(19) United States
(12) Patent Application Publication
(10) Pub. No.: US 2021/0130339 A1
(43) Pub. Date: May 6, 2021

(54) COMPOUNDS

(71) Applicant: GLAXOSMITHKLINE INTELLECTUAL PROPERTY DEVELOPMENT LIMITED, Brentford (GB)

(72) Inventors: Feng REN, Shanghai (CN); Yingxia SANG, Shanghai (CN); Baowei ZHAO, Shanghai (CN)

(21) Appl. No.: 16/480,839

(22) PCT Filed: Jan. 19, 2018
(86) PCT No.: PCT/CN2018/073462

§ 371 Date: Jul. 25, 2019

(30) Foreign Application Priority Data
Jan. 25, 2017 (CN) PCT/CN2017/072590

(51) Int. Cl. C07D 413/14; C07D 405/14

(52) U.S. Cl. CPC C07D 413/14; C07D 405/14

(57) ABSTRACT

Provided are novel compounds that inhibit LRRK2 kinase activity, processes for their preparation, compositions containing them and their use in the treatment of or prevention of diseases associated with or characterized by LRRK2 kinase activity, for example Parkinson's disease, Alzheimer's disease and amyotrophic lateral sclerosis (ALS).

### FIELD OF THE INVENTION

The present invention relates to novel compounds that inhibit LRRK2 kinase activity, processes for their preparation, compositions containing them and their use in the treatment of diseases associated with or characterized by LRRK2 kinase activity, for example, Parkinson's disease, Alzheimer's disease and amyotrophic lateral sclerosis (ALS).

### BACKGROUND OF THE INVENTION

Parkinson's disease (PD) is a neurodegenerative disorder characterized by selective degeneration and cell death of dopaminergic neurons in the substantial nigra region of the brain. Parkinson's disease was generally considered to be sporadic and of unknown etiology, but, in the last 15 years, there has been an important development of the understanding of the genetic basis of this disease and associated pathogenic mechanisms. One area of the development is the understanding of leucine rich repeat kinase 2 (LRRK2) protein. A number of mis-sense mutations in the LRRK2 gene have been strongly linked with autosomal dominant Parkinson's disease in familial studies (See WO2006068492 and WO2006045392; Trinh and Farrer 2013, Nature Reviews in Neurology 9: 445-454; Paisan-Ruiz et al., 2013, J. Parkinson's Disease 3: 85-103). The G2019S mutation in LRRK2 is the most frequent mis-sense mutation and is associated with a clinical phenotype that closely resembles sporadic Parkinson's disease. The LRRK2 G2019S mutation is also present in approximately 1.5% of sporadic Parkinson's disease cases (See Gilks et al., 2005, Lancet, 365: 415-416). In addition to the known pathogenic coding mutations in LRRK2, additional amino acid coding variants of LRRK2 have been identified that are also associated with risk of developing Parkinson's disease (See Ross et al., 2011 Lancet Neurology 10: 898-908). Furthermore, genome-wide association studies (GWAS) have identified LRRK2 as a Parkinson's disease susceptibility locus, which indicates that LRRK2 may be also relevant to sporadic Parkinson's disease cases without mutations that cause amino acid substitutions in the LRRK2 protein. (See Satake et al., 2009 Nature Genetics 41:1303-1307; Simon-Sanchez et al 2009 Nature Genetics 41: 1308-1312)

LRRK2 is a member of the ROCO protein family and all members of this family share five conserved domains. The most common pathogenic mutation G2019S occurs in the highly conserved kinase domain of LRRK2. This mutation confers an increase in the LRRK2 kinase activity in in vitro enzyme assays of recombinant LRRK2 proteins (See Jaleel et al., 2007, Biochem J, 405: 307-317) and in LRRK2 proteins purified from G2019S PD patient-derived cells (See Dzamko et al., 2010 Biochem. J. 430: 405-413). A less frequent LRRK2 pathogenic mutation that confers amino acid substitution at a different residue, R1441, has also been shown to elevate LRRK2 kinase activity by decreasing the rate of GTP hydrolysis by the GTPase domain of LRRK2 (See Guo et al., 2007 Exp Cell Res. 313: 3658-3670; West et al., 2007 Hum. Mol Gen. 16: 223-232). Moreover, phosphorylation of Rab protein physiologic substrates of LRRK2 has been shown to be increased by a range of Parkinson's disease pathogenic mutations of LRRK2 (See Steger et al., 2016 eLife 5 e12813). Therefore, the evidence indicates that the kinase and GTPase activities of LRRK2 are important for pathogenesis, and that the LRRK2 kinase domain may regulate overall LRRK2 function (See Cookson, 2010 Nat. Rev. Neurosci. 11: 791-797).

There is evidence to show that the increased LRRK2 kinase activity is associated with neuronal toxicity in cell culture models (See Smith et al., 2006 Nature Neuroscience 9: 1231-1233) and kinase inhibitor compounds protect against LRRK2-mediated cell death (See Lee et al., 2010 Nat. Med. 16: 998-1000). LRRK2 has also been reported to act as a negative regulator of microglial-mediated clearance of alpha-synuclein (See Maekawa et al., 2016 BMC Neuroscience 17:77), suggesting a possible utility of LRRK2 inhibitors in promoting clearance of neurotoxic forms of alpha-synuclein in the treatment of Parkinson's disease.

Induced pluripotent stem cells (iPSCs) derived from LRRK2 G2019S Parkinson's disease patients have been found to exhibit defects in neurite outgrowth and increased susceptibility to rotenone, that may be ameliorated by either genetic correction of the G2019S mutation or treatment of cells with small molecule inhibitors of LRRK2 kinase activity (See Reinhardt et al., 2013 Cell Stem Cell 12: 354-367). Mitochondrial DNA damage has been reported as a molecular marker of vulnerable dopamine neurons in substantia nigra of postmortem Parkinson's disease specimens (See Sanders et al 2014 Neurobiol. Dis. 70: 214-223). Increased levels of such mitochondrial DNA damage associated with LRRK2 G2019S mutation in iSPCs is blocked by genetic correction of the G2019S mutation (See Sanders et al., 2014 Neurobiol. Dis. 62: 381-386).

Additional evidence links LRRK2 function and dysfunction with autophagy-lysosomal pathways (See Manzoni and Lewis, 2013 Faseb J. 27:3234-3429). LRRK2 proteins confer defects in chaperone-mediated autophagy that negatively impact the ability of cells to degrade alpha-synuclein (Orenstein et al., 2013 Nature Neurosci. 16 394-406). In other cell models, selective LRRK2 inhibitors have been shown to stimulate macroautophagy (See Manzoni et al., 2013 BBA Mol. Cell Res. 1833: 2900-2910). These data suggest that small molecule inhibitors of LRRK2 kinase activity may have utility in the treatment of diseases characterized by defects in cellular proteostasis that result from aberrant autophagy/lysosomal degradation pathways including forms of Parkinson's disease associated with GBA mutations (See Swan and Saunders-Pullman 2013 Curr. Neurol. Neurosci Rep. 13: 368), other alpha-synucleinopathies, tauopathies, Alzheimer's disease (See Li et al., 2010 Neurodegen. Dis. 7: 265-271) and other neurodegenerative diseases (See Nixon 2013 Nat. Med. 19: 983-997) and Gaucher disease (See Westbroek et al., 2011 Trends. Mol. Med. 17: 485-493). As promoters of autophagy, small molecule inhibitors of LRRK2 kinase may also have utility in treatment of other diseases including diabetes, obesity, motor neuron disease, epilepsy and some cancers (See Rubinsztein et al., 2012 Nat. Rev. Drug Discovery 11: 709-730), pulmonary diseases such as chronic obstructive pulmonary disease and idiopathic pulmonary fibrosis (See Araya et al., 2013 Intern. Med. 52: 2295-2303) and autoimmune diseases such as systemic lupus erythematosus (See Martinez et al., 2016 Nature 533: 115-119). As promoters of autophagy and phagocytic processes, small molecule inhibitors of LRRK2 kinase may also have utility in augmenting host responses in treatment of a range of intracellular bacterial infections, parasitic infections and viral infections, including diseases such as tuberculosis (See Rubinsztein et al., 2012 Nat. Rev. Drug Discovery 11: 709-730; Araya et al., 2013 Intern. Med. 52: 2295-2303; Gutierrez, Biochemical Society Conference; Leucine rich repeat kinase 2: ten years along the road to therapeutic intervention, Henley Business School, UK 12 Jul. 2016), HIV, West Nile Virus and chikungunya virus (see Shoji-Kawata et al., 2013 Nature 494: 201-206). LRRK2 inhibitors may have utility in treatment of such diseases alone, or in combination with drugs that directly target the infectious agent. Further, significantly elevated levels of LRRK2 mRNA have also been observed in fibroblasts of Niemann-Pick Type C (NPC) disease patients compared with fibroblasts of normal subjects, which indicates that aberrant LRRK2 function may play a role in lysosomal disorders (See Reddy et al., 2006 PLOS One 1 (1):e19 doi: 10.1371/journal.pone.0000019—supporting information Dataset S1). This observation suggests that LRRK2 inhibitors may have utility for treatment of NPC.

The PD-associated G2019S mutant form of LRRK2 has also been reported to enhance phosphorylation of tubulin-associated Tau (See Kawakami et al., 2012 PLoS ONE 7: e30834, doi 10.1371), and disease models have been proposed in which LRRK2 acts upstream of the pathogenic effects of Tau and alpha-synuclein (See Taymans & Cookson, 2010, BioEssays 32: 227-235). In support of this, LRRK2 expression has been associated with increased aggregation of insoluble Tau, and increased Tau phosphorylation, in a transgenic mouse model (See Bailey et al., 2013 Acta Neuropath. 126:809-827). Over-expression of the PD pathogenic mutant protein LRRK2 R1441G is reported to cause symptoms of Parkinson's disease and hyperphosphorylation of Tau in transgenic mouse models (See Li, Y. et al. 2009, Nature Neuroscience 12: 826-828). Therefore, these data suggest that LRRK2 inhibitors of kinase catalytic activity may be useful for the treatment of tauopathy diseases characterized by hyperphosphorylation of Tau such as argyrophilic grain disease, Pick's disease, corticobasal degeneration, progressive supranuclear palsy and inherited frontotemporal dementia and parkinsonism linked to chromosome 17 (FTDP-17) (See Goedert, M and Jakes, R (2005) Biochemica et Biophysica Acta 1739, 240-250). In addition, LRRK2 inhibitors may have utility in treatment of other diseases characterized by diminished dopamine levels such as withdrawal symptoms/relapse associated with drug addiction (See Rothman et al., 2008, Prog. Brain Res, 172: 385).

Other studies have also shown that overexpression of the G2019S mutant form of LRRK2 confers defects in subventricular zone (SVZ) neuroprogenitor cell proliferation and migration in transgenic mouse models (See Winner et al., 2011 Neurobiol. Dis. 41: 706-716) and reduces neurite length and branching cell culture models (See Dachsel et al., 2010 Parkinsonism & Related Disorders 16: 650-655). Moreover, it was reported that agents that promote SVZ neuroprogenitor cell proliferation and migration also improve neurological outcomes following ischemic injury in rodent models of stroke (See Zhang et al., 2010 J. Neurosci. Res. 88: 3275-3281). These findings suggest that compounds that inhibit aberrant activity of LRRK2 may have utility for the treatments designed to stimulate restoration of CNS functions following neuronal injury, such as ischemic stroke, traumatic brain injury, spinal cord injury.

Mutations in LRRK2 have also been identified that are clinically associated with the transition from mild cognitive impairment (MCI) to Alzheimer's disease (See WO2007149798). These data suggest that inhibitors of LRRK2 kinase activity may be useful for the treatment diseases such as Alzheimer's disease, other dementias and related neurodegenerative disorders.

Aberrant regulation of normal LRRK2 proteins is also observed in some disease tissues and models of disease. Normal mechanisms of translational control of LRRK2 by miR-205 are perturbed in some sporadic PD cases, where significant decreases in miR-205 levels in PD brain samples concur with elevated LRRK2 protein levels in those samples (See Cho et al., (2013) Hum. Mol. Gen. 22: 608-620). Therefore, LRRK2 inhibitors may be used in treatment of sporadic PD patients who have elevated levels of normal LRRK2 proteins.

In an experimental model of Parkinson's disease in marmosets, an elevation of LRRK2 mRNA is observed in a manner that correlates with the level of L-Dopa induced dyskinesia (See Hurley, M. J et al., 2007 Eur. J. Neurosci. 26: 171-177). This suggests that LRRK2 inhibitors may have a utility in amelioration of such dyskinesias.

Significantly elevated levels of LRRK2 mRNA have been reported in ALS patient muscle biopsy samples (See Shtilbans et al., 2011 Amyotrophic Lateral Sclerosis 12: 250-256) It is suggested that elevated levels of LRRK2 kinase activity may be a characteristic feature of ALS. Therefore, this observation indicated that LRRK2 inhibitor may have utility for treatment of ALS.

There is also evidence indicating that LRRK2 kinase activity may play a role in mediating microglial proinflammatory responses (See Moehle et al., 2012, J. Neuroscience 32: 1602-1611). This observation suggests a possible utility of LRRK2 inhibitors for treatment of aberrant neuroinflammatory mechanisms that contribute a range of neurodegenerative diseases, including Parkinson's disease, Alzheimer's disease, multiple sclerosis, HIV-induced dementia, amyotrophic lateral sclerosis, ischemic stroke, traumatic brain injury and spinal cord injury. Some evidence also indicates that LRRK2 plays a role in regulating neuronal progenitor differentiation in vitro (See Milosevic, J. et al., 2009 Mol. Neurodegen. 4: 25). This evidence suggests that inhibitors of LRRK2 may have a utility in production of neuronal progenitor cells in vitro for consequent therapeutic application in cell based-treatment of CNS disorders.

It has been reported that Parkinson's disease patients bearing LRRK2 G2019S mutation display increased frequency of non-skin cancers, including renal, breast, lung, prostate cancers as well as acute myelogenous leukemia (AML). Since there is evidence to show that G2019S mutation in LRRK2 increases catalytic activity of the LRRK2 kinase domain, small molecule inhibitors of LRRK2 may have a utility in treatment of cancers, for example kidney cancer, breast cancer, lung cancer, prostate cancer (e.g. solid tumors) and blood cancer (See. AML; Saunders-Pullman et al., 2010, Movement Disorders, 25:2536-2541; Inzelberg et al., 2012 Neurology 78: 781-786). Amplification and over-expression of LRRK2 has also been reported in papillary renal and thyroid carcinomas, where co-operativity between LRRK2 and the MET oncogene may promote tumor cell growth and survival (See Looyenga et al., 2011 PNAS 108: 1439-1444.) Some studies suggested that genetic association of common LRRK2 variants with susceptibility to ankylosing spondylitis (See Danoy P, et al., 2010. PLoS Genet.; 6(12):e1001195; and leprosy infection. (See Zhang F R, et al. 2009, N Engl J Med. 361:2609-18.) These findings suggest that inhibitors of LRRK2 may have a utility in the treatment of ankylosing spondylitis and leprosy infection.

Meta-analysis of three genome wide associated scans for Crohn's disease identified a number of loci associated with the disease, including the locus containing the LRRK2 gene (See Barrett et al., 2008, Nature Genetics, 40: 955-962). Evidence has also emerged that LRRK2 is an IFN-γ target gene that may be involved in signaling pathways relevant to Crohn's disease pathogenesis (See Gardet et al., 2010, J. Immunology, 185: 5577-5585). These findings suggest that inhibitors of LRRK2 may have utility in the treatment of Crohn's disease.

As an IFN-γ target gene, LRRK2 may also play a role in T cell mechanisms that underlie other diseases of the immune system such as multiple sclerosis and rheumatoid arthritis. Further potential utility of LRRK2 inhibitors comes from the reported finding that B lymphocytes constitute a major population of LRRK2 expressing cells (See Maekawa et al. 2010, BBRC 392: 431-435). This suggests that LRRK2 inhibitors may be effective in treatment of diseases of the immune system for which B cell depletion is, or may be, effective in diseases such as lymphomas, leukemias, multiple sclerosis (See Ray et al., 2011 J. Immunol. 230: 109), rheumatoid arthritis, systemic lupus erythematosus, autoimmune hemolytic anemia, pure red cell aplasia, idiopathic thrombocytopenic purpura (ITP), Evans syndrome, vasculitis, bullous skin disorders, type 1 diabetes mellitus, Sjogren's syndrome, Devic's disease and inflammatory myopathies (See Engel et al., 2011 Pharmacol. Rev. 63: 127-156; Homam et al., 2010 J. Clin. Neuromuscular Disease 12: 91-102).

WO2016036586 and WO2017012576 disclose a series of compounds described as inhibitors of LRRK2 kinase and their use in the treatment of diseases, including, inter alia, Parkinson's disease. Unmet needs exist for new treatments that will halt or slow disease progression both in terms of motor (e.g. control of gait dysfunction, freezing, and postural imbalance) and non-motor symptoms (e.g. PD-associated dementia), reducing the need for escalating use of symptomatic medications and associated long-term adverse effects of currently available treatment (e.g. dyskinesia and on/off fluctuations) maintaining independence for longer.

### SUMMARY OF THE INVENTION

The present invention provides, in a first aspect, compounds of Formula (I) and salts thereof:

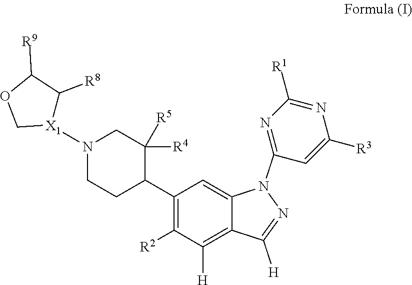

wherein
X₁ is CR⁶ wherein R⁶ is H or C₁₋₃alkyl, which alkyl group is optionally substituted with one or more substituents independently selected from the group consisting of hydroxyl, halo and C₁₋₃alkoxyl;
R¹ is selected from the group consisting of CN, C₁₋₃ alkyl, C₁₋₃ alkoxy, C₁₋₃haloalkyl, and C₃ cycloalkyl;
R² is selected from the group consisting of H, halo, CN, C₁₋₃alkyl and C₁₋₃haloalkyl;
R³ is selected from the group consisting of:
a) an N-linked 4-6 membered heterocyclyl ring optionally substituted with one or two substituents independently selected from the group consisting of:
   oxo,
   halo,
   hydroxyl,
   C₁₋₆alkyl, which alkyl group is optionally substituted with one or two substituents independently selected from the group consisting of: halo, hydroxyl, C₁₋₃alkoxy and cyclopropyl, and
   C₁₋₆ alkoxyl, which alkoxyl group isoptionally substituted with one or two substitutents independently selected from halo, hydroxyl and C₁₋₃ alkoxyl, wherein when the N-linked 4-6 membered heterocyclyl ring contains a substitutable nitrogen atom, the group of substituents also includes a 4-6 membered heterocyclyl ring which is optionally substituted with one, two or three substitutents independently selected from halo, hydroxyl, and C₁₋₃ alkoxyl with the proviso that the 4-6 membered heterocyclyl ring is attached to said substitutable nitrogen atom;
b) NHR⁷;
c) OR⁷;
R⁴ and R⁵ are independently selected from the group consisting of H, hydroxyl and halo;
R⁷ is independently selected from the group consisting of:
C₄₋₆ cycloalkyl, which cycloalkyl is optionally substituted with one, two or three substituents independently selected from halo, hydroxyl, C₁₋₃ alkoxyl and C₁₋₃ alkyl, which alkyl group is optionally substituted with one two or three halo or hydroxyl groups, and
a nitrogen or oxygen containing 4-6 membered heterocyclyl optionally substituted with one or more substitutents independently selected from halo, hydroxyl, C₁₋₃ alkoxyl and C₁₋₃ alkyl, which alkyl group is optionally substituted with one two or three halo or hydroxyl groups; and
R⁸ and R⁹ are independently selected from the group consisting of H, halo, methyl, ethyl, methoxyl and hydroxyl.

In a further aspect of the invention, the invention provides a pharmaceutical composition comprising a compound of Formula (I) or a pharmaceutically acceptable salt thereof and a pharmaceutically acceptable carrier.

A further aspect of the invention provides a compound of Formula (I) or a pharmaceutically acceptable salt thereof for use in the treatment or prevention of Parkinson's disease, Alzheimer's disease, or amyotrophic lateral sclerosis (ALS).

### DETAILED DESCRIPTION OF THE INVENTION

The foregoing and other aspects of the present invention will now be described in more detail with respect to the description and methodologies provided herein. It should be appreciated that the invention can be embodied in different forms and should not be construed as limited to the embodiments set forth herein. Rather, these embodiments are provided so that this disclosure will be thorough and complete, and will Fully convey the scope of the invention to those skilled in the art.

The terminology used in the description of the invention herein is for the purpose of describing particular embodiments only and is not intended to be limiting of the invention. As used in the description of the embodiments of the invention and the appended claims, the singular forms “a”, “an” and “the” are intended to include the plural forms as well, unless the context clearly indicates otherwise. Also, as used herein, “and/or” refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

Generally, the nomenclature used herein and the laboratory procedures in organic chemistry, medicinal chemistry, biology described herein are those well known and commonly employed in the art. Unless defined otherwise, all technical and scientific terms used herein generally have the same meaning as commonly understood by one of ordinary skill in the art to which this disclosure belongs. In the event that there is a plurality of definitions for a term used herein, those in this section prevail unless stated otherwise.

### A. Definitions

As used herein, “alkyl” refers to a monovalent, saturated hydrocarbon chain having a specified number of carbon atoms. For example, C₁₋₃ alkyl refers to an alkyl group having from 1 to 3 carbon atoms. Alkyl groups may be straight or branched. In some embodiments, branched alkyl groups may have one, two, or three branches. Exemplary alkyl groups include, but are not limited to, methyl, ethyl, and propyl (n-propyl and isopropyl).

As used herein, “alkoxy” refers to the group —O-alkyl. For example, C₁₋₆ alkoxy groups contain from 1 to 6 carbon atoms. C₁₋₃ alkoxy groups contain from 1 to 3 carbon atoms. Exemplary alkoxy groups include, but are not limited to, methoxy, ethoxy, propoxy, butoxyl, pentyloxy, and hexyloxy.

As used herein, “cycloalkyl” refers to a saturated monocyclic hydrocarbon ring having a specified number of carbon atoms. For example, C₃₋₆ cycloalkyl contains 3 to 6 carbon atoms as member atoms in the ring. Examples of C₃₋₆ cycloalkyl include cyclopropyl, cyclobutyl, cyclopentyl and cyclohexyl.

As used herein, “halogen” refers to fluorine (F), chlorine (Cl), bromine (Br), or iodine (I). “Halo” refers to the halogen radicals: fluoro (—F), chloro (—Cl), bromo (—Br), or iodo (—I).

As used herein, “haloalkyl” refers to an alkyl group, as defined above, having one or more halogen atoms selected from F, Cl, Br, or I, which are substituted on any or all of the carbon atoms of the alkyl group by replacing hydrogen atoms attached to the carbon atoms and which may be the same or different. For example, C₁₋₃haloalkyl refers to a C₁₋₃alkyl group substituted with one or more halogen atoms. In some embodiments, “haloalkyl” refers to an alkyl group substituted with one or more halogen atoms independently selected from F or Cl. Exemplary haloalkyl groups include, but are not limited to, chloromethyl, bromoethyl, trifluoromethyl, and dichloromethyl.

As used herein, “heterocyclyl” or “herterocyclyl ring” is a monovalent radical derived by removal of a hydrogen atom from a saturated monocyclic ring, which ring consists of ring carbon atoms and 1 or more ring heteroatoms independently selected from nitrogen, oxygen or sulphur. In one embodiment, the ring consists of ring carbon atoms and 1 to 3 ring heteroatoms independently selected from nitrogen, oxygen or sulphur. In one embodiment, the ring-heteroatoms are independently selected from nitrogen or oxygen. The number of ring atoms may be specified. For example, a “4-6 membered heterocyclyl” a heterocyclyl as defined above consisting of 4-6 ring atoms. The term N-linked 4-6 membered heterocyclyl refers to a 4-6 membered heterocyclyl ring as defined above that contains at least one nitrogen ring atom through which it is linked to the core. Other ring heteroatoms (nitrogen, oxygen or sulphur) may additionally be present. The term nitrogen containing heterocyclyl refers to heterocyclyl ring as defined above that contains at least one nitrogen ring atom. Other ring heteroatoms (nitrogen, oxygen or sulphur) may additionally be present. The term oxygen containing heterocyclyl should be construed in an analogous manner. Examples of herterocyclyl rings include, but are not limited to, azetidinyl, tetrahydrofuranyl (including, for example, tetrahydrofuran-2-yl and tetrahydrofuran-3-yl), pyrrolidinyl (including, for example, pyrrolidin-1-yl and pyrrolidin-3-yl), piperidinyl (including, for example, piperidin-3-yl and piperidin-4-y), morpholinyl (including, for example, morpholin-2-yl and morpholin-4-yl).

As used herein, “substituted” in reference to a group indicates that one or more hydrogen atom attached to a member atom (e.g., carbon atom) within the group is replaced with a substituent selected from the group of defined substituents. It should be understood that the term “substituted” includes the implicit provision that such substitution is in accordance with the permitted valence of the substituted atom and the substituent and that the substitution results in a stable compound (i.e. one that does not spontaneously undergo transformation such as by rearrangement, cyclization, or elimination and that is sufficiently robust to survive isolation from a reaction mixture). When it is stated that a group may contain one or more substituent, one or more (as appropriate) member atom within the group may be substituted. In addition, a single member atom within the group may be substituted with more than one substituent as long as such substitution is in accordance with the permitted valence of the atom. Examples of substituted heterocyclyl rings rings include, but are not limited to,

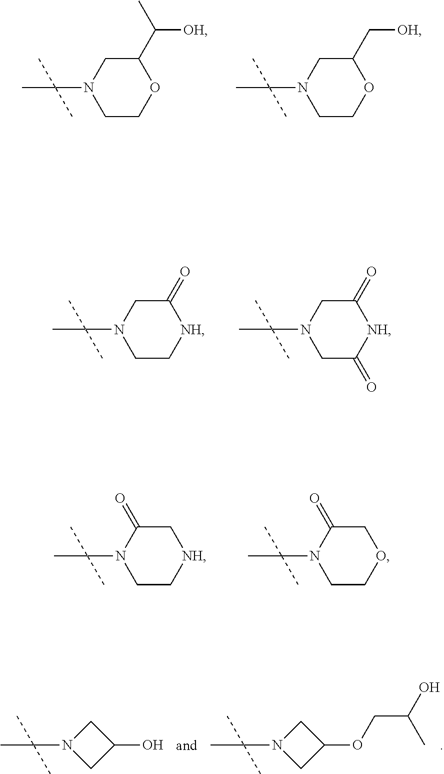

As used herein, “optionally substituted” indicates that a particular group may be unsubstituted, or may be substituted as further defined.

As used herein, the term “disease” refers to any alteration in state of the body or of some of the organs, interrupting or disturbing the performance of the functions and/or causing symptoms such as discomfort, dysfunction, distress, or even death to the person afflicted or those in contact with a person. A disease can also include a distemper, ailing, ailment, malady, disorder, sickness, illness, complain, interdisposition and/or affectation.

As used herein, “treat”, “treating” or “treatment” in reference to a disease means: (1) to ameliorate the disease or one or more of the biological manifestations of the disease, (2) to interfere with (a) one or more points in the biological cascade that leads to or is responsible for the disease or (b) one or more of the biological manifestations of the disease, (3) to alleviate one or more of the symptoms or effects associated with the disease, (4) to slow the progression of the disease or one or more of the biological manifestations of the disease, and/or (5) to diminish the likelihood of severity of a disease or biological manifestations of the disease. Symptomatic treatment refers to treatment as referred to in point (1), (3) and (5). Disease modifying treatment refers to treatment as defined in point (2) and (4).

As used herein, “prevent”, “preventing” or “prevention” means the prophylactic administration of a drug to diminish the likelihood of the onset of or to delay the onset of a disease or biological manifestation thereof.

As used herein, “subject” means a mammalian subject (e.g., dog, cat, horse, cow, sheep, goat, monkey, etc.), and human subjects including both male and female subjects, and including neonatal, infant, juvenile, adolescent, adult and geriatric subjects, and further including various races and ethnicities including, but not limited to, white, black, Asian, American Indian and Hispanic.

As used herein, “pharmaceutically acceptable salt(s)” refers to salt(s) that retain the desired biological activity of the subject compound and exhibit minimal undesired toxicological effects. These pharmaceutically acceptable salts may be prepared in situ during the final isolation and purification of the compound, or by separately reacting the purified compound in its free acid or free base form with a suitable base or acid, respectively.

As used herein, “therapeutically effective amount” in reference to a compound of the invention or other pharmaceutically-active agent means an amount of the compound sufficient to treat or prevent the patient's disease but low enough to avoid serious side effects (at a reasonable benefit/risk ratio) within the scope of sound medical judgment. A therapeutically effective amount of a compound will vary with the particular compound chosen (e.g. consider the potency, efficacy, and half-life of the compound); the route of administration chosen; the disease being treated; the severity of the disease being treated; the age, size, weight, and physical disease of the patient being treated; the medical history of the patient to be treated; the duration of the treatment; the nature of concurrent therapy; the desired therapeutic effect; and like factors, but can nevertheless be routinely determined by the skilled artisan.

### B. Compounds

This invention provides, in a first aspect, a compound of Formula (I) and salts thereof:

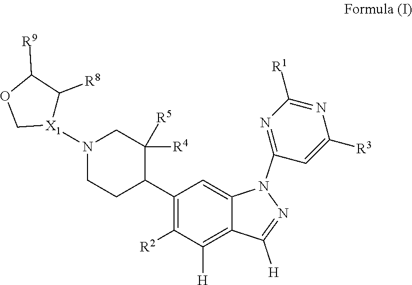

wherein
X₁ is CR⁶ wherein R⁶ is H or C₁₋₃alkyl, which alkyl group is optionally substituted with one or more substituents independently selected from the group consisting of hydroxyl, halo and C₁₋₃alkoxyl;
R¹ is selected from the group consisting of CN, C₁₋₃ alkyl, C₁₋₃ alkoxy, C₁₋₃haloalkyl, and C₃ cycloalkyl;
R² is selected from the group consisting of H, halo, CN, C₁₋₃alkyl and C₁₋₃haloalkyl;
R³ is selected from the group consisting of:
a) an N-linked 4-6 membered heterocyclyl ring optionally substituted with one or two substituents independently selected from the group consisting of:
   oxo,
   halo,
   hydroxyl,
   C₁₋₆alkyl, which alkyl group is optionally substituted with one or two substituents independently selected from the group consisting of: halo, hydroxyl, C₁₋₃alkoxy and cyclopropyl, and
   C₁₋₆ alkoxyl, which alkoxyl group is optionally substituted with one or two substitutents independently selected from halo, hydroxyl and C₁₋₃ alkoxyl, wherein when the N-linked 4-6 membered heterocyclyl ring contains a substitutable nitrogen atom, the group of substituents also includes a 4-6 membered heterocyclyl ring which is optionally substituted with one, two or three substitutents independently selected from halo, hydroxyl, and C₁₋₃ alkoxyl with the proviso that the 4-6 membered heterocyclyl ring is attached to said substitutable nitrogen atom;
b) NHR⁷; and
c) OR⁷
R⁴ and R⁵ are independently selected from the group consisting of H, hydroxyl and halo;
R⁷ is independently selected from the group consisting of:
C₄₋₆ cycloalkyl, which cycloalkyl is optionally substituted with one, two or three substituents independently selected from halo, hydroxyl, C₁₋₃ alkoxyl and C₁₋₃ alkyl, which alkyl group is optionally substituted with one two or three halo or hydroxyl groups, and
a nitrogen or oxygen containing 4-6 membered heterocyclyl optionally substituted with one or more substitutents independently selected from halo, hydroxyl, C₁₋₃ alkoxyl and C₁₋₃ alkyl, which alkyl group is optionally substituted with one two or three halo or hydroxyl groups; and
R⁸ and R⁹ are independently selected from the group consisting of H, halo, methyl, ethyl, methoxyl and hydroxyl.

In one embodiment, R¹ is selected from the group consisting of C₁₋₃alkyl and C₁₋₃alkoxyl. In one embodiment, R¹ is selected from the group consisting of methyl or methoxy. In one embodiment, R¹ is methyl.

In one embodiment, R² is selected from the group consisting of H, halo and C₁₋₃alkyl. In one embodiment, R² is selected from the group consisting of C₁₋₃alkyl. In one embodiment, R² is selected from the group consisting of H, halo and methyl. In one embodiment, R² is selected from the group consisting of H, fluoro, chloro and methyl. In one embodiment, R² is selected from the group consisting of H, chloro and methyl. In one embodiment, R² is selected from the group consisting of chloro and methyl. In one embodiment, R² is methyl.

In one embodiment R³ is an N-linked 4-6 membered heterocyclyl ring optionally substituted with one or two substituents independently selected from the group consisting of:
halo,
hydroxyl,
C₁₋₆alkyl, which alkyl group is optionally substituted with one or two substituents independently selected from the group consisting of: halo, hydroxyl, C₁₋₃alkoxy and cyclopropyl, and
C₁₋₆ alkoxyl, which alkoxyl group is optionally substituted with one or two substitutents independently selected from halo, hydroxyl and C₁₋₃ alkoxyl, wherein when the N-linked 4-6 membered heterocyclyl ring contains a substitutable nitrogen atom, the group of substituents also includes a 4-6 membered heterocyclyl ring which is optionally substituted with one, two or three substitutents independently selected from halo, hydroxyl, and C₁₋₃ alkoxyl, with the proviso that the 4-6 membered heterocyclyl ring is attached to said substitutable nitrogen atom.

In one embodiment R³ is an N-linked 4-6 membered heterocyclyl ring optionally substituted with one or two substituents independently selected from the group consisting of:
oxo,
halo,
hydroxyl,
C₁₋₃alkyl, which alkyl group is optionally substituted with one or two substituents independently selected from the group consisting of: halo, hydroxyl and C₁₋₃alkoxy, and
C₁₋₃ alkoxyl, which alkoxyl group is optionally substituted with one or two substitutents independently selected from halo, hydroxyl and C₁₋₃ alkoxyl.

In one embodiment R³ is an N-linked 4-6 membered heterocyclyl ring selected from the group consisting of morpholinyl, azetidinyl, pyrrolidinyl and piperazinyl, optionally substituted with one or two substituents independently selected from the group consisting of:
halo,
hydroxyl,
C₁₋₃alkyl, which alkyl group is optionally substituted with one or two substituents independently selected from the group consisting of: halo, hydroxyl and C₁₋₃ alkoxy, and
C₁₋₃ alkoxyl, which alkoxyl group is optionally substituted with one or two substitutents independently selected from halo, hydroxyl and C₁₋₃ alkoxyl.

In one embodiment R³ is an N-linked 4-6 membered heterocyclyl ring selected from the group consisting of morpholinyl, azetidinyl, pyrrolidinyl and piperazinyl, optionally substituted with one or two substituents independently selected from the group consisting of:
hydroxyl,
C₁₋₃alkyl, which alkyl group is optionally substituted with one or two substituents independently selected from the group consisting of: halo, hydroxyl and C₁₋₃alkoxy, and
C₁₋₃ alkoxyl, which alkoxyl group is optionally substituted with one or two substitutents independently selected from halo, hydroxyl and C₁₋₃ alkoxyl.

In one embodiment R³ is an N-linked morpholinyl ring optionally substituted with one or two substituents independently selected from the group consisting of:
hydroxyl,
C₁₋₃alkyl, which alkyl group is optionally substituted with one or two substituents independently selected from the group consisting of: halo, hydroxyl and C₁₋₃ alkoxy, and
C₁₋₃ alkoxyl, which alkoxyl group is optionally substituted with one or two substitutents independently selected from halo, hydroxyl and C₁₋₃ alkoxyl.

In one embodiment R³ is an N-linked 4-6 membered heterocyclyl ring selected from the group consisting of morpholinyl, azetidinyl, pyrrolidinyl and piperazinyl.

In one embodiment, R³ is (2-hydroxymethyl)-morpholin-4-yl.

In one embodiment, R³ is (2-hydroxyethyl)-morpholin-4-yl.

In one embodiment, R³ is (2-hydroxymethyl)-6-methyl-morpholin-4-yl.

In one embodiment, R³ is 3-methyl-morpholin-4-yl.

In one embodiment, R³ is 3-hydroxyl 3-methyl azetidin-1-yl

In one embodiment, R³ is 3-hydroxyl pyrrolidin-1-yl.

In one embodiment, R³ is

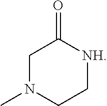

In one embodiment R³ is an N-linked 4-6 membered heterocyclyl ring containing a substitutable nitrogen atom, substituted with a further 4-6 membered heterocyclyl ring which is optionally substituted with one, two or three substitutents independently selected from halo, hydroxyl, and C₁₋₃ alkoxyl, and with the proviso that the further 4-6 membered heterocyclyl ring is attached to said substitutable nitrogen atom.

In one embodiment R³ is an N-linked 4-6 membered heterocyclyl ring containing a substitutable nitrogen atom, substituted with an oxetanyl group on said substitutable nitrogen atom.

In one embodiment, R⁴ and R⁵ are independently selected from the group consisting of H and halo. In one embodiment, R⁴ and R⁵ are independently selected from the group consisting of H and fluoro. In one embodiment, R⁴ and R⁵ are both hydrogen.

In one embodiment, R⁶ is H or unsubstituted C₁₋₃alkyl. In one embodiment, R⁶ is H or methyl.

In one embodiment, R⁶ is H.

In one embodiment, both R⁸ and R⁹ are H.

In one embodiment, the invention provides a compound of Formula (I) or a salt thereof wherein R¹, R², R⁴, R⁵, X₁, R⁶, R⁸ and R⁹ are as defined above, and R³ is an N-linked 4-6 membered heterocyclyl ring optionally substituted with one or two substituents independently selected from the group consisting of: halo, hydroxyl, C₁₋₃alkyl (which alkyl group is optionally substituted with one or two substituents independently selected from the group consisting of: halo, hydroxyl and C₁₋₃alkoxy) and C₁₋₃ alkoxyl (which alkoxyl group is optionally substituted with one or two substitutents independently selected from halo, hydroxyl and C₁₋₃ alkoxyl). In this embodiment, R¹, R², R⁴, R⁵, X₁, R⁶, R⁸ and R⁹ may be further defined as in any of the preceding embodiments. For example, R¹ may be selected from the group consisting of C₁₋₃ alkyl and C₁₋₃ alkoxyl and/or R² may be selected from the group consisting of H, halo and C₁₋₃alkyl and/or R⁶ may be H, and/or Ra and R⁹ may be H.

In one embodiment, the invention provides a compound of formula (I) or a pharmaceutically acceptable salt thereof that is a compound of any one of examples 1 to 156, or a pharmaceutically acceptable salt thereof.

In one embodiment, this invention relates to a compound selected from

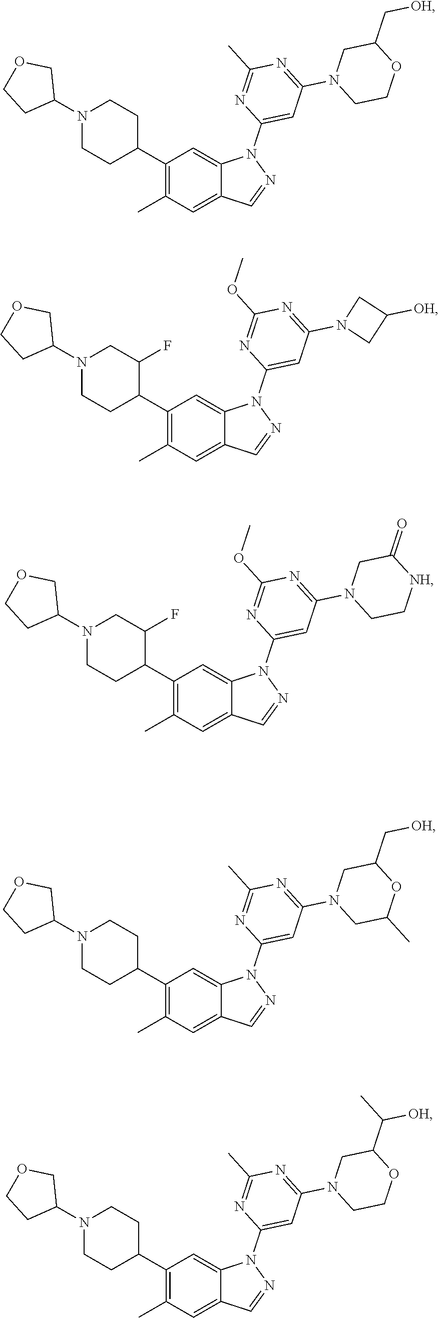 [SMILES: Cc1nc(N2CCOC(CO)C2)cc(-n2ncc3cc(C)c(C4CCN(C5CCOC5)CC4)cc32)n1 ; COc1nc(N2CC(O)C2)cc(-n2ncc3cc(C)c(C4CCN(C5CCOC5)CC4F)cc32)n1 ; COc1nc(N2CCNC(=O)C2)cc(-n2ncc3cc(C)c(C4CCN(C5CCOC5)CC4F)cc32)n1 ; Cc1nc(N2CC(C)OC(CO)C2)cc(-n2ncc3cc(C)c(C4CCN(C5CCOC5)CC4)cc32)n1 ; Cc1nc(N2CCOC(C(C)O)C2)cc(-n2ncc3cc(C)c(C4CCN(C5CCOC5)CC4)cc32)n1]

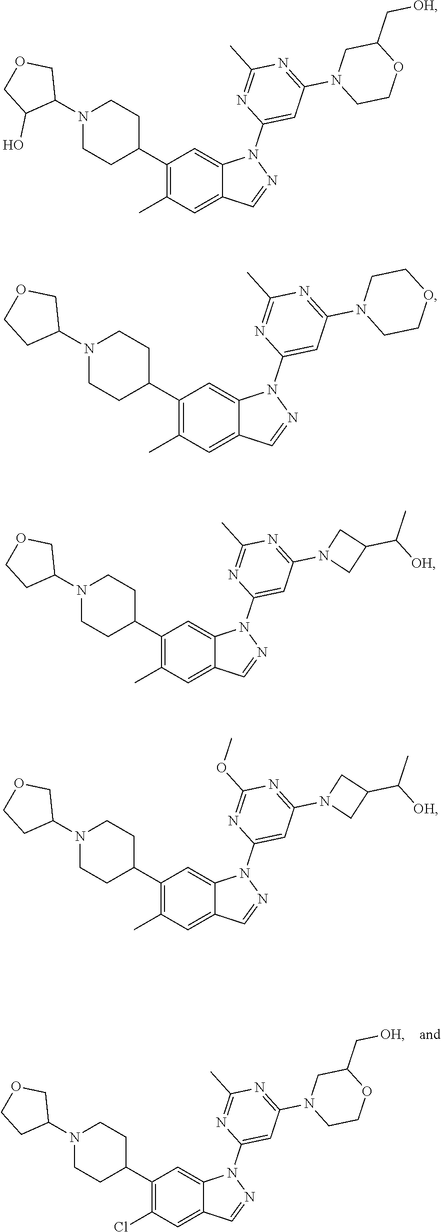 [SMILES: Cc1nc(N2CCOC(CO)C2)cc(-n2ncc3cc(C)c(C4CCN(C5COCC5O)CC4)cc32)n1 ; Cc1nc(N2CCOCC2)cc(-n2ncc3cc(C)c(C4CCN(C5CCOC5)CC4)cc32)n1 ; Cc1nc(N2CC(C(C)O)C2)cc(-n2ncc3cc(C)c(C4CCN(C5CCOC5)CC4)cc32)n1 ; COc1nc(N2CC(C(C)O)C2)cc(-n2ncc3cc(C)c(C4CCN(C5CCOC5)CC4)cc32)n1 ; Cc1nc(N2CCOC(CO)C2)cc(-n2ncc3cc(Cl)c(C4CCN(C5CCOC5)CC4)cc32)n1]

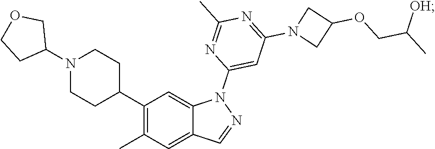 [SMILES: Cc1nc(N2CC(OCC(C)O)C2)cc(-n2ncc3cc(C)c(C4CCN(C5CCOC5)CC4)cc32)n1]

or a pharmaceutically acceptable salt thereof.

In one embodiment the invention provides ((2R)-4-(2-methyl-6-(5-methyl-6-(1-(tetrahydrofuran-3-yl)piperidin-4-yl)-1H-indazol-1-yl)pyrimidin-4-yl)morpholin-2-yl)methanol.

In one embodiment the invention provides a compound selected from
(4-(2-Methyl-6-(5-methyl-6-(1-(tetrahydrofuran-3-yl)piperidin-4-yl)-1H-indazol-1-yl)pyrimidin-4-yl)morpholin-2-yl)methanol,
1-(6-(6-(3-Fluoro-1-(tetrahydrofuran-3-yl)piperidin-4-yl)-5-methyl-1H-indazol-1-yl)-2-methoxypyrimidin-4-yl)azetidin-3-ol, 4-(6-(6-(3-Fluoro-1-(tetrahydrofuran-3-yl)piperidin-4-yl)-5-methyl-1H-indazol-1-yl)-2-methoxypyrimidin-4-yl)piperazin-2-one,
(6-Methyl-4-(2-methyl-6-(5-methyl-6-(1-(tetrahydrofuran-3-yl)piperidin-4-yl)-1H-indazol-1-yl)pyrimidin-4-yl)morpholin-2-yl)methanol,
1-(4-(2-methyl-6-(5-methyl-6-(1-(tetrahydrofuran-3-yl)piperidin-4-yl)-1H-indazol-1-yl)pyrimidin-4-yl)morpholin-2-yl)ethanol, and
4-(4-(1-(6-((S)-2-(hydroxymethyl)morpholino)-2-methylpyrimidin-4-yl)-5-methyl-1H-indazol-6-yl)piperidin-1-yl)tetrahydrofuran-3-ol,
4-(2-methyl-6-(5-methyl-6-(1-(tetrahydrofuran-3-yl)piperidin-4-yl)-1H-indazol-1-yl)pyrimidin-4-yl)morpholine,
1-(1-(2-methyl-6-(5-methyl-6-(1-(tetrahydrofuran-3-yl)piperidin-4-yl)-1H-indazol-1-yl)pyrimidin-4-yl)azetidin-3-yl)ethanol,
1-(1-(2-methoxy-6-(5-methyl-6-(1-(tetrahydrofuran-3-yl)piperidin-4-yl)-1H-indazol-1-yl)pyrimidin-4-yl)azetidin-3-yl)ethanol,
(4-(6-(5-chloro-6-(1-(tetrahydrofuran-3-yl)piperidin-4-yl)-1H-indazol-1-yl)-2-methylpyrimidin-4-yl)morpholin-2-yl)methanol, and
1-((1-(2-methyl-6-(5-methyl-6-(1-(tetrahydrofuran-3-yl)piperidin-4-yl)-1H-indazol-1-yl)pyrimidin-4-yl)azetidin-3-yl)oxy)propan-2-ol;
   or a pharmaceutically acceptable salt thereof.

In one embodiment the invention provides (4-(2-Methyl-6-(5-methyl-6-(1-(tetrahydrofuran-3-yl)piperidin-4-yl)-1H-indazol-1-yl)pyrimidin-4-yl)morpholin-2-yl)methanol,

or a pharmaceutically acceptable salt thereof.

In one embodiment the invention provides 1-(6-(6-(3-Fluoro-1-(tetrahydrofuran-3-yl)piperidin-4-yl)-5-methyl-1H-indazol-1-yl)-2-methoxypyrimidin-4-yl)azetidin-3-ol, or a pharmaceutically acceptable salt thereof.

In one embodiment, the invention provides 4-(6-(6-(3-Fluoro-1-(tetrahydrofuran-3-yl)piperidin-4-yl)-5-methyl-1H-indazol-1-yl)-2-methoxypyrimidin-4-yl)piperazin-2-one, or a pharmaceutically acceptable salt thereof.

In one embodiment, the invention provides (6-Methyl-4-(2-methyl-6-(5-methyl-6-(1-(tetrahydrofuran-3-yl)piperidin-4-yl)-1H-indazol-1-yl)pyrimidin-4-yl)morpholin-2-yl)methanol, or a pharmaceutically acceptable salt thereof.

In one embodiment, the invention provides (6-Methyl-4-(2-methyl-6-(5-methyl-6-(1-(tetrahydrofuran-3-yl)piperidin-4-yl)-1H-indazol-1-yl)pyrimidin-4-yl)morpholin-2-yl)methanol, or a pharmaceutically acceptable salt thereof.

In one embodiment, the invention provides 4-(4-(1-(6-((S)-2-(hydroxymethyl)morpholino)-2-methylpyrimidin-4-yl)-5-methyl-1H-indazol-6-yl)piperidin-1-yl)tetrahydrofuran-3-ol, or a pharmaceutically acceptable salt thereof.

In one embodiment, the invention provides 4-(2-methyl-6-(5-methyl-6-(1-(tetrahydrofuran-3-yl)piperidin-4-yl)-1H-indazol-1-yl)pyrimidin-4-yl)morpholine, or a pharmaceutically acceptable salt thereof.

In one embodiment, the invention provides 1-(1-(2-methyl-6-(5-methyl-6-(1-(tetrahydrofuran-3-yl)piperidin-4-yl)-1H-indazol-1-yl)pyrimidin-4-yl)azetidin-3-yl)ethanol, or a pharmaceutically acceptable salt thereof.

In one embodiment, the invention provides 1-(1-(2-methoxy-6-(5-methyl-6-(1-(tetrahydrofuran-3-yl)piperidin-4-yl)-1H-indazol-1-yl)pyrimidin-4-yl)azetidin-3-yl)ethanol, or a pharmaceutically acceptable salt thereof.

In one embodiment, the invention provides (4-(6-(5-chloro-6-(1-(tetrahydrofuran-3-yl)piperidin-4-yl)-1H-indazol-1-yl)-2-methylpyrimidin-4-yl)morpholin-2-yl)methanol, or a pharmaceutically acceptable salt thereof.

In one embodiment, the invention provides 1-((1-(2-methyl-6-(5-methyl-6-(1-(tetrahydrofuran-3-yl)piperidin-4-yl)-1H-indazol-1-yl)pyrimidin-4-yl)azetidin-3-yl)oxy)propan-2-ol, or a pharmaceutically acceptable salt thereof.

In one embodiment, the invention provides ((R)-4-(2-methyl-6-(5-methyl-6-(1-((R)-tetrahydrofuran-3-yl)piperidin-4-yl)-1H-indazol-1-yl)pyrimidin-4-yl)morpholin-2-yl)methanol.

In one embodiment, the invention provides ((R)-4-(2-methyl-6-(5-methyl-6-(1-((R)-tetrahydrofuran-3-yl)piperidin-4-yl)-1H-indazol-1-yl)pyrimidin-4-yl)morpholin-2-yl)methanol or a pharmaceutically acceptable salt thereof.

In one embodiment, the invention provides ((R)-4-(2-methyl-6-(5-methyl-6-(1-((S)-tetrahydrofuran-3-yl)piperidin-4-yl)-1H-indazol-1-yl)pyrimidin-4-yl)morpholin-2-yl)methanol.

In one embodiment, the invention provides ((R)-4-(2-methyl-6-(5-methyl-6-(1-((S)-tetrahydrofuran-3-yl)piperidin-4-yl)-1H-indazol-1-yl)pyrimidin-4-yl)morpholin-2-yl)methanol or a pharmaceutically acceptable salt thereof.

In one embodiment, the compound of formula (I) or a pharmaceutically acceptable salt thereof is a compound of any one of Examples 1 to 156 or a pharmaceutically acceptable salt thereof. In one embodiment, the compound of formula (I) is a compound of any one of Examples 1 to 156. In one embodiment, the invention provides a compound or a pharmaceutically acceptable salt thereof of any one of Examples 1 to 156.

In addition to the free base form of the compounds described herein, the salt form of the compounds is also within the scope of the present invention. The salts or pharmaceutically-acceptable salts of the compounds described herein may be prepared in situ during the final isolation and purification of the compound, or by separately reacting the purified compound in its free base form with a suitable base or acid, respectively. For reviews on suitable pharmaceutical salts see Berge et al, J. Pharm, Sci., 66, 1-19, 1977; P L Gould, International Journal of Pharmaceutics, 33 (1986), 201-217; and Bighley et al, Encyclopedia of Pharmaceutical Technology, Marcel Dekker Inc, New York 1996, Volume 13, page 453-497.

Certain compounds of formula (I) contain a basic group and are therefore capable of forming pharmaceutically-acceptable acid addition salts by treatment with a suitable acid. Suitable acids include pharmaceutically-acceptable inorganic acids and pharmaceutically-acceptable organic acids. Exemplary pharmaceutically-acceptable acid addition salts include hydrochloride, hydrobromide, nitrate, methylnitrate, sulfate, bisulfate, sulfamate, phosphate, acetate, hydroxyacetate, phenylacetate, propionate, butyrate, isobutyrate, valerate, maleate, hydroxymaleate, acrylate, fumarate, malate, tartrate, citrate, salicylate, p-aminosalicyclate, glycollate, lactate, heptanoate, phthalate, oxalate, succinate, benzoate, o-acetoxybenzoate, chlorobenzoate, methylbenzoate, dinitrobenzoate, hydroxybenzoate, methoxybenzoate, mandelate, tannate, formate, stearate, ascorbate, palmitate, oleate, pyruvate, pamoate, malonate, laurate, glutarate, glutamate, estolate, methanesulfonate (mesylate), ethanesulfonate (esylate), 2-hydroxyethanesulfonate, benzenesulfonate (besylate), p-aminobenzenesulfonate, p-toluenesulfonate (tosylate), and napthalene-2-sulfonate. In some embodiments, the pharmaceutically acceptable salts include the L-tartrate, ethanedisulfonate (edisylate), sulfate, phosphate, p-toluenesulfonate (tosylate), hydrochloride salt, methanesulfonate, citrate, fumarate, benzenesulfonate, maleate, hydrobromate, L-lactate, malonate, and S-camphor-10-sulfonate. In certain embodiments, some of these salts form solvates. In certain embodiments, some of these salts are crystalline.

Certain compounds of Formula (I) or salts thereof may exist in stereoisomeric forms (e.g., they may contain one or more asymmetric carbon atoms). The individual stereoisomers (enantiomers and diastereomers) and mixtures of these are included within the scope of the present invention. The different isomeric forms may be separated or resolved one from the other by conventional methods, or any given isomer may be obtained by conventional synthetic methods or by stereospecific or asymmetric syntheses.

Certain compounds of Formula (I) are capable of existing in tautomeric forms. For example, certain compounds exhibit keto-enol tautomerism. In some cases, only one of a pair of tautomeric forms fall within Formula (I). Such alternative tautomers also form part of the invention.

The invention also includes isotopically-labelled compounds and salts, which are identical to compounds of Formula (I) or salts thereof, but for the fact that one or more atoms are replaced by an atom having an atomic mass or mass number different from the atomic mass or mass number most commonly found in nature. Examples of isotopes that can be incorporated into compounds of Formula (I) or salts thereof isotopes of hydrogen, carbon, nitrogen, fluorine, such as ³H, ¹¹C, ¹⁴C and ¹⁸F. Such isotopically-labelled compound of Formula (I) or salts thereof are useful in drug and/or substrate tissue distribution assays. For example, ¹¹C and ¹⁸F isotopes are useful in PET (positron emission tomography). PET is useful in brain imaging. Isotopically-labelled compounds of Formula (I) and salts thereof can generally be prepared by carrying out the procedures disclosed below, by substituting a readily available isotopically-labelled reagent for a non-isotopically labelled reagent. In one embodiment, compounds of Formula (I) or salts thereof are not isotopically labelled.

Certain compounds of Formula (I) or salts thereof may exist in solid or liquid form. In the solid state, compounds of Formula (I) or salts may exist in crystalline or noncrystalline form, or as a mixture thereof. For compounds of Formula (I) or salts that are in crystalline form, the skilled artisan will appreciate that pharmaceutically-acceptable solvates may be formed wherein solvent molecules are incorporated into the crystalline lattice during crystallization. Solvates may involve nonaqueous solvents such as ethanol, isopropanol, DMSO, acetic acid, ethanolamine, and ethyl acetate, or they may involve water as the solvent that is incorporated into the crystalline lattice. Solvates wherein water is the solvent that is incorporated into the crystalline lattice are typically referred to as “hydrates.” Hydrates include stoichiometric hydrates as well as compositions containing variable amounts of water.

The skilled artisan will further appreciate that certain compounds of Formula (I), pharmaceutically acceptable salts or solvates thereof that exist in crystalline form, including the various solvates thereof, may exhibit polymorphism (i.e. the capacity to occur in different crystalline structures). These different crystalline forms are typically known as “polymorphs.” Polymorphs have the same chemical composition but differ in packing, geometrical arrangement, and other descriptive properties of the crystalline solid state. Polymorphs, therefore, may have different physical properties such as shape, density, hardness, deformability, stability, and dissolution properties. Polymorphs typically exhibit different melting points, IR spectra, and X-ray powder diffraction patterns, which may be used for identification. The skilled artisan will appreciate that different polymorphs may be produced, for example, by changing or adjusting the reaction conditions or reagents, used in making the compound. For example, changes in temperature, pressure, or solvent may result in polymorphs. In addition, one polymorph may spontaneously convert to another polymorph under certain conditions.

The skilled artisan also appreciates that this invention may contain various deuterated forms of compounds of Formula (I), or pharmaceutically acceptable salts thereof. Each available hydrogen atom attached to a carbon atom may be independently replaced with a deuterium atom. A person of ordinary skill in the art will know how to synthesize deuterated forms of compounds of Formula (I), or pharmaceutically acceptable salts thereof. Commercially available deuterated starting materials may be employed in the preparation of deuterated forms of compounds of Formula (I) or pharmaceutically acceptable salts thereof, or they may be synthesized using conventional techniques employing deuterated reagents (e.g. lithium aluminum deuteride).

### C. Methods of Use

Compounds of Formula (I) or pharmaceutically acceptable salts thereof are inhibitors of LRRK2 kinase activity and are thus believed to be of potential use in the treatment of or prevention of the following neurological diseases: Parkinson's disease, Alzheimer's disease, dementia (including Lewy body dementia and vascular dementia, HIV-induced dementia), amyotrophic lateral sclerosis (ALS), age related memory dysfunction, mild cognitive impairment, argyrophilic grain disease, Pick's disease, corticobasal degeneration, progressive supranuclear palsy, inherited frontotemporal dementia and parkinsonism linked to chromosome 17 (FTDP-17), withdrawal symptoms/relapse associated with drug addiction, L-Dopa induced dyskinesia, ischemic stroke, traumatic brain injury, spinal cord injury and multiple sclerosis. Other diseases potentially treatable by inhibition of LRRK2 include, but are not limited to, lysosomal disorders (for example, Niemann-Pick Type C disease, Gaucher disease), Crohn's disease, cancers (including thyroid, renal (including papillary renal), breast, lung and prostate cancers, leukemias (including acute myelogenous leukemia (AML)) and lymphomas), rheumatoid arthritis, systemic lupus erythematosus, autoimmune hemolytic anemia, pure red cell aplasia, idiopathic thrombocytopenic purpura (ITP), Evans syndrome, vasculitis, bullous skin disorders, type 1 diabetes mellitus, obesity, epilepsy, pulmonary diseases such as chronic obstructive pulmonary disease, idiopathic pulmonary fibrosis, Sjogren's syndrome, Devic's disease, inflammatory myopathies, ankylosing spondylitis, bacterial infections (including leprosy), viral infections (including tuberculosis, HIV, West Nile virus and chikungunya virus) and parasitic infections.

One aspect of the invention provides a compound of Formula (I) or a pharmaceutically acceptable salt thereof for use in therapy. In one embodiment, the invention provides a compound of Formula (I) or a pharmaceutically acceptable salt thereof for use in the treatment of or prevention of the above disorders (i.e. the neurological diseases and other diseases listed above). In one embodiment, the invention provides a compound of Formula (I) or a pharmaceutically acceptable salt thereof for use in the treatment of or prevention of Parkinson's disease. In one embodiment, the invention provides a compound of Formula (I) or a pharmaceutically acceptable salt thereof for use in the treatment of Parkinson's disease. In another embodiment, the invention provides a compound of Formula (I) or a pharmaceutically acceptable salt thereof for use in the treatment of or prevention of Alzheimer's disease. In one embodiment, the invention provides a compound of Formula (I) or a pharmaceutically acceptable salt thereof for use in the treatment of Alzheimer's disease. In another embodiment, the invention provides a compound of Formula (I) or a pharmaceutically acceptable salt thereof for use in the treatment of amyotrophic lateral sclerosis (ALS).

In one embodiment, the invention provides ((R)-4-(2-methyl-6-(5-methyl-6-(1-((S)-tetrahydrofuran-3-yl)piperidin-4-yl)-1H-indazol-1-yl)pyrimidin-4-yl)morpholin-2-yl)methanol or a pharmaceutically acceptable salt thereof for use in the treatment or prevention of Parkinson's disease, Alzheimer's disease or amyotrophic lateral sclerosis (ALS).

In another embodiment, the invention provides ((R)-4-(2-methyl-6-(5-methyl-6-(1-((S)-tetrahydrofuran-3-yl)piperidin-4-yl)-1H-indazol-1-yl)pyrimidin-4-yl)morpholin-2-yl)methanol or a pharmaceutically acceptable salt thereof for use in the treatment of Parkinson's disease.

A further aspect of the invention provides the use of a compound of Formula (I) or a pharmaceutically acceptable salt thereof in the manufacture of a medicament for the treatment or prevention of the above disorders (i.e. the neurological diseases and other diseases listed above). A further aspect of the invention provides the use of a compound of Formula (I) or a pharmaceutically acceptable salt thereof in the manufacture of a medicament for the treatment of or prevention of Parkinson's disease. A further aspect of the invention provides the use of a compound of Formula (I) or a pharmaceutically acceptable salt thereof in the manufacture of a medicament for the treatment of Parkinson's disease. In another embodiment, the invention provides the use of a compound of Formula (I) or a pharmaceutically acceptable salt thereof in the manufacture of a medicament for the treatment or prevention of Alzheimer's disease. In one embodiment, the invention provides the use of a compound of Formula (I) or a pharmaceutically acceptable salt thereof in the manufacture of a medicament for the treatment of Alzheimer's disease. In another embodiment, the invention provides use of a compound of Formula (I) or a pharmaceutically acceptable salt thereof in the manufacture of a medicament for the treatment of amyotrophic lateral sclerosis (ALS).

In one embodiment, the invention provides the use of ((R)-4-(2-methyl-6-(5-methyl-6-(1-((S)-tetrahydrofuran-3-yl)piperidin-4-yl)-1H-indazol-1-yl)pyrimidin-4-yl)morpholin-2-yl)methanol or a pharmaceutically acceptable salt thereof in the manufacture of a medicament for the treatment or prevention of Parkinson's disease, Alzheimer's disease or amyotrophic lateral sclerosis (ALS).

In another embodiment, the invention provides the use of ((R)-4-(2-methyl-6-(5-methyl-6-(1-((S)-tetrahydrofuran-3-yl)piperidin-4-yl)-1H-indazol-1-yl)pyrimidin-4-yl)morpholin-2-yl)methanol or a pharmaceutically acceptable salt thereof in the manufacture of a medicament for the treatment or prevention of Parkinson's disease.

In yet another embodiment, the invention provides the use of ((R)-4-(2-methyl-6-(5-methyl-6-(1-((S)-tetrahydrofuran-3-yl)piperidin-4-yl)-1H-indazol-1-yl)pyrimidin-4-yl)morpholin-2-yl)methanol or a pharmaceutically acceptable salt thereof in the manufacture of a medicament for the treatment of Parkinson's disease.

A further aspect of the invention provides a method of treatment or prevention of a disorder listed above (i.e. selected from the neurological diseases and other diseases listed above), which comprises administering to a subject in need thereof a therapeutically effective amount of a compound of Formula (I) or a pharmaceutically acceptable salt thereof. A further aspect of the invention provides a method of treatment or prevention of Parkinson's disease, which comprises administering to a subject in need thereof a therapeutically effective amount of a compound of Formula (I) or a pharmaceutically acceptable salt thereof. A further aspect of the invention provides a method of treatment of Parkinson's disease, which comprises administering to a subject in need thereof a therapeutically effective amount of a compound of Formula (I) or a pharmaceutically acceptable salt thereof. A further aspect of the invention provides a method of treatment or prevention of Alzheimer's disease, which comprises administering to a subject in need thereof a therapeutically effective amount of a compound of Formula (I) or a pharmaceutically acceptable salt thereof. A further aspect of the invention provides a method of treatment of Alzheimer's disease, which comprises administering to a subject in need thereof a therapeutically effective amount of a compound of Formula (I) or a pharmaceutically acceptable salt thereof. A further aspect of the invention provides a method of treatment of tuberculosis, which comprises administering to a subject in need thereof a therapeutically effective amount of a compound of Formula (I) or a pharmaceutically acceptable salt thereof. In an embodiment, the subject is human.

In one embodiment, the invention provides a method of treatment of Parkinson's disease, Alzheimer's disease or amyotrophic lateral sclerosis (ALS), which comprises administering to a subject in need thereof a therapeutically effective amount of a compound of Formula (I) or a pharmaceutically acceptable salt thereof.

In one embodiment, the invention provides a method of treatment of Parkinson's disease, Alzheimer's disease or amyotrophic lateral sclerosis (ALS), which comprises administering to a human in need thereof a therapeutically effective amount of a compound of Formula (I) or a pharmaceutically acceptable salt thereof.

In one embodiment, the invention provides a method of treatment of Parkinson's disease, which comprises administering to a subject in need thereof a therapeutically effective amount of a compound of Formula (I) or a pharmaceutically acceptable salt thereof.

In one embodiment, the invention provides a method of treatment of Parkinson's disease, which comprises administering to a human in need thereof a therapeutically effective amount of a compound of Formula (I) or a pharmaceutically acceptable salt thereof.

In one embodiment, the invention provides a method of treatment of Parkinson's disease, which comprises administering to a human in need thereof a therapeutically effective amount of a compound selected from

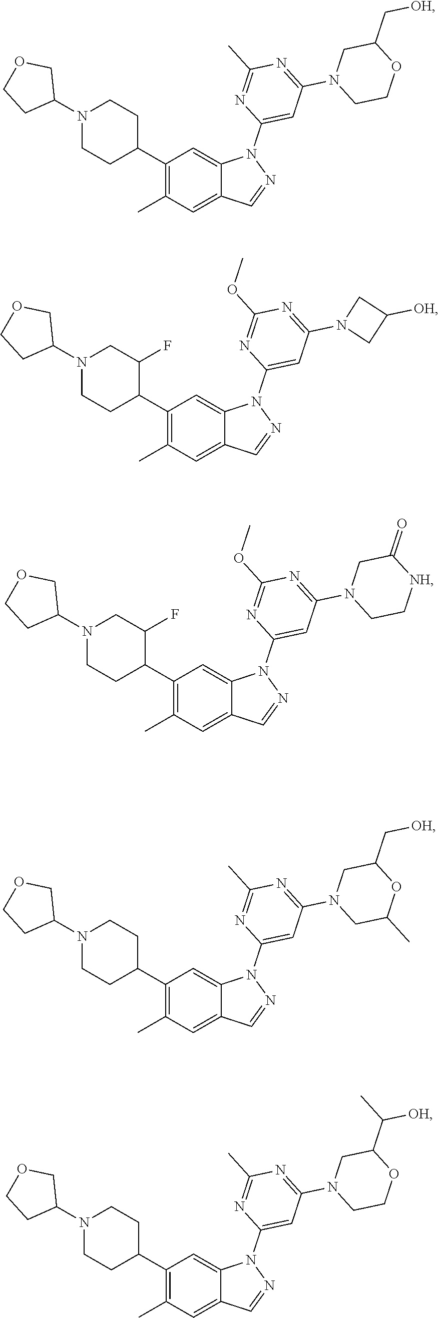 [SMILES: Cc1nc(N2CCOC(CO)C2)cc(-n2ncc3cc(C)c(C4CCN(C5CCOC5)CC4)cc32)n1 ; COc1nc(N2CC(O)C2)cc(-n2ncc3cc(C)c(C4CCN(C5CCOC5)CC4F)cc32)n1 ; COc1nc(N2CCNC(=O)C2)cc(-n2ncc3cc(C)c(C4CCN(C5CCOC5)CC4F)cc32)n1 ; Cc1nc(N2CC(C)OC(CO)C2)cc(-n2ncc3cc(C)c(C4CCN(C5CCOC5)CC4)cc32)n1 ; Cc1nc(N2CCOC(C(C)O)C2)cc(-n2ncc3cc(C)c(C4CCN(C5CCOC5)CC4)cc32)n1]

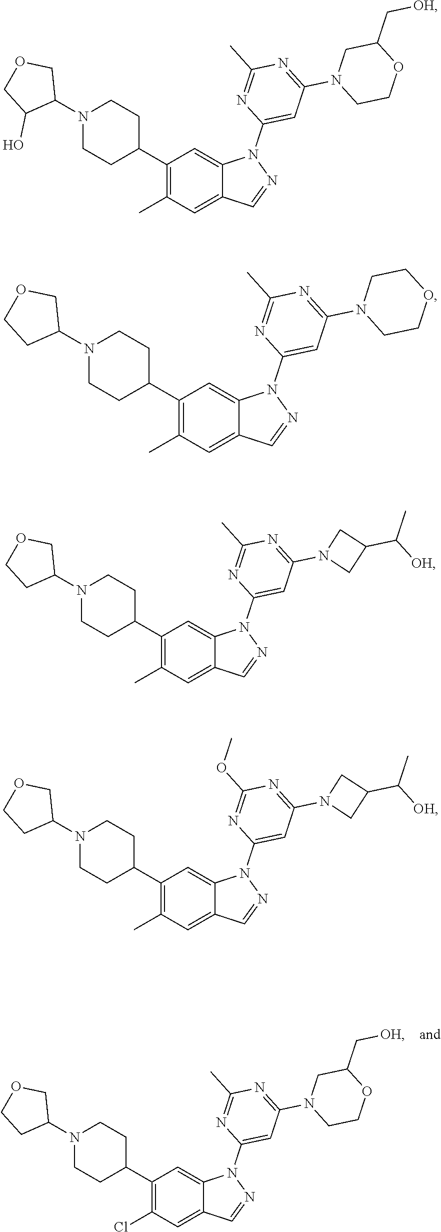 [SMILES: Cc1nc(N2CCOC(CO)C2)cc(-n2ncc3cc(C)c(C4CCN(C5COCC5O)CC4)cc32)n1 ; Cc1nc(N2CCOCC2)cc(-n2ncc3cc(C)c(C4CCN(C5CCOC5)CC4)cc32)n1 ; Cc1nc(N2CC(C(C)O)C2)cc(-n2ncc3cc(C)c(C4CCN(C5CCOC5)CC4)cc32)n1 ; COc1nc(N2CC(C(C)O)C2)cc(-n2ncc3cc(C)c(C4CCN(C5CCOC5)CC4)cc32)n1 ; Cc1nc(N2CCOC(CO)C2)cc(-n2ncc3cc(Cl)c(C4CCN(C5CCOC5)CC4)cc32)n1]

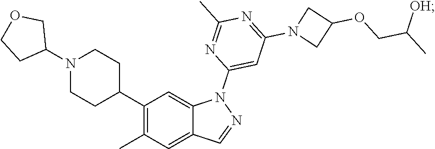 [SMILES: Cc1nc(N2CC(OCC(C)O)C2)cc(-n2ncc3cc(C)c(C4CCN(C5CCOC5)CC4)cc32)n1]

or a pharmaceutically acceptable salt thereof.

In one embodiment, the invention provides a method of treatment of Parkinson's disease, which comprises administering to a human in need thereof a therapeutically effective amount of 6-(1-((R)-tetrahydrofuran-3-yl)piperidin-4-yl)-1H-indazol-1-yl)pyrimidin-4-yl)morpholin-2-yl)methanol.

In one embodiment, the invention provides a method of treatment of Parkinson's disease, which comprises administering to a human in need thereof a therapeutically effective amount of 6-(1-((R)-tetrahydrofuran-3-yl)piperidin-4-yl)-1H-indazol-1-yl)pyrimidin-4-yl)morpholin-2-yl)methanol or a pharmaceutically acceptable salt thereof.

In one embodiment, the invention provides a method of treatment of Parkinson's disease, which comprises administering to a human in need thereof a therapeutically effective amount of ((R)-4-(2-methyl-6-(5-methyl-6-(1-((S)-tetrahydrofuran-3-yl)piperidin-4-yl)-1H-indazol-1-yl)pyrimidin-4-yl)morpholin-2-yl)methanol.

In one embodiment, the invention provides a method of treatment of Parkinson's disease, which comprises administering to a human in need thereof a therapeutically effective amount of ((R)-4-(2-methyl-6-(5-methyl-6-(1-((S)-tetrahydrofuran-3-yl)piperidin-4-yl)-1H-indazol-1-yl)pyrimidin-4-yl)morpholin-2-yl)methanol or a pharmaceutically acceptable salt thereof.

In the context of the present invention, treatment of Parkinson's disease refers to the treatment of sporadic Parkinson's disease, and/or familial Parkinson's disease. In one embodiment, treatment of Parkinson's disease refers to treatment of familial Parkinson's disease. Familial Parkinson's disease patients are those expressing one or more of the following LRRK2 kinase mutations: G2019S mutation, N1437H mutation, R1441G mutation, R1441C mutation, R1441H mutation, Y1699C mutation, S1761R mutation, or I2020T mutation. In another embodiment, familial Parkinson's disease patients express other coding mutations (such as G2385R) or non-coding single nucleotide polymorphisms at the LRRK2 locus that are associated with Parkinson's disease In a more particular embodiment, familial Parkinson's disease includes patients expressing the G2019S mutation or the R1441G mutation in LRRK2 kinase. In one embodiment, treatment of Parkinson's disease refers to the treatment of familial Parkinson's disease includes patients expressing LRRK2 kinase bearing G2019S mutation. In another embodiment, familial Parkinson's disease patients express aberrantly high levels of normal LRRK2 kinase.

In one embodiment, the invention provides a method of treatment of Parkinson's disease, which comprises administering to a human expressing the G2019S mutation in LRRK2 kinase in need thereof a therapeutically effective amount of a compound of Formula (I) or a pharmaceutically acceptable salt thereof.

In one embodiment, the invention provides a method of treatment of Parkinson's disease, which comprises testing in a human for the G2019S mutation in LRRK2 kinase and administering to the human expressing the G2019S mutation in LRRK2 kinase in need thereof a therapeutically effective amount of a compound of Formula (I) or a pharmaceutically acceptable salt thereof.

Treatment of Parkinson's disease may be symptomatic or may be disease modifying. In one embodiment, treatment of Parkinson's disease refers to symptomatic treatment. In one embodiment, treatment of Parkinson's disease refers to disease modifying treatment.

Compounds of the present invention may also be useful in treating patients identified as susceptible to progression to severe Parkinsonism by means of one or more subtle features associated with disease progression such as family history, olfaction deficits, constipation, cognitive defects, gait or biological indicators of disease progression gained from molecular, biochemical, immunological or imaging technologies. In this context, treatment may be symptomatic or disease modifying.

In the context of the present invention, treatment of Alzheimer's disease refers to the treatment of sporadic Alzheimer's disease and/or familial Alzheimer's disease. Treatment of Alzheimer's disease may be symptomatic or may be disease modifying. In one embodiment, treatment of Alzheimer's disease refers to symptomatic treatment.

In the context of the present invention, treatment of dementia (including Lewy body dementia and vascular dementia, HIV-induced dementia), amyotrophic lateral sclerosis (ALS), age related memory dysfunction, mild cognitive impairment, argyrophilic grain disease, Pick's disease, corticobasal degeneration, progressive supranuclear palsy, inherited frontotemporal dementia and parkinsonism linked to chromosome 17 (FTDP-17), multiple sclerosis, lysosomal disorders (for example, Niemann-Pick Type C disease, Gaucher disease), Crohn's disease, cancers (including thyroid, renal (including papillary renal), breast, lung and prostate cancers, leukemias (including acute myelogenous leukemia (AML)) and lymphomas), rheumatoid arthritis, systemic lupus erythematosus, autoimmune hemolytic anemia, pure red cell aplasia, idiopathic thrombocytopenic purpura (ITP), Evans syndrome, vasculitis, bullous skin disorders, type 1 diabetes mellitus, obesity, epilepsy, pulmonary diseases such as chronic obstructive pulmonary disease, idiopathic pulmonary fibrosis, Sjogren's syndrome, Devic's disease, inflammatory myopathies, ankylosing spondylitis, may be symptomatic or disease modifying. In certain embodiments, treatment of these disorders refers to symptomatic treatment.

The invention also provides the use of inhibitors of LRRK2 in the production of neuronal progenitor cells in vitro for consequent therapeutic application in cell based-treatment of CNS disorders.

When a compound of Formula (I) or a pharmaceutically acceptable salt thereof is intended for use in the treatment of Parkinson's disease, it may be used in combination with medicaments alleged to be useful as symptomatic treatments of Parkinson's disease. Suitable examples of such other therapeutic agents include L-dopa, and dopamine agonists (e.g. pramipexole, ropinirole).

When a compound of Formula (I) or a pharmaceutically acceptable salt thereof is intended for use in the treatment of Alzheimer's disease, it may be used in combination with medicaments claimed to be useful as either disease modifying or symptomatic treatments of Alzheimer's disease. Suitable examples of such other therapeutic agents may be symptomatic agents, for example those known to modify cholinergic transmission such as M1 muscarinic receptor agonists or allosteric modulators, M2 muscarinic antagonists, acetylcholinesterase inhibitors (such as tetrahydroaminoacridine, donepezil hydrochloride rivastigmine, and galantamine), nicotinic receptor agonists or allosteric modulators (such as α7 agonists or allosteric modulators or α4β2 agonists or allosteric modulators), PPAR agonists (such as PPARγ agonists), 5-HT₄ receptor partial agonists, 5-HT₆ receptor antagonists e.g. SB-742457 or 5HT1A receptor antagonists and NMDA receptor antagonists or modulators, or disease modifying agents such as β or γ-secretase inhibitors e.g semagacestat, mitochondrial stabilizers, microtubule stabilizers or modulators of Tau pathology such as Tau aggregation inhibitors (e.g. methylene blue and REMBER™) NSAIDS, e.g. tarenflurbil, tramiprosil; or antibodies for example bapineuzumab or solanezumab; proteoglycans for example tramiprosate.

When a compound of Formula (I) or a pharmaceutically acceptable salt thereof is intended for use in the treatment of bacterial infections, parasitic infections or viral infections, it may be used in combination with medicaments alleged to be useful as symptomatic treatments that directly target the infectious agent.

When a compound of Formula (I) or a pharmaceutically acceptable salt thereof is used in combination with other therapeutic agents, the compound may be administered either sequentially or simultaneously by any convenient route.

The invention also provides, in a further aspect, a combination comprising a compound of Formula (I) or a pharmaceutically acceptable salt thereof together with one or more further therapeutic agent or agents.

The combinations referred to above may conveniently be presented for use in the form of a pharmaceutical formulation and thus pharmaceutical formulations comprising a combination as defined above together with a pharmaceutically acceptable carrier or excipient comprise a further aspect of the invention. The individual components of such combinations may be administered either sequentially or simultaneously in separate or combined pharmaceutical formulations.

When a compound of Formula (I) or a pharmaceutically acceptable salt thereof is used in combination with a second therapeutic agent active against the same disease state the dose of each compound may differ from that when the compound is used alone. Appropriate doses will be readily appreciated by those skilled in the art.

### D. Composition

Compounds of Formula (I) or pharmaceutically acceptable salts thereof may be formulated into pharmaceutical compositions prior to administration to a subject. According to one aspect, the invention provides a pharmaceutical composition comprising a compound of Formula (I) or a pharmaceutically acceptable salt thereof and a pharmaceutically acceptable excipient. According to another aspect, the invention provides a process for the preparation of a pharmaceutical composition comprising admixing a compound of Formula (I) or a pharmaceutically acceptable salt thereof, with a pharmaceutically acceptable excipient.

Pharmaceutical compositions may be presented in unit dose forms containing a predetermined amount of active ingredient per unit dose. Such a unit may contain, for example, 0.1 mg, 0.5 mg, or 1 mg to 50 mg, 100 mg, 200 mg, 250 mg, 500 mg, 750 mg or 1 g of a compound of the present invention, depending on the disease being treated, the 35 route of administration and the age, weight and condition of the subject, or pharmaceutical compositions may be presented in unit dose forms containing a predetermined amount of active ingredient per unit dose. In other embodiments, the unit dosage compositions are those containing a daily dose or sub-dose as described herein, or an appropriate fraction thereof, of an active ingredient. Furthermore, such pharmaceutical compositions may be prepared by any of the methods well-known to one skilled in the art.

A therapeutically effective amount of a compound of Formula (I) will depend upon a number of factors including, for example, the age and weight of the intended recipient, the precise condition requiring treatment and its severity, the nature of the formulation, and the route of administration, and will ultimately be at the discretion of the attendant prescribing the medication. However, a therapeutically effective amount of a compound of formula (I) for the treatment of diseases described in the present invention will generally be in the range of 0.1 to 100 mg/kg body weight of recipient per day and more usually in the range of 1 to 10 mg/kg body weight per day. Thus, for a 70 kg adult mammal, the actual amount per day would usually be from 70 to 700 mg and this amount may be given in a single dose per day or in a number of sub-doses per day as such as two, three, four, five or six doses per day. Or the dosing can be done intermittently, such as once every other day, once a week or once a month. A therapeutically effective amount of a pharmaceutically acceptable salt or solvate, etc., may be determined as a proportion of the therapeutically effective amount of the compound of Formula (I) per se. It is envisaged that similar dosages would be appropriate for treatment of the other diseases referred to above.

The pharmaceutical compositions of the invention may contain one or more compounds of Formula (I) or a pharmaceutically acceptable salt thereof. In some embodiments, the pharmaceutical compositions may contain more than one compound of the invention. For example, in some embodiments, the pharmaceutical compositions may contain two or more compounds of Formula (I) or a pharmaceutically acceptable salt thereof. In addition, the pharmaceutical compositions may optionally further comprise one or more additional active pharmaceutical ingredients (APIs).

As used herein, “pharmaceutically acceptable excipient” means a pharmaceutically acceptable material, composition or vehicle involved in giving form or consistency to the pharmaceutical composition. Each excipient may be compatible with the other ingredients of the pharmaceutical composition when commingled such that interactions which would substantially reduce the efficacy of the compound of the invention when administered to a subject and interactions which would result in pharmaceutical compositions that are not pharmaceutically acceptable are avoided.

The compounds of the invention and the pharmaceutically-acceptable excipient or excipients may be formulated into a dosage form adapted for administration to the subject by the desired route of administration. For example, dosage forms include those adapted for (1) oral administration (including buccal or sublingual) such as tablets, capsules, caplets, pills, troches, powders, syrups, elixers, suspensions, solutions, emulsions, sachets, and cachets; (2) parenteral administration (including subcutaneous, intramuscular, intravenous or intradermal) such as sterile solutions, suspensions, and powders for reconstitution; (3) transdermal administration such as transdermal patches; (4) rectal administration such as suppositories; (5) nasal inhalation such as dry powders, aerosols, suspensions, and solutions; and (6) topical administration (including buccal, sublingual or transdermal) such as creams, ointments, lotions, solutions, pastes, sprays, foams, and gels. Such compositions may be prepared by any methods known in the art of pharmacy, for example by bringing into association a compound of Formula (I) with the carrier(s) or excipient(s).

Pharmaceutical compositions adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

Suitable pharmaceutically-acceptable excipients may vary depending upon the particular dosage form chosen. In addition, suitable pharmaceutically-acceptable excipients may be chosen for a particular function that they may serve in the composition. For example, certain pharmaceutically-acceptable excipients may be chosen for their ability to facilitate the production of uniform dosage forms. Certain pharmaceutically-acceptable excipients may be chosen for their ability to facilitate the production of stable dosage forms. Certain pharmaceutically acceptable excipients may be chosen for their ability to facilitate carrying or transporting the compound or compounds of the invention once administered to the subject from an organ, or a portion of the body, to another organ, or a portion of the body. Certain pharmaceutically-acceptable excipients may be chosen for their ability to enhance patient compliance.

Suitable pharmaceutically acceptable excipients include the following types of excipients: diluents, fillers, binders, disintegrants, lubricants, glidants, granulating agents, coating agents, wetting agents, solvents, co-solvents, suspending agents, emulsifiers, sweeteners, flavoring agents, flavor masking agents, coloring agents, anticaking agents, hemectants, chelating agents, plasticizers, viscosity increasing agents, antioxidants, preservatives, stabilizers, surfactants, and buffering agents. The skilled artisan will appreciate that certain pharmaceutically-acceptable excipients may serve more than one function and may serve alternative functions depending on how much the excipient is present in the formulation and what other ingredients are present in the formulation.

Skilled artisans possess the knowledge and skill in the art to enable them to select suitable pharmaceutically-acceptable excipients in appropriate amounts for use in the invention. In addition, there are a number of resources that are available to the skilled artisan which describe pharmaceutically-acceptable excipients and may be useful in selecting suitable pharmaceutically-acceptable excipients. Examples include Remington's Pharmaceutical Sciences (Mack Publishing Company), The Handbook of Pharmaceutical Additives (Gower Publishing Limited), and The Handbook of Pharmaceutical Excipients (the American Pharmaceutical Association and the Pharmaceutical Press).

The pharmaceutical compositions of the invention are prepared using techniques and methods known to those skilled in the art. Some of the methods commonly used in the art are described in Remington's Pharmaceutical Sciences (Mack Publishing Company).

In one aspect, the invention is directed to a solid oral dosage form such as a tablet or capsule comprising a therapeutically effective amount of a compound of the invention and a diluent or filler. Suitable diluents and fillers include lactose, sucrose, dextrose, mannitol, sorbitol, starch (e.g. corn starch, potato starch, and pre-gelatinized starch), cellulose and its derivatives (e.g. microcrystalline cellulose), calcium sulfate, and dibasic calcium phosphate. The oral solid dosage form may further comprise a binder. Suitable binders include starch (e.g. corn starch, potato starch, and pre-gelatinized starch), gelatin, acacia, sodium alginate, alginic acid, tragacanth, guar gum, povidone, and cellulose and its derivatives (e.g. microcrystalline cellulose). The oral solid dosage form may further comprise a disintegrant. Suitable disintegrants include crospovidone, sodium starch glycolate, croscarmelose, alginic acid, and sodium carboxymethyl cellulose. The oral solid dosage form may further comprise a lubricant. Suitable lubricants include stearic acid, magnesium stearate, calcium stearate, and talc.

In certain embodiments, the present invention is directed to a pharmaceutical composition comprising 0.01 to 1000 mg of one or more of a compound of Formula (I) or a pharmaceutically acceptable salt thereof and 0.01 to 5 g of one or more pharmaceutically acceptable excipients.

In another embodiment, the present invention is directed to a pharmaceutical composition for the treatment of a neurodegeneration disease comprising a compound of formula (I) or a pharmaceutically acceptable salt thereof and a pharmaceutically acceptable excipient. In another embodiment, the present invention is directed to a pharmaceutical composition for the treatment of Parkinson's disease comprising a compound of formula (I) or a pharmaceutically acceptable salt thereof and a pharmaceutically acceptable excipient.

### E. Process of Preparing Compounds

The process to be utilized in the preparation of compounds of formula (I) or salts thereof described herein depends upon the desired compounds. Such factors as the selection of the specific substituent and various possible locations of the specific substituent all play a role in the path to be followed in the preparation of the specific compounds of this invention. Those factors are readily recognized by one of ordinary skill in the art.

In general, the compounds of the present invention may be prepared by standard techniques known in the art and by known processes analogous thereto. General methods for preparing compounds of formula (I) are set forth below. All starting material and reagents described in the below general experimental schemes are commercially available or can be prepared by methods known to one skilled in the art.

The skilled artisan will appreciate that if a substituent described herein is not compatible with the synthetic methods described herein, the substituent may be protected with a suitable protecting group that is stable to the reaction conditions. The protecting group may be removed at a suitable point in the reaction sequence to provide a desired intermediate or target compound. Suitable protecting groups and the methods for protecting and de-protecting different substituents using such suitable protecting groups are well known to those skilled in the art; examples of which may be found in T. Greene and P. Wuts, Protecting Groups in Chemical Synthesis (3rd ed.), John Wiley & Sons, NY (1999). In some instances, a substituent may be specifically selected to be reactive under the reaction conditions used. Under these circumstances, the reaction conditions convert the selected substituent into another substituent that is either useful as an intermediate compound or is a desired substituent in a target compound.

General Scheme 1 provides exemplary processes of synthesis for preparing compounds of the present invention.

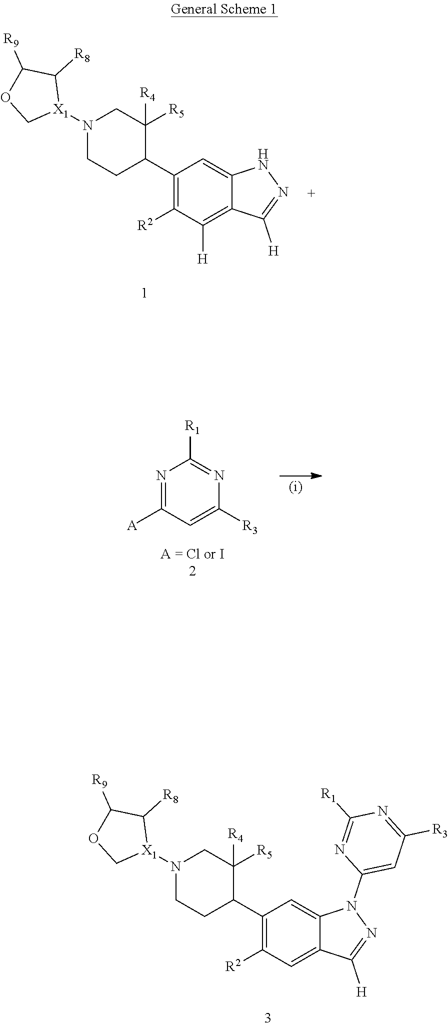

General Scheme 1 provides an exemplary synthesis for preparing compound 3 which represents compounds of Formula (I). In General Scheme 1, R₁, R₂, R₃, R₄, R₅, R₈, R₉ and X₁ are as defined in Formula I.

Step (i) may be a substitution reaction by reacting compound 1 with compound 2 using appropriate base such as Cs₂CO₃ in an appropriate solvent such as N, N-dimethylformamide (DMF) under suitable temperature such as about 100° C. to provide compound 3.

Step (i) may alternatively be a coupling reaction using appropriate reagents such as CuI and N,N′-dimethyl-cyclohexane-1,2-diamine in the presence of a suitable base such as K₃PO₄ in a suitable solvent such as toluene at suitable temperature such as reflux condition to provide compound 3.

Step (i) may alternatively be a coupling reaction using appropriate reagents such as Pd₂dba₃ and di-tert-butyl(2′,4′,6′-triisopropyl-[1,1′-biphenyl]-2-yl)phosphine in the presence of suitable base such as sodium tert-butoxide in a suitable solvent such as toluene at suitable temperature such as 100° C. to provide compound 3.

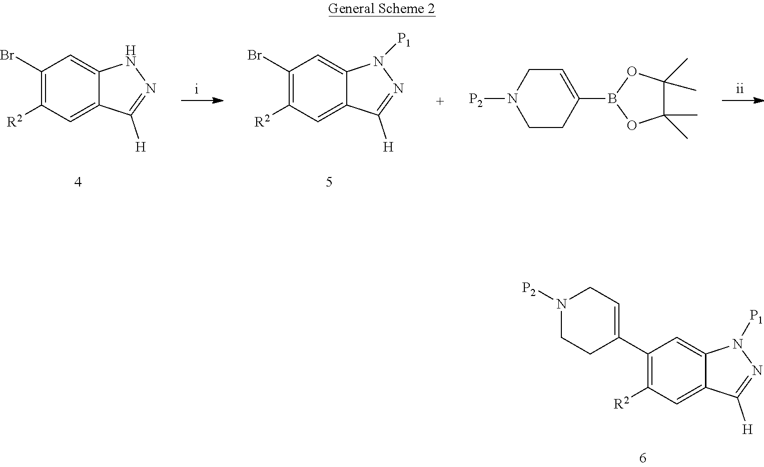

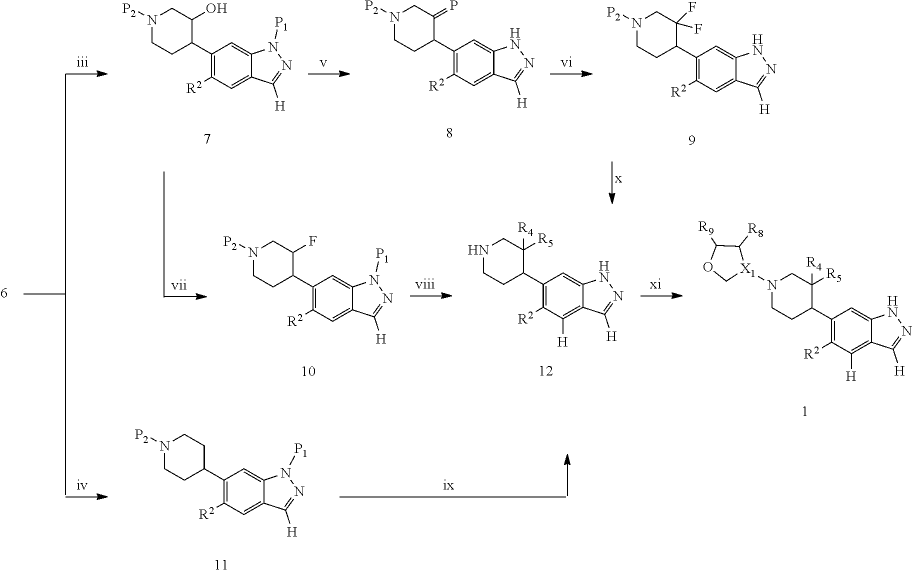

General Scheme 2 provides an exemplary synthesis for preparing intermediate 1. The protecting group, P₁, can be any suitable protecting groups for example, tetrahydro-2H-pyran-2-yl (THP), (trimethylsilyl)ethoxy)methyl (SEM) or Acetyl (Ac).

Intermediate 5 can be obtained in step (i) by reacting starting material 4 with suitable reagents such as DHP in the presence of suitable acids such as TsOH in appropriate solvents such as DCM under suitable temperatures such as 20° C. to 40° C.

Step (ii) is a cross-coupling reaction between intermediate 5 and boronic acid or esters using appropriate palladium catalysts such as Pd(dppf)Cl₂ in the presence of suitable bases such as Na₂CO₃ in appropriate solvents such as 1,4-dioxane at suitable temperatures such as 60° C. to 100° C.

Step (iii) involves reaction with suitable oxidation reagents such as H₂O₂ in a suitable solvent such as THE under suitable temperatures such as −60° C. to −10° C. to provide intermediate 7.

Step (iv) is a reaction with a suitable reducing reagent such as hydrogen in the presence of suitable catalysts such Pd/C in polar solvents such as MeOH at appropriate temperatures such as 25° C. to 8000.

Step (v) may be an oxidation reaction with oxidants such as DMP in suitable solvents such as DCM under suitable temperatures such as 0° C. to 25° C. to give intermediate 8.

Steps (vi) and (vii) involve reaction with a fluridizer such as DAST in suitable solvents such as DCM under suitable temperatures such as −78° C. to 0° C.

Steps (viii) (ix) and (x) are de-protection reactions. Typically, the intermediate is reacted with suitable acids such as HCl in suitable solvents such as 1,4-dioxane under suitable temperatures such as 25° C. to 40° C. to give intermediate 1.

Step (xi) involves reaction with dihydrofuran-3(2H)-one or substituted dihydrofuran-3(2H)-one under suitable reductant such as NaBH₃CN in a suitable solvent such as MeOH and CH₂Cl₂ at suitable temperature such as room temperature.

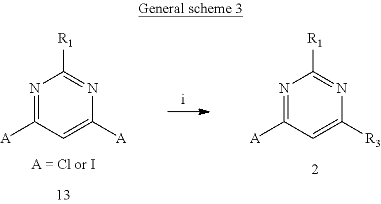

General Scheme 3 provides an exemplary synthesis for preparing intermediates 2.

When R₃ is an N-linked 4-6 membered heterocyclyl ring or NHR⁷; step (i) can be a reaction with different amines using appropriate bases such as TEA in appropriate solvents such as EtOH under suitable temperatures such as 25° C. to 100° C. to provide intermediate 2.

When R₃ is OR⁷, step (i) is a coupling reaction. The alcohol (R⁷OH) is deprotonated by a suitable base such as sodium hydride in suitable solvent such as THE at suitable temperature such as 0° C. to give the transitional intermediate. Then intermediate 13 is reacted with the transitional intermediate in suitable solvent such as THF at suitable temperature such as room temperature.

### EXAMPLES

General Experimental Procedures

The following descriptions and examples illustrate the invention. These examples are not intended to limit the scope of the present invention, but rather to provide guidance to the skilled chemist to prepare and use the compounds, compositions and methods of the present invention. While particular embodiments of the present invention are described, the skilled chemist will appreciate that various changes and modifications can be made without departing from the spirit and scope of the invention.

The chemical names of compounds described in the present application were generally created from ChemDraw Ultra (ChambridgeSoft) and/or generally follow the principle of IUPAC nomenclature.

Heating of reaction mixtures with microwave irradiations was carried out on a Smith Creator (purchased from Personal Chemistry, Forboro/MA, now owned by Biotage), an Emrys Optimizer (purchased from Personal Chemistry) or an Explorer (provided by CEM Discover, Matthews/NC) microwave.

Conventional techniques may be used herein for work up of reactions and purification of the products of the Examples.

References in the Examples below relating to the drying of organic layers or phases may refer to drying the solution over magnesium sulfate or sodium sulfate and filtering off the drying agent in accordance with conventional techniques. Products may generally be obtained by removing the solvent by evaporation under reduced pressure.

Purification of the compounds in the examples may be carried out by conventional methods such as chromatography and/or re-crystallization using suitable solvents. Chromatographic methods are known to the skilled person and include e.g. column chromatography, flash chromatography, HPLC (high performance liquid chromatography), and MDAP (mass directed auto-preparation, also referred to as mass directed LCMS purification). MDAP is described in e.g. W. Goetzinger et al, Int. J. Mass Spectrom., 2004, 238, 153-162.

Analtech Silica Gel GF and E. Merck Silica Gel 60 F-254 thin layer plates were used for thin layer chromatography. Both flash and gravity chromatography were carried out on E. Merck Kieselgel 60 (230-400 mesh) silica gel. Preparative HPLC were performed using a Gilson Preparative System using a Luna 5u C18(2) 100A reverse phase column eluting with a 10-80 gradient (0.1% FA in acetonitrile/0.1% aqueous FA) or a 10-80 gradient (acetonitrile/water). The CombiFlash system used for purification in this application was purchased from Isco, Inc. CombiFlash purification was carried out using a pre-packed SiO₂ column, a detector with UV wavelength at 254 nm and mixed solvents.

The terms “CombiFlash”, “Biotage”, “Biotage 75” and “Biotage SP4®” when used herein refer to commercially available automated purification systems using pre-packed silica gel cartridges.

Final compounds were characterized with LCMS (conditions listed below) or NMR. ¹H NMR or ¹⁹FNMR spectra were recorded using a Bruker Avance 400 MHz spectrometer. CDCl₃ is deuteriochloroform, DMSO-d₆ is hexadeuteriodimethylsulfoxide, and CD₃OD is tetradeuteriomethanol. Chemical shifts are reported in parts per million (ppm) downfield from the internal standard tetramethylsilane (TMS) or the NMR solvent. Abbreviations for NMR data are as follows: s=singlet, d=doublet, t=triplet, q=quartet, m=multiplet, dd=doublet of doublets, dt=doublet of triplets, app=apparent, br=broad. J indicates the NMR coupling constant measured in Hertz.

All temperatures are reported in degrees Celsius. All other abbreviations are as described in the ACS Style Guide (American Chemical Society, Washington, D C, 1986).

Absolute stereochemistry can be determined by methods known to one skilled in the art, for example X-ray or Vibrational Circular Dichroism (VCD).

When an enantiomer or a diasteroisomer is described and the absolute stereochemistry of a chiral center is not known, the use of “*” at the chiral centre denotes that the absolute stereochemistry of the chiral center is not known, i.e. the compound as drawn may be either a single R enantiomer or a single S enantiomer. Where the absolute stereochemistry at a chiral center of an enantiomer or a diasteroisomer is known, a bold wedge symbol ( [symbol] ) or a hashed wedge symbol ( [symbol] ) is used as appropriate, without the use of “*” at the chiral centre.

When a geometric or cis-trans isomer is described and the absolute configuration of the isomer is not known, the use of “*” at one of the atoms relevant to the geometric or cis-trans isomerism denotes that the absolute configuration at or around that atom is not known, i.e. the compound as drawn may be either a single cis isomer or a single trans enantiomer.

In the procedures that follow, after each starting material, reference to an intermediate is typically provided. This is provided merely for assistance to the skilled chemist. The starting material may not necessarily have been prepared from the batch referred to.

LCMS Conditions:

1) Acidic Method:

a. Instruments: HPLC: Waters UPC2 and MS: Qda

Mobile phase: water containing 0.1% FA/0.1% MeCN

Column: ACQUITY UPLC BEH C₁₈ 1.7 μm 2.1×50 mm and 1.7 μm 2.1×100 mm

Detection: MS and photodiode array detector (PDA)

b. Instruments: HPLC: Shimadzu and MS: 2020

Mobile phase: water containing 0.1% FA/0.1% MeCN

Column: Sunfire C₁₈ 5 μm 50×4.6 mm and Sunfire C₁₈ 5 μm 150×4.6 mm

Detection: MS and photodiode array detector (PDA)

2) Basic Conditions:

Instruments: HPLC: Agilent 1260 and MS: 6120

Mobile phase: 0.1% NH₄OH in H₂O/0.1% NH₄OH in ACN

Column: Xbridge C₁₈ 5 μm 50×4.6 mm and Xbridge C₁₈ 5 μm 150×4.6 mm

Detection: MS and photodiode array detector (DAD)

Prep-HPLC conditions

Instrument: Waters instrument

Column: Xbridge Prep C₁₈ column OBD (10 μm, 19×250 mm), Xbridge prep C₁₈ 10 μm OBD™ 19×150 mm, Sunfire Prep C₁₈ 10×25 0 mm 5 μm, XBRIDGE Prep C₁₈ 10×150 mm 5 μm, etc

Acidic Method:

Mobile phase: water containing 0.1% TFA/acetonitrile.

Basic Method:

Mobile phase: water containing 0.1% NH₄OH/acetonitrile.

Chiral prep-HPLC:

Thar SFC Prep 80 (TharSFC ABPR1, TharSFC SFC Prep 80 CO₂ Pump, TharSFC Co-Solvent Pump, TharSFC Cooling Heat Exchanger and Circulating Bath, TharSFC Mass Flow Meter, TharSFC Static Mixer, TharSFC Injection Module, Gilson UV Detector, TharSFC Fraction Collection Module

Chiral-HPLC analysis:

Instrument: Thar SFC Prep 80 (TharSFC ABPR1, TharSFC SFC Prep 80 CO₂Pump, TharSFC Co-Solvent Pump, TharSFC Cooling Heat Exchanger and Circulating Bath, TharSFC Mass Flow Meter, TharSFC Static Mixer, TharSFC Injection Module, Gilson UV Detector, TharSFC Fraction Collection Module

Column and mobile phase: are described in below examples.

Abbreviations and Resource Sources

The following abbreviations and resources are used herein below:

Ac—acetyl

MeCN—acetonitrile

Atm—atmosphere

Aq.—aqueous

BINAP-2,2′-bis(diphenylphosphino)-1,1′-binaphthyl

Boc—tert-butyloxycarbonyl

Boc₂O—di-tert-butyl dicarbonate

Bn—benzyl

t-Bu—tert-butyl

conc.—concentrated

DAST—N,N-diethylaminosulfur trifluoride

DCE—1,2-dichloroethane

DCM—dichloromethane

DEA—diethanolamine

DMEDA—N,N′-Dimethylethylenediamine

Dess-Martin—1,1,1-Tris(acetyloxy)-1,1-dihydro-1,2-benziodoxol-3-(1H)-one

DHP—3,4-dihydro-2H-pyran

DIBAL-H—diisobutylaluminum hydride

DIEA—N,N-diisopropylethylamine

DIPEA—N, N-diisopropylethylamine

DMA—N, N-dimethylacetamide

DMAP—4-dimethylaminopyridine

DMEDA—N,N′-dimethylethylenediamine

DMF—N, N-dimethylformamide

DMP—Dess-Martin periodinane

DMSO—dimethyl sulfoxide

DPPF—1,1′-bis(diphenylphosphino)ferrocene

EA—ethyl acetate

EDC—1-ethyl-3-(3-dimethylaminopropyl)carbodiimide hydrochloride

EDCl—3-(ethyliminomethyleneamino)-N,N-dimethylpropan-1-amine

EtOH/EtOH—ethanol

Et₂O—diethyl ether

EtOAc—ethyl acetate

Et₃N—triethylamine

FA—formic acid

HEP—heptane

Hex—hexane

HOAc-acetic acid

HATU—2-(1H-7-azabenzotriazol-1-yl)-1,1,3,3-tetramethyl uranium hexafluorophosphate

HOBT—hydroxybenzotriazole

IPA—isopropyl alcohol

ⁱPrOH/iPrOH—isopropyl alcohol

m-CPBA—meta-chloroperoxybenzoic acid

MOMCl—monochlorodimethyl ether

Me—methyl

MeOH—methanol

MsCl—methanesulfonyl chloride

NaHMDS—sodium bis(trimethylsilyl)amide

NIS—N-iodosuccinimide

NMP—1-methyl-2-pyrrolidone

NMO—4-methylmorpholine 4-oxide

PE—petroleum ether

PMB—p-methoxybenzyl

Pd₂(dba)₃—Tris(dibenzylideneacetone)dipalladium

Pd(dppf)Cl₂—1,1′-Bis(diphenylphosphino)ferrocenepalladium(II)dichloride

dichloromethane complex

Ph₃P—triphenylphosphine

PhNTf₂—N,N-bis-(Trifluoromethanesulfonyl)aniline

PPTS—pyridinium p-toluenesulfonate

PTSA—p-toluenesulfonic acid

rt/RT—room temperature

Rt—retention time

sat. —saturated

SEM-Cl—2-(trimethylsilyl)ethoxymethyl chloride

SFC—Supercritical Fluid Chromatography

TBAI—Tetrabutylammonium iodide

TBDPSCl—tert-Butyl(chloro)diphenylsilane

TEA—triethylamine

TFA—trifluoroacetic acid

TFAA—trifluoroacetic anhydride

THE—tetrahydrofuran

TLC—thin layer chromatography

TsCl—4-toluenesulfonyl chloride

TsOH—p-toluenesulfonic acid

Description 1

### (S)-Morpholin-2-ylmethanol hydrochloride (D1)

To a solution of (S)-tert-butyl 2-(hydroxymethyl)morpholine-4-carboxylate (500 mg, 2.30 mmol) in dioxane (4 mL) was added HCl/dioxane (4 M, 5 mL) and stirred at rt for 2 hrs. TLC showed that the reaction was completed. The reaction mixture was concent-rated to give the title compound (crude, 430 mg, yield >100%) as a white solid.

Description 2

### 4,6-Diiodo-2-methylpyrimidine (D2)

To a solution of NaI (11.9 g, 79.7 mmol) in HI (55%, 50 mL) was added 4,6-dichloro-2-methylpyrimidine (10.0 g, 61.3 mmol) in portions. The resulting suspension was heated to 40° C. and stirred for 1 hour. The reaction mixture was cooled and filtered. The solid was washed with water and then washed with methanol (50 mL). The mixture was filtered to give the title compound (9.0 g, yield 42%) as a white solid.

¹H NMR (400 MHz, CDCl₃): δ 8.07 (s, 1H), 2.67 (s, 3H).

LCMS: (mobile phase: 5-95% acetonitrile in 2.5 min), Rt=1.59 min, MS Calcd: 346; MS Found: 347 [M+H]+.

In a separate batch, to a solution of NaI (40 g, 26.8 mmol) in HI (55%, 200 mL) was added 4,6-dichloro-2-methylpyrimidine (33 g, 20.6 mmol). The resulting suspension was stirred at 40° C. for 24 hrs, then poured into ice water (500 mL) and filtered. The filtered cake was washed with ice water three times to give the crude product (67.3 g, yield: 96%) as a yellow solid.

¹H NMR (400 MHz, CDCl₃) δ 8.07 (s, 1H), 2.67 (s, 3H).

Description 3

### (S)-(4-(6-Iodo-2-methylpyrimidin-4-yl)morpholin-2-yl)methanol (D3)

To a solution of (S)-morpholin-2-ylmethanol hydrochloride (430 mg crude, 2.80 mmol) in CH₃OH (5 mL) was added 4,6-diiodo-2-methylpyrimidine (1.10 g, 3.10 mmol) and TEA (850 mg, 8.40 mmol). The resulting mixture was warmed to 60° C. for 2 hrs. TLC showed the reaction was completed. The reaction mixture was diluted with water (20 mL) and extracted EtOAc (20 mL×2). The combined organic layers were concentrated. The crude was purified by gel silico column (PE:EA=5:1) to give the title compound (760 mg, yield 81%) as a white solid.

¹H NMR (300 MHz, CDCl₃) δ 6.79 (s, 1H), 4.18-4.01 (m, 3H), 3.79-3.58 (m, 4H), 3.08-2.99 (m, 1H), 2.92-2.84 (m, 1H), 2.46 (s, 3H), 1.97-1.90 (m, 1H).

Description 4

### (2S)-4-(6-Iodo-2-methylpyrimidin-4-yl)-2-(((tetrahydro-2H-pyran-2-yl)oxy)methyl)-morpholine (D4)

To a solution of (S)-(4-(6-iodo-2-methylpyrimidin-4-yl)morpholin-2-yl)methanol (760 mg, 2.30 mmol) in DCM (20 mL) was added DHP (774 mg, 9.20 mmol) and TsOH (396 mg, 2.30 mmol). The resulting mixture was stirred at 50° C. overnight. TLC showed the reaction was completed. The mixture was washed with water (20 mL) and the aqueous part was extracted with DCM (20 mL×2). The combined organic layers were washed with brine, dried over Na₂SO₄, filtered and concentrated. The crude was purified by column (PE:EA=5:1) to give the title compound (750 mg, yield 78%) as a light yellow oil.

¹H NMR (300 MHz, CDCl₃) δ 6.79 (s, 1H), 4.63-4.61 (m, 1H), 4.15-4.00 (m, 3H), 3.901-3.77 (m, 2H), 3.73-3.51 (m, 4H), 3.11-2.78 (m, 2H), 2.46 (s, 3H), 1.88-1.48 (m, 6H).

Description 5

### 6-Bromo-5-methyl-1H-indazole (D5)

To a solution of 5-bromo-2,4-dimethylaniline (15.0 g, 75.0 mmol) in chloroform (150 mL) were added Ac₂O (15.0, 150 mmol), KOAc (8.00 g, 82.5 mmol), 18-crown-6 (10.0 g, 37.5 mmol) and isoamyl nitrite (26.3 g, 225 mmol) under ice bath. The reaction mixture was refluxed for 36 hrs, then concentrated to remove solvent. The residue was dissolved in EtOAc (500 mL), washed with water (100 mL), dried over Na₂SO₄, filtered and concentrated. The residue was dissolved in THF (100 mL) and NaOH (4 M, 40.0 mL, 160 mmol) was added. The mixture was stirred at rt for 1 h. The solvent was removed under vacuum and the residue was partitioned between EtOAc (400 mL) and water (200 mL). The organic layer was washed with brine, dried over Na₂SO₄, filtered and concentrated. The crude was purified by column chromatography (PE:EtOAc from 10:1 to 5:1) to give the title compound (5.1 g, yield 32%) as an orange solid.

¹H NMR (300 MHz, CDCl₃): δ 10.20 (br s, 1H), 7.99 (s, 1H), 7.75 (s, 1H), 7.61 (s, 1H), 2.50 (s, 3H).

Alternatively, to a solution of 5-bromo-2,4-dimethylaniline (242 g, 1.25 mol) in chloroform (5 L) was added Ac₂O (510 g, 5.0 mol). The mixture was stirred for 4 h and charged with KOAc (245 g, 2.5 mol) and 18-crown-6 (99 g, 0.375 mol). Isoamyl nitrite (293 g, 2.5 mol) was slowly added into the reaction mixture under N₂ protection. The reaction mixture was kept for reflux overnight, concentrated and redissolved in EtOAc, washed with water (3 L) and aq. NaCl (1 L). The solution was dried over Na₂SO₄ and concentrated. The crude was combined with other batch (250 g) and stirred in PE/EtOAc (1 L/200 mL). The precipitate was filtered, washed with PE/EA (5/1) to afford the solid (300 g). The mother liquid was stirred in PE/EA (5/1) solution and 125 g of product was obtained.

¹H NMR (400 MHz, CDCl₃) δ 8.70 (s, 1H), 8.02 (s, 1H), 7.57 (s, 1H), 2.77 (s, 3H), 2.52 (s, 3H).

To a solution of above intermediate (33.0 g, 130.4 mmol) in THE (330 mL) was added dropwise aq. NaOH (5.0 M, 130 mL) at 0-5° C. The resulting mixture was then stirred at rt for 2 hrs, then diluted with EtOAc (400 mL). The separated organic part was washed with brine (400 mL) and water (400 mL), dried over Na₂SO₄ and concentrated to afford the title product (27.5 g) as a yellow solid.

LC-MS [mobile phase: from 30% water (0.1% FA) and 70% MeCN (0.1% FA) to 5% water (0.1% FA) and 95% MeCN (0.1% FA) in 2.0 min] Rt=0.32 min; MS Calcd.: 211.06, MS Found: 213.2 [M+2H]⁺.

Description 6

### 6-Bromo-5-methyl-1-(tetrahydro-2H-pyran-2-yl)-1H-indazole (D6)

To a solution of 6-bromo-5-methyl-1H-indazole (5.10 g, 24.2 mmol) in dry DCM (120 mL) was added DHP (4.10 g, 48.4 mmol), TsOH (0.800 g, 4.80 mmol) and Mg₂SO₄ (5.0 g) at rt. The reaction mixture was heated to 35° C. and stirred for an hour. The reaction mixture was filtered and the filtrate was washed with a solution of Na₂CO₃ (10%, 100 mL), dried over Na₂SO₄, filtered and concentrated. The crude was purified by column chromatography (PE:EtOAc=50/1 to 20/1) to give the title compound (6.0 g, yield 84%) as an orange solid.

¹H NMR (300 MHz, CDCl₃): δ 7.90 (s, 1H), 7.84 (s, 1H), 7.55 (s, 1H), 5.63 (dd, J=9.6, 3.0 Hz, 1H), 4.05-4.00 (m, 1H), 3.78-3.70 (m, 1H), 2.58-2.44 (m, 4H), 2.20-2.02 (m, 2H), 1.78-1.65 (m, 3H).

LCMS: (mobile phase: 5-95% CH₃CN), Rt=2.19 min in 3 min; MS Calcd: 294; MS Found: 295 [M+1]⁺.

Alternatively, to a solution of 6-bromo-5-methyl-1H-indazole (27.0 g, 127.9 mmol) in DCM (405 mL) was added DHP (21.5 g, 255.8 mmol) and TsOH H₂O (4.86 g, 25.58 mmol) at rt. The reaction mixture was heated at 45° C. and stirred for 3 hrs. The reaction mixture was quenched with aq. NaHCO₃ to pH˜9. The aqueous phase was separated and extracted with DCM (200 mL×2). The combined organic layers were washed with brine (300 mL) and water (300 mL), dried over Na₂SO₄ and concentrated. The crude was purified by column chromatography (PE:EtOAc from 40:1 to 20:1) to give the title product (25.0 g, yield: 66.2%) as a yellow solid.

¹H NMR (400 MHz, CDCl₃) δ 7.84 (s, 1H), 7.77 (s, 1H), 7.48 (s, 1H), 5.57 (dd, J=9.2, 2.4 Hz, 1H), 3.96-3.94 (m, 1H), 3.70-3.65 (m, 1H), 2.46-2.39 (m, 4H), 2.09-1.97 (m, 2H), 1.71-1.47 (m, 3H).

Description 7

### tert-Butyl 4-(5-methyl-1-(tetrahydro-2H-pyran-2-yl)-1H-indazol-6-yl)-5,6-dihydropyrid-ine-1(2H)-carboxylate (D7)

To a suspension of 6-bromo-5-methyl-1-(tetrahydro-2H-pyran-2-yl)-1H-indazole (5.50 g, 18.6 mmol), tert-butyl 4-(4,4,5,5-tetramethyl-1,3,2-dioxaborolan-2-yl)-5,6-dihydropyridine-1(2H)-carboxylate (6.90 g, 22.3 mmol) and Na₂CO₃ (4.90 g, 46.5 mmol) in dioxane (150 mL) and water (130 mL) was added Pd(dppf)Cl₂ (658 mg, 0.900 mmol). The mixture was degassed with N₂ for 3 times and then stirred at 80° C. overnight. The solvent was removed under vacuum and the residue was partitioned between EtOAc (300 mL) and water (200 mL). The separated organic layer was washed with brine, dried over Na₂SO₄, filtered and concentrated. The crude was purified by column chromatography (PE:EtOAc=10:1) to give the title compound (7.3 g, yield 99%) as a slight brown solid.

¹H NMR (400 MHz, CDCl₃): δ 7.92 (s, 1H), 7.48 (s, 1H), 7.28 (s, 1H), 5.67 (dd, J=9.6, 2.8 Hz, 1H), 5.63 (br s, 1H), 4.07-4.01 (m, 3H), 3.78-3.70 (m, 1H), 3.67-3.64 (m, 2H), 2.62-2.53 (m, 1H), 2.45-2.39 (m, 2H), 2.34 (s, 3H), 2.18-2.12 (m, 1H), 2.07-2.02 (m, 1H), 1.81-1.73 (m, 2H), 1.69-1.61 (m, 1H), 1.52 (s, 9H).

Alternatively, to a suspension of 6-bromo-5-methyl-1-(tetrahydro-2H-pyran-2-yl)-1H-indazole (25.09 g, 84.7 mmol), tert-butyl 4-(4,4,5,5-tetramethyl-1,3,2-dioxaborolan-2-yl)-5,6-dihydropyridine-1(2H)-carboxylate (28.8 g, 93.2 mmol) and Na₂CO₃ (22.4 g, 211.7 mmol) in dioxane (375 mL) and water (60 mL) was added Pd(dppf)Cl₂ (3.89 g, 4.23 mmol) at 28° C. The resulting mixture was degassed with Ar₂ for 3 times and then stirred at 80° C. for 16 hrs. The reaction mixture cooled to rt, then diluted with EtOAc (250 mL) and water (300 mL). The aqueous phase was separated and extracted with EtOAc (250 mL). The combined organic phase was washed with brine (300 mL), dried over Na₂SO₄ and concentrated. The crude was purified by column chromatography (PE:EtOAc from 30:1 to 10:1) to give the title product (27.0 g, yield: 80.2%) as a light yellow gum.

LC-MS [mobile phase: from 30% water (0.1% FA) and 70% MeCN (0.1% FA) to 5% water (0.1% FA) and 95% MeCN (0.1% FA) in 2.0 min] Rt=0.69 min; MS Calcd.: 397.5, MS Found: 398.5 [M+H]⁺.

Description 8

### tert-Butyl 4-(5-methyl-1-(tetrahydro-2H-pyran-2-yl)-1H-indazol-6-yl)piperidine-1-carboxylate (D8)

To a solution of tert-butyl 4-(5-methyl-1-(tetrahydro-2H-pyran-2-yl)-1H-indazol-6-yl)-5,6-dihydropyridine-1(2H)-carboxylate (80 g, crude) in MeOH (2 L) under H₂ was added Pd/C (10 g, 12%/W). The reaction mixture was degassed for 3 times, stirred at r.t for 2 d, filtered and concentrated to give the crude product as a white solid. (65.8 g)

LC-MS [mobile phase: mobile phase: from 30% water (0.1% FA) and 70% CH₃CN (0.1% FA) to 5% water (0.1% FA) and 95% CH₃CN (0.1% FA) in 2.0 min], Rt=0.63 min; MS Calcd.:399.2, MS Found: 400.5 [M+H]⁺.

Alternatively, to a solution of tert-butyl 4-(5-methyl-1-(tetrahydro-2H-pyran-2-yl)-1H-indazol-6-yl)-5,6-dihydropyridine-1(2H)-carboxylate (27.0 g, 67.8 mmol) in MeOH (540 mL) was added Pd/C (4.05 g, 15%/W) under Ar₂ The reaction mixture was degassed with H₂ for 3 times. Then the mixture was stirred at r.t for 16 hrs. The reaction mixture was filtered through celite and concentrated to give the title product (25.3 g, yield: 93.5%) as a white solid.

LC-MS [mobile phase: from 30% water (0.1% FA) and 70% MeCN (0.1% FA) to 5% water (0.1% FA) and 95% MeCN (0.1% FA) in 2.0 min] Rt=0.65 min; MS Calcd.: 399.2, MS Found: 400.5 [M+H]⁺.

Description 9

### 5-Methyl-6-(piperidin-4-yl)-1H-indazole (D9)

To a solution of tert-butyl 4-(5-methyl-1-(tetrahydro-2H-pyran-2-yl)-1H-indazol-6-yl)piperidine-1-carboxylate (55.4 g, 139 mmol) in MeOH (150 mL) was added HCl/MeOH (5M, 200 mL). The reaction mixture was stirred at rt overnight, then concentrated, treated with Na₂CO₃aq. and basified with NaOH aq. to pH>12. The mixture was filtered to give the desired product as a white solid. (29.3 g, yield=98%)

LC-MS [mobile phase: mobile phase: from 90% water (0.1% FA) and 10% CH₃CN (0.1% FA) to 5% water (0.1% FA) and 95% CH₃CN (0.1% FA) in 2.0 min], Rt=0.85 min; MS Calcd.:215, MS Found: 216 [M+H]⁺.

Alternatively, to a solution of tert-butyl 4-(5-methyl-1-(tetrahydro-2H-pyran-2-yl)-1H-indazol-6-yl)piperi-dine-1-carboxylate (25.3 g, 63.3 mmol) in DCM (250 mL) was added dropwise HCl/MeOH (5 M, 200 mL) at 0° C. The reaction mixture was stirred at rt for 16 hrs. TLC (DCM/MeOH=10/1) showed the reaction was completed. The reaction mixture was concentrated to give a white solid (18.0 g). The hydrochloride salt (12 g) was dissolved into water (50 mL) and NaOH (3.2 g) was added slowly to the solution. The mixture was stirred at rt for 30 min and filtered to give the title product (8.0 g, yield: 58.7%) as a white solid.

¹H NMR (400 MHz, DMSO-d₆) δ 12.8 (br, 1H), 8.48 (s, 1H), 7.90 (s, 1H), 7.28 (s, 1H), 3.18 (d, J=12 Hz, 2H), 2.96 (t, J=21.6 Hz, 1H), 2.80 (t, J=12.4 Hz, 2H), 2.39 (s, 1H), 1.79-1.68 (m, 4H)

Description 10

### 5-Methyl-6-(1-(tetrahydrofuran-3-yl)piperidin-4-yl)-1H-indazole (D10)

To a stirred mixture of 5-methyl-6-(piperidin-4-yl)-1H-indazole_(900 mg, 4.18 mmol), dihydrofuran-3(2H)-one (900 mg, 10.5 mmol), 4 Å molecular sieves (747 mg) in MeOH/CH₂Cl₂ (9 mL/36 mL) at 0° C. were added AcOH (88.0 mg, 1.46 mmol) and NaBH₃CN (525 mg, 8.36 mmol). The reaction mixture was warmed to room temperature and stirred overnight, then filtered. The filtrate was washed with aqueous NaHCO₃ (10 mL), dried, filtered and concentrated. The purification by column chromatography (eluent: PE:EtOAc=1:1, followed by CH₂Cl₂:MeOH=20:1) afforded the desired product as a white solid (1.13 g, yield: 94%).

LC-MS [mobile phase: from 90% water (0.1% FA) and 10% MeCN (0.1% FA) to 5% water (0.1% FA) and 95% MeCN (0.1% FA) in 2.0 min]: Rt=0.29 min; MS Calcd: 285; MS Found: 286 [M+H]⁺.

¹H NMR (400 MHz, DMSO-d₆) δ 12.7 (br, 1H), 7.79 (s, 1H), 7.40 (s, 1H), 7.19 (s, 1H), 3.74-3.50 (m, 6H), 3.02-2.71 (m, 3H), 2.40 (s, 3H), 1.65-1.57 (m, 6H)

Description 11

### (R)-Morpholin-2-ylmethanol hydrochloride (D11)

To a solution of (R)-tert-butyl 2-(hydroxymethyl)morpholine-4-carboxylate (500 mg, 2.30 mmol) was added HCl/dioxane (4 M, 10 mL) and stirred for 1 h at rt. TLC showed that the reaction was completed. The reaction was concentrated to give the title compound (420 mg, yield >100%) as a white solid.

¹H NMR (300 MHz, DMSO-d₆) δ 9.67 (s, 1H), 9.38 (s, 1H), 3.94-3.88 (m, 1H), 3.77-3.67 (m, 2H), 3.45-3.33 (m, 2H), 3.13 (t, J=12.6 Hz, 2H), 2.95-2.87 (m, 1H), 2.78-2.67 (m, 1H).

Description 12

### (R)-(4-(6-Iodo-2-methylpyrimidin-4-yl)morpholin-2-yl)methanol (D12)

To a solution of (R)-morpholin-2-ylmethanol hydrochloride (423 mg crude, 2.30 mmol) in CH₃OH (10 mL) was added 4,6-diiodo-2-methylpyrimidine (954 mg, 2.75 mmol) and TEA (835 mg, 8.25 mmol). The resulting mixture was warmed to 70° C. and stirred for 2 hrs. LCMS showed that the reaction was completed. The reaction mixture was concentrated to remove solvent, poured into water (40 mL) and extracted with EtOAc (40 mL×2). The combined organic layers were washed with brine, dried over Na₂SO₄ and concentrated. The residue was purified by column (PE:EA=2:1) to give the title compound (639 mg, yield 83%) as a white solid.

¹H NMR (300 MHz, CDCl₃) δ 6.79 (s, 1H), 4.22-4.01 (m, 3H), 3.79-3.56 (m, 4H), 3.08-2.98 (m, 1H), 2.88-2.84 (m, 1H), 2.46 (s, 3H), 2.09-2.04 (m, 1H).

Alternatively, to a solution of (R)-morpholin-2-ylmethanol hydrochloride (355 mg crude, 2.31 mmol) and 4,6-diiodo-2-methylpyrimidine (800 mg, 2.31 mmol) in EtOH/THF (10 mL/10 mL) was added DIEA (1.49 g, 11.6 mmol). The resulting mixture was stirred at rt for 2 days, then concentrated and purified by column (PE:EtOAc=2:1) to give the title product as a white solid (387 mg, yield: 50%)

LC-MS [mobile phase: from 50% water (0.1% FA) and 50% MeCN (0.1% FA) to 5% water (0.1% FA) and 95% MeCN (0.1% FA) in 2.0 min]: Rt=0.28 min; MS Calcd:335.01; MS Found: 336.2 [M+H]⁺.

Descriptions 13 and 14

### trans-tert-Butyl 3-hydroxy-4-(5-methyl-1-(tetrahydro-2H-pyran-2-yl)-1H-indazol-6-yl)piperidine-1-carboxylate (D13) and tert-Butyl 4-(5-methyl-1-(tetrahydro-2H-pyran-2-yl)-1H-indazol-6-yl)piperidine-1-carboxylate (D14)

To a solution of tert-butyl 4-(5-methyl-1-(tetrahydro-2H-pyran-2-yl)-1H-indazol-6-yl)-5,6-dihydropyridine-1(2H)-carboxylate (21.0 g, 52.8 mmol) in dry THF (200 mL) was added BH₃-THF solution (1 M, 211 mL, 211 mmol) under N₂ and below 5° C. with internal temperature. The mixture was warmed to rt and stirred overnight. TLC showed the starting material was consumed up. A solution of NaOH (2 M, 79 mL, 158 mmol) was carefully added dropwise below 10° C. (internal temperature) and then H₂O₂ (30%, 20.0 mL, 151 mmol) was added dropwise under same temperature. The mixture was stirred at rt. for an hour, then quenched with 150 mL of 10% Na₂S2O₃ solution under ice bath and stirred for 20 min. The solvent was removed and the residue was extracted with EtOAc (200 mL×2). The combined organic layers were washed with brine, dried over Na₂SO₄ and concentrated. The residue was purified by column chromatography (PE:EtOAc from 10:1 to 2:1) to give the title compound (16.5 g, yield 75%) as a white solid.

¹H NMR (300 MHz, CDCl₃) δ 7.92 (s, 1H), 7.53 (s, 1H), 7.42 (s, 1H), 5.70-5.67 (m, 1H), 4.49-4.44 (m, 1H), 4.30-4.17 (m, 1H), 4.05-3.91 (m, 2H), 3.82-3.72 (m, 1H), 3.04-2.96 (m, 1H), 2.86-2.72 (m, 2H), 2.63-2.53 (m, 1H), 2.47 (s, 3H), 2.21-2.16 (m, 1H), 2.07-2.02 (m, 1H), 1.99-1.67 (m, 6H), 1.52 (s, 9H).

Description 15

### (cis)-tert-Butyl 3-fluoro-4-(5-methyl-1-(tetrahydro-2H-pyran-2-yl)-1H-indazol-6-yl) piperidine-1-carboxylate (D15)

To a solution of (trans)-tert-Butyl 3-hydroxy-4-(5-methyl-1-(tetrahydro-2H-pyran-2-yl)-1H-indazol-6-yl)piperidine-1-carboxylate (24.5 g, 59.0 mmol) in dry DCM (200 mL) was added DAST (38.0 g, 236 mmol) under N₂ at −65° C. The mixture was gradually warmed to rt and stirred for 2 hrs. The reaction mixture was care Fully poured into Na₂CO₃ aqueous solution (10%, 300 mL) and stirred for 20 min. The organic layer was separated and the aqueous was extracted with DCM (250 mL×2). The combined organic layers were washed with brine, dried over Na₂SO₄ and evaporated. The crude was purified by column chromatography (PE:EtOAc=10:1) to give the title compound (11.8 g, yield 48%) as a white solid.

¹H NMR (400 MHz, CDCl₃) δ 7.92 (s, 1H), 7.52 (s, 1H), 7.41 (s, 1H), 5.74-5.67 (m, 1H), 4.80-4.59 (m, 2H), 4.21 (br s, 1H), 4.07-3.99 (m, 1H), 3.80-3.71 (m, 1H), 3.25-3.19 (m, 1H), 2.89-2.79 (m, 2H), 2.65-2.51 (m, 1H), 2.45 (s, 3H), 2.19-2.15 (m, 1H), 2.15-2.04 (m, 1H), 1.93-1.88 (m, 1H), 1.80-1.74 (m, 5H), 1.52 (s, 9H).

LCMS [5-95% MeCN]: Rt=2.25 min in 3 min; MS Calcd: 417; MS Found: 418 [M+H]⁺.

Description 16

### (cis)-6-(3-Fluoropiperidin-4-yl)-5-methyl-1H-indazole hydrochloride (D16)

A mixture of (cis)-tert-butyl 3-fluoro-4-(5-methyl-1-(tetrahydro-2H-pyran-2-yl)-1H-indazol-6-yl) piperidine-1-carboxylate (2.50 g, 6.00 mmol) in HCl/dioxane (6 M, 40 mL) was stirred at rt for 6 hrs. The reaction mixture was cooled to 0° C. and filtered. The solid was washed with cold 1,4-dioxane (5 mL) to get the title compound (1.4 g, yield 100%) as a white solid which was used for next step directly.

LC-MS [5-95% MeCN]: Rt=1.73 min; MS Calcd.:233, MS Found: 234 [M+H]⁺.

Description 17

### (cis)-tert-Butyl 3-fluoro-4-(5-methyl-1H-indazol-6-yl)piperidine-1-carboxylate (D17)

To a solution of (cis)-6-(3-fluoropiperidin-4-yl)-5-methyl-1H-indazole hydrochloride (500 mg, 2.14 mmol) in CH₃OH (5 mL) and H₂O (1 mL) was added KOH (242 mg, 4.29 mmol) and (Boc)₂O (700 mg, 3.21 mmol) under ice bath. The reaction mixture was stirred at rt for 2 hrs. The reaction mixture was diluted with water (30 mL) and extracted with EtOAc (3×20 mL). The combined organic layers were concentrated. The residue was purified by column chromatograph (PE:EtOAc=20:1) to give the title compound (180 mg, yield: 25%) as a colorless oil.

¹H NMR (300 MHz, CDCl₃) δ 9.98 (s, 1H), 7.96 (s, 1H), 7.56 (s, 1H), 7.39 (s, 1H), 4.76-4.54 (m, 2H), 4.27-4.10 (m, 1H), 3.25-3.14 (m, 1H), 2.91-2.76 (m, 2H), 2.48 (s, 3H), 1.97-1.84 (m, 1H), 1.71-1.62 (m, 1H), 1.51 (s, 9H).

Descriptions 18 and 19

### (cis)-tert-Butyl 3-fluoro-4-(5-methyl-1H-indazol-6-yl)piperidine-1-carboxylate (single cis isomer 1) (D18) and (cis)-tert-Butyl 3-fluoro-4-(5-methyl-1H-indazol-6-yl)piperidine-1-carboxylate (Single Cis Isomer 2) (019)

(cis)-tert-Butyl 3-fluoro-4-(5-methyl-1H-indazol-6-yl)piperidine-1-carboxylate (140 mg, 0.420 mmol) was separated by chiral prep. HPLC with the method (Chiralpak IB 5 um, 20×250 nm, Supercritical CO₂:i-PrOH=80:20, Flow rate: 20 mL/min, 205 nm, Temperature: 30° C.) to give (cis)-tert-butyl 3-fluoro-4-(5-methyl-1H-indazol-6-yl)piperidine-1-carboxylate (single cis isomer 1) (D18) (68 mg, yield 48%) as a white solid and (cis)-tert-Butyl 3-fluoro-4-(5-methyl-1H-indazol-6-yl)piperidine-1-carboxylate (single cis isomer 2) (D19) (47 mg, yield 33%) as a white solid.

Single Cis Isomer 1 (D18)

LCMS [mobile phase: 5-95% MeCN in 2.5 min]: Rt=1.64 min; MS Calcd: 333, MS Found: 332 [M−H]⁻.

¹H NMR (300 MHz, CDCl₃) δ 10.07 (s, 1H), 7.97 (s, 1H), 7.56 (s, 1H), 7.39 (s, 1H), 4.78-4.53 (m, 2H), 4.32-4.12 (m, 1H), 3.26-3.13 (m, 1H), 2.93-2.75 (m, 2H), 2.47 (s, 3H), 1.94-1.79 (m, 1H), 1.69-1.60 (m, 1H), 1.49 (s, 9H).

Chiral HPLC [Chiralpak IB 5 μm 4.6×250 mm, Phase:Hex/IPA=80/20, flowrate: 1 mL/min, temperature: 30° C.]: Rt: 6.142 min, 100% ee.

Single Cis Isomer 2 (19)

LCMS [mobile phase: 5-95% MeCN in 2.5 min]: Rt=1.64 min; MS Calcd: 333 MS Found: 332 [M−H]⁻.

¹H NMR (300 MHz, CDCl₃) δ 10.45 (s, 1H), 7.97 (s, 1H), 7.56 (s, 1H), 7.39 (s, 1H), 4.75-4.55 (m, 2H), 4.26-4.16 (m, 1H), 3.24-3.17 (m, 1H), 2.90-2.74 (m, 2H), 2.46 (s, 3H), 1.93-1.87 (m, 1H), 1.70-1.61 (m, 1H), 1.50 (s, 9H).

Chiral HPLC [Chiralpak IB 5 μm 4.6×250 mm, Phase: Hex/IPA=80/20, flow rate: 1 mL/min, temperature: 30° C.]: Rt: 7.671 min, 100% ee

Description 20

### 6-((3S,4R)-3-Fluoropiperidin-4-yl)-5-methyl-1H-indazole (D20)

To a solution of (cis)-tert-butyl 3-fluoro-4-(5-methyl-1H-indazol-6-yl)piperidine-1-carboxylate (D18, 100 mg, 0.30 mmol) in MeOH (1.5 mL) was added HCl/MeOH (5 M, 1 mL) at 0° C.

The reaction mixture was warmed to room temperature, stirred overnight, concentrated to remove solvent, neutralized with Na₂CO₃ solution (5 mL) and extracted with EtOAc for 3 times. The combined organic phase was dried, filtered and concentrated to give the crude product as a white solid.

LC-MS [mobile phase: from 90% water (0.1% FA) and 10% MeCN (0.1% FA) to 5% water (0.1% FA) and 95% MeCN (0.1% FA) in 2.0 min]: Rt=0.49 min; MS Calcd.:233, MS Found: 234 [M+H]⁺.

Description 21

### 6-((3S,4R)-3-Fluoropiperidin-4-yl)-5-methyl-1H-indazole (D21)

The title compound was prepared by a procedure similar to that described for D20 starting from a suspension of (cis)-tert-butyl 3-fluoro-4-(5-methyl-1H-indazol-6-yl)piperidine-1-carboxylate (D19).

LC-MS [mobile phase: from 90% water (0.1% FA) and 10% MeCN (0.1% FA) to 5% water (0.1% FA) and 95% MeCN (0.1% FA) in 2.0 min]: Rt=0.29 min; MS Calcd.:279.1, MS Found: 280.2 [M+H]⁺.

Description 22

### 1-(6-Iodo-2-methylpyrimidin-4-yl)azetidin-3-ol (D22)

A suspension of 4,6-diiodo-2-methylpyrimidine (2.00 g, 5.80 mmol), azetidin-3-ol hydrochloride (700 mg, 6.38 mmol) and TEA (1.76 g, 17.4 mmol) in i-PrOH (12 mL) was heated to 75° C. and stirred for 1 h. The reaction mixture was concentrated and the residue was triturated with water (50 mL), filtered and dried to give the title compound (1.2 g, yield 71%) as a white solid.

¹H NMR (400 MHz, DMSO-d₆) δ 6.69 (s, 1H), 5.79 (d, J=6.4 Hz, 1H), 4.59-4.52 (m, 1H), 4.22-4.18 (m, 2H), 3.72 (dd, J=9.6, 4.4 Hz, 2H), 2.29 (s, 3H).

LCMS [mobile phase: 5-95% MeCN in 2.5 min]:Rt=1.18 min, MS Calcd: 291; MS Found: 292 [M+H]⁺.

Description 23

### 4,6-Diiodo-2-methoxypyrimidine (D23)

To a solution of NaI (1.10 g, 7.34 mmol) in HI (55%, 7.5 mL) was added 4,6-dichloro-2-methoxypyrimidine (1.00 g, 5.59 mmol). The reaction mixture was heated to 40° C. and stirred for 10 h, then poured into ice water (50 mL) and filtered to give the crude solid. The residue was purified by column chromatography (PE:EtOAc=10:1) to give the title product (640 mg, yield 31.7%) as a white solid.

¹H NMR (400 MHz, CDCl₃) δ 7.85 (s, 1H), 4.00 (s, 3H).

Description 24

### 1-(6-iodo-2-methoxypyrimidin-4-yl)azetidin-3-ol (D24)

The title compound was prepared by a procedure similar to that described for D22 starting from a suspension of 4,6-diiodo-2-methoxypyrimidine, azetidin-3-ol hydrochloride and TEA in i-PrOH at 85° C.

LCMS (5-95% MeCN in 2.5 min) Rt=1.27 min, [M+H]⁺=216.

¹H NMR (400 MHz, CDCl₃) δ 5.86 (s, 1H), 4.84-4.79 (m, 1H), 4.34-4.30 (m, 2H), 3.98-3.95 (m, 2H), 3.92 (s, 3H), 3.13 (br s, 1H).

Description 25

### (R)-(4-(6-Iodo-2-methoxypyrimidin-4-yl)morpholin-2-yl)methanol (D25)

The title compound was prepared by a procedure similar to that described for D3 starting from a solution of 4,6-diiodo-2-methoxypyrimidine and (R)-morpholin-2-ylmethanol hydrochloride in ⁱPrOH and DIPEA at rt.

LC-MS [mobile phase: from 50% water (0.1% FA) and 50% MeCN (0.1% FA) to 5% water (0.1% FA) and 95% MeCN (0.1% FA) in 2.6 min]: Rt=0.92 min; MS Calcd: 351.1, MS Found: 352.0 [M+H]⁺.

Description 26

### tert-Butyl 4-(5-methyl-1H-indazol-6-yl)piperidine-1-carboxylate (D26)

To a stirred solution of 5-methyl-6-(piperidin-4-yl)-1H-indazole (1.00 g, 4.64 mmol) and Et₃N (930 mg, 9.20 mmol) in CH₂Cl₂ (80 mL) was added Boc₂O (1.00 g, 4.60 mmol). The reaction mixture was stirred at room temperature for 3 h. LC-MS showed the reaction was completed. The reaction mixture was concentrated to dryness. The residue was purified by silica gel chromatography eluted with PE:EtOAc=3:1 to afford the desired product as a white solid (900 mg, yield: 61%).

¹H NMR (400 MHz, DMSO-d₆) δ 12.77 (s, 1H), 7.89 (s, 1H), 7.50 (s, 1H), 7.28 (s, 1H), 4.12-4.07 (m, 2H), 3.17 (s, 1H), 2.94-2.84 (m, 2H), 2.40 (s, 3H), 1.77 (d, J=12.0 Hz, 2H), 1.55-1.47 (m, 2H), 1.43 (s, 9H).

Description 27

### 6-Bromo-5-nitro-1H-indazole (D27)

To a solution of 1-(6-bromo-5-nitro-1H-indazol-1-yl)ethanone (2.2 g, 7.8 mmol) in THF (10 mL) was added aqueous NaOH (5 M, 6 mL). The resulting mixture was stirred at rt for 1 h. DCM (100 mL) was added to extract the desired compound. The organic solution was washed with water (30 mL) and brine, dried over Na₂SO₄ and concentrated to give the title compound (1.0 g, yield: 53%) as a brown solid which was used for next step directly.

¹H NMR (300 MHz, DMSO-d₆) δ 13.74 (s, 1H), 8.63 (s, 1H), 8.35 (s, 1H), 8.07 (s, 1H).

Description 28

### 6-Bromo-5-nitro-1-(tetrahydro-2H-pyran-2-yl)-1H-indazole (D28)

To a suspension of 6-bromo-5-nitro-1H-indazole (1.03 g, 4.26 mmol) and DHP (717 mg, 8.54 mmol) in DCM (10 mL) was added TsOH H₂O (146 mg, 0.77 mmol) at rt. The resulting mixture was stirred at rt (25° C.) for 20 min. The reaction mixture was diluted with DCM (50 mL) and then washed with sat.Na₂CO₃ (30 mL) and brine, dried over MgSO₄ and concentrated. The crude product was purified by column chromatography (PE:EtOAc=5:1) to give the title compound (1.08 g, yield: 78%) as an orange solid.

¹H NMR (300 MHz, CDCl₃) δ 8.35 (s, 1H), 8.14 (s, 1H), 8.00 (s, 1H), 5.75-5.71 (m, 1H), 4.04-3.99 (m 1H), 3.82-3.74 (m, 1H), 2.54-2.41 (m, 1H), 2.21-2.08 (m, 2H), 1.85-1.66 (m, 3H).

Description 29

### tert-Butyl 4-(5-nitro-1-(tetrahydro-2H-pyran-2-yl)-1H-indazol-6-yl)-5,6-dihydropyridine-1(2H)-carboxylate (D29)

To a suspension of 6-bromo-5-nitro-1-(tetrahydro-2H-pyran-2-yl)-1H-indazole (1.08 g, 3.31 mmol), tert-butyl 4-(4,4,5,5-tetramethyl-1,3,2-dioxaborolan-2-yl)-5,6-dihydropyridine-1(2H)-carboxylate (1.08 g, 3.48 mmol) and Na₂CO₃ (878 mg, 8.28 mmol) in 1,4-dioxane (12 mL) and water (2.5 mL) was added Pd(dppf)Cl₂ (121 mg, 0.166 mmol) at room temperature. The resulting mixture was stirred at 100° C. under N₂ atmosphere overnight. The reaction mixture was cooled and filtered. The filtrate was concentrated and the crude product was purified by column chromatography (PE:EtOAc=5:1) to give the title compound (1.2 g, yield: 85%) as an orange solid.

¹H NMR (300 MHz, CDCl₃) δ 8.48 (s, 1H), 8.17 (s, 1H), 7.43 (s, 1H), 5.76-5.61 (m, 2H), 4.13-4.01 (m 3H), 3.83-3.74 (m, 1H), 3.72-3.65 (m, 2H), 2.58-2.45 (m, 1H), 2.41-2.28 (m, 2H), 2.22-2.06 (m, 2H), 1.85-1.65 (m, 3H), 1.51 (s, 9H).

Description 30

### tert-Butyl 4-(5-amino-1-(tetrahydro-2H-pyran-2-yl)-1H-indazol-6-yl)piperidine-1-carboxylate (D30)

To a solution of tert-butyl 4-(5-nitro-1-(tetrahydro-2H-pyran-2-yl)-1H-indazo-6-yl)-5,6-dihydropyridine-1(2H)-carboxylate (1.0 g, 2.3 mmol) in MeOH (15 mL) was added Pd/C (10%, 100 mg) at room temperature. The resulting mixture was stirred at 50° C. under H₂ atmosphere (1 atm) for 3 hrs. The reaction mixture was cooled and filtered. The filtrate was concentrated to give the title compound (876 mg, yield: 94%) as a white solid.

¹H NMR (300 MHz, CDCl₃) δ 7.82 (s, 1H), 7.28 (s, 1H), 6.98 (s, 1H), 5.66-5.62 (m, 1H), 4.41-4.24 (m, 2H), 4.07-4.01 (m, 1H), 3.79-3.71 (m, 1H), 3.57 (s, 2H), 2.92-2.75 (m, 3H), 2.64-2.48 (m, 1H), 2.20-2.10 (m, 1H), 2.07-1.93 (m, 3H), 1.83-1.63 (m, 5H), 1.50 (s, 9H).

Description 31

### 5-Chloro-6-(piperidin-4-yl)-1H-indazole (D31)

A solution of NaNO₂ (165 mg, 2.39 mmol) in water (5 mL) was added dropwise to a solution of tert-butyl 4-(5-amino-1-(tetrahydro-2H-pyran-2-yl)-1H-indazol-6-yl)piperidine-1-carboxylate (870 mg, 2.17 mmol) in conc. HCl (3 mL) under ice bath (0° C.-5° C.). Then the resulting mixture was stirred for additional 15 min under ice bath. Then the mixture was added to a suspension of CuCl (387 mg, 3.91 mmol) in water (5 mL) at 60° C. in one portion. The resulting mixture was stirred for 30 min at 60° C., cooled and gradually treated with sat. Na₂CO₃ (50 mL) and stirred for 15 min. Aq. ammonia (30%, 5 mL) was added to the mixture and stirred for 5 min. Then the mixture was extracted with EtOAc (30 mL×3) and the combined organic layers were washed with brine, dried over MgSO₄ and concentrated to give the title compound (400 mg, yield: 78%) as a pale yellow solid.

¹H NMR (300 MHz, DMSO-d₆) δ 13.15 (br s, 1H), 8.01 (s, 1H), 7.86 (s, 1H), 7.43 (s, 1H), 3.10-3.06 (m, 3H), 2.69-2.62 (m, 2H), 1.81-1.77 (m, 2H), 1.62-1.47 (m, 2H).

Description 32

### 5-Chloro-6-(1-(tetrahydrofuran-3-yl)piperidin-4-yl)-1H-indazole (D32)

To a solution of 5-chloro-6-(piperidin-4-yl)-1H-indazole (410 mg, 1.74 mmol) in MeOH/CH₂Cl₂ (2 mL/10 mL) were added dihydrofuran-3(2H)-one (300 mg, 3.48 mmol), AcOH (35 mg, 0.52 mmol), 4 A molecular sieve (0.500 g) and NaBH₃CN (220 mg, 3.48 mmol). The reaction mixture was stirred at room temperature for 2 days. LC-MS showed the reaction was completed. The reaction mixture was quenched with water (50 mL) and extracted with CH₂Cl₂ (3×50 mL). The combined organic layers were washed with brine (2×100 mL), dried over anhydrous Na₂SO₄, filtered and concentrated to afford the desired crude product as a yellow solid (540 mg).

LC-MS [mobile phase: from 50% water (0.1% FA) and 50% MeCN (0.1% FA) to 5% water (0.1% FA) and 95% MeCN (0.1% FA) in 2.6 min]: Rt=0.76 min; MS Calcd: 305.80, MS Found: 306.1 [M+H]⁺.

Description 33

### cis-6-(3-Fluoro-1-(tetrahydrofuran-3-yl)piperidin-4-yl)-5-methyl-1H-indazole (D33)

To a solution of 6-((3S,4R)-3-fluoropiperidin-4-yl)-5-methyl-1H-indazole (D20) (200 mg, 0.86 mmol) in MeOH/CH₂Cl₂ (40 mL/8 mL) were added dihydrofuran-3(2H)-one (120 mg, 1.37 mmol), AcOH (12 mg, 0.2 mmol), 4A molecular sieve (0.5 g) and NaBH₃CN (90 mg, 1.37 mmol). The reaction mixture was stirred at room temperature overnight, then poured into water (50 mL) and extracted with CH₂Cl₂ (2×50 mL). The combined organic layers were washed with water (30 mL), brine (30 mL), dried over anhydrous Na₂SO₄ and filtered. The filtrate was concentrated to give the desired product as a white solid (180 mg, yield: 69%).

LC-MS [mobile phase: from 50% water (0.1% FA) and 50% MeCN (0.1% FA) to 5% water (0.1% FA) and 95% MeCN (0.1% FA) in 2.6 min]: Rt=0.72 min; MS Calcd: 303.37, MS Found: 304.2 [M+H]⁺.

Description 34

### cis-6-(3-Fluoro-1-(tetrahydrofuran-3-yl)piperidin-4-yl)-5-methyl-1H-indazole (D34)

The title compound was prepared by a procedure similar to that described for D33 starting from 6-((3S,4R)-3-fluoropiperidin-4-yl)-5-methyl-1H-indazole (D21) in MeOH/CH₂Cl₂, dihydrofuran-3(2H)-one, AcOH, 4A molecular sieve and NaBH₃CN.

Description 35:

### 1-(6-Iodo-2-methoxypyrimidin-4-yl)-3-methylazetidin-3-ol (D35)

To a solution of 4,6-diiodo-2-methoxypyrimidine (1.50 g, 4.14 mmol) in i-PrOH (12 mL) was added 3-methylazetidin-3-ol (616 mg, 4.97 mmol) and TEA (1.25 g, 12.4 mmol). The reaction mixture was stirred at room temperature for 5 hour, diluted with H₂O (30 mL) and extracted with EtOAc (30 mL×2). The combined organic layers were dried over Na₂SO₄, filtered and concentrated. The residue was purified by silica gel chromatography column (petroleum ether/EtOAc=1/1) to give the title compound (1.2 g, 92%) as a white solid.

¹HNMR (400 MHz, CDCl₃) δ 6.33 (s, 1H), 4.01-3.99 (m, 4H), 3.89 (s, 3H), 2.48 (s, 1H), 1.64-1.59 (m, 3H).

Description 36:

### tert-Butyl 4-(1-(6-(3-hydroxy-3-methylazetidin-1-yl)-2-methoxypyrimidin-4-yl)-5-methyl-1H-indazol-6-yl)piperidine-1-carboxylate (D36)

A mixture of tert-butyl 4-(5-methyl-1H-indazol-6-yl)piperidine-1-carboxylate (250 mg, 0.790 mmol), 1-(6-iodo-2-methoxypyrimidin-4-yl)-3-methylazetidin-3-ol (306 mg, 0.950 mmol), N,N′-dimethylcyclohexane-1,2-diamine (224 mg, 1.58 mmol), CuI (150 mg, 0.790 mmol) and K₃PO₄ (335 mg, 1.58 mmol) in toluene (3 mL) was stirred at 100° C. for 2 hrs, then diluted with EtOAc (30 mL), washed with brine (30 mL), dried over Na₂SO₄, filtered and concentrated. The residue was purified by silica gel chromatography column (petroleum ether/EtOAc=2:1) to give the title compound (310 mg, 77%) as a yellow oil.

LCMS [column: C₁₈; column size: 4.6×30 mm 5 μm; Dikwa Diamonsil plus; mobile phase: B (MeCN) A1 (0.02% NH₄Ac+5% MeCN); gradient (B %) in 4 mins. 10-95-POS; flow rate: 1.5 mL/min]: Rt=2.647 min; MS Calcd.: 508, MS Found: 509 [M+H]⁺.

Description 37:

### 1-(2-Methoxy-6-(5-methyl-6-(piperidin-4-yl)-1H-indazol-1-yl)pyrimidin-4-yl)-3-methylazetidin-3-ol (D37)

To a solution of tert-butyl 4-(1-(6-(3-hydroxy-3-methylazetidin-1-yl)-2-methoxypyrimidin-4-yl)-5-methyl-1H-indazol-6-yl)piperidine-1-carboxylate (310 mg, 0.610 mmol) in MeOH (6 mL) was added HCl/dioxane (6 M, 3 mL). The mixture was stirred at room temperature for 2 hrs, then diluted with sat. NaHCO₃ (30 mL), extracted with DCM (30 mL×2), dried over Na₂SO₄, filtered and concentrated to give the title product (227 mg, 91%) as a yellow oil.

LCMS [column: C₁₈; column size: 4.6×30 mm 5 μm; Dikwa Diamonsil plus; mobile phase: B (MeCN) A₁ (0.02% NH₄Ac+5% MeCN); gradient (B %) in 4 mins. 10-95-POS; flow rate: 1.5 mL/min.]: Rt=1.811 min; MS Calcd.: 408, MS Found: 409 [M+H]⁺.

Description 38:

### (S)-1-(6-Iodo-2-methoxypyrimidin-4-yl)pyrrolidin-3-ol (D38)

A solution of 4,6-diiodo-2-methylpyrimidine (550 mg, 1.52 mmol), (S)-pyrrolidin-3-ol hydrochloride (145 mg, 1.67 mmol) and TEA (460 mg, 4.56 mmol) in i-PrOH (12 mL) was stirred at room temperature for 18 hrs, then concentrated. The residue was purified by silica gel chromatography column (petroleum ether/EtOAc=1/1) to give the title compound (358 mg, 73%) as a white solid.

¹H NMR (400 MHz, CDCl₃) δ 6.45 (s, 1H), 4.60 (s, 1H), 3.89 (s, 3H), 3.72-3.50 (m, 5H), 2.10-2.04 (m, 2H).

Description 39:

### (S)-tert-Butyl 4-(1-(6-(3-hydroxypyrrolidin-1-yl)-2-methoxypyrimidin-4-yl)-5-methyl-1H-indazol-6-yl)piperidine-1-carboxylate (D39)

The title compounds was prepared by a procedure similar to that described for D36 starting from a mixture of tert-butyl-4-(5-methyl-1H-indazol-6-yl)piperidine-1-carboxylate, (S)-1-(6-iodo-2-methoxypyrimidin-4-yl)pyrrolidin-3-ol, CuI, K₃PO₄ and N,N′-dimethylcyclohexane-1,2-diamine in toluene at 100° C.

¹H NMR (400 MHz, CDCl₃) δ 8.74 (s, 1H), 8.07 (s, 1H), 7.51 (s, 1H), 6.62 (s, 1H), 4.64 (s, 1H), 4.32-4.21 (br s, 2H), 4.15 (s, 3H), 3.72 (b rs, 4H), 3.01-2.95 (m, 1H), 2.87 (br s, 2H), 2.47 (s, 3H), 2.16-2.12 (m, 2H), 1.89-1.86 (m, 2H), 1.72-1.62 (m, 2H), 1.60 (s, 9H).

Description 40:

### (S)-1-(2-Methoxy-6-(5-methyl-6-(piperidin-4-yl)-1H-indazol-1-yl)pyrimidin-4-yl)pyrrolidin-3-ol (D40)

A solution of (S)-tert-butyl 4-(1-(6-(3-hydroxypyrrolidin-1-yl)-2-methoxypyrimidin-4-yl)-5-methyl-1H-indazol-6-yl)piperidine-1-carboxylate (124 mg, 0.240 mmol) in HCl/Et₂O (4 M, 1 mL) and MeOH (1 mL) was stirred at room temperature overnight, then concentrated to give the title product (99 mg, 100%) as a yellow solid.

LCMS [column: C₁₈; column size: 2.1×50 mm; Waters ACQUITY UPLC BEH; mobile phase: B (MeCN); A (0.02% NH₄Ac+5% MeCN); flow rate: 0.5 mL/min; gradient (B %) in 3 mins]: Rt=1.37 min; MS Calcd.:408, MS Found: 409 [M+H]⁺.

Description 41:

### (R)-1-(6-iodo-2-methoxypyrimidin-4-yl)pyrrolidin-3-ol (D41)

The title compound was prepared by a procedure similar to that described for D3 starting from a solution of 4,6-diiodo-2-methylpyrimidine, (R)-pyrrolidin-3-ol hydrochloride and TEA in i-PrOH.

¹H NMR (400 MHz, CDCl₃) δ 6.43 (s, 1H), 4.60 (s, 1H), 3.89 (s, 3H), 3.77-3.36 (m, 4H), 2.16-2.03 (m, 2H), 1.77 (br s, 1H).

Description 42:

### (R)-tert-Butyl 4-(1-(6-(3-hydroxypyrrolidin-1-yl)-2-methoxypyrimidin-4-yl)-5-methyl-1H-indazol-6-yl)piperidine-1-carboxylate (D42)

The title compound was prepared by a procedure similar to that described for D36 starting from a mixture of tert-butyl-4-(5-methyl-1H-indazol-6-yl)piperidine-1-carboxylate, (R)-1-(6-iodo-2-methoxypyrimidin-4-yl)pyrrolidin-3-ol, CuI, K₃PO₄ and N,N′-dimethylcyclohexane-1,2-diamine in toluene at 100° C.

¹H NMR (400 MHz, CDCl₃) δ 8.74 (s, 1H), 8.07 (s, 1H), 7.52 (s, 1H), 6.62 (s, 1H), 4.64 (s, 1H), 4.29-4.25 (br s, 2H), 4.11 (s, 3H), 3.77-3.66 (m, 4H), 2.99-2.95 (m, 1H), 2.85 (br s, 2H), 2.47 (s, 3H), 2.13 (br s, 2H), 1.89-1.86 (m, 2H), 1.70-1.65 (m, 2H), 1.59 (s, 9H).

Description 43:

### (R)-1-(2-Methoxy-6-(5-methyl-6-(piperidin-4-yl)-1H-indazol-1-yl)pyrimidin-4-yl)pyrrolidin-3-ol (D43)

The title compound was prepared by a procedure similar to that described for D37 starting from a solution of (R)-tert-butyl 4-(1-(6-(3-hydroxypyrrolidin-1-yl)-2-methoxypyrimidin-4-yl)-5-methyl-1H-indazol-6-yl)piperidine-1-carboxylate in HCl/Et₂O and MeCOH.

LCMS [column: C₁₈; column size: 2.1×50 mm; Waters ACQUITY UPLC BEH; mobile phase: B (MeCN); A (0.02% NH₄Ac+5% MeCN); flow rate: 0.5 mL/min; gradient (B %) in 3 mins.]: Rt=1.37 min; MS Calcd.:408, MS Found: 409 [M+H]⁺.

Description 44:

### 4-Benzyl-N-methoxy-N-methylmorpholine-2-carboxamide (D44)

A mixture of 4-benzylmorpholine-2-carboxylic acid (10.00 g, 45.25 mmol), 4-methylmorpholine (13.24 g, 135.8 mmol) and N,O-dimethylhydroxylamine hydrochloride (13.24 g, 135.8 mmol) in DCM (250 mL) was treated with EDCI (26.00 g, 135.8 mmol) at room temperature. The reaction mixture was stirred at room temperature for 18 hrs, then poured into sat.NaHCO₃ (200 mL) solution and extracted with DCM (200 mL×2). The combined organic layers were dried over Na₂SO₄, filtered and concentrated to give the title compound (11.74 g, 98%) as a yellow oil.

¹H NMR (400 MHz, CD₃Cl) δ 7.32-7.23 (m, 5H), 4.02-3.99 (m, 1H), 3.99-3.77 (m, 2H), 3.68 (s, 3H), 3.58-3.50 (m, 2H), 3.17 (s, 3H), 2.94-2.89 (m, 1H), 2.68-2.63 (m, 1H), 2.34-2.19 (m, 2H).

Description 45:

### 1-(4-Benzylmorpholin-2-yl)ethanone (D45)

To a solution of 4-benzyl-N-methoxy-N-methylmorpholine-2-carboxamide (11.7 g, 44.5 mmol) in THE (300 mL) was added a solution of CH₃MgBr (45.00 mL, 133.4 mmol, 3.0 M in ether).

The reaction mixture was stirred for at 0° C. 1 hour and at room temperature for 5 h rs, then cooled to 0° C. and quenched with sat.NH₄C (200 mL). The mixture was extracted with EtOAc (200 mL×2). The combined organic layers were washed with brine, dried over Na₂SO₄, filtered and concentrated. The residue was purified by silica gel chromatography column (petroleum ether:EtOAc=5:1) to give the title compound (5.83 g, 60%) as a yellow oil.

¹H NMR (400 MHz, CD₃Cl) δ 7.34-7.23 (m, 5H), 4.01 (dd, J=10.0, 2.8 HZ, 1H), 3.96-3.91 (m, 1H), 3.72-3.66 (m, 1H), 3.52 (q, J=13.2 HZ, 2H), 3.99 (td, J=11.6, 4.0 HZ, 1H), 2.64 (td, J=14.4, 2.0 HZ, 1H), 2.17 (s, 3H), 2.23-2.15 (m, 1H), 2.04-1.65 (m, 1H).

Description 46:

### 1-(4-Benzylmorpholin-2-yl)ethanol (D46)

To a solution of 1-(4-benzylmorpholin-2-yl)ethanone (5.83 g, 26.6 mmol) in MeCOH (60 mL) was added NaBH₄ (1.52 g, 39.9 mmol) in portions at 0° C. The reaction mixture was stirred at room temperature for 1 hour, then quenched with H₂O (80 mL), concentrated to remove solvent and extracted with EtOAc (100 mL×3). The combined organic layers were washed with brine (80 mL), dried over Na₂SO₄, filtered and concentrated to give the title compound (5.66 g, 96%) as a yellow oil.

¹H NMR (400 MHz, CD₃Cl) δ 7.35-7.23 (m, 5H), 3.92-3.83 (m, 1H), 3.71-3.66 (m, 2H), 3.56-3.54 (m, 3H), 2.80 (m, 1H), 2.63 (d, J=11.2 Hz, 1H), 2.62-2.01 (m, 3H), 1.13 (d J=6.4 Hz, 3H).

Description 47:

### 1-(morpholin-2-yl)ethanol hydrochloride (D47)

A mixture of 1-(4-benzylmorpholin-2-yl)ethanol (1.44 g, 6.51 mmol) and Pd/C (1.10 g) in MeOH (40 mL) was added dropwise conc.HCl (10 drops). The reaction mixture was stirred at room temperature under H₂ overnight, then filtered and concentrated to give the title compound (900 mg, 82%) as a green oil.

¹H-NMR (300 MHz, DMSO-d₆) δ 3.85-3.78 (m, 1H), 3.57-3.07 (m, 4H), 2.90-2.55 (m, 3H), 1.06-0.99 (m, 3H).

Description 48:

### 1-(4-(6-Iodo-2-methoxypyrimidin-4-yl)morpholin-2-yl)ethanol (D48)

To a solution of 4,6-diiodo-2-methoxypyrimidine (1.07 g, 2.95 mmol) and 1-(morpholin-2-yl)ethanol hydrochloride (450 mg, 2.68 mmol) in i-PrOH (30 mL) was added TEA (814 mg, 8.06 mmol). The reaction mixture was stirred at room temperature overnight, diluted with water (100 mL) and extracted with EtOAc (50 mL×3). The combined organic layers were washed with brine (30 mL), dried over Na₂SO₄, filtered and concentrated. The residue was purified by silica gel chromatography column (petroleum ether:EtOAc=3/1) to give the title compound (800 mg, 82%) as a colorless oil.

LCMS [column: C₁₈; column size: 4.6×30 mm 5 μm; Dikwa Diamonsil plus; mobile phase: B(MeCN)A (0.02% NH₄A c+5% MeCN); gradient (B %) in 4 min-5-95-POS; flow 1.5 mL/min, stop time 4 mins]: Rt=1.934 min; MS Calcd.: 365, MS Found: 366 [M+H]⁺.

Descriptions 49 and 50:

### 1-(4-(6-iodo-2-methoxypyrimidin-4-yl)morpholin-2-yl)ethanol (Isomer A. D₄₉ and Isomer B, D₅₀

1-(4-(6-iodo-2-methoxypyrimidin-4-yl)morpholin-2-yl)ethanol (D48, 800 mg) was separated by chiral-HPLC to afford the geometric isomer A (D49, 335 mg, 42%) as a colorless oil

Chiral Separation:

Method: column: Chiralpak IC; 5 μm 250 mm×4.6 mm; Phase: IC, Supercritical CO₂:EtOH=70:30; Flow Rate: 15 mL/min, Wave Length: 230 nm.

¹H-NMR (400 MHz, CDCl₃) δ 6.62-6.15 (m, 1H), 4.32-3.82 (m, 6H), 3.62-3.22 (m, 2H), 3.04-1.92 (m, 3H), 1.25-1.22 (m, 3H).

Chiral-HPLC [Chiralpak IC 5 μm 4.6×250 mm; Phase: Hex:EtOH=70:30; Flow rate: 1.0 mL/min; Wave Length: 230 nm; Temperature: 30° C.]: Rt=8.658 min. (isomer A)

Description 51:

### tert-Butyl 4-(1-(6-(2-(1-hydroxyethyl)morpholino)-2-methoxypyrimidin-4-yl)-5-methyl-1H-indazol-6-yl)piperidine-1-carboxylate (D51)

The title compound was prepared by the procedure similar to D36 starting from a mixture of tert-butyl 4-(5-methyl-1H-indazol-6-yl)piperidine-1-carboxylate, 1-(4-(6-iodo-2-methoxypyrimidin-4-yl)morpholin-2-yl)ethanol (isomer A, D49), N,N′-dimethylcyclohexane-1,2-diamine, CuI and K₃PO₄ in toluene at 100° C.

LCMS [column: C₁₈; column size: 4.6×30 mm 5 μm; Dikwa Diamonsil plus; mobile phase: B (MeCN), A₁ (0.1% FA); gradient(B %) in 4 mins-5-95-POS; flow rate: 1.5 mL/min.]: Rt=2.752 min; MS Calcd.:552, MS Found:553 [M+H]⁺.

Description 52:

### 1-(4-(2-Methoxy-6-(5-methyl-6-(piperidin-4-yl)-1H-indazol-1-yl)pyrimidin-4-yl)morpholin-2-yl)ethanol (D52)

A mixture of tert-butyl 4-(1-(6-(2-(1-hydroxyethyl)morpholino)-2-methoxypyrimidin-4-yl)-5-methyl-1H-indazol-6-yl)piperidine-1-carboxylate (D51) (140 mg, 0.25 mmol) in HCl (g)/MeOH (2 M, 2 mL) was stirred at room temperature for 2 hrs and concentrated. The residue was dissolved in MeOH (15 mL), treated with Amberlyst A-21 resin (1 g) at room temperature for 0.5 hour, filtered and concentrated to afford the title compound (116 mg, 100%) as a colorless oil.

LCMS [column: C₁₈; column size: 4.6×30 mm 5 μm; Dikwa Diamonsil plus; mobile phase: B (MeCN) A1 (0.1% FA); gradient (B %) in 4 mins-5-95-POS; flow rate: 1.5 mL/min]: Rt=1.884 min; MS Calcd.:452, MS Found: 453 [M+H]⁺.

Description 53:

### 6-(1-(3-Deuteriumtetrahydrofuran-3-yl)piperidin-4-yl)-5-methyl-1H-indazole (D53)

To a mixture of 5-methyl-6-(piperidin-4-yl)-1H-indazole (430 mg, 2.00 mmol), dihydrofuran-3(2H)-one (860 mg, 10.0 mmol) in DCM (8 mL) was added NaBD₃CN (264 mg, 4.0 mmol) and 4 drops of HOAc. The mixture was stirred at rt for 2 hrs, filtered and concentrated. The residue was purified by prep-HPLC to give title compound (148 mg, 26%) as a yellow solid.

¹H NMR (400 MHz, MeOD) δ 8.26 (s, 1H), 7.69 (s, 1H), 7.43 (s, 1H), 4.07-3.88 (m, 4H), 3.79-3.73 (m, 1H), 3.47-3.44 (m, 1H), 3.18-3.12 (m, 1H), 2.89-2.82 (m, 2H), 2.49 (s, 3H), 2.36-2.28 (m, 1H), 2.12-1.87 (m, 6H).

¹H NMR (400 MHz, MeOD) δ 8.30 (s, 1H), 7.73 (s, 1H), 7.44 (s, 1H), 4.21 (d, J=11.2 Hz, 1H), 4.13-4.07 (m, 1H), 3.88 (d, J=11.2 Hz, 1H), 3.76 (dd, J=7.6, 16 Hz, 1H), 3.70 (d, J=12.8 Hz, 1H), 3.61 (d, J=12.4 Hz, 1H), 3.35-3.27 (m, 3H), 2.52 (s, 3H), 2.46-2.42 (m, 1H), 2.27-2.18 (m, 3H), 2.20-1.97 (m, 2H)

Description 54:

### 1-(6-Iodo-2-methylpyrimidin-4-yl)-3-methylazetidin-3-ol (D54)

To a solution of 4,6-diiodo-2-methylpyrimidine (1.00 mg, 2.89 mmol), 3-methylazetidin-3-ol (430 mg, 3.47 mmol) in DMSO (12 mL) was added TEA (876 mg, 8.67 mmol). The mixture was stirred at 60° C. for 4 hrs, diluted with H₂O (20 mL) and extracted with EtOAc (30 mL×2).

The combined organic layers were filtered and concentrated. The residue was purified by chromatography column on silica gel (petroleum ether/EtOAc=1/1) to give the title compound (842 mg, 95%) as a yellow oil.

¹HNMR (400 MHz, CDCl₃) δ 6.49 (s, 1H), 3.98 (s, 4H), 2.61 (s, 1H), 2.45 (s, 3H), 1.59 (s, 3H).

Description 55:

### tert-Butyl 3-(methoxy(methyl)carbamoyl)azetidine-1-carboxylate (D55)

To a stirred solution of 1-(tert-butoxycarbonyl)azetidine-3-carboxylic acid (5.00 g, 24.9 mmol) in DMF (50 mL) was added HATU (11.4 g, 29.8 mmol) at rt. After 30 min, N,O-dimethylhydroxylamine hydrochloride (2.40 g, 24.8 mmol) and DIEA (12.8 g, 99.5 mmol) were respectively added dropwise at rt. The reaction mixture was stirred at rt for 16 h. TLC showed the reaction was completed. The mixture was poured into water (100 mL) and extracted with EtOAc (3×100 mL). The combined organic layers were washed with brine (2×150 mL), dried over anhydrous Na₂SO₄, filtered and concentrated to afford the crude product as a light yellow oil (6.0 g).

¹H NMR (400 MHz, CDCl₃) δ 4.13˜4.11 (m, 2H), 4.07˜4.02 (m, 2H), 3.66 (s, 4H), 3.20 (s, 3H), 1.47 (s, 9H).

Description 56:

### tert-Butyl 3-acetylazetidine-1-carboxylate (D56)

To a solution of tert-butyl 3-(methoxy(methyl)carbamoyl)azetidine-1-carboxylate (6.0 g, 24.6 mmol) in THF (50 mL) was added dropwise MeMgBr (3 M in THF, 16 mL, 49.1 mmol) at −78° C. The reaction mixture was stirred at rt for 16 h. TLC showed the reaction was completed. Then the mixture was quenched by water (100 mL) and extracted with EtOAc (3×100 mL). The combined organic layers were washed with brine (2×150 mL), dried over anhydrous Na₂SO₄, filtered and concentrated. The residue was purified by silica gel chromatography eluted with PE/EtOAc=5/1 to give the desired product as a colorless oil (3.8 g, yield: 77%).

¹H NMR (400 MHz, CDCl₃) δ 4.06˜4.04 (m, 4H), 4.07˜4.02 (t, 1H), 2.18 (s, 3H), 1.43 (s, 9H).

Description 57:

### tert-Butyl 3-(1-hydroxyethyl)azetidine-1-carboxylate (D57)

To a solution of tert-butyl 3-acetylazetidine-1-carboxylate (3.80 g, 19.1 mmol) in MeOH (50 mL) was added NaBH₄ (1.40 g, 38.1 mmol) in three portions at rt. The mixture was stirred at rt for 2.0 hrs. TLC showed the reaction was completed. The mixture was quenched by ice-water (100 mL) and extracted with EtOAc (3×100 mL). The combined organic layers were washed with brine (2×150 mL), dried over anhydrous Na₂SO₄, filtered and concentrated to dryness to afford the desired product as a colorless oil (3.8 g, yield: 98%).

¹H NMR (400 MHz, CDCl₃) δ 3.94˜3.81 (m, 4H), 3.66˜3.62 (m, 1H), 2.66 (s, 1H), 2.48 (s, 1H), 1.43 (s, 9H), 1.14˜1.13 (d, J=6 Hz, 3H).

Description 58:

### 1-(Azetidin-3-yl)ethanol (D58)

To a solution of tert-butyl 3-(1-hydroxyethyl)azetidine-1-carboxylate (2.30 g, 11.4 mmol) in CH₂Cl₂ (30 mL) was added TFA (20 mL) at rt. The mixture was stirred at rt for 16 hrs. TLC (PE/EtOAc=1/1) showed the reaction was completed. The reaction mixture was concentrated to dryness to afford the product as a yellow oil which was used in the next step without further purification (5.7 g).

Description 59:

### 1-(1-(6-Iodo-2-methylpyrimidin-4-yl)azetidin-3-yl)ethanol (059)

To a solution of 1-(azetidin-3-yl)ethanol (5.70 g, 23.5 mmol) in isopropanol (60 mL) were added 4,6-diiodo-2-methylpyrimidine (4.0 mg, 11.42 mmol) and DIEA (20 mL, 235.3 mmol). The mixture was stirred at room temperature for 16 h. LC-MS showed the reaction was completed. The reaction mixture was concentrated and purified by silica gel chromatography eluted with PE/EtOAc=10/1˜EtOAc to afford the desired product as a white solid (2.2 g, yield: 61%).

LC-MS [mobile phase: from 50% water (0.1% FA) and 50% MeCN (0.1% FA) to 5% water (0.1% FA) and 95% MeCN (0.1% FA) in 2.6 min]: Rt=0.80 min; MS Calcd: 319.14, MS Found: 320.0 [M+H]⁺.

Description 60:

### tert-Butyl 3-(2-(methoxy(methyl)amino)-2-oxoethyl)azetidine-1-carboxylate (D60)

The title compound was prepared by a procedure similar that described for D55 starting from a solution of 2-(1-(tert-butoxycarbonyl)azetidin-3-yl)acetic acid in DMF, HATU, N,O-dimethylhydroxylamine hydrochloride and DIEA.

Description 61:

### tert-Butyl 3-(2-oxopropyl)azetidine-1-carboxylate (D61)

The title compound was prepared by a procedure similar that described for D56 starting from a mixture of tert-butyl 3-(2-(methoxy(methyl)amino)-2-oxoethyl)azetidine-1-carboxylate in THE at −78° C. and MeMgBr.

¹H NMR (400 MHz, CDCl₃) 4.09 (t, J=8.4 Hz, 2H), 3.53-3.50 (m, 2H), 2.88-2.77 (m, 3H), 2.14 (s, 3H), 1.42 (s, 9H).

Description 62:

### tert-Butyl 3-(2-hydroxypropyl)azetidine-1-carboxylate (D62)

The title compound was prepared by a procedure similar that described for D57 starting from a mixture of tert-butyl 3-(2-oxopropyl)azetidine-1-carboxylate in MeOH at 0° C. and NaBH₄.

Description 63:

### 1-(Azetidin-3-yl)propan-2-ol (D63)

A solution of tert-butyl 3-(2-hydroxypropyl)azetidine-1-carboxylate (0.980 g, 4.55 mmol) in TFA (5 mL) was stirred at rt for 16 h. The solvent was removed under reduced pressure to give the desired product as a light yellow oil (0.75 g) which was directly used into next step without purification.

Description 64:

### 1-(1-(6-Iodo-2-methylpyrimidin-4-yl)azetidin-3-yl)propan-2-ol (D64)

The title compound was prepared by a procedure similar that described for D3 starting from a mixture of 1-(azetidin-3-yl)propan-2-ol, 4,6-diiodo-2-methylpyrimidine and DIEA in ⁱPrOH.

LC-MS [mobile phase: from 90% water (0.1% FA) and 10% MeCN (0.1% FA) to 50% water (0.1% FA) and 50% MeCN (0.1% FA) in 3.0 min]: Purity: 98% @ 254 nm; Rt=0.76 min; MS Calcd: 334.0, MS Found: 334.1 [M+H]⁺.

Description 65:

### tert-Butyl 3-(2-(methoxy(methyl)amino)-2-oxoethyl)azetidine-1-carboxylate (D65)

The title compound was prepared by a procedure similar that described for D55 starting from a mixture of 2-(1-(tert-butoxycarbonyl)azetidin-3-yl)acetic acid in DMF, N,O dimethylhyd-roxyl-amine hydrochloride, HOBt, EDCI and DIPEA.

¹H NMR (400 MHz, CDCl₃) δ 4.14-4.09 (m, 2H), 3.70 (s, 3H), 3.62-3.58 (m, 2H), 3.16 (s, 3H), 2.95-2.75 (m, 2H), 1.43 (s, 9H).

Description 66:

### tert-Butyl 3-(2-oxopropyl)azetidine-1-carboxylate (D66)

The title compound was prepared by a procedure similar that described for D56 starting from a solution of tert-butyl 3-(2-(methoxy(methyl)amino)-2-oxoethyl)azetidine-1-carboxylate in THE and CH₃MgBr.

¹H NMR (400 MHz, CDCl₃) δ 4.12-4.07 (m, 2H), 3.54-3.50 (m, 2H), 2.88-2.77 (m, 3H), 2.14 (s, 3H), 1.42 (s, 9H).

Description 67:

### tert-Butyl 3-(2-hydroxypropyl)azetidine-1-carboxylate (D67)

The title compound was prepared by a procedure similar that described for D57 starting from a solution of tert-butyl 3-(2-oxopropyl)azetidine-1-carboxylate in MeOH (20 mL) and NaBH₄.

¹H NMR (400 MHz, CDCl₃) δ 4.13-4.03 (m, 2H), 3.62.3.58 (m, 2H), 2.69-2.67 (m, 1H), 1.78-1.73 (m, 2H), 1.43 (s, 9H), 1.28-1.18 (m, 3H).

Description 68:

### 1-(azetidin-3-yl)propan-2-ol 2,2,2-trifluoroacetate (D68)

The title compound was prepared by a procedure similar that described for D58 starting from a solution of tert-butyl 3-(2-hydroxypropyl)azetidine-1-carboxylate in DCM and CF₃COOH.

¹H NMR (400 MHz, CDCl₃) δ 4.21-3.73 (m, 5H), 1.40-1.25 (m, 5H).

Description 69:

### 1-(1-(6-Iodo-2-methoxypyrimidin-4-yl)azetidin-3-yl)propan-2-ol (D69)

The title compound was prepared by a procedure similar that described for D3 starting from a mixture of 1-(azetidin-3-yl)propan-2-ol 2,2,2-trifluoroacetate, 4,6-diiodo-2-methoxypyrimidine and DIPEA in EtOH/THF.

LC-MS [mobile phase: from 80% water (0.1% FA) and 20% MeCN (0.1% FA) to 5% water (0.1% FA) and 95% MeCN (0.1% FA) in 2 min]: Rt=1.32 min; MS Calcd: 349, MS Found: 350 [M+H]⁺.

Description 70

### cis-1-(6-Chloro-2-methoxypyrimidin-4-yl)-6-(3-fluoro-1-(tetrahydrofuran-3-yl)piperidin-4-yl)-5-methyl-1H-indazole (D70)

A mixture of cis-6-(3-fluoro-1-(tetrahydrofuran-3-yl)piperidin-4-yl)-5-methyl-1H-indazole (D33) (69.0 mg, 0.230 mmol), 4,6-dichloro-2-methoxypyrimidine (45.0 mg, 0.250 mmol) and Cs₂CO₃ (225 mg, 0.690 mmol) in DMF (5 mL) was stirred at 40° C. overnight, then poured into water (50 mL) and extracted with EtOAc (30 mL×3). The combined organic layers were dried over Na₂SO₄, filtered and concentrated to give the crude as a yellow solid. (100 mg).

LC-MS [mobile phase: from 80% water (0.1% FA) and 20% MeCN (0.1% FA) to 5% water (0.1% FA) and 95% MeCN (0.1% FA) in 2.6 min]: Rt=1.159 min; MS Calcd: 445; MS Found: 446 [M+H]⁺.

Description 71

### cis-1-(6-Chloro-2-methoxypyrimidin-4-yl)-6-(3-fluoro-1-(tetrahydrofuran-3-Yl)piperidin-4-yl)-5-methyl-1H-indazole (D71)

The title compound was prepared by a procedure similar that described for D70 starting from a mixture of cis-6-(3-fluoro-1-(tetrahydrofuran-3-yl)piperidin-4-yl)-5-methyl-1H-indazole (D34), 4,6-dichloro-2-methoxypyrimidine and Cs₂CO₃ in DMF at 40° C.

LC-MS [mobile phase: from 80% water (0.1% FA) and 20% MeCN (0.1% FA) to 5% water (0.1% FA) and 95% MeCN (0.1% FA) in 2.6 min]: Rt=1.149 min; MS Calcd: 445; MS Found: 446 [M+H]⁺.

Description 72

### 1-(Azetidin-3-yloxy)propan-2-ol hydrochloride (D72)

A mixture of tert-butyl 3-(2-hydroxypropoxy)azetidine-1-carboxylate (1.00 g, 4.33 mmol) in HCl/MeOH (3 M, 6 mL) was stirred at room temperature for 2 hrs and concentrated to give the title compound (567 mg, 100%) as a yellow oil.

¹HNMR (400 MHz, CDCl₃) δ 4.49-3.90 (m, 6H), 3.46-3.31 (m, 2H), 1.28-1.06 (m, 3H).

Description 73

### 1-((1-(6-Iodo-2-methylpyrimidin-4-yl)azetidin-3-yl)oxy)propan-2-ol (D73)

The title compound was prepared by a procedure similar that described for D3 starting from a mixture of 4,6-diiodo-2-methylpyrimidine, 1-(azetidin-3-yloxy)propan-2-ol hydrochloride and TEA in DMSO at 60° C.

¹H-NMR (CDCl₃, 400 MHz) δ 6.49 (s, 1H), 4.48-4.44 (m, 1H), 4.26-4.22 (m, 2H), 4.01-3.94 (m, 3H), 3.45-3.41 (m, 1H), 3.27-3.23 (m, 1H), 2.46 (s, 3H), 2.32 (br s, 1H), 1.18 (d, J=6.4 MHz, 3H).

Descriptions 74 and 75

### 1-((1-(6-Iodo-2-methylpyrimidin-4-yl)azetidin-3-yl)oxy)propan-2-ol (Single Unknown Enantiomer 1, D74 and Single Unknown Enantiomer 2. D75)

1-((1-(6-iodo-2-methylpyrimidin-4-yl)azetidin-3-yl)oxy)propan-2-ol (D73) (756 mg, 2.16 mmol) was separated by prep-HPLC to give single unknown enatiomer 1 (303 mg, 40%) and single unknown enatiomer isomer 2 (315 mg, 42%).

Chiral pre-HPLC: column: Chiralpak IC 5 μm 20×150 mm; Phase: Supercritical CO₂:EtOH=80:20, Flow rate: 20 mL/min; Wave length: 230 nm.

Single Unknown Enantiomer 1 (D74)

Chiral-HPLC [Column: Chiralpak IC 250 mm×4.6 mm 5 um; Mobile phase: Hex:EtOH=80:20; Flow rate:1 mL/min; Temperature: 30° C.]: Rt=9.448 min.

LCMS [column: C₁₈; column size: 4.6×30 mm 5 μm; Dikwa Diamonsil plus; mobile phase: B (MeCN) A1 (0.02% NH₄Ac+5% MeCN); gradient(B %) in 4 mins. 10-95-POS; flow rate: 1.5 mL/min]: Rt=1.527 min; MS Calcd.:349, MS Found: 350 [M+H]⁺.

Single Unknown Enantiomer 2 (D75)

Chiral-HPLC [Column: Chiralpak IC 250 mm×4.6 mm 5 um; Mobile phase: Supercritical CO₂:EtOH=80:20; Flow rate: 1 mL/min; Temperature: 30° C.]: Rt=11.255 min.

LCMS [column: C₁₈; column size: 4.6×30 mm 5 μm; Dikwa Diamonsil plus; mobile phase: B (MeCN) A1 (0.02% NH₄Ac+5% MeCN); gradient (B %) in 4 mins. 10-95-POS; flow rate: 1.5 mL/min]: Rt=1.527 min; MS Calcd.: 349, MS Found: 350 [M+H]⁺.

Description 76

### tert-Butyl 4-(1-(6-(3-(2-hydroxypropoxy)azetidin-1-yl)-2-methylpyrimidin-4-yl)-5-methyl-1H-indazol-6-yl)piperidine-1-carboxylate (D76)

A mixture of tert-butyl 4-(5-methyl-1H-indazol-6-yl)piperidine-1-carboxylate (130 mg, 0.410 mmol), 1-((1-(6-iodo-2-methylpyrimidin-4-yl)azetidin-3-yl)oxy)propan-2-ol (single unknown enantiomer 1, D74) (172 mg, 0.49 mmol), N,N′-dimethylcyclohexane-1,2-diamine (116 mg, 0.820 mmol), CuI (78.0 mg, 0.410 mmol) and K₃PO₄ (174 mg, 0.820 mmol) in toluene (3 mL) was stirred at 100° C. for 2 hrs. The mixture was diluted with EtOAc (30 mL), washed with brine (30 mL), dried over Na₂SO₄, filtered and concentrated. The residue was purified by silica gel chromatography column (petroleum ether/EtOAc=1:1) to give the title compound (143 mg, 65%) as a yellow oil.

¹H-NMR (CDCl₃, 400 MHz) δ 8.76 (s, 1H), 8.06 (s, 1H), 7.50 (s, 1H), 6.60 (s, 1H), 4.51-4.48 (m, 1H), 4.38-4.27 (m, 4H), 4.15-4.01 (m, 4H), 3.47-3.44 (m, 1H), 3.30-3.24 (m, 1H), 2.97-2.84 (m, 3H), 2.62 (s, 3H), 2.47-2.42 (m, 3H), 2.25 (br s, 1H), 2.05 (s, 1H), 1.90-1.86 (m, 2H), 1.52 (s, 9H), 1.19 (d, J=6.4 MHz, 3H).

Description 77

### 1-((1-(2-Methyl-6-(5-methyl-6-(piperidin-4-yl)-1H-indazol-1-yl)pyrimidin-4-yl)azetidin-3-yl)oxy)propan-2-ol (D77)

To a solution of tert-butyl 4-(1-(6-(3-(2-hydroxypropoxy)azetidin-1-yl)-2-methylpyrimidin-4-yl)-5-methyl-1H-indazol-6-yl)piperidine-1-carboxylate (D76, 143 mg, 0.270 mmol) in MeOH (4 mL) was added HCl/dioxane (2 mL). The mixture was stirred at room temperature for 2 hour, then concentrated to give the compound (110 mg, 94%) as a yellow oil.

LCMS [column: C₁₈; column size: 4.6×30 mm 5 μm; Dikwa Diamonsil plus; mobile phase: B (MeCN) A1 (0.02% NH₄Ac+5% MeCN); gradient (B %) in 4 mins. 10-95-POS; flow rate: 1.5 mL/min]: Rt=1.744 min; MS Calcd.: 436, MS Found: 437 [M+H]⁺.

Description 78

### tert-Butyl 4-(1-(6-(3-(2-hydroxypropoxy)azetidin-1-yl)-2-methylpyrimidin-4-yl)-5-methyl-1H-indazol-6-yl)piperidine-1-carboxylate (D78)

The title compound was prepared by a procedure similar that described for D76 starting from a mixture of tert-butyl 4-(5-methyl-1H-indazol-6-yl)piperidine-1-carboxylate, 1-((1-(6-iodo-2-methylpyrimidin-4-yl)azetidin-3-yl)oxy)propan-2-ol (single unknown enatiomer 2, D75), N,N′-dimethylcyclohexane-1,2-diamine, CuI and K₃PO₄ in toluene at 100° C.

¹H-NMR (CDCl₃, 400 MHz) δ 8.76 (s, 1H), 8.06 (s, 1H), 7.51 (s, 1H), 6.60 (s, 1H), 4.51-4.50 (m, 1H), 4.36-4.32 (m, 4H), 4.13-4.00 (m, 4H), 3.47-3.44 (m, 1H), 3.29-3.24 (m, 1H), 2.97-2.84 (m, 3H), 2.60 (s, 3H), 2.47-2.42 (m, 3H), 2.24 (br s, 1H), 2.05 (s, 1H), 1.90-1.85 (m, 2H), 1.50 (s, 9H), 1.20 (d, J=8.4 Hz, 3H).

Description 79

### 1-((1-(2-Methyl-6-(5-methyl-6-(piperidin-4-yl)-1H-indazol-1-yl)pyrimidin-4-yl)azetidin-3-yl)oxy)propan-2-ol (D79)

The title compound was prepared by a procedure similar that described for D77 starting from tert-butyl 4-(1-(6-(3-(2-hydroxypropoxy)azetidin-1-yl)-2-methylpyrimidin-4-yl)-5-methyl-1H-indazol-6-yl)piperidine-1-carboxylate (78) in MeOH and HCl/dioxane.

LCMS [column: C₁₈; column size: 4.6×30 mm 5 μm; Dikwa Diamonsil plus; mobile phase: B (MeCN) A₁ (0.02% NH₄Ac+5% MeCN); gradient(B %) in 4 mins. 10-95-POS; flow rate: 1.5 mL/min]: Rt=1.743 min; MS Calcd.:436, MS Found: 437 [M+H]⁺.

Descriptions 80 and 81

### 4-(6-Iodo-2-methylpyrimidin-4-yl)-6-methylmorpholin-2-yl)methanol (D80 and D81)

The title compound was prepared by a procedure similar that described for D₃ starting from a solution of (6-methylmorpholin-2-yl)methanol and 4,6-diiodo-2-methylpyrimidine in ⁱPrOH and DIEA.

The residue was purified by silica gel chromatography eluted with PE/EtOAc=5/1 to give the two desired products as white solids (isomer 1, D80: 510 mg, yield: 38% and isomer 2, D81: 320 mg, yield: 24%).

Isomer 1 (D80)

LC-MS [mobile phase: from 90% water (0.1% FA) and 10% MeCN (0.1% FA) to 5% water (0.1% FA) and 95% MeCN (0.1% FA) in 2.6 min]: Rt=1.240 min; MS Calcd: 349.17, MS Found: 350.0 [M+H]⁺

Isomer 2 (D81)

LC-MS [mobile phase: from 90% water (0.1% FA) and 10% MeCN (0.1% FA) to 5% water (0.1% FA) and 95% MeCN (0.1% FA) in 2.6 min]: Rt=1.166 min; MS Calcd: 349.17, MS Found: 350.0 [M+H]⁺.

Description 82

### (R)-4-(6-Iodo-2-methoxypyrimidin-4-yl)-3-methylmorpholine (D82)

The title compound was prepared by a procedure similar that described for D3 starting from 4,6-diiodo-2-methoxypyrimidine and (S)-3-methylmorpholine.

Description 83

### (S)-4-(6-Iodo-2-methoxypyrimidin-4-yl)-3-methylmorpholine (D83)

The title compound was prepared by a procedure similar that described for D3 starting from 4,6-diiodo-2-methoxypyrimidine and (S)-3-methylmorpholine.

Description 84

### (R)-4-(6-Iodo-2-methylpyrimidin-4-yl)-3-methylmorpholine (D84)

To a mixture of 4,6-diiodo-2-methylpyrimidine (700 mg, 2.1 mmol) and (R)-3-methylmorpholine (280 mg, 2.77 mmol) in ⁱPrOH (10 mL) was added DIPEA (1 mL). The reaction mixture was heated to 85° C., stirred overnight and concentrated. The residue was purified by silica gel chromatography eluted with PE:EtOAc=20:1-5:1 to afford the desired product as a colorless oil (420 mg, yield: 66%).

¹H NMR (400 MHz, CDCl₃) δ 6.74 (s, 1H), 4.26 (d, J=4.0 Hz, 1H), 3.99 (dd, J=11.6 Hz, 3.6 Hz, 2H), 3.78-3.65 (m, 2H), 3.53 (td, J=11.6 Hz, 3.2 Hz, 1H), 3.20 (td, J=13.2 Hz, 4.0 Hz, 1H), 2.46 (s, 3H), 1.28 (d, J=6.8 Hz, 3H).

Description 85

### (S)-4-(6-Iodo-2-methylpyrimidin-4-yl)-3-methylmorpholine (D85)

The title compound was prepared by a procedure similar that described for D3 starting from a solution of 4,6-diiodo-2-methylpyrimidine and (S)-3-methylmorpholine and DIPEA in iPrOH and THF.

LC-MS [mobile phase: from 60% water (0.1% FA) and 40% MeCN (0.1% FA) to 5% water (0.1% FA) and 95% MeCN (0.1% FA) in 2.0 min]: Rt=0.99 min; MS Calcd.:319.0, MS Found: 320.2 [M+H]⁺.

Description 86

### 1-(6-Chloro-2-methylpyrimidin-4-yl)-5-methyl-6-(1-(tetrahydrofuran-3-yl)piperidin-4-yl)-1H-indazole (D86)

A mixture of 5-methyl-6-(1-(tetrahydrofuran-3-yl)piperidin-4-yl)-1H-indazole (1.0 g, 3.5 mol), 4,6-dichloro-2-methylpyrimidine (570 mg, 3.5 mmol) and Cs₂CO₃ (3.42 g, 10.5 mmol) in DMF (20 mL) was stirred at 50° C. for 5 h, then diluted with water (50 mL) and extracted with EtOAc (2×50 mL). The combined organic layers were washed with water (3×50 mL), brine (50 mL), dried, filtered and concentrated. The residue was purified by silica gel chromatography eluted with EtOAc:MeOH=1:1 to give the crude product. The crude product was recrystallized from MeOH/CH₂Cl₂=7/1 to give the title product as a white solid (390 mg, 27% yield).

LC-MS [mobile phase: from 90% water (0.1% FA) and 10% MeCN (0.1% FA) to 5% water (0.1% FA) and 95% MeCN (0.1% FA) in 10 min]: Rt=5.18 min; MS Calcd: 411.9, MS Found: 412.2[M+H]⁺

Description 87

### 4-(6-Iodo-2-methoxypyrimidin-4-yl)morpholine (D87)

The title compound was prepared by a procedure similar that described for D3 starting from a mixture of 4,6-diiodo-2-methoxypyrimidine and morpholine in Et₃N and EtOH.

LC-MS [mobile phase: from 80% water (0.1% FA) and 20% MeCN (0.1% FA) to 5% water (0.1% FA) and 95% MeCN (0.1% FA) in 2.0 min]: Rt=0.89 min; MS Calcd.:321.0, MS Found: 322.2 [M+H]⁺.

Description 88

### (E)-((But-2-en-1-yloxy)methyl)benzene(D88)

To a stirred solution of NaH (4.0 g, 100 mmol) in DMF (50 mL) was added (E)-but-2-en-1-ol (6.0 g, 85.4 mmol) at 0° C. After 30 min BnBr (15.6 g, 91.6 mmol) in DMF (10 mL) was added at 0° C. dropwise and the reaction mixture was allowed to room temperature and stirred overnight. TLC showed the reaction was completed. The reaction mixture was diluted with water (100 mL) and extracted with EtOAc (3×100 mL). The combined organic layers were washed with water (3×100 mL) and brine (100 mL), dried over anhydrous Na₂SO₄, filtered and concentrated to give a residue. The residue was purified by silica gel column chromatography (PE:EtOAc=100:1) to give the title compound (12.5 g, yield: 92.5%) as a colorless oil.

¹H NMR (400 MHz, CDCl₃) δ 7.34˜7.27 (m, 5H), 5.76˜5.69 (m, 1H), 5.66˜5.60 (m, 1H), 4.49 (s, 2H), 3.96˜3.95 (m, 2H), 1.73˜1.71 (m, 3H).

Description 89

### 2-((Benzyloxy)methyl)-3-methyloxirane (D89)

To a solution of (E)-((but-2-en-1-yloxy)methyl)benzene (12.50 g, 76.90 mmol) in CH₂Cl₂ (100 mL) was added m-CPBA (20.00 g, 115.4 mmol) in three portions. The mixture was stirred at room temperature overnight. TLC showed the reaction was completed. The reaction mixture was filtered and the fitrate was washed with Na₂S₂O₃ (2×50 mL) and brine (100 mL). The organic layer was dried over anhydrous Na₂SO₄, filtered and concentrated. The residue was purified by silica gel column chromatography (PE) to give the title compound (13.2 g, yield: 96.3%) as a pale yellow oil.

¹H NMR (400 MHz, CDCl₃) δ 7.36˜7.27 (m, 5H), 4.62˜4.53 (m, 2H), 3.71˜3.67 (m, 1H), 3.51˜3.47 (m, 2H), 2.92˜2.89 (m, 2H), 1.33˜1.32 (d, J=4.8 Hz, 3H).

Description 90

### 3-Amino-1-(benzyloxy)butan-2-ol (D90)

To a solution of 2-((benzyloxy)methyl)-3-methyloxirane (13.0 g, 72.8 mmol) in MeCOH (40 mL) was added NH₃.H₂O (25 mL). The reaction mixture was stirred at 95° C. overnight. LC-MS showed the reaction was completed. The reaction mixture was diluted with water (200 mL) and extracted with EtOAc (3×250 mL). The combined organic layers were washed with brine (300 mL), dried over anhydrous Na₂SO₄, filtered and concentrated to give the crude product (12 g) as a white solid.

LC-MS [mobile phase: from 55% water (0.1% FA) and 55% MeCN (0.1% FA) to 5% water (0.1% FA) and 95% MeCN (0.1% FA) in 2.6 min]: Rt=0.678 min; MS Calcd: 195.13, MS Found: 196.2 [M+H]⁺.

Description 91

### N-(4-(Benzyloxy)-3-hydroxybutan-2-yl)-2-chloroacetamide (D91)

To a stirred solution of 3-amino-1-(benzyloxy)butan-2-ol (12.0 g, 61.5 mmol) in anhydrous THE (300 mL) was added Et₃N (9.50 g, 93.8 mmol) at 0° C. After 10 min, 2-chloroacetyl chloride (7.00 g, 61.9 mmol) was added at 0° C. and stirred for 10 min. The reaction mixture was allowed to room temperature and stirred for 2 h. LC-MS showed the reaction was completed. The reaction mixture was quenched with a solution of sat. NH₄Cl (100 mL) and extracted with EtOAc (3×100 mL). The combined organic layers were washed with brine (200 mL), dried over anhydrous Na₂SO₄, filtered and concentrated to give a residue. The residue was purified by silica gel column chromatography (PE:EtOAc=5:1˜2:1) to give the title compound (12.0 g, yield: 72%) as a colorless oil.

LC-MS [mobile phase: from 50% water (0.1% FA) and 50% MeCN (0.1% FA) to 5% water (0.1% FA) and 95% MeCN (0.1% FA) in 2.6 min]: Rt=1.010 min; MS Calcd: 271.10, MS Found: 272.1 [M+H]⁺.

Description 92

### cis-6-((Benzyloxy)methyl)-5-methylmorpholin-3-one (D92)

To a solution of N-(4-(benzyloxy)-3-hydroxybutan-2-yl)-2-chloroacetamide (9.0 g, 44.3 mmol) a in t-BuOH (250 mL) was added t-BuOK (3.70 g, 44.3 mmol) in three portions. The reaction mixture was stirred at room temperature overnight under N₂. LC-MS showed the reaction was completed. The reaction mixture was diluted with water (200 mL) and extracted with CH₂Cl₂ (3×300 mL). The combined organic layers were washed with brine (300 mL), dried over anhydrous Na₂SO₄, filtered and concentrated to give a residue. The residue was purified by silica gel column chromatography (PE:EtOAc=1:1) to give the title compound (5.0 g, yield: 64%) as a white solid.

¹H NMR (400 MHz, CDCl₃) δ 7.38˜7.30 (m, 5H), 6.75 (m, 1H), 4.62˜4.49 (m, 2H), 4.28˜4.11 (m, 2H), 4.01˜3.97 (m, 1H), 3.61˜3.52 (m, 2H), 3.47˜3.41 (m, 1H), 1.17˜1.16 (m, 3H).

LC-MS [mobile phase: from 50% water (0.1% FA) and 50% MeCN (0.1% FA) to 5% water (0.1% FA) and 95% MeCN (0.1% FA) in 2.0 min]: Rt=0.948 min; MS Calcd: 235.12, MS Found: 236.2 [M+H]⁺.

Description 93

### cis-2-((Benzyloxy)methyl)-3-methylmorpholine (D93)

To a solution of cis-6-((benzyloxy)methyl)-5-methylmorpholin-3-one (3.20 g, 13.2 mmol) in anhydrous THE (80 mL) was added dropwise BH₃-THF (40 mL, 40 mmol) at 0° C. The reaction mixture was allowed to room temperature and stirred overnight under N₂. TLC showed the reaction was completed. The reaction mixture was quenched with water (50 mL) and extracted with CH₂Cl₂ (3×200 mL). The combined organic layers were washed with brine (400 mL), dried over anhydrous Na₂SO₄, filtered and concentrated to give a residue. The residue was dissolved in MeOH (80 mL) and HCl (5 mL) and stirred at reflux for 2 h. The reaction mixture was concentrated to give a residue. The residue was purified by silica gel column chromatography (EtOAc:MeOH=10:1) to give the title compound (2.4 g, yield: 80%) as a yellow oil.

Description 94

### cis-(3-Methylmorpholin-2-yl)methanol (D94)

To a solution of cis-2-((benzyloxy)methyl)-3-methylmorpholine (2.2 g, 10 mmol) a in MeOH (80 mL) was added HCl (5 mL, 5 mmol) and Pd/C (200 mg). The reaction mixture was stirred at room temperature under H₂ overnight. LC-MS showed the reaction was completed. The reaction mixture was filtered and concentrated to give crude product. (1.71 g) as a yellow oil.

LC-MS [mobile phase: from 95% water (0.1% FA) and 5% MeCN (0.1% FA) to 5% water (0.1% FA) and 95% MeCN (0.1% FA) in 2.6 min]: Rt=0.322 min; MS Calcd: 131.09, MS Found: 132.2 [M+H]⁺.

Description 95

### (R)-(4-(6-Iodo-2-methoxypyrimidin-4-yl)morpholin-2-yl)methanol (D95)

The title compound was prepared by a procedure similar to that described for D3 starting from a solution of 4,6-diiodo-2-methoxypyrimidine and (R)-morpholin-2-ylmethanol hydrochloride in ⁱPrOH and DIPEA.

LC-MS [mobile phase: from 50% water (0.1% FA) and 50% MeCN (0.1% FA) to 5% water (0.1% FA) and 95% MeCN (0.1% FA) in 2.6 min]: Rt=0.92 min; MS Calcd: 351.1, MS Found: 352.0 [M+H]⁺.

Description 96

### (S)-(4-(6-Iodo-2-methoxypyrimidin-4-yl)morpholin-2-yl)methanol (D96)

The title compound was prepared by a procedure similar to that described for D3 starting from a solution of 4,6-diiodo-2-methoxypyrimidine and (S)-morpholin-2-ylmethanol hydrochloride in ⁱPrOH and DIPEA.

LC-MS [mobile phase: from 50% water (0.1% FA) and 50% MeCN (0.1% FA) to 5% water (0.1% FA) and 95% MeCN (0.1% FA) in 2.6 min]: Rt=0.91 min; MS Calcd: 351.1, MS Found: 352.0 [M+H].

Description 97

### (S)-(4-(6-Iodo-2-methoxypyrimidin-4-yl)morpholin-3-yl)methanol (D97)

The title compound was prepared by a procedure similar to that described for D3 starting from a solution of (S)-morpholin-3-ylmethanol hydrochloride and 4,6-diiodo-2-methylpyrimidine in EtOH/THF and DIEA.

LC-MS [mobile phase: from 90% water (0.1% FA) and 10% MeCN (0.1% FA) to 5% water (0.1% FA) and 95% MeCN (0.1% FA) in 2.6 min]: purity 96%, Rt=1.400 min; MS Calcd: 351.14, MS Found: 351.9 [M+H]⁺.

Description 98

### (R)-(4-(6-iodo-2-methoxypyrimidin-4-yl)morpholin-3-yl)methanol (D₉₈)

The title compound was prepared by a procedure similar to that described for D3 starting from a solution of (S)-morpholin-3-ylmethanol hydrochloride and 4,6-diiodo-2-methylpyrimidine in DMF and DIEA at 70° C.

LC-MS [mobile phase: from 50% water (0.1% FA) and 50% MeCN (0.1% FA) to 5% water (0.1% FA) and 95% MeCN (0.1% FA) in 2.6 min]: purity 98%, Rt=0.925 min; MS Calcd: 351.14, MS Found: 351.9 [M+H]⁺.

Description 99

### tert-butyl 4-(5-methyl-1H-indazol-6-yl)piperidine-1-carboxylate

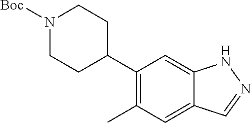 [SMILES: Cc1cc2cn[nH]c2cc1C1CCN(C(=O)OC(C)(C)C)CC1]

To a stirred solution of 5-methyl-6-(piperidin-4-yl)-1H-indazole (D9, 1.00 g, 4.64 mmol) and Et₃N (930 mg, 9.20 mmol) in CH₂Cl₂ (80 mL) was added Boc₂O (1.00 g, 4.60 mmol). The reaction mixture was stirred at room temperature for 3 h. LC-MS showed the reaction was completed. The mixture was concentrated to dryness and purified by silica gel chromatography eluted with PE:EtOAc=3:1 to afford the desired product as a white solid (900 mg, yield: 61%).

¹H NMR (400 MHz, DMSO-d₆) δ 12.77 (s, 1H), 7.89 (s, 1H), 7.50 (s, 1H), 7.28 (s, 1H), 4.12-4.07 (m, 2H), 3.17 (s, 1H), 2.94-2.84 (m, 2H), 2.40 (s, 3H), 1.77 (d, J=12.0 Hz, 2H), 1.55-1.47 (m, 2H), 1.43 (s, 9H).

Description 100

### tert-butyl 4-(5-methyl-2-((2-(trimethylsilyl)ethoxy)methyl)-2H-indazol-6-yl)piperidi-ne-1-carboxylate

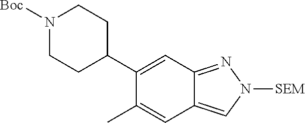 [SMILES: Cc1cc2cn(COCC[Si](C)(C)C)nc2cc1C1CCN(C(=O)OC(C)(C)C)CC1]

To a solution of tert-butyl 4-(5-methyl-1H-indazol-6-yl)piperidine-1-carboxylate (D99, 7.00 g, 22.2 mmol) in THE (100 mL) was added dropwise N-cyclohexyl-N-methylcyclohexanamine (5.60 g, 28.9 mmol) at 0° C. under N₂. After the reaction mixture was stirred at 0° C. for 15 min 2-(trimethylsilyl)ethoxymethyl chlorid (4.40 g, 26.6 mmol) was added dropwise. The reaction mixture was stirred at room temperature overnight, quenched with water and concentrated. The residue was diluted with EtOAc (100 mL) and washed with brine (100 mL). The organic solution was dried over anhydrous Na₂SO₄ and concentrated. The residue was purified by silica gel chromatography (PE:EtOAc=5:1) to give the title product (7.50 g, yield: 76.0%) as a colorless oil.

¹H NMR (400 MHz, CDCl₃): δ 7.98 (s, 1H), 7.55 (s, 1H), 7.48 (s, 1H), 5.71 (s, 2H), 4.33-4.29 (m, 2H), 3.64 (t, J=8.4 Hz, 2H), 2.91-2.85 (m, 3H), 2.46 (s, 3H), 1.90-1.87 (m, 2H), 1.75-1.62 (m, 2H), 1.53 (s, 9H), 0.96 (t, J=8.4 Hz, 2H), 0.00 (s, 9H).

Description 101

### tert-butyl 4-(3-deuterium-5-methyl-2-((2-(trimethylsilyl)ethoxy)methyl)-2H-indazol-6-Y)piperidine-1-carboxylate

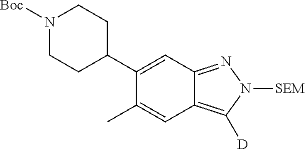 [SMILES: [2H]c1c2cc(C)c(C3CCN(C(=O)OC(C)(C)C)CC3)cc2nn1COCC[Si](C)(C)C]

To a solution of tert-butyl 4-(5-methyl-2-((2-(trimethylsilyl)ethoxy)methyl)-2H-indazol-6-yl)piperidine-1-carboxylate (D100, 7.50 g, 16.8 mmol) in dry THF (80.0 mL) was added n-BuLi (1.6 M in hexane, 15.8 mL, 25.2 mmol) at −65° C. under N₂. The reaction mixture was stirred at −65° C.˜−40° C. for 5 h, then quenched with D₂O (2 mL), diluted with EtOAc (200 mL), washed with sat. NH₄Cl (200 mL) and brine (200 mL), dried over anhydrous Na₂SO₄ and concentrated. The residue was purified by silica gel chromatography (PE:EtOAc=5:1) to give the title product (5.40 g) as a yellow oil.

¹H NMR showed the afforded oil was not fully deuterated. The residue was reacted with n-BuLi and quenched with D₂O one more time to give the title product (2.5 g, yield: 33%) as a yellow oil.

¹H NMR (400 MHz, CDCl₃): δ 7.98 (s, 0.06H), 7.55 (s, 1H), 7.48 (s, 1H), 5.71 (s, 2H), 4.33˜4.29 (m, 2H), 3.64 (t, J=8.0 Hz, 2H), 2.92˜2.85 (m, 3H), 2.46 (s, 3H), 1.90˜1.87 (m, 2H), 1.74˜1.62 (m, 2H), 1.53 (s, 9H), 0.96 (t, J=8.4 Hz, 2H), 0.00 (s, 9H).

Description 102

### 3-deuterium-5-methyl-6-(piperidin-4-yl)-1H-indazole

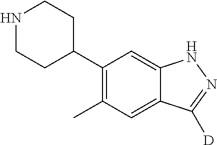 [SMILES: [2H]c1n[nH]c2cc(C3CCNCC3)c(C)cc12]

A mixture of tert-butyl 4-(3-deuterium-5-methyl-2-((2-(trimethylsilyl)ethoxy)methyl)-2H-indazol-6-yl)piperidine-1-carboxylate (D101) in HCl (g)/MeOH (15.0 mL) was stirred at 35° C. for 5 h, then concentrated. The residue was diluted with EtOAc (30.0 mL) and stirred at room temperature overnight. The resulting suspension was filtered to give the title product with a HCl salt (950 mg) as a white solid. The solid 3-deuterium-5-methyl-6-(piperidin-4-yl)-1H-indazole HCl salt (550 mg) was dissolved in MeOH (50.0 mL) and K₂CO₃ (1.50 g) was added. The resulting suspension was stirred at room temperature for 60 min., diluted with CH₂Cl₂ (300 mL), washed with brine (50.0 mL×2), dried over anhydrous Na₂SO₄ and concentrated to give the title product (1.10 g) as a pale yellow solid.

¹H NMR (400 MHz, DMSO-d6): δ 12.78 (s, 1H), 7.89 (s, 0.01H), 7.49 (s, 1H), 7.29 (s, 1H), 4.11 (S, 1H), 3.07 (d, J=12.4 Hz, 2H), 2.86˜2.80 (m, 1H), 2.66˜2.61 (m, 2H), 2.38 (s, 3H), 1.71 (d, J=12.4 Hz, 2H), 1.57˜1.46 (m, 2H).

Description 103

### 3-deuterium-5-methyl-6-(1-(tetrahydrofuran-3-yl)piperidin-4-yl)-1H-indazole

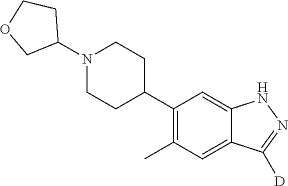 [SMILES: [2H]c1n[nH]c2cc(C3CCN(C4CCOC4)CC3)c(C)cc12]

To a solution of 3-deuterium-5-methyl-6-(piperidin-4-yl)-1H-indazole (1.00 g, 4.62 mmol) in CH₂Cl₂ (20.0 mL) and MeOH (20.0 mL) were added dihydrofuran-3(2H)-one (796 mg, 9.25 mmol), acetic acid (83.0 mg, 1.39 mmol) and 4 Å molecular sieve (500 mg) followed by NaBH₃CN (581 mg, 9.25 mmol). The reaction mixture was stirred at room temperature overnight, quenched with sat. NH₄Cl and filtered. The filtrate was concentrated and purified by silica gel chromatography (CH₂Cl₂:MeOH=20:1) to give the title product (1.20 g, yield: 91.0%) as a pale yellow solid.

LC-MS [mobile phase: from 90% water (0.1% FA) and 10% MeCN (0.1% FA) to 5% water (0.1% FA) and 95% MeCN (0.1% TFA) in 2.0 min]: Rt=1.08 min; MS Calcd.: 286.2, MS Found: 287.2 [M+H]⁺.

Descriptions 104 and 105

### (S)-3-deuterium-5-methyl-6-(1-(tetrahydrofuran-3-yl)piperidin-4-yl)-1H-indazole and (R)-3-deuterium-5-methyl-6-(1-(tetrahydrofuran-3-yl)piperidin-4-yl)-1H-indazole

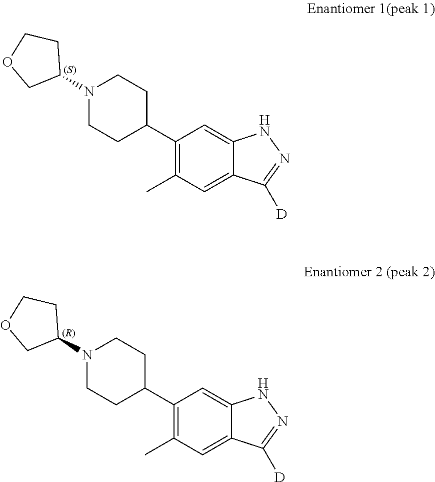

The compound 5-methyl-6-(1-(tetrahydrofuran-3-yl)piperidin-4-yl)-1H-indazole (D103, 1.00 g, 3.49 mmol) was purified by SFC to give the title products which were identified by known chiral intermediates via chiral HPLC system: (S)-3-deuterium-5-methyl-6-(1-(tetrahydrofuran-3-yl) piperidin-4-yl)-1H-indazole (peak 1, D104, 400 mg, yield: 40%) as a white solid and (R)-3-deuterium-5-methyl-6-(1-(tetrahydrofuran-3-yl)piperidin-4-yl)-1H-indazole (peak 2, D105, 350 mg, yield: 35%) as a white solid.

### Peak 1 (D104): (S)-3-deuterium-5-methyl-6-(1-(tetrahydrofuran-3-yl)piperidin-4-yl)-1H-indazole

¹H NMR (400 MHz, CDCl₃): δ 10.70 (br, 1H), 7.95 (s, 0.02H), 7.52 (s, 1H), 7.38 (s, 1H), 4.03˜3.93 (m, 2H), 3.86˜3.71 (m, 2H), 3.21-3.17 (m, 1H), 3.13˜3.05 (m, 1H), 2.99˜2.96 (m, 1H), 2.88˜2.80 (m, 1H), 2.44 (s, 3H), 2.31˜2.20 (m, 2H), 2.17˜2.08 (m, 1H), 2.02˜1.80 (m, 5H).

Chiral HPLC [method: Column: OJ, Column size: 3×100 mm, 3 um (Daicel) (UPC). Injection: 1 μl, Mobile phase: CO₂/MeOH/DEA: 90/10/0.01, Flow rate: 2.0 mL/min, Wave length: UV 254 nm, Temperature: 35° C.]: Rt=1.749 min, ee: 99.44%

### Peak 2 (D105): (R)-3-deuterium-5-methyl-6-(1-(tetrahydrofuran-3-yl)piperidin-4-yl)-1H-in-dazole

¹H NMR (400 MHz, CDCl₃): δ 10.76 (br, 1H), 7.95 (s, 0.02H), 7.52 (s, 1H), 7.38 (s, 1H), 4.03˜3.93 (m, 2H), 3.86˜3.71 (m, 2H), 3.21-3.18 (m, 1H), 3.13˜3.06 (m, 1H), 2.99˜2.96 (m, 1H), 2.88˜2.80 (m, 1H), 2.44 (s, 3H), 2.31˜2.21 (m, 2H), 2.16˜2.08 (m, 1H), 2.02˜1.80 (m, 5H).

Chiral HPLC [method: Column: OJ, Column size: 3×100 mm, 3 μm (Daicel) (UPC). Injection: 1 μl, Mobile phase: Supercritical CO₂/MeOH/NH₃.H₂O=90/10/0.01, Flow rate: 2.0 mL/min, Wave length: UV 254 nm, Temperature: 35° C.]: Rt=1.507 min, ee: 97.88%

Description 106

### 1-benzylpiperidin-2,2,6,6-d4-4-ol

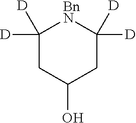 [SMILES: [2H]C1([2H])CC(O)CC([2H])([2H])N1Cc1ccccc1]

A solution of BnNH₂.TFA (9.00 g, 40.7 mmol) in CD₂O (20% in D₂O) (10.7 mL) was stirred at r.t for 10 min before allyltrimethylsilane (6.90 g, 61.1 mmol) was added. The

reaction mixture was stirred at 40° C. overnight under N₂, diluted with H₂O (10 mL), basified with K₂CO₃ to pH=10 and extracted with EtOAc three times. The combined organic phase was dried over Na₂SO₄ and concentrated. The crude product was purified by silica gel column (DCM/MeOH=10/1) to give the title product as a light yellow oil (5.40 g, yield 68.0%) which was used to next step directly.

Description 107

### tert-butyl 4-hydroxypiperidine-1-carboxylate-2,2,6,6-d4

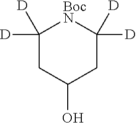 [SMILES: [2H]C1([2H])CC(O)CC([2H])([2H])N1C(=O)OC(C)(C)C]

To a mixture of 1-benzylpiperidin-2,2,6,6-d4-4-ol (5.40 g, 27.7 mmol) in EtOAc (100 mL) were added (Boc)₂O (7.20 g, 33.2 mmol) and Pd/C (600 mg). The reaction mixture was stirred at rt overnight under H₂, filtered and concentrated. The residue was purified by silica gel column (PE/EtOAc=3/1) to give the title product as a colorless oil (4.30 g, yield: 75.0%).

¹H NMR (400 MHz, CDCl₃): δ 3.86-3.81 (m, 1H), 1.85-1.81 (m, 2H), 1.71 (br, 2H), 1.45 (s, 9H).

Description 108

### tert-butyl 4-oxopiperidine-1-carboxylate-2,2,6,6-d₄

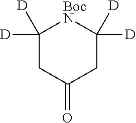 [SMILES: [2H]C1([2H])CC(=O)CC([2H])([2H])N1C(=O)OC(C)(C)C]

To a solution of tert-butyl 4-hydroxypiperidine-1-carboxylate-2,2,6,6-d₄ (6.40 g, 31.2 mmol) in dicholormethane (DCM) (300 mL) was added Dess-martin (19.8 g, 46.8 mmol). The mixture was stirred at rt for 2 hrs, filtered and concentrated. The residue was purified by silica gel column (PE/EtOAc=4/1) to give the title product as a slightly yellow solid (6.00 g, yield: 94.0%).

¹H NMR (400 MHz, CDCl₃): δ 2.43 (s, 4H), 1.49 (s, 9H).

Description 109

### tert-butyl 4-(((trifluoromethyl)sulfonyl)oxy)-3,6-dihydropyridine-1(2H)-carboxylate-2,2,6,6-d₄

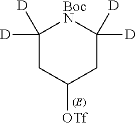 [SMILES: [2H]C1([2H])CC(OS(=O)(=O)C(F)(F)F)CC([2H])([2H])N1C(=O)OC(C)(C)C]

To a solution of tert-butyl 4-oxopiperidine-1-carboxylate-2,2,6,6-d₄ (270 mg, 1.33 mmol) in THF (8 mL) was added 1,1,1-trifluoro-N-phenyl-N-((trifluoromethyl)sulfonyl)meth-anesulfonamide (521 mg, 1.46 mmol). The mixture was cooled to −78° C. and LiHMDS (1.00 M, 1.60 mL) was added dropwise at the same temperature. The reaction mixture was stirred at r.t overnight under N₂, quenched with sat. NH₄Cl, extracted with EtOAc three times, dried and concentrated. The residue was purified by silica gel column (PE/EtOAc=20/1) to give the title product as a colorless oil (490 mg, crude).

¹H NMR (400 MHz, CDCl₃): δ 5.74 (s, 1H), 2.42 (s, 2H), 1.46 (s, 9H).

Description 110

### 5-methyl-1-(tetrahydro-2H-pyran-2-yl)-6-(4,4,5,5-tetramethyl-1,3,2-dioxaborolan-2-yl)-1H-indazole

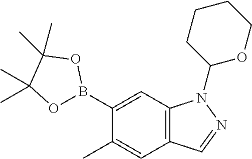 [SMILES: Cc1cc2cnn(C3CCCCO3)c2cc1B1OC(C)(C)C(C)(C)O1]

To a solution of 6-bromo-5-methyl-1-(tetrahydro-2H-pyran-2-yl)-1H-indazole (10.0 g, 33.9 mmol) in 1,4-dioxane (100 mL) was added 4,4,4′,4′,5,5,5′,5′-octamethyl-2,2′-bi(1,3,2-dioxaborolane) (10.3 g, 40.6 mmol), KOAc (10.0 g, 101.7 mmol) and Pd(dppf)Cl₂ (2.40 g, 3.40 mmol). The reaction mixture was stirred at 100° C. for 2 hrs under N₂. TLC (EtOAc:Petroleum Ether=1:10) showed the reaction was completed. The reaction mixture was poured into water (400 mL), extracted with EtOAc (100 mL×3). The combined organic layers were washed with brine (100 mL), dried over anhydrous Na₂SO₄, filtered and concentrated to dryness. The residue was purified by silica gel chromatography (EtOAc:Petroleum Ether=1:20 to 1:10) to give the title product 5-methyl-1-(tetrahydro-2H-pyran-2-yl)-6-(4,4,5,5-tetramethyl-1,3,2-dioxaborolan-2-yl)-1H-indazole (8.50 g, 73.3% yield) as a white solid.

LC-MS [mobile phase: from 90% water (0.1% FA) and 10% MeCN (0.1% T FA) to 5% water (0.1% FA) and 95% MeCN (0.1% FA) in 2.0 min]: Rt=0.79 min; MS Calcd.: 342.2; MS Found: 343.2 [M+H]⁺.

Description 111

### tert-butyl 4-(5-methyl-1-(tetrahydro-2H-pyran-2-yl)-1H-indazol-6-yl)-3,6-dihydrop-yridine-1(2H)-carboxylate-2,2,6,6-d4

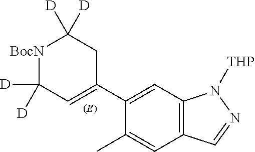

To a mixture of tert-butyl 4-(((trifluoromethyl)sulfonyl)oxy)-3,6-dihydropyridine-1(2H)-carboxylate-2,2,6,6-d4 (D110, 490 mg, 1.46 mmol) in dioxane (10 mL)/H₂O (2 mL) were added 5-methyl-1-(tetrahydro-2H-pyran-2-yl)-6-(4,4,5,5-tetramethyl-1,3,2-dioxaborolan-2-yl)-1H-indazole (D6, 500 mg, 1.46 mmol), PdCl₂(dppf) (107 mg, 0.150 mmol) and K₃PO₄ (620 mg, 2.92 mmol). The reaction mixture was stirred at 85° C. overnight under N₂, then filtered and the filtrate was extracted with EtOAc three times. The combined organic solution was dried and concentrated. The crude product was purified by silica gel column (PE/EtOAc=6/1) to give the title product as a white solid (183 mg, yield: 34%).

LC-MS [mobile phase: from 50% water (0.1% FA) and 50% MeCN (0.1% FA) to 5% water (0.1% FA) and 95% MeCN (0.1% FA) in 2 min]: Rt=1.40 min; MS Calcd.:401.2, MS Found: 402.5 [M+H]⁺.

Description 112

### tert-butyl 4-(5-methyl-1-(tetrahydro-2H-pyran-2-yl)-1H-indazol-6-yl)piperidine-1-carboxylate-2,2,6,6-d4

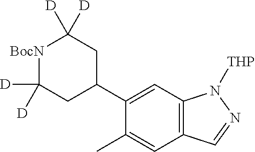

To a mixture of tert-butyl 4-(5-methyl-1-(tetrahydro-2H-pyran-2-yl)-1H-indazol-6-yl)-3,6-dihydropyridine-1(2H)-carboxylate-2,2,6,6-d₄ (D111, 183 mg, 0.456 mmol) in MeOH (10 mL) was added Pd/C (20 mg). The mixture was stirred at rt overnight under H₂ and filtered. The filtrate was concentrated to give the title product as a white solid (180 mg, yield: 98.0%) which was directly used into next step.

LC-MS [mobile phase: from 50% water (0.1% FA) and 50% MeCN (0.1% FA) to 5% water (0.1% FA) and 95% MeCN (0.1% FA) in 2 min]: Rt=1.37 min; MS Calcd.: 403.2, MS Found: 404.5 [M+H]⁺.

Description 113

### 5-methyl-6-(piperidin-4-yl-2,2,6,6-d4)-1H-indazole

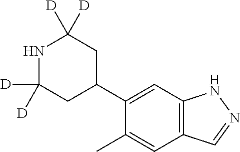 [SMILES: [2H]C1([2H])CC(c2cc3[nH]ncc3cc2C)CC([2H])([2H])N1]

To a solution of tert-butyl 4-(5-methyl-1-(tetrahydro-2H-pyran-2-yl)-1H-indazol-6-yl)piperidine-1-carboxylate-2,2,6,6-d4 (D112, 180 mg, 0.450 mmol) in DCM (4.00 mL) was added dropwise TFA (2.00 mL) at r.t. The reaction mixture was stirred at rt for 6 hrs, then concentrated, dissolved in MeOH (15 mL), basified with K₂CO₃ to pH=8, filtered and concentrated. The crude product was purified by silica gel column (DCM/MeOH=8/1) to give the title product as a light yellow solid (100 mg, yield: 97.0%).

LC-MS [mobile phase: from 90% water (0.1% FA) and 10% MeCN (0.1% FA) to 5% water (0.1% FA) and 95% MeCN (0.1% FA) in 2 min]: Rt=0.70 min; MS Calcd.:219.1, MS Found: 220.4 [M+H]⁺.

Description 114

### tert-butyl 4-(5-methyl-1H-indazol-6-yl)piperidine-1-carboxylate-2,2,6,6-d₄

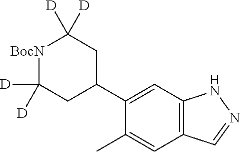 [SMILES: [2H]C1([2H])CC(c2cc3[nH]ncc3cc2C)CC([2H])([2H])N1C(=O)OC(C)(C)C]

To a mixture of 5-methyl-6-(piperidin-4-yl-2,2,6,6-d4)-1H-indazole (D113, 160 mg, 0.73 mmol) in MeOH (20 mL) was added a solution of NaOH (58.4 mg, 1.46 mmol) in H₂O (5 mL). Then (Boc)₂O (318 mg, 1.46 mmol) was added dropwise. The mixture was stirred at rt overnight, extracted with EtOAc three times. The combined organic layers were dried and concentrated. The residue was purified by silica gel chromatography (PE/EtOAc=3/1) to give the title product as a white solid (140 mg, yield: 62.0%).

¹H NMR (400 MHz, CDCl₃): δ 7.95 (s, 1H), 7.52 (s, 1H), 7.30 (s, 1H), 2.97˜2.91 (m, 1H), 2.44 (s, 3H), 1.83˜1.79 (m, 2H), 1.64˜1.58 (m, 2H), 1.51 (s, 9H).

Description 115

### tert-butyl (R)-4-(1-(6-(2-(hydroxymethyl)morpholino)-2-methylpyrimidin-4-yl)-5-m-ethyl-1H-indazol-6-yl)piperidine-1-carboxylate-2,2,6,6-d₄

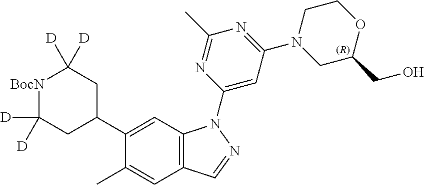

A mixture of tert-butyl 4-(5-methyl-1H-indazol-6-yl)piperidine-1-carboxylate-2,2,6,6-d4 (D114, 140 mg, 0.440 mmol), (R)-(4-(6-iodo-2-methylpyrimidin-4-yl)morpholin-2-yl)methanol (D12, 161 mg, 0.480 mmol), DMEDA (58.0 mg, 0.660 mmol), CuI (100 mg, 0.520 mmol) and K₃PO₄ (140 mg, 0.660 mmol) in toluene (10 mL) was stirred at 90° C. for 3 hrs under N₂, then concentrated. The residue was purified by silico gel chromatography column (PE/EtOAc=2:1) to give the title product as a light yellow solid (132 mg, yield: 57.0%).

LC-MS [mobile phase: from 90% water (0.1% FA) and 10% MeCN (0.1% FA) to 5% water (0.1% FA) and 95% MeCN (0.1% FA) in 2 min]: Rt=1.78 min; MS Calcd.:526.3, MS Found: 527.5 [M+H]⁺.

Description 116

### (R)-(4-(2-methyl-6-(5-methyl-6-(piperidin-4-yl-2,2,6,6-d4)-1H-indazol-1-Yl)pyrimi-din-4-yl)morpholin-2-yl)methanol

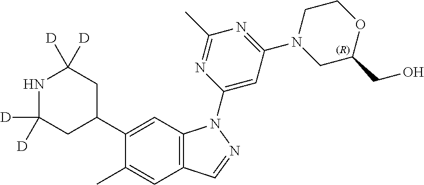

To a solution of tert-butyl (R)-4-(1-(6-(2-(hydroxymethyl)morpholino)-2-methylpy-rimidin-4-yl)-5-methyl-1H-indazol-6-yl)piperidine-1-carboxylate-2,2,6,6-d₄ (D115, 132 mg, 0.250 mmol) in DCM (3.00 mL) was added dropwise TFA (1.00 mL) at rt. The reaction mixture was stirred at rt for 30 min, concentrated, dissolved in MeOH, basified with K₂CO₃ to pH-8, filtered and concentrated. The crude product was purified by prep-HPLC (Waters 2767, Inertsil ODS-3 20×250 mm, 10 μM, mobile phase: MeCN/H₂O (10 mM NH₄HCO₃), from 19:81 to 95:5, flow rate: 20 mL/min, 254 nm) to give the title product as a white solid (65 mg, yield: 57%).

¹H NMR (400 MHz, CDCl₃): δ 8.78 (s, 1H), 8.05 (s, 1H), 7.50 (s, 1H), 6.95 (s, 1H), 4.33˜4.27 (m, 2H), 4.07˜4.04 (m, 1H), 3.80˜3.66 (m, 4H), 3.15˜3.08 (m, 1H), 2.98˜2.92 (m, 2H), 2.63 (s, 3H), 2.46 (s, 3H), 2.04 (br, 1H), 1.84-1.78 (m, 3H).

LC-MS [mobile phase: from 90% water (0.1% FA) and 10% MeCN (0.1% FA) to 5% water (0.1% FA) and 95% MeCN (0.1% FA) in 2 min]: Rt=1.11 min; MS Calcd.:426.2, MS Found: 427.5 [M+H]⁺.

Description 117

### 1-(1-(6-iodo-2-methoxypyrimidin-4-yl)azetidin-3-yl)ethanol

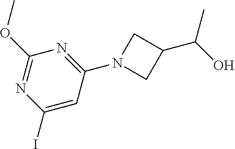 [SMILES: COc1nc(I)cc(N2CC(C(C)O)C2)n1]

A mixture of 3-methylazetidin-3-ol 2,2,2-trifluoroacetate (1.70 g, 8.00 mmol), 4,6-diiodo-2-methoxypyrimidine (2.88 g, 8.00 mmol) and DIPEA (2.06 g, 16.0 mmol) in EtOH/THF (10 mL/10 mL) was stirred at r.t. overnight, then concentrated. The residue was purified by silica gel chromatography (PE:EtOAc=5:1) to give the title product as a white solid (1.00 g, 38.0% yield).

LC-MS [mobile phase: from 80% water (0.1% FA) and 20% MeCN (0.1% FA) to 5% water (0.1% FA) and 95% MeCN (0.1% FA) in 2 min]: Rt=1.25 min; MS Calcd: 335, MS Found: 336 [M+H]⁺.

Description 118

### 4-(6-iodo-2-methylpyrimidin-4-yl)morpholine

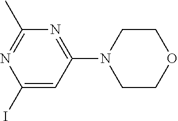

A mixture of 4,6-diiodo-2-methylpyrimidine (1.00 g, 2.90 mol), morpholine (870 mg, 10.0 mmol) and Et₃N (1.00 mL) in i-PrOH (20 mL) and THE (5 mL) was stirred at 40° C. overnight and concentrated. The residue was purified by silica gel chromatography (PE:EtOAc=5:1) to give the title product as an off-white solid (720 mg, 82% yield).

LC-MS [mobile phase: from 70% water (0.1% FA) and 30% MeCN (0.1% FA) to 5% water (0.1% FA) and 95% MeCN (0.1% FA) in 2.0 min]: Rt=0.45 min; MS Calcd.: 305, MS Found: 306.2 [M+H]⁺.

Description 119

### (R)-(4-(6-iodo-2-methylpyrimidin-4-yl)morpholin-3-yl)methanol

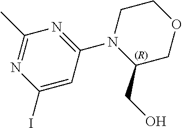

To a solution of 4,6-diiodo-2-methylpyrimidine (471 mg, 1.30 mmol) and (R)-morpholin-3-ylmethanol hydrochloride (200 mg, 1.30 mmol) in ⁱPrOH (10.0 mL) was added DIPEA (504 mg, 3.90 mmol) at rt. The reaction mixture was stirred at 70° C. for 48 h, then concentrated to dryness. The residue was purified by silica gel chromatography eluted with PE:EtOAc (2:1) to afford the title product as a white solid (280 mg, yield: 64%) which was directly used into next step

Description 120

### (S)-(4-(6-iodo-2-methylpyrimidin-4-yl)morpholin-3-yl)methanol

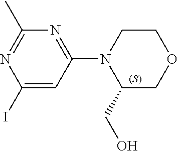

To a solution of 4,6-diiodo-2-methylpyrimidine (400 mg, 1.20 mmol) and (S)-morpholin-3-ylmethanol hydrochloride (184 mg, 1.20 mmol) in THF/EtOH=1/1 (30.0 mL) was added DIEA (465 mg, 3.60 mmol) at rt. The reaction mixture was stirred at 70° C. for 24 h, then concentrated to dryness. The residue was purified by silica gel chromatography eluted with PE:EtOAc=1:1 to afford the title product as a white solid (230 mg, yield: 59%).

LC-MS [mobile phase: from 50% water (0.1% FA) and 50% MeCN (0.1% FA) to 5% water (0.1% FA) and 95% MeCN (0.1% FA) in 2.6 min]: Rt=0.80 min; MS Calcd: 335.0, MS Found: 336.0 [M+H]⁺.

Description 121

### Trans-3-((6-iodo-2-methylpyrimidin-4-yl)amino)cyclobutanol

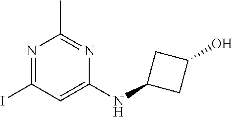

A mixture of trans-3-aminocyclobutanol hydrochloride (328 mg, 2.67 mmol), 4,6-diiodo-2-methylpyrimidine (923 mg, 2.67 mmol) and K₂CO₃ (1.10 g, 8.01 mmol) in DMF (20 mL) was stirred at 35° C. for 16 hours, then concentrated in vacuo. The residue was purified by column chromatography on silica gel (DCM to DCM/MeOH=20/1) to give the title product (690 mg, 85.0%) as a yellow oil.

¹H NMR (400 MHz, CDCl₃): δ 6.53 (s, 1H), 5.11-5.07 (m, 1H), 4.57-4.54 (m, 1H), 4.19-4.13 (m, 1H), 3.76-3.69 (m, 1H), 2.46 (s, 3H), 2.44-2.39 (m, 2H), 2.29-2.22 (m, 2H).

Description 122

### tert-butyl 4-(1-(6-((trans-3-hydroxycyclobutyl)amino)-2-methylpyrimidin-4-yl)-5-m-ethy-I-1H-indazol-6-yl)piperidine-1-carboxylate

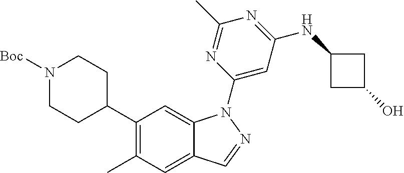 [SMILES: Cc1nc(N[C@H]2C[C@H](O)C2)cc(-n2ncc3cc(C)c(C4CCN(C(=O)OC(C)(C)C)CC4)cc32)n1]

A mixture of tert-butyl 4-(5-methyl-1H-indazol-6-yl)piperidine-1-carboxylate (206 mg, 0.655 mmol), trans-3-((6-iodo-2-methylpyrimidin-4-yl)amino)cyclobutanol (200 mg, 0.655 mmol), N,N′-dimethylcyclohexane-1,2-diamine (93.0 mg, 0.655 mmol), CuI (62.0 mg, 0.327 mmol) and K₃PO₄ (278 mg, 1.31 mmol) in toluene (4 mL) was stirred at 100° C. for 2 hours, then diluted with EtOAc(60 mL), washed with water (20 mL), ammonium hydroxide (2.00 mL) and brine (20.0 mL). The organic layer was dried over Na₂SO₄, filtered and concentrated. The residue was purified by column chromatography on silica gel (DCM to DCM/MeOH=30/1) to give the title product (150 mg, 46%) as a yellow solid.

¹H NMR (400 MHz, CDCl₃) δ 8.72 (s, 1H), 8.07 (s, 1H), 7.51 (s, 1H), 6.64 (s, 1H), 5.20 (s, 1H), 4.61-4.58 (m, 1H), 4.33-4.27 (m, 3H), 3.00-2.86 (m, 2H), 2.58 (s, 3H), 2.54-2.42 (m, 5H), 2.34-2.31 (m, 2H), 1.93-1.85 (m, 4H), 1.71-1.64 (m, 2H), 1.50 (s, 9H).

Description 123

### trans-3-((2-methyl-6-(5-methyl-6-(piperidin-4-yl)-1H-indazol-1-yl)pyrimidin-4-yl)amino)cyclobutanol

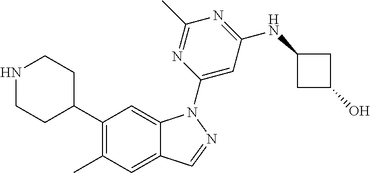 [SMILES: Cc1nc(N[C@H]2C[C@H](O)C2)cc(-n2ncc3cc(C)c(C4CCNCC4)cc32)n1]

To a solution of tert-butyl 4-(1-(6-((trans-3-hydroxycyclobutyl)amino)-2-methylpyrimi-din-4-yl)-5-methyl-1H-indazol-6-yl)piperidine-1-carboxylate (150 mg, 0.305 mmol) in DCM (4 mL) was added TFA (1 mL). The reaction mixture was stirred at room temperature for 1 hour, diluted with DCM (100 mL) and adjusted to pH>7 with sat.NaHCO₃. The separated aqueous layer was extracted with DCM/MeOH=10/1 (60 mL×3). The combined organic layers were dried over Na₂SO₄, filtered and concentrated to afford the title product (120 mg, 100%) as a yellow solid.

¹H NMR (400 MHz, DMSO-d₆) δ 8.95 (s, 1H), 8.28 (s, 1H), 7.75 (s, 1H), 7.60 (s, 1H), 5.16-5.06 (m, 1H), 4.33-4.06 (m, 1H), 3.38-3.35 (m, 2H), 3.15-3.11 (m, 2H), 2.97-2.90 (m, 1H), 2.76-2.67 (m, 2H), 2.49 (s, 3H), 2.42 (s, 3H), 2.38-2.36 (m, 1H), 2.21-2.15 (m, 4H), 1.79-1.74 (m, 2H), 1.63-1.55 (m, 2H).

Description 124

### cis-3-((6-iodo-2-methylpyrimidin-4-yl)amino)cyclobutanol

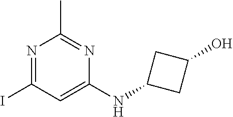

A mixture of cis-3-((6-iodo-2-methylpyrimidin-4-yl)amino)cyclobutanol (215 mg, 1.73 mmol), 4,6-diiodo-2-methylpyrimidine (500 mg, 1.44 mmol) and TEA (436 mg, 4.32 mmol) in DMSO (10.0 mL) was stirred at 6° C. for 5 hours, then diluted with H₂O (30.0 mL), extracted with EtOAc (30 mL×2), dried over Na₂SO₄, filtered and concentrated. The residue was purified by column chromatography on silica gel (petroleum ether/EtOAc=1:1) to give the title product containing DMSO (569 mg, 100%) as a yellow oil.

¹H NMR (400 MHz, CDCl₃): δ 6.58 (s, 1H), 5.73 (s, 1H), 4.09-4.13 (m, 1H), 3.69-3.49 (m, 1H), 2.87-2.80 (m, 2H), 2.42 (s, 3H), 1.91-1.84 (m, 2H).

Description 125

### tert-butyl 4-(1-(6-((cis-3-hydroxycyclobutyl)amino)-2-methylpyrimidin-4-yl)-5-meth-yl-1H-indazol-6-yl)piperidine-1-carboxylate

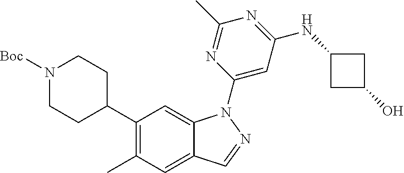 [SMILES: Cc1nc(N[C@H]2C[C@@H](O)C2)cc(-n2ncc3cc(C)c(C4CCN(C(=O)OC(C)(C)C)CC4)cc32)n1]

A mixture of tert-butyl 4-(5-methyl-1H-indazol-6-yl)piperidine-1-carboxylate (D99, 227 mg, 0.720 mmol), cis-3-((6-iodo-2-methylpyrimidin-4-yl)amino)cyclobutanol (220 mg, 0.720 mmol), N,N′-dimethylcyclohexane-1,2-diamine (102 mg, 0.720 mmol), CuI (68.0 mg, 0.360 mmol) and K₃PO₄ (305 mg, 1.44 mmol) in toluene (3.00 mL) was stirred at 100° C. for 3 hours, then diluted with EtOAc (60 mL), washed with NH₃H₂O (30 mL) and brine (30 mL). The organic layer was dried over Na₂SO₄, filtered and concentrated. The residue was purified by silica gel chromatography column (petroleum ether/EtOAc=1:1) to give the title product (277 mg, 78.0%) as a yellow oil.

LCMS [column: C₁₈; column size: 4.6×30 mm 5 μm; Dikwa Diamonsil plus; mobile phase: B (MeCN), A(0.02% NH₄Ac+5% MeCN in water); gradient (B %) in 4 mins. 10-95-POS; flow rate: 1.5 ml/min]: Rt=2.426 min; MS Calcd.:492, MS Found: 493 [M+H]⁺.

Description 126

### cis-3-((2-methyl-6-(5-methyl-6-(piperidin-4-yl)-1H-indazol-1-yl)pyrimidin-4-yl)amin-o)cyclobutanol

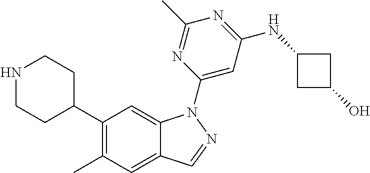 [SMILES: Cc1nc(N[C@H]2C[C@@H](O)C2)cc(-n2ncc3cc(C)c(C4CCNCC4)cc32)n1]

To a solution of tert-butyl 4-(1-(6-((cis-3-hydroxycyclobutyl)amino)-2-methylpyrimidin-4-yl)-5-methyl-1H-indazol-6-yl)piperidine-1-carboxylate (277 mg, 0.560 mmol) in DCM (4 mL) was added TFA (1 mL). The mixture was stirred at room temperature for 2 hrs, then adjusted to pH=9˜10 with Sat. NaHCO₃, diluted with H₂O (30 mL) and extracted with DCM (30 mL×2). The combined organic layers were dried over Na₂SO₄, filtered and concentrated to give the title product (209 mg, 95%) as a yellow oil.

¹H NMR (400 MHz, CDCl₃) δ 8.80 (s, 1H), 8.08 (s, 1H), 7.53 (s, 1H), 6.70 (s, 1H), 4.17-4.08 (m, 1H), 3.64-3.60 (m, 2H), 3.16-3.06 (m, 4H), 3.00-2.94 (m, 1H), 2.61 (s, 3H), 2.46 (s, 3H), 2.25-2.05 (m, 7H), 1.98-1.85 (m, 3H).

Description 127

### 1-(4-(6-iodo-2-methylpyrimidin-4-yl)morpholin-2-yl)ethanone

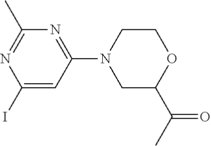 [SMILES: CC(=O)C1CN(c2cc(I)nc(C)n2)CCO1]

To a solution of 4,6-diiodo-2-methylpyrimidine (2.1 g, 6.0 mmol) and 2-acetylmorpholin-4-ium chloride (1.0 g, 6.0 mmol) in THF/EtOH=1/1 (30 mL/30 mL) was added DIEA (3.10 g, 24.0 mmol) at rt. The reaction mixture was stirred at room temperature for 5 h and concentrated to dryness. The residue was purified by silica gel chromatography eluted with PE:EtOAc=10:1 to afford the title product as a white solid (1.50 g, yield: 71.0%).

LC-MS [mobile phase: from 50% water (0.1% FA) and 50% MeCN (0.1% FA) to 5% water (0.1% FA) and 95% MeCN (0.1% FA) in 2.6 min]: Rt=1.04 min; MS Calcd: 347.0, MS Found: 348.0 [M+H]⁺.

Description 128

### 1-(4-(2-methyl-6-(5-methyl-6-(1-(tetrahydrofuran-3-yl)piperidin-4-yl)-1H-indazol-1-yl)pyrimidin-4-yl)morpholin-2-yl)ethanone

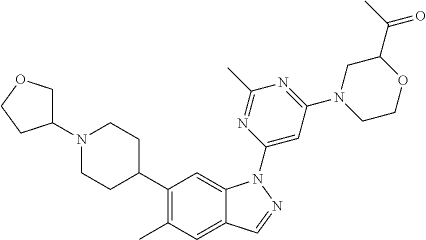 [SMILES: CC(=O)C1CN(c2cc(-n3ncc4cc(C)c(C5CCN(C6CCOC6)CC5)cc43)nc(C)n2)CCO1]

To a stirred solution of 1-(4-(6-iodo-2-methylpyrimidin-4-yl)morpholin-2-yl)ethanone (1.20 g, 3.50 mmol) and 5-methyl-6-(1-(tetrahydrofuran-3-yl)piperidin-4-yl)-1H-indazole (1.00 g, 3.50 mmol) in toluene (50.0 ml) were added CuI (1.00 g, 5.30 mmol), K₃PO₄.3H₂O (1.90 g, 7.00 mmol) and N,N′-dimethylethylenediamine (617 mg, 7.00 mmol). The reaction mixture was stirred at 100° C. for 5 h and concentrated. The residue was purified by silica gel chromatography eluted with PE:EtOAc (1:2) to give the title product as a pale yellow solid (570 mg, yield: 32.0%).

LC-MS [mobile phase: from 50% water (0.1% FA) and 50% MeCN (0.1% FA) to 5% water (0.1% FA) and 95% MeCN (0.1% T FA) in 2.6 min]: Rt=0.83 min; MS Calcd: 504.6, MS Found: 505.3 [M+H]⁺.

Description 129

### (R)-6-iodo-2-methyl-N-(tetrahydrofuran-3-yl)pyrimidin-4-amine

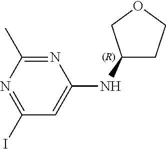

To a solution of 4,6-diiodo-2-methylpyrimidine (1.99 g, 5.75 mmol) and (R)-tetrahydrofuran-3-amine (500 mg, 1.44 mmol) in DMSO (15.0 mL) was added TEA (1.74 g, 17.3 mmol). The reaction mixture was stirred at 60° C. overnight, diluted with H₂O (60 mL) and extracted with EtOAc (100 mL×3). The combined organic layers were dried over Na₂SO₄, filtered and concentrated. The residue was purified by column chromatography on silica gel (petroleum ether/EtOAc=5/1) to afford the title product as a yellow solid. (1.35 g, 77.0%)

¹H NMR (400 MHz, CDCl₃): δ 6.64 (s, 1H), 5.00-4.94 (m, 1H), 4.42-3.33 (m, 1H), 3.97-3.84 (m, 3H), 3.83-3.68 (m, 1H), 2.48 (s, 3H), 2.39-2.27 (m, 1H), 1.89-1.81 (m, 1H).

Description 130

### (R)-tert-butyl 4-(5-methyl-1-(2-methyl-6-((tetrahydrofuran-3-yl)amino)pyrimidin-4yl)-1H-indazol-6-yl)piperidine-1-carboxylate

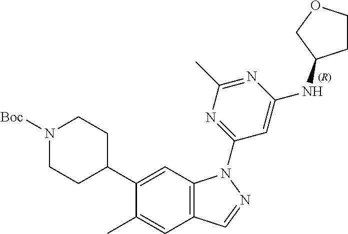

A mixture of tert-butyl 4-(5-methyl-1H-indazol-6-yl)piperidine-1-carboxylate (200 mg, 0.630 mmol), (R)-6-iodo-2-methyl-N-(tetrahydrofuran-3-yl)pyrimidin-4-amine (210 mg, 0.690 mmol), CuI (60.0 mg, 0.320 mmol), K₃PO₄ (267 mg, 1.26 mmol) and N,N′-dimethylcyclohexane-1,2-diamine (89.0 mg, 0.630 mmol) in toluene (3.00 mL) was stirred at 100° C. for 3 hours under N₂, diluted with EtOAc (50.0 mL), washed with NH₃.H₂O (30 mL×3). The combined organic layers were dried over Na₂SO₄, filtered and concentrated. The residue was purified by silica gel chromatography column (petroleum ether/EtOAc=1/1) to give the title product (295 mg, 95%) as a white solid.

¹H NMR (400 MHz, CDCl₃): δ 8.75 (s, 1H), 8.07 (s, 1H), 7.51 (s, 1H), 6.77 (s, 1H), 5.14-5.13 (m, 1H), 4.44-4.32 (m, 3H), 4.15-4.10 (m, 2H), 4.03-3.85 (m, 1H), 3.77-3.73 (m, 1H), 3.00-2.83 (m, 3H), 2.60 (s, 3H), 2.47 (s, 3H), 2.05 (s, 2H), 1.96-1.87 (m, 4H), 1.51 (s, 9H).

Description 131

### (R)-2-methyl-6-(5-methyl-6-(piperidin-4-yl)-1H-indazol-1-yl)-N-(tetrahydrofuran-3-Y)pyrimidin-4-amine

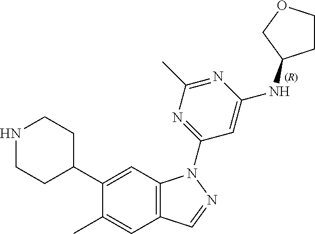

To a solution of (R)-tert-butyl 4-(5-methyl-1-(2-methyl-6-((tetrahydrofuran-3-yl)amino)pyrimidin-4-yl)-1H-indazol-6-yl)piperidine-1-carboxylate (D132, 295 mg, 0.600 mmol) in DCM (4.00 mL) was added TFA (1.00 mL). The mixture was stirred at room temperature for 1 hour, adjusted to pH>7 with sat.NaHCO₃, diluted with H₂O (50 mL) and extracted with EtOAc (30 mL×3). The combined organic layers were dried over Na₂SO₄, filtered and concentrated to give the title product (235 mg, 100%) as a white solid.

¹H NMR (400 MHz, CDCl₃): δ 8.82 (s, 1H), 8.08 (s, 1H), 7.53 (s, 1H), 6.77 (s, 1H), 5.19-5.17 (m, 1H), 4.45 (s, 1H), 4.03-3.86 (m, 3H), 3.77-3.68 (m, 1H), 3.55-3.49 (m, 2H), 3.09-3.03 (m, 3H), 2.61 (s, 3H), 2.47 (s, 3H), 2.10-1.92 (m, 7H).

Description 132

### (S)-6-iodo-2-methyl-N-(tetrahydrofuran-3-yl)pyrimidin-4-amine

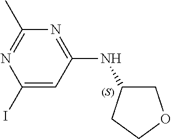

The title product was prepared by a procedure similar to that described for D129 starting from a solution of (S)-tetrahydrofuran-3-amine, 4,6-diiodo-2-meth-ylpyrimidine and TEA in DMSO.

LCMS [column: C₁₈; column size: 4.6×30 mm 5 μm; Dikwa Diamonsil plus; mobile phase: B (MeCN), A (0.02% NH₄Ac+5% MeCN in water); gradient (B %) in 4 min-5-95-POS; flow 1.5 mL/min, stop time 4 mins]: Rt=1.801 min]: MS Calcd.: 305, MS Found: 306 [M+H]⁺.

Description 133

### (S)-tert-butyl 4-(5-methyl-1-(2-methyl-6-((tetrahydrofuran-3-yl)amino)pyrimidin-4-yl)-1H-indazol-6-yl)piperidine-1-carboxylate

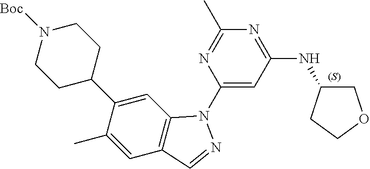

The title product was prepared by a procedure similar to that described for D130 starting from a mixture of tert-butyl 4-(5-methyl-1H-indazol-6-yl)piperidine-1-carboxylate (D99), (S)-6-iodo-2-methyl-N-(tetrahydrofuran-3-yl)pyrimidin-4-amine (D132), N,N′-dimethylc-yclohexane-1,2-dia-mine, CuI and K₃PO₄ in toluene.

¹HNMR (400 MHz, CDCl₃): δ 8.74 (s, 1H), 8.06 (s, 1H), 7.51 (s, 1H), 5.13 (br s, 1H), 4.03-3.88 (m, 4H), 3.77-3.73 (m, 1H), 2.97-2.85 (m, 4H), 2.59 (s, 3H), 2.47 (s, 3H), 2.39-2.33 (m, 2H), 1.94-1.88 (m, 5H), 1.50 (s, 9H).

Description 134

### (S)-2-methyl-6-(5-methyl-6-(piperidin-4-yl)-1H-indazol-1-yl)-N-(tetrahydrofuran-3-yl)pyrimidin-4-amine

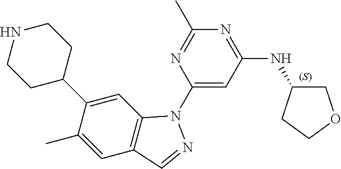

The title product was prepared by a procedure similar to that described for D133 starting from a solution of (S)-tert-butyl 4-(5-methyl-1-(2-methyl-6-((tetrahydrofuran-3-yl)amino)pyrimidin-4-yl)-1H-indazol-6-yl)piperidine-1-carboxylate (D135) in DCM.

LCMS [column: C₁₈; column size: 4.6×30 mm 5 μm; Dikwa Diamonsil plus; mobile phase: B (MeCN), (0.02% NH₄Ac+5% MeCN in water); gradient (B %) in 4 min-10-95-POS; flow 1.5 mL/min, stop time 4 mins]: Rt=1.644 min; MS Calcd.: 392, MS Found: 393 [M+H]⁺.

Description 135

### 3-(benzyloxy)cyclobutanol

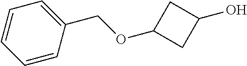 [SMILES: OC1CC(OCc2ccccc2)C1]

To a mixture of 3-(benzyloxy)cyclobutanone (5.00 g, 28.4 mmol) in MeOH (30.0 mL) was added NaBH₄ (3.20 g, 85.1 mmol). The reaction mixture was stirred at room temperature overnight, diluted with sat.NH₄Cl (100 mL) and extracted with EtOAc (100 mL×3). The combined organic layers were dried over Na₂SO₄, filtered and concentrated to give title product (4.98 g, 98.0%) as a yellow oil.

¹HNMR (400 MHz, CDCl₃): δ 7.36-7.28 (m, 5H), 4.41 (s, 2H), 3.90-3.86 (m, 1H), 3.65-3.58 (m, 1H), 2.73-2.67 (m, 2H), 2.19-2.17 (m, 1H), 1.96-1.89 (m, 2H).

Description 136

### 4-(3-(benzyloxy)cyclobutoxy)-6-iodo-2-methylpyrimidine

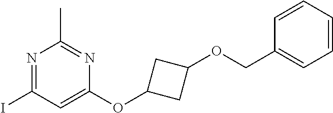 [SMILES: Cc1nc(I)cc(OC2CC(OCc3ccccc3)C2)n1]

To a solution of 3-(benzyloxy)cyclobutanol (2.00 g, 11.0 mmol) in THE (16.0 mL) was added NaH (560 mg, 14.0 mmol, 60.0% in mineral oil). After 1 hour at 0° C., 4,6-diiodo-2-methoxypyrimidine (3.20 g, 9.30 mmol) was added. The reaction mixture was gradually allowed to room temperature and stirred for 3 hours, then quenched with 10 drops of sat. NH₄Cl and extracted with EtOAc (30 mL×3). The combined organic layers were washed with water (100 mL) and concentrated. The residue was purified by silica gel chromatography column (petroleum ether/EtOAc=5/1) to give the title product (2.70 g, 72.0%) as a colorless oil.

¹HNMR (400 MHz, CDCl₃): δ 7.37-7.28 (m, 5H), 7.00 (s, 1H), 4.86-4.83 (m, 1H), 4.44 (s, 2H), 3.82-3.78 (m, 1H), 2.89-2.83 (m, 2H), 2.54 (s, 3H), 2.19-2.12 (m, 2H).

Description 137

### tert-butyl 4-(1-(6-(3-(benzyloxy)cyclobutoxy)-2-methylpyrimidin-4-yl)-5-methyl-1H-indazol-6-yl)piperidine-1-carboxylate

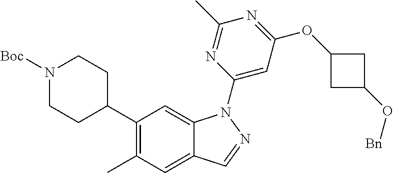 [SMILES: Cc1nc(OC2CC(OCc3ccccc3)C2)cc(-n2ncc3cc(C)c(C4CCN(C(=O)OC(C)(C)C)CC4)cc32)n1]

A mixture of tert-butyl 4-(5-methyl-1H-indazol-6-yl)piperidine-1-carboxylate (D99, 945 mg, 3.00 mmol), 4-(3-(benzyloxy)cyclobutoxy)-6-iodo-2-methylpyrimidine (D136, 1.30 g, 3.30 mmol), N,N′-dimethylcyclohexane-1,2-diamine (426 mg, 3.00 mmol), CuI (285 mg, 1.50 mmol) and K₃PO₄ (1.30 g, 6.00 mmol) in toluene (4.00 mL) was stirred at 100° C. for 2 hours, diluted with EtOAc (50 mL) and washed with NH₃.H₂O (30×3 mL). The combined organic layers were dried over Na₂SO₄, filtered and concentrated. The residue was purified by silica gel chromatography column (petroleum ether/EtOAc=1:1) to give the title product (1.55 g, 88.0%) as a colorless oil.

¹HNMR (400 MHz, CDCl₃): δ 8.72 (s, 1H), 8.08 (s, 1H), 7.51 (s, 1H), 7.36-7.35 (m, 5H), 7.06 (s, 1H), 4.92-4.87 (m, 1H), 4.47 (s, 3H), 4.33 (br s, 2H), 3.86-3.83 (m, 1H), 3.01-2.83 (m, 6H), 2.67 (s, 3H), 2.47 (s, 3H), 2.25-2.22 (m, 2H), 1.89-1.86 (m, 2H), 1.51 (s, 9H).

Description 138

### 1-(6-(3-(benzyloxy)cyclobutoxy)-2-methylpyrimidin-4-yl)-5-methyl-6-(piperidin-4-yl)-1H-indazole

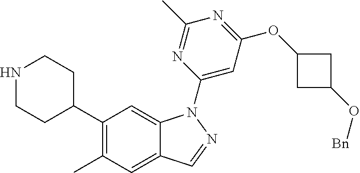 [SMILES: Cc1nc(OC2CC(OCc3ccccc3)C2)cc(-n2ncc3cc(C)c(C4CCNCC4)cc32)n1]

To a solution of tert-butyl 4-(1-(6-(3-(benzyloxy)cyclobutoxy)-2-methylpyrimidin-4-yl)-5-methyl-1H-indazol-6-yl)piperidine-1-carboxylate (D137, 1.55 g, 2.70 mmol) in DCM (6.00 mL) was added TFA (4.00 mL). The reaction mixture was stirred at room temperature for 2 hours, adjusted to pH>7 with sat.NaHCO₃, diluted with H₂O (50 mL) and extracted with EtOAc (30 mL×3). The combined organic layers were dried over Na₂SO₄, filtered and concentrated to give the title product (1.28 g, 100%) as a colorless oil.

LCMS [column: C₁₈; column size: 4.6×30 mm 5 μm; Dikwa Diamonsil plus; mobile phase: B (MeCN), A (0.02% NH₄Ac+5% MeCN in water); gradient (B %) in 2.5 mins. 70-95-POS; flow rate: 1.5 ml/min]: Rt=1.59 min; MS Calcd.:483, MS Found: 484 [M+H]⁺.

Description 139

### 3-((2-methyl-6-(5-methyl-6-(piperidin-4-yl)-1H-indazol-1-yl)pyrimidin-4-yl)oxy)cyclobutanol

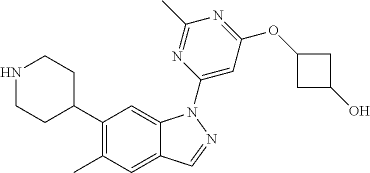 [SMILES: Cc1nc(OC2CC(O)C2)cc(-n2ncc3cc(C)c(C4CCNCC4)cc32)n1]

A mixture of 1-(6-(3-(benzyloxy)cyclobutoxy)-2-methylpyrimidin-4-yl)-5-methyl-6-(pi-peridin-4-yl)-1H-indazole (1.28 g, 2.60 mmol) and Pd(OH)₂ (256 mg, 20.0% W) in MeCOH (50.0 mL) was hydrogenated at 50° C. under 50 Psi for 4 days, then filtered and concentrated to give the title product (1.00 g, 96.0%) as a white solid.

LCMS [column: C₁₈; column size: 4.6×30 mm 5 μm; Dikwa Diamonsil plus; mobile phase: B (MeCN), A (0.02% NH₄Ac+5% MeCN in water); gradient (B %) in 4 mins. 05-95-POS; flow rate: 1.5 ml/min]: Rt=1.671 min; MS Calcd.:393, MS Found: 394 [M+H]⁺.

Description 140

### 4-(4-(5-methyl-1-(tetrahydro-2H-pyran-2-yl)-1H-indazol-6-yl)piperidin-1-yl)tetrahydrofuran-3-ol

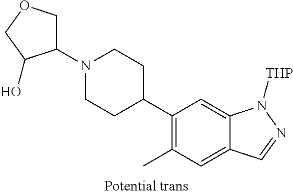

To a solution of 5-methyl-6-(piperidin-4-yl)-1-(tetrahydro-2H-pyran-2-yl)-1H-indazole (1.20 g, 4.01 mmol) in DMF (15.0 mL) were added Cs₂CO₃ (3.90 g, 12.0 mmol) and 3,6-dioxabicyclo[3.1.0]hexane (1.38 g, 16.0 mmol). The reaction mixture was stirred at 80° C. for 18 hours, cooled, diluted with water (150 mL) and extracted with EtOAc (40.0 mL×3). The combined organic solution was washed with brine (100 mL×2), dried over Na₂SO₄ and concentrated. The residue was purified by chromatography (MeOH/DCM=1/100˜1/10) to give the title product as a yellow solid (660 mg, 43.0% yield).

LC-MS [mobile phase: from 60% water (0.1% FA) and 40% MeCN (0.1% FA) to 5% water (0.1% FA) and 95% MeCN (0.1% FA) in 2.0 min]: Rt=0.52 min; MS Calcd.:385.24, MS Found: 386.4 [M+H]⁺.

Description 141

### 4-(4-(5-methyl-1H-indazol-6-yl)piperidin-1-yl)tetrahydrofuran-3-ol

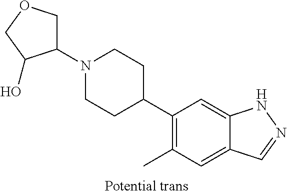 [SMILES: Cc1cc2cn[nH]c2cc1C1CCN(C2COCC2O)CC1]

To a solution of 4-(4-(5-methyl-1-(tetrahydro-2H-pyran-2-yl)-1H-indazol-6-yl)piperidin-1-yl)tetrahydrofuran-3-ol (D140, 400 mg, 1.04 mmol) in CH₂Cl₂ (10.0 mL) was added dropwise TFA (2.00 mL). The reaction mixture was stirred at room temperature overnight, concentrated, diluted with EtOAc (20.0 mL), washed with sat. NaHCO₃ (20.0 mL×2) and brine (20.0 mL), dried over anhydrous Na₂SO₄ and concentrated. The residue was purified by silica gel column (CH₂Cl₂:MeOH=10:1) to give the title product (250 mg, yield: 80.0%) as a pale yellow solid.

LC-MS [mobile phase: from 70% water (0.1% FA) and 30% MeCN (0.1% FA) to 5% water (0.1% FA) and 95% MeCN (0.1% FA) in 9.0 min]: Rt=3.48 min; MS Calcd: 301.2, MS Found: 302.2 [M+H]⁺.

Description 142

### 6-(1-(4-fluorotetrahydrofuran-3-yl)piperidin-4-yl)-5-methyl-1-(tetrahydro-2H-pyran-2-yl)-1H-indazole

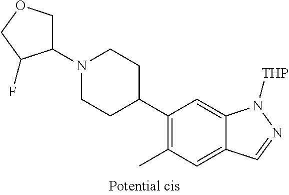

To a solution of 4-(4-(5-methyl-1-(tetrahydro-2H-pyran-2-yl)-1H-indazol-6-yl)piperidin-1-yl)tetrahydrofuran-3-ol (600 mg, 1.57 mmol) in DCM (15.0 mL) was added DAST (756 mg, 4.70 mmol) at −70° C. The reaction mixture was warmed to 10° C. and then 30° C. for 1 hour, poured into sat. NaHCO₃ (50 mL) and extracted with DCM (20 mL×3). The combined organic layers were washed with brine, dried and concentrated. The residue was purified by chromatography (MeOH/DCM=1/100 1/30) to give the title product (350 mg, 58% yield).

LC-MS [mobile phase: from 40% water (0.1% FA) and 60% MeCN (0.1% FA) to 5% water (0.1% FA) and 95% MeCN (0.1% FA) in 2.0 min]: Rt=0.25 min; MS Calcd.:387.23, MS Found: 388.4 [M+H]⁺.

Description 143

### 6-(1-(4-fluorotetrahydrofuran-3-yl)piperidin-4-yl)-5-methyl-1H-indazole

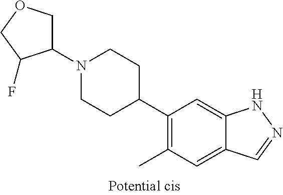 [SMILES: Cc1cc2cn[nH]c2cc1C1CCN(C2COCC2F)CC1]

To a solution of 6-(1-(4-fluorotetrahydrofuran-3-yl)piperidin-4-yl)-5-methyl-1-(tetrah-ydro-2H-pyran-2-yl)-1H-indazole (D142, 350 mg, 0.900 mmol) in DCM (6.00 mL) was added TFA (3.00 mL). The reaction mixture was stirred at 5˜10° C. for 18 hours, concentrated, re-dissolved in DCM (10 mL), treated with sat. NaHCO₃ (30 mL) and extracted with DCM (15 mL×3). The combined organic layers were washed with brine, dried and concentrated to give the crude product (280 mg) as a yellow solid which was used to next step without further purification.

Description 144

### 4-(4-(5-methyl-1-(tetrahydro-2H-pyran-2-yl)-1H-indazol-6-yl)piperidin-1-yl)dihydr-ofuran-3(2H)-one

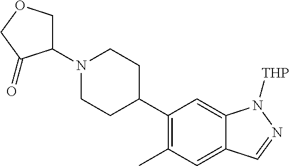

To a solution of DMSO (1.50 g, 19.5 mmol) in CH₂Cl₂ (30.0 mL) was added dropwise a solution of oxalyl chlorie (1.20 g, 9.34 mmol) in CH₂Cl₂ (5.00 mL) at −65° C. under N₂. After the reaction mixture was stirred at −65° C.˜−60° C. for 20 min, a solution of 4-(4-(5-methyl-1-(tetrahydro-2H-pyran-2-yl)-1H-indazol-6-yl)piperidin-1-yl)tetrahydrofuran-3-ol (D140, 3.00 g, 7.78 mmol) in CH₂Cl₂ (5.00 mL) was added dropwise. After 20 min at −60° C.−55° C., Et₃N (3.90 g, 38.9 mmol) was added dropwise. The reaction mixture was stirred at −55° C. for 20 min, then quenched with water, diluted with CH₂Cl₂ (60 mL) and washed with brine (2×60 mL). The organic layer was dried over anhydrous Na₂SO₄ and concentrated. The residue was purified by silica gel column (CH₂Cl₂: MeOH=80:1) to give the title product (2.09 g, yield: 67.0%) as a pale yellow solid.

LC-MS [mobile phase: from 70% water (0.1% FA) and 30% MeCN (0.1% FA) to 5% water (0.1% FA) and 95% MeCN (0.1% FA) in 2.0 min]: Rt=0.96 min; MS Calcd: 383.2, MS Found: 384.4 [M+H]⁺.

Description 145

### 4-(4-(5-methyl-1-(tetrahydro-2H-pyran-2-yl)-1H-indazol-6-yl)piperidin-1-yl)tetra-hydrofuran-3-ol

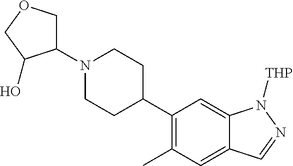

To a solution of 4-(4-(5-methyl-1-(tetrahydro-2H-pyran-2-yl)-1H-indazol-6-yl)piperidin-1-yl)dihydrofuran-3(2H)-one (D144, 2.00 g, 5.22 mmol) in MeOH (20.0 mL) was added NaBH₄ (592 mg, 15.7 mmol). The reaction mixture was stirred at room temperature for 60 min, then quenched with aq. 1N HCl, diluted with CH₂Cl₂ (100 mL), washed with sat. NaHCO₃ (100 mL) and brine (100 mL), dried over anhydrous Na₂SO₄, filtered and concentrated. The residue was purified by silica gel column (CH₂Cl₂: MeOH=10:1) to give the title product (1.60 g, yield: 80.0%) as a white solid.

LC-MS [mobile phase: from 70% water (0.1% FA) and 30% MeCN (0.1% FA) to 5% water (0.1% FA) and 95% MeCN (0.1% FA) in 2.0 min]: Rt=0.68 min; MS Calcd: 385.2, MS Found: 386.5[M+H]⁺.

Description 146

### methyl 4-oxotetrahydrofuran-3-carboxylate

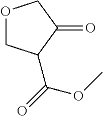

To a suspension of NaH (3.30 g, 85.5 mmol) in DMF (5.00 mL) was added dropwise methyl 2-hydroxyacetate (7.50 g, 85.5 mmol) at 0° C. After the reaction mixture was stirred at 0° C. for 30 min, methyl acrylate (8.30 mL, 93.0 mmol) was added dropwise at 0° C. The reaction mixture was allowed to room temperature and stirred overnight, diluted with water (200 mL) and extracted with EtOAc (200 mL×3). The combined organic layers were washed with water (400 mL×3) and brine (400 mL), concentrated and purified by silica gel column chromatography (PE:EtOAc=10:1) to give the title product as a colorless oil (2.20 g, 18.0 yield).

LC-MS [mobile phase: from 60% water (0.1% FA) and 40% MeCN (0.1% FA) to 5% water (0.1% FA) and 95% MeCN (0.1% FA) in 2.6 min]: Rt=0.78 min; MS Calcd.: 144.04, MS Found: 145.4 [M+H]⁺.

Description 147

### methyl 3-methyl-4-oxotetrahydrofuran-3-carboxylate

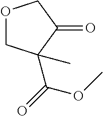

To a solution of methyl 4-oxotetrahydrofuran-3-carboxylate (1.90 g, 13.2 mmol) in DMF (20.0 mL) was added NaH (633 mg, 15.8 mmol) in two portions at 0° C. After 30 min Mel (1.67 mL, 26.4 mmol) was added dropwise at 0° C. The reaction mixture was allowed to room temperature and stirred overnight, quenched with sat. NH₄Cl and extracted with EtOAc (100 mL×3). The combined organic layers were washed with water (150 mL×3) and brine (150 mL), dried over anhydrous Na₂SO₄, filtered, concentrated and purified by silica gel column chromatography (PE:EtOAc=15:1) to give the title product as a colorless oil (750 mg, 36.1% yield).

LC-MS [mobile phase: from 60% water (0.1% FA) and 40% MeCN (0.1% FA) to 5% water (0.1% FA) and 95% MeCN (0.1% FA) in 2.6 min]: Rt=0.97 min; MS Calcd.:158.06, MS Found: 159.3 [M+H]⁺.

¹H NMR (400 MHz, CDCl₃): δ 4.62˜4.59 (d, J=9.6 Hz, 1H), 4.16˜4.12 (d, J=17.2 Hz, 1H), 4.04˜3.99 (d, J=17.2 Hz, 1H), 3.97˜3.95 (d, J=9.6 Hz, 1H), 3.76 (s, 3H), 1.42 (s, 3H).

Description 148

### 4-methyldihydrofuran-3(2H)-one

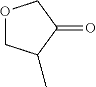

A mixture of methyl 3-methyl-4-oxotetrahydrofuran-3-carboxylate (200 mg, 1.26 mmol) in 10% H₂SO₄ (5.00 mL) was stirred at 100° C. for 1 h, diluted with water (10 mL) and extracted with EtOAc (20 mL×3). The combined organic layers were washed with brine (20 mL), dried over anhydrous Na₂SO₄ and concentrated to give the crude product (150 mg)

Description 149

### 5-methyl-6-(1-(4-methyltetrahydrofuran-3-yl)piperidin-4-yl)-1H-indazole

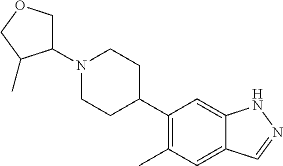 [SMILES: Cc1cc2cn[nH]c2cc1C1CCN(C2COCC2C)CC1]

To a solution of 4-methyldihydrofuran-3(2H)-one (150 mg, 1.50 mmol) and 5-methyl-6-(piperidin-4-yl)-1H-indazole (D9, 322 mg, 1.50 mmol) in CH₂Cl₂ (20.0 mL) and MeOH (5.00 mL) were added AcOH (54.0 mg, 0.900 mmol) and NaBH₃CN (235 mg, 3.80 mmol). The reaction mixture was stirred at 45° C. overnight, diluted with water (20.0 mL) and extracted with CH₂Cl₂ (20.0 mL×3). The combined organic layers were washed with brine (30.0 mL), dried, concentrated and purified by silica gel column chromatography (PE:EtOAc=1:5) to give the title product as a white solid (45.0 mg, 10.0% yield) LC-MS [mobile phase: from 70% water (0.1% FA) and 30% MeCN (0.1% FA) to 5% water (0.1% FA) and 95% MeCN (0.1% FA) in 2.6 min]: Rt=0.65 min; MS Calcd.:299.20, MS Found: 300.4 [M+H]⁺.

¹H NMR (400 MHz, CDCl₃): δ 10.11 (brs, 1H), 7.95 (s, 1H), 7.52 (s, 1H), 7.38 (s, 1H), 4.00˜3.94 (m, 2H), 3.74˜3.68 (m, 2H), 3.13˜3.10 (m, 1H), 2.86˜2.77 (m, 3H), 2.44 (s, 3H), 2.36˜2.32 (m, 1H), 2.21˜2.17 (m, 1H), 2.10˜2.07 (m, 1H), 1.86˜1.81 (m, 4H), 1.07˜1.06 (d, J=6.8 Hz, 3H).

Description 150

### N-(2-hydroxyethyl)-4-methylbenzenesulfonamide

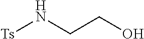 [SMILES: Cc1ccc(S(=O)(=O)NCCO)cc1]

To a solution of 2-aminoethanol (3.52 g, 57.6 mmol) and TEA (13.2 g, 131 mmol) in DCM (250 mL) was added dropwise TsCl (10.0 g, 52.4 mmol) in DCM (50 mL) at 0° C. The reaction mixture was stirred at room temperature overnight and diluted with H₂O (200 mL). The organic layer was washed with 1 N HCl (150 mL) and brine (100 mL), dried over Na₂SO₄, filtered and concentrated to give the title product (10.0 g, 89.0%) as a colorless oil.

¹H NMR (400 MHz, CDCl₃): δ 7.76 (d, J=8.0 Hz, 2H), 7.32 (d, J=7.6 Hz, 2H), 4.91 (t, J=5.8 Hz, 1H), 3.70 (t, J=3.6 Hz, 2H), 3.12-3.08 (m, 2H), 2.43 (s, 3H), 1.90 (s, 1H).

Description 151

### N-(2-hydroxyethyl)-4-methyl-N-(2-methylallyl)benzenesulfonamide

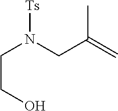 [SMILES: C=C(C)CN(CCO)S(=O)(=O)c1ccc(C)cc1]

A mixture of N-(2-hydroxyethyl)-4-methylbenzenesulfonamide (9.00 g, 41.9 mmol), 3-bromo-2-methylprop-1-ene (6.78 g, 50.2 mmol) and K₂CO₃ (11.6 g, 83.7 mmol) in acetone (100 mL) was refluxed overnight, diluted with H₂O (150 mL) and extracted with EtOAc (100 mL×2). The combined organic layers were washed with brine (50 mL), dried over Na₂SO₄ and concentrated to give the title product (10.7 g, 95%) as a white solid.

¹H NMR (400 MHz, CDCl₃): δ 7.72 (d, J=8.4 Hz, 2H), 7.32 (d, J=8.4 Hz, 2H), 4.91 (d, J=15.6 Hz, 2H), 3.73 (s, 2H), 3.72-3.68 (m, 2H), 3.20 (t, J=5.6 Hz, 2H), 2.43 (s, 3H), 2.28 (t, J=6.0 Hz, 1H), 1.74 (s, 3H).

Description 152

### N-(2-hydroxyethyl)-4-methyl-N-((2-methyloxiran-2-yl)methyl)benzenesulfonamide

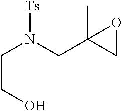 [SMILES: Cc1ccc(S(=O)(=O)N(CCO)CC2(C)CO2)cc1]

To a solution of N-(2-hydroxyethyl)-4-methyl-N-(2-methylallyl)benzenesulfonamide (10.5 g, 39.0 mmol) in DCM (150 mL) at 0° C. was added m-CPBA (10.1 g, 58.6 mmol). The reaction mixture was stirred at room temperature for 5 hours, quenched with saturated Na₂SO₃ (100 mL) and extracted with DCM (100 mL×2). The combined organic layers were washed with saturated Na₂CO₃ (50 mL×2) and brine (100 mL), dried over Na₂SO₄ and concentrated to give the title product (11.1 g, 100%) as a yellow oil.

¹H NMR (400 MHz, CDCl₃): δ 7.69 (d, J=8.0 Hz, 2H), 7.33 (d, J=8.0 Hz, 2H), 3.81-3.71 (m, 2H), 3.44-3.38 (m, 2H), 3.29-3.20 (m, 2H), 3.07-3.01 (m, 1H), 2.98 (d, J=4.4 Hz, 1H), 2.72 (d, J=4.4 Hz, 1H), 2.44 (s, 3H), 1.40 (s, 3H).

Description 153

### (2-methyl-4-tosylmorpholin-2-yl)methanol

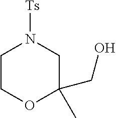 [SMILES: Cc1ccc(S(=O)(=O)N2CCOC(C)(CO)C2)cc1]

To a solution of N-(2-hydroxyethyl)-4-methyl-N-((2-methyloxiran-2-yl)methyl)benzenesulfonamide (11.1 g, 38.9 mmol) in DCM (100 mL) at 0° C. was added dropwise BF₃.Et₂O (6.63 g, 46.7 mmol). The reaction mixture was allowed to room temperature and stirred for 2 hrs, then concentrated and purified by silica gel chromatography column (Petroleum ether/EtOAc=1/1) to give the title product (11.1 g, 100%) as a yellow oil.

¹H NMR (400 MHz, CDCl₃): δ 7.62 (d, J=8.0 Hz, 2H), 7.34 (d, J=8.0 Hz, 2H), 3.93-3.87 (m, 1H), 3.79-3.74 (m, 1H), 3.58-3.50 (m, 2H), 3.27-3.22 (m, 1H), 3.01 (d, J=12.4 Hz, 1H), 2.78 (d, J=11.2 Hz, 1H), 2.69-2.62 (m, 1H), 2.44 (s, 3H), 1.28 (s, 3H).

Description 154

### (2-methylmorpholin-2-yl)methanol

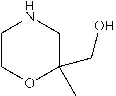

A mixture of (2-methyl-4-tosylmorpholin-2-yl)methanol (D153, 2.00 g, 7.02 mmol) and Mg powder (3.37 g, 14.0 mmol) in MeOH (100 mL) was sonicated for 3 hrs, then filtered and washed with MeOH (20 mL×2). The filtrate was concentrated to give the title product (crude, 8.00 g) as a white solid, which was used directly for next step.

LCMS [column: C₁₈; column size: 4.6×30 mm 5 μm; Dikwa Diamonsil plus; mobile phase: B (MeCN), A (0.02% NH₄Ac+5% MeCN in water); gradient (B %) in 4 mins. 05-95-POS; flow rate: 1.5 ml/min]: Rt=0.603 min; MS Calcd.:131, MS Found: 132 [M+H]⁺.

Description 155

### (4-(6-iodo-2-methylpyrimidin-4-yl)-2-methylmorpholin-2-yl)methanol

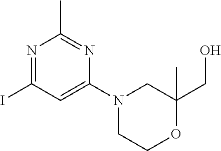 [SMILES: Cc1nc(I)cc(N2CCOC(C)(CO)C2)n1]

A mixture of 4,6-diiodo-2-methylpyrimidine (1.54 g, 6.32 mmol), (2-methylmorpholin-2-yl)methanol (D154, 8.00 g, crude) and TEA (2.13 g, 21.1 mmol) in DMSO (20.0 mL) was stirred at 60° C. for 4 hrs, diluted with H₂O (100 mL) and extracted with EtOAc (50 mL×3). The combined organic layers were washed with brine (50 mL), dried over Na₂SO₄ and concentrated. The residue was purified by silica gel chromatography column (petroleum ether/EtOAc=5/1 to 2/1) to give the title product (1.00 g, 45.0% yield for two steps) as a yellow oil.

¹H NMR (400 MHz, CDCl₃): δ 6.78 (s, 1H), 3.83-3.79 (m, 2H), 3.76-3.66 (m, 2H), 3.58 (d, J=11.6 Hz, 1H), 3.50-3.46 (m, 2H), 3.43-3.37 (m, 1H), 2.46 (s, 3H), 1.22 (s, 3H).

Description 156

### tert-butyl 4-(1-(6-(2-(hydroxymethyl)-2-methylmorpholino)-2-methylpyrimidin-4-yl)-5-methyl-1H-indazol-6-yl)piperidine-1-carboxylate

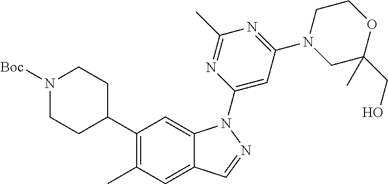 [SMILES: Cc1nc(N2CCOC(C)(CO)C2)cc(-n2ncc3cc(C)c(C4CCN(C(=O)OC(C)(C)C)CC4)cc32)n1]

A mixture of tert-butyl 4-(5-methyl-1H-indazol-6-yl)piperidine-1-carboxylate (D99, 904 mg, 2.87 mmol), (4-(6-iodo-2-methylpyrimidin-4-yl)-2-methylmorpholin-2-yl)methanol (D155, 1.00 g, 2.87 mmol), N,N′-dimethylcyclohexane-1,2-diamine (80.0 mg, 0.570 mmol), CuI (55.0 mg, 0.290 mmol) and K₃PO₄ (1.21 g, 5.73 mmol) in toluene (10 mL) was stirred at 100° C. for 4 hrs, diluted with EtOAc (100 mL), washed with NH₃.H₂O (30 mL×2) and brine (30 mL), dried over Na₂SO₄ and concentrated. The residue was purified by silica gel chromatography column (Petroleum ether/EtOAc=1/1) to give the title product (1.10 g, 71.0%) as a yellow oil.

¹H NMR (400 MHz, CDCl₃): δ 8.74 (s, 1H), 8.05 (s, 1H), 7.51 (s, 1H), 6.93 (s, 1H), 4.35-4.29 (m, 1H), 4.00 (d, J=12.4 Hz, 1H), 3.86-3.78 (m, 3H), 3.64-3.58 (m, 2H), 3.49-3.44 (m, 2H), 3.02-2.85 (m, 3H), 2.61 (s, 3H), 2.47 (s, 3H), 1.89-1.64 (m, 5H), 1.50 (s, 9H), 1.27 (s, 3H).

Description 157

### (2-methyl-4-(2-methyl-6-(5-methyl-6-(piperidin-4-yl)-1H-indazol-1-yl)pyrimidin-4-yl)morpholin-2-yl)methanol

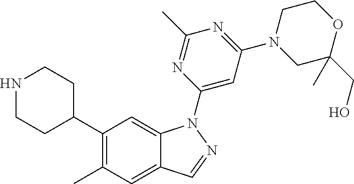 [SMILES: Cc1nc(N2CCOC(C)(CO)C2)cc(-n2ncc3cc(C)c(C4CCNCC4)cc32)n1]

To a solution of tert-butyl 4-(1-(6-(2-(hydroxymethyl)-2-methylmorpholino)-2-methylpyrimidin-4-yl)-5-methyl-1H-indazol-6-yl)piperidine-1-carboxylate (D156, 1.10 g, 1.87 mmol) in DCM (8.00 mL) was added TFA (4.00 mL) at 0° C. The reaction mixture was stirred at room temperature for 1 hour, poured into saturated Na₂CO₃ (30.0 mL) and extracted with DCM (50.0 mL×3). The combined organic layers were concentrated and purified by silica gel chromatography column (DCM/MeOH=15/1) to give the title product (700 mg, 86.0%) as a light yellow solid.

¹H NMR (400 MHz, CDCl₃): δ 8.80 (s, 1H), 8.06 (s, 1H), 7.51 (s, 1H), 6.94 (s, 1H), 4.00 (d, J=10.8 Hz, 1H), 3.85-3.78 (m, 3H), 3.63 (d, J=11.7 Hz, 2H), 3.49-3.44 (m, 2H), 3.34 (d, J=12.4 Hz, 2H), 3.01-2.95 (m, 1H), 2.92-2.85 (m, 2H), 2.61 (s, 3H), 2.47 (s, 3H), 1.94-1.83 (m, 4H), 1.27 (s, 3H).

Description 158

### 5-bromo-2,4-dimethylaniline

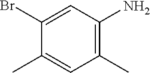

To a solution of 1-bromo-2,4-dimethyl-5-nitrobenzene (540 g, 2.35 mol) in THF (4 L) which was fiercely stirred was added Fe powder (1600 g, 17.9 mol) and con. HCl (500 ml). The reaction mixture was stirred at rt for 4 hrs, then quenched with K₂CO₃ and filtered. The filtered cake was washed with EtOAc. The organic solutions were combined and dried over Na₂SO₄ and concentrated to give the title product (crude, 457 g, yield: 97.3%) as a light yellow solid.

¹H NMR (400 MHz, CDCl₃): δ 6.88 (s, 1H), 6.85 (s, 1H), 3.49 (s, 2H), 2.25 (s, 3H), 2.07 (s, 3H).

### Examples 1 and 2

### ((2S)-4-(2-Methyl-6-(5-methyl-6-(1-(tetrahydrofuran-3-yl)piperidin-4-yl)-1H-indazol-1-yl)pyrimidin-4-yl)morpholin-2-yl)methanol (Single Unknown Isomer 1, E1 and Single Unknown Isomer 2, E2)

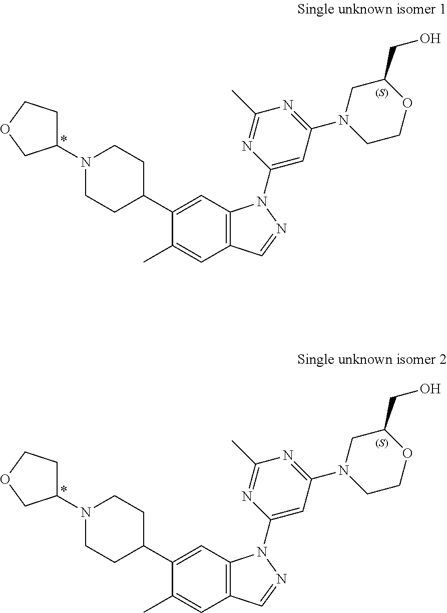

To a mixture of 5-methyl-6-(1-(tetrahydrofuran-3-yl)piperidin-4-yl)-1H-indazole (150 mg, 0.526 mmol), (S)-(4-(6-iodo-2-methylpyrimidin-4-yl)morpholin-2-yl)methanol (178 mg, 0.531 mmol), CuI (101 mg, 0.53 mmol) and K₃PO₄ (225 mg, 1.06 mmol) in dry toluene (4 mL) was added N,N′-dimethylethylenediamine (93 mg, 1.06 mmol). The suspension was degassed with Ar and refluxed at 95° C. for 4 hrs. TLC showed the reaction was completed. The cooled reaction mixture was filtered and the filter cake was washed with CH₂Cl₂. The combined filtrate was concentrated and the residue was purified by column chromatography (eluent: PE:EtOAc=1:1, followed by CH₂Cl₂:MeOH=50:1 to 25:1) to afford the desired mixture as a yellow solid (144 mg, yield: 55%).

LC-MS [mobile phase: from 95% water (0.1% FA) and 5% MeCN (0.1% FA) to 5% water (0.1% FA) and 95% MeCN (0.1% FA) in 12.0 min]: Rt=5.74 min; MS Calcd: 492.28; MS Found: 493.7 [M+H]⁺.

Chiral separation of racemic ((2S)-4-(2-methyl-6-(5-methyl-6-(1-(tetrahydrofuran-3-yl)piperidin-4-yl)-1H-indazol-1-yl)pyrimidin-4-yl)morpholin-2-yl)methanol by chiral prep-HPLC (Method: Column: AD-H; Column size: 0.46 cm I.D.×15 cm L; Mobile phase: Supercritical CO₂:EtOH (0.1% NH₃H₂O)=60:40; Flow rate: 0.5 mL; Wave length: UV 254 nm; Temperature: 25° C.; Sample solution in EtOH) afforded the title product (Single unknown isomer 1) as a yellow solid (71.19 mg, yield: 49%) and another isomer (Single unknown isomer 2) as a yellow solid (74.34 mg, yield: 51%).

Single Unknown Isomer 1

¹H NMR (400 MHz, CDCl₃) δ8.78 (s, 1H), 8.05 (s, 1H), 7.50 (s, 1H), 6.94 (s, 1H), 4.31-4.29 (m, 2H), 4.06 (d, J=5.2 Hz, 1H), 4.00-3.93 (m, 2H), 3.84-3.68 (m, 6H), 3.22-2.82 (m, 6H), 2.63 (s, 3H), 2.46 (s, 3H), 2.29-2.10 (m, 3H), 2.01-1.94 (m, 5H).

LC-MS [mobile phase: from 90% water (0.1% FA) and 10% MeCN (0.1% FA) to 5% water (0.1% FA) and 95% MeCN (0.1% FA) in 12 min]: Purity 98%; Rt=5.40 min; MS Calcd: 492.28, MS Found: 493.8 [M+H]⁺.

Single Unknown Isomer 2

¹H NMR (400 MHz, CDCl₃) 8.78 (s, 1H), 8.05 (s, 1H), 7.50 (s, 1H), 6.95 (s, 1H), 4.32-4.29 (m, 2H), 4.06 (d, J=5.2 Hz, 1H), 4.00-3.95 (m, 2H), 3.84-3.68 (m, 6H), 3.22-2.82 (m, 6H), 2.63 (s, 3H), 2.46 (s, 3H), 2.29-2.10 (m, 4H), 2.01-1.93 (m, 5H).

LC-MS [mobile phase: from 90% water (0.1% FA) and 10% MeCN (0.1% FA) to 5% water (0.1% FA) and 95% MeCN (0.1% FA) in 12 min]: Rt=5.40 min; MS Calcd: 492.28, MS Found: 493.9 [M+H]⁺.

### Examples 3 and 4

### ((2R)-4-(2-Methyl-6-(5-methyl-6-(1-(tetrahydrofuran-3-yl)piperidin-4-yl)-1H-indazol-1-yl)pyrimidin-4-yl)morpholin-2-yl)methanol (Single Unknown Isomer 1, E3 and Single Unknown Isomer 2, E4)

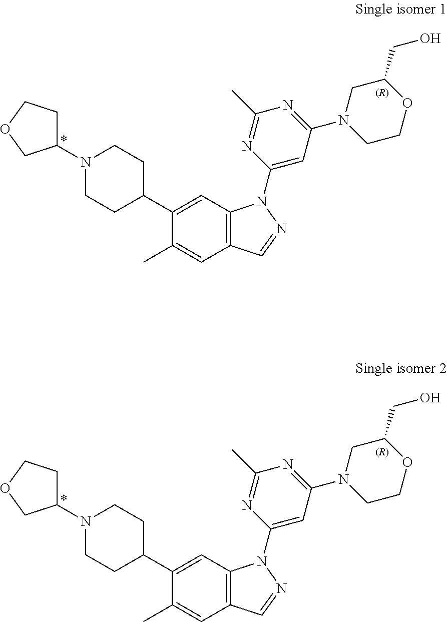

To a mixture of 5-methyl-6-(1-(tetrahydrofuran-3-yl)piperidin-4-yl)-1H-indazole (150 mg, 0.526 mmol), (R)-(4-(6-iodo-2-methylpyrimidin-4-yl)morpholin-2-yl)methanol (178 mg, 0.530 mmol), CuI (101 mg, 0.530 mmol) and K₃PO₄ (225 mg, 1.06 mmol) in dry toluene (4 mL) was added N,N′-dimethylethylenediamine (93.0 mg, 1.06 mmol). The suspension was degassed with Ar and refluxed at 95° C. for 3.5 hrs. The cooled reaction mixture was filtered and the filtered cake was washed with CH₂Cl₂. The combined filtrate was concentrated and purified by column chromatography (PE:EtOAc=1:1, followed by CH₂Cl₂:MeOH=50:1 to 25:1) to afford the mixture as a yellow solid, which was the chiral mixture (134 mg, yield: 51%).

LC-MS [mobile phase: from 95% water (0.1% FA) and 5% MeCN (0.1% FA) to 5% water (0.1% FA) and 95% MeCN (0.1% FA) in 12.0 min]: Rt=5.76 min; MS Calcd: 492.28; MS Found: 493.8 [M+H]⁺.

The mixture (134 mg) was analyzed by HPLC (Method: Column: AD-H; Column size: 0.46 cm I.D.×15 cm L; Injection: 2 μl; Mobile phase: HEP:ETOH (0.1% DEA)=60:40; Flow rate: 0.5 ml; Wave length: UV 254 nm; Temperature: 25° C.; Sample solution: X mg/ml in ETOH) and separated by chiral prep-HPLC (Column: ChiralPak AD-H Daicel chemical Industries, Ltd, 250×30 mm I.D., 5 μm; Mobile phase A: Supercritical CO₂, Mobile phase B: Ethanol (0.1% NH₃H₂O); A:B=60:40 at 50 mL/min; Column Temp: 38° C.; Nozzle Pressure: 100 Bar; Nozzle Temp:60° C.; Evaporator Temp: 20° C.; Trimmer Temp:25° C.; Wavelength: 220 nm) to afford the single unknown isomer 1 as a brown solid (61.6 mg, yield: 45%) and the single unknown isomer 2 as a brown solid (59.4 mg, yield: 44%).

Single Isomer 1

¹H NMR (400 MHz, CDCl₃) δ8.78 (s, 1H), 8.05 (s, 1H), 7.50 (s, 1H), 6.94 (s, 1H), 4.31-4.29 (m, 2H), 4.06 (d, J=5.2 Hz, 1H), 4.00-3.93 (m, 2H), 3.84-3.68 (m, 6H), 3.22-2.82 (m, 6H), 2.63 (s, 3H), 2.46 (s, 3H), 2.29-2.10 (m, 3H), 2.01-1.94 (m, 5H).

LC-MS [mobile phase: from 90% water (0.1% FA) and 10% MeCN (0.1% FA) to 5% water (0.1% FA) and 95% MeCN (0.1% FA) in 12 min]: Purity 98%; Rt=5.40 min; MS Calcd: 492.28, MS Found: 493.8 [M+H]⁺.

Chiral HPLC [Column: OD 4.6×250 mm, 5 μm (Daicel) (CA-HPLC-182), Mobile phase: hexane/EtOH (0.2% DEA)=90/10, Flow rate: 1 mL/min, Temperature: 35° C.]: Rt=27.170 min, purity: 100%.

Further analyses characterized the structure of isomer 1 to be ((R)-4-(2-methyl-6-(5-methyl-6-(1-((R)-tetrahydrofuran-3-yl)piperidin-4-yl)-1H-indazol-1-yl)pyrimidin-4-yl)morpholin-2-yl)methanol.

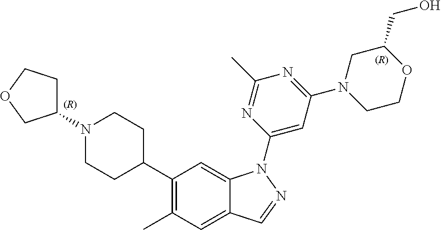

Single Isomer 2

¹H NMR (400 MHz, CDCl₃) δ 8.78 (s, 1H), 8.05 (s, 1H), 7.50 (s, 1H), 6.95 (s, 1H), 4.32-4.29 (m, 2H), 4.06 (d, J=5.2 Hz, 1H), 4.00-3.95 (m, 2H), 3.84-3.68 (m, 6H), 3.22-2.82 (m, 6H), 2.63 (s, 3H), 2.46 (s, 3H), 2.29-2.10 (m, 4H), 2.01-1.93 (m, 5H).

LC-MS [mobile phase: from 80% water (0.1% FA) and 20% MeCN (0.1% FA) to 5% water (0.1% FA) and 95% MeCN (0.1% FA) in 12 min]: Purity 96%; Rt=4.32 min; MS Calcd: 492.28, MS Found: 493.6 [M+H]⁺.

Chiral HPLC [Column: OD 4.6×250 mm, 5 μm (Daicel) (CA-HPLC-182), Mobile phase: hexane/EtOH (0.2% DEA)=90/10, Flow rate: 1 mL/min, Temperature: 35° C.]: Rt=25.022 min, purity: 100%.

Further analyses characterized the structure of isomer 2 to be ((R)-4-(2-methyl-6-(5-methyl-6-(1-((S)-tetrahydrofuran-3-yl)piperidin-4-yl)-1H-indazol-1-yl)pyrimidin-4-yl)morpholin-2-yl)methanol.

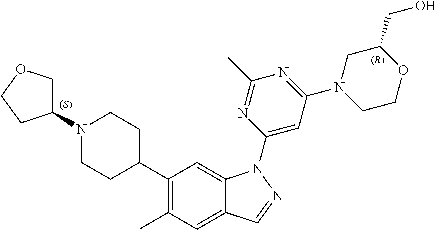

### Examples 5 and 6

### cis-1-(6-(6-(3-Fluoro-1-(tetrahydrofuran-3-yl)piperidin-4-yl)-5-methyl-1H-indazol-1-yl)-2-methoxypyrimidin-4-yl)azetidin-3-ol (Single Unknown Isomer 1, E5 and Single Unknown Isomer 2, E6)

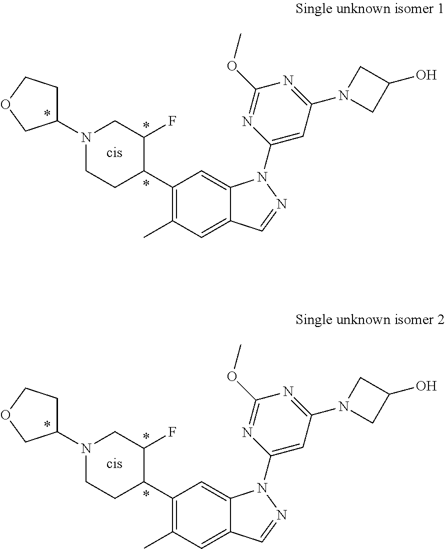 [SMILES: COc1nc(N2CC(O)C2)cc(-n2ncc3cc(C)c(C4CCN(C5CCOC5)CC4F)cc32)n1 ; COc1nc(N2CC(O)C2)cc(-n2ncc3cc(C)c(C4CCN(C5CCOC5)CC4F)cc32)n1]

The title compounds were prepared by a procedure similar to that described for E1 and E2 starting from a solution of cis-6-(3-fluoro-1-(tetrahydrofuran-3-yl)piperidin-4-yl)-5-methyl-1H-indazole (D34) and 1-(6-iodo-2-methoxypyrimidin-4-yl)azetidin-3-ol in toluene, CuI, K₃PO₄ and N,N′-dimethylethy-lenediamine at 100° C.

Chiral Separation:

Method: Column: AD-H; Column size: 0.46 cm I.D.×15 cm L; Mobile phase: Supercritical CO₂:IPA (0.1% NH₃H₂O)=60:40; Flow rate: 0.5 mL; Wave length: UV 254 nm; Temperature: 25° C.; Sample solution in EtOH

Single Unknown Isomer 1

¹H NMR (400 MHz, CDCl₃) δ 8.86 (s, 1H), 8.07 (s, 1H), 7.53 (s, 1H), 6.47 (s, 1H), 4.88 4.75 (m, 2H), 4.44˜4.39 (m, 2H), 4.11 (s, 3H), 4.02˜3.92 (m, 3H), 3.90˜3.88 (m, 1H), 3.82 3.80 (m, 1H), 3.69˜3.60 (m, 1H), 3.44˜3.40 (m, 1H), 3.17˜3.08 (m, 2H), 2.82˜2.79 (m, 1H), 2.47 (s, 3H), 2.25˜2.19 (m, 3H), 2.11˜2.05 (m, 1H), 1.95˜1.91 (m, 2H), 1.89 1.77 (m, 1H).

¹⁹F NMR (376 MHz, CDCl₃) δ 301.70 (s)

LC-MS [mobile phase: from 90% water (0.1% FA) and 10% MeCN (0.1% FA) to 5% water (0.1% FA) and 95% MeCN (0.1% FA) in 9 min]: Rt=4.76 min; MS Calcd: 482.55, MS Found: 483.3 [M+H]⁺.

Chiral HPLC [Column: AD-H; Column size: 0.46 cm I.D.×15 cm L; Injection: 2 μl; Mobile phase: HEP:IPA (0.1% DEA)=60:40; Flow rate: 0.5 m/min; Wave length: UV 254 nm; Temperature: 25° C.]: Rt: 1.712 min, ee: 100%

Single Unknown Isomer 2

¹H NMR (400 MHz, CDCl₃) 8.86 (s, 1H), 8.07 (s, 1H), 7.53 (s, 1H), 6.47 (s, 1H), 4.86 4.73 (m, 2H), 4.43˜4.39 (m, 2H), 4.11 (s, 3H), 4.03˜3.97 (m, 3H), 3.90˜3.88 (m, 1H), 3.81˜3.77 (m, 1H), 3.72˜3.68 (m, 1H), 3.24˜3.15 (m, 2H), 3.10˜3.08 (m, 1H), 3.02˜2.99 (m, 1H), 2.47 (s, 3H), 2.23˜2.17 (m, 3H), 2.15˜2.07 (m, 1H), 1.98˜1.93 (m, 2H), 1.89˜1.79 (m, 1H).

¹⁹F NMR (376 MHz, CDCl₃) δ 301.80 (s)

LC-MS [mobile phase: from 90% water (0.1% FA) and 0% MeCN (0.1% FA) to 5% water (0.1% FA) and 95% MeCN (0.1% FA) in 9 min]: Rt=4.74 min; MS Calcd: 482.55, MS Found: 483.3 [M+H]⁺.

Chiral HPLC [Column: AD-H; Column size: 0.46 cm I.D.×15 cm L; Injection: 2 μl; Mobile phase: HEP:IPA (0.1% DEA)=60:40; Flow rate: 0.5 ml/min; Wave length: UV 254 nm; Temperature: 25° C.]: Rt: 2.542 min, ee: 100%

### Examples 7 and 8

### cis-1-(6-(6-(3-Fluoro-1-(tetrahydrofuran-3-yl)piperidin-4-yl)-5-methyl-1H-indazol-1-yl)-2-methoxypyrimidin-4-yl)azetidin-3-ol(Single Unknown Isomer 1, E7 and Single Unknown Isomer 1, E8)

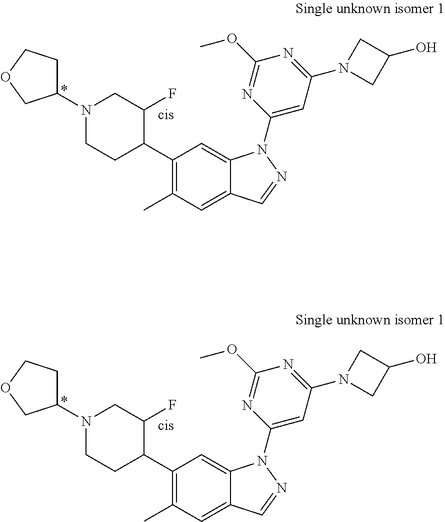 [SMILES: COc1nc(N2CC(O)C2)cc(-n2ncc3cc(C)c(C4CCN(C5CCOC5)CC4F)cc32)n1 ; COc1nc(N2CC(O)C2)cc(-n2ncc3cc(C)c(C4CCN(C5CCOC5)CC4F)cc32)n1]

The title compounds were prepared by a procedure similar to that described for E1 and E2 starting from a solution of cis-6-(3-fluoro-1-(tetrahydrofuran-3-yl)piperidin-4-yl)-5-methyl-1H-indazole (D33), 1-(6-iodo-2-methoxypyrimidin-4-yl)azetidin-3-ol in Toluene, CuI, K₃PO₄ and N,N′-dimethylethylenediamine.

Chiral Separation:

Method: Column: AD-H; Column size: 0.46 cm I.D.×15 cm L; Injection: 2 μl; Mobile phase: Supercritical CO₂::EtOH (0.1% NH₃H₂O)=60:40; Flow rate: 0.5 mL; Wave length: UV 254 nm; Temperature: 25° C.; Sample solution in EtOH

Single Unknown Isomer 1

¹H NMR (400 MHz, MeOD) 8.75 (s, 1H), 8.19 (s, 1H), 7.67 (s, 1H), 6.51 (s, 1H), 5.15 5.11 (m, 2H), 4.71˜4.68 (m, 2H), 4.38˜4.36 (m, 2H), 4.29˜4.27 (m, 1H), 4.12 (br, 2H), 4.07 (s, 3H), 3.93˜3.90 (m, 4H), 3.88˜3.86 (m, 1H), 3.78˜3.75 (m, 2H), 3.37˜3.35 (m, 1H), 2.65 (s, 3H), 2.52˜2.47 (m, 1H), 2.46˜2.44 (m, 2H), 2.04˜2.00 (m, 1H).

¹⁹F NMR (376 MHz, MeOD) 77.06 (s), 185 (s), TFA salt

LC-MS [mobile phase: from 90% water (0.1% FA) and 10% MeCN (0.1% FA) to 5% water (0.1% FA) and 95% MeCN (0.1% FA) in 9.0 min]: Rt=4.89 min; MS Calcd: 482.55, MS Found: 483.3 [M+H]⁺.

Chiral HPLC [Column: AD-H; Column size: 0.46 cm I.D.×15 cm L; Injection: 2 μl; Mobile phase: HEP:EtOH (0.1% DEA)=60:40; Flow rate: 0.5 ml; Wave length: UV 254 nm; Temperature: 25° C.]: Rt: 1.913 min, ee 100%;

Single Unknown Isomer 2

¹H NMR (400 MHz, MeOD) 8.71 (s, 1H), 8.19 (s, 1H), 7.66 (s, 1H), 6.49 (s, 1H), 5.14 5.01 (m, 2H), 4.88˜4.86 (m, 2H), 4.38˜4.36 (m, 2H), 4.35˜4.30 (m, 1H), 4.27˜4.12 (m, 2H), 4.09 (s, 3H), 3.93˜3.85 (m, 4H), 3.77˜3.75 (m, 1H), 3.60˜3.57 (m, 2H), 3.40˜3.37 (m, 1H), 2.52 (s, 3H), 2.47˜2.45 (m, 1H), 2.34˜2.30 (m, 2H), 2.05˜2.01 (m, 1H).

¹⁹F NMR (376 MHz, MeOD) δ 77.12 (s), 185 (s), TFA salt

LC-MS [mobile phase: from 90% water (0.1% FA) and 0% MeCN (0.1% FA) to 5% water (0.1% FA) and 95% MeCN (0.1% FA) in 9.0 min]: Rt=4.26 min; MS Calcd: 482.55, MS Found: 483.3 [M+H]⁺.

Chiral HPLC [Column: AD-H; Column size: 0.46 cm I.D.×15 cm L; Injection: 2 μl; Mobile phase: HEP:EtOH (0.1% DEA)=60:40; Flow rate: 0.5 ml/min; Wave length: UV 254 nm; Temperature: 25° C.]: Rt: 3.046 min, ee 99%

### Examples 9 and 10

### 1-(2-Methoxy-6-(5-methyl-6-(1-(tetrahydrofuran-3-yl)piperidin-4-yl)-1H-indazol-1-yl)pyrimidin-4-yl)azetidin-3-ol (Single Unknown Enantiomer 1, E9 and Single Unknown Enantiomer 2, E10)

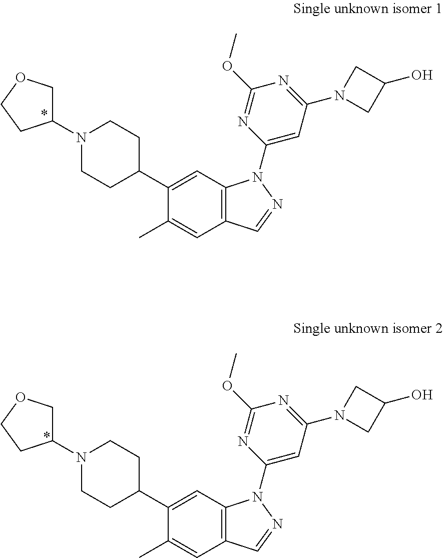 [SMILES: COc1nc(N2CC(O)C2)cc(-n2ncc3cc(C)c(C4CCN(C5CCOC5)CC4)cc32)n1 ; COc1nc(N2CC(O)C2)cc(-n2ncc3cc(C)c(C4CCN(C5CCOC5)CC4)cc32)n1]

The title compounds were prepared by a procedure similar to that described for E1 and E2 starting from a solution of 5-methyl-6-(1-(tetrahydrofuran-3-yl)piperidin-4-yl)-1H-indazole, and 1-(6-iodo-2-methoxypyrimidin-4-yl)azetidin-3-ol in toluene/THF, DMEDA, CuI and K₃PO₄ at 90° C.

Chiral Separation:

Method: AD-H, 0.46 cm I.D.×15 cm L, Mobile phase: Supercritical CO₂:IPA (0.1% NH₃H₂O)=60:40, Flow rate: 0.5 mL/min, 254 nm, Temperature: 25° C.

Single Unknown Enantiomer 1

¹H NMR (400 MHz, CDCl₃) δ 8.74 (s, 1H), δ8.06 (s, 1H), 7.50 (s, 1H), δ 6.47 (s, 1H), δ 4.84 (s, 1H), δ 4.41˜4.39 (m, 2H), δ 4.12 (s, 3H), δ 4.02˜3.92 (m, 4H), δ 3.81˜3.70 (m, 1H), δ 3.68˜3.64 (m, 1H), δ 3.15˜3.12 (d, J=12.8 Hz, 1H), δ 3.06-3.02 (m, 1H), δ 2.97-2.97 (d, J=6.4 Hz, 1H), δ 2.83˜2.80 (m, 1H), δ 2.45 (s, 3H), δ 2.24˜2.21 (m, 2H), δ 2.08˜2.05 (m, 1H), δ 1.90˜1.84 (m, 6H).

LC-MS [mobile phase: From 90% water (0.1% FA) and 10% MeCN (0.1% FA) to 5% water (0.1% FA) and 95% MeCN (0.1% FA) in 9 min]: purity 100%, Rt=4.44 min; MS Calcd.: 464.5, MS Found: 465.3 [M+H]⁺.

Chiral HPLC [AD-H, 0.46 cm I.D.×15 cm L, Mobile phase: HEP:APA (0.1% DEA)=60:40, Flow rate: 0.5 mL/min, 254 nm, Temperature: 25° C.]: Rt=1.345 min, ee: 100%

Single Unknown Isomer 2

¹H NMR (400 MHz, CDCl₃) δ 8.74 (s, 1H), δ8.06 (s, 1H), δ 7.50 (s, 1H), δ 6.47 (s, 1H), δ 4.83 (s, 1H), δ 4.44˜4.40 (m, 2H), δ 4.12 (s, 3H), δ 4.02˜3.91 (m, 4H), δ 3.83˜3.81 (m, 1H), δ 3.71˜3.66 (m, 1H), δ 3.15˜3.13 (d, J=12.8 Hz, 1H), δ 3.03˜3.01 (m, 1H), δ 2.93˜2.91 (d, J=6.4 Hz, 1H), δ 2.83˜2.81 (m, 1H), δ 2.46 (s, 3H), δ 2.24˜2.18 (m, 2H), δ 2.08˜2.06 (m, 1H), δ 1.93˜1.81 (m, 6H).

LC-MS [mobile phase: From 90% water (0.1% FA) and 10% MeCN (0.1% FA) to 5% water (0.1% FA) and 95% MeCN (0.1% FA) in 9 min]: purity 100%, Rt=4.42 min; MS Calcd.:464.5, MS Found: 465.3 [M+H]⁺.

Chiral HPLC [AD-H, 0.46 cm I.D.×15 cm L, Mobile phase: HEP:IPA (0.1% DEA)=60:40, Flow rate: 0.5 mL/min, Wave length: 254 nm, Temperature: 25° C.]: Rt=2.193 min, ee: 100%

### Examples 11 and 12

### 1-(2-Methoxy-6-(5-methyl-6-(1-(tetrahydrofuran-3-yl)piperidin-4-yl)-1H-indazol-1-yl)pyrimidin-4-yl)-3-methylazetidin-3-ol (single unknown enantiomer 1, E11 and Single Unknown Enantiomer 2, E12)

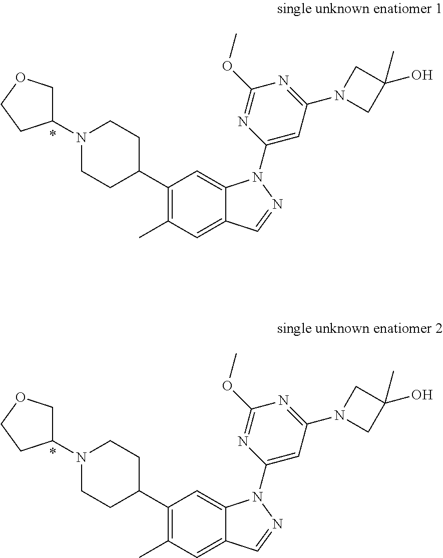 [SMILES: COc1nc(N2CC(C)(O)C2)cc(-n2ncc3cc(C)c(C4CCN(C5CCOC5)CC4)cc32)n1 ; COc1nc(N2CC(C)(O)C2)cc(-n2ncc3cc(C)c(C4CCN(C5CCOC5)CC4)cc32)n1]

To a solution of 1-(2-methoxy-6-(5-methyl-6-(piperidin-4-yl)-1H-indazol-1-yl)pyrimidin-4-yl)-3-methylazetidin-3-ol (227 mg, 0.560 mmol), dihydrofuran-3(2H)-one (239 mg, 2.78 mmol) and AcOH (1 drop) in DCE (8 mL) was added NaBH₃CN (70.0 mg, 1.11 mmol). The mixture was stirred at room temperature for 20 hrs, then quenched with a solution of sat. NaHCO₃ (3 drops) and concentrated. The purification via silica gel chromatography column (DCM/MeOH=15:1) afforded the title compound (127 mg, 47%) as a white solid.

¹HNMR (400 MHz, CDCl₃) δ 8.63 (s, 1H), 8.33 (s, 1H), 7.66 (s, 1H), 6.44 (s, 1H), 5.76 (s, 1H), 4.11-4.09 (m, 1H), 4.00-3.89 (m, 10H), 3.80-3.78 (m, 2H), 3.67-3.65 (m, 1H), 3.17 (s, 3H), 2.45-2.33 (m, 6H), 1.91 (s, 2H), 1.45 (s, 4H).

Chiral Separation

Method: column: Chiralpak IF; 5 μm 20×150 mm; Phase: Supercritical CO₂:EtOH=70:30; Flow rate:12 mL/min, Wave length: 230 nm.

Single Unknown Enantiomer 1:

¹H NMR (400 MHz, CDCl₃) δ 8.72 (s, 1H), 8.05 (s, 1H), 7.49 (s, 1H), 6.45 (s, 1H), 4.43 (br s, 1H), 4.07 (s, 6H), 3.99-3.90 (m, 2H), 3.85-3.79 (m, 1H), 3.72 (t, J=7.6 Hz, 1H), 3.48 (s, 1H), 3.18-3.15 (m, 1H), 3.08-3.05 (m, 1H), 2.96-2.93 (m, 1H), 2.86-2.81 (m, 1H), 2.44 (s, 3H), 2.27-2.21 (m, 2H), 2.14-2.07 (m, 1H), 1.98-1.89 (m, 5H), 1.61 (s, 3H).

Chiral-HPLC [column: chiral pak IF, 5 μm 250 mm×4.6 mm; mobile phase: Hex:EtOH=70:30; flow rate: 1 mL/min; wave length: 230 nm; Temperature: 30° C.]: Rt=11.319 min.

LC-MS [column: C₁₈; column size: 4.6×50 mm; mobile phase: B (MeCN) A (0.02% NH₄AC); gradient (B %)]: Rt=3.477 min, MS Calcd.: 478, MS Found: 479 [M+H]⁺.

Single Unknown Enantiomer 2:

¹H NMR (400 MHz, CDCl₃) δ 8.71 (s, 1H), 8.04 (s, 1H), 7.48 (s, 1H), 6.44 (s, 1H), 4.45 (br s, 1H), 4.07 (s, 6H), 4.01-3.90 (m, 2H), 3.85-3.79 (m, 1H), 3.71 (t, J=8.0 Hz, 1H), 3.48 (s, 1H), 3.17-3.14 (m, 1H), 3.07-3.04 (m, 1H), 2.95-2.92 (m, 1H), 2.84-2.78 (m, 1H), 2.44 (s, 3H), 2.28-2.21 (m, 2H), 2.12-2.07 (m, 1H), 1.96-1.82 (m, 5H), 1.60 (s, 3H).

Chiral-HPLC [column: chiral pak IF, 5 μm 250 mm×4.6 mm; mobile phase: Hex:EtOH=70:30; flow rate: 1 mL/min; wave length: 230 nm; Temperature: 30° C.]: Rt=14.219 min.

LC-MS [column: C₁₈; column size: 4.6×50 mm; mobile phase: B (MeCN) A (0.02% NH₄AC); gradient (B %)]: Rt=3.489 min, MS Calcd.: 478, MS Found: 479 [M+H]⁺.

### Examples 13 and 14

### cis-1-(6-(6-(3-Fluoro-1-(tetrahydrofuran-3-yl)piperidin-4-yl)-5-methyl-1H-indazol-1-yl)-2-methylpyrimidin-4-yl)azetidin-3-ol (Single Unknown Isomer 1, E13 and Single Unknown Isomer 2, E14)

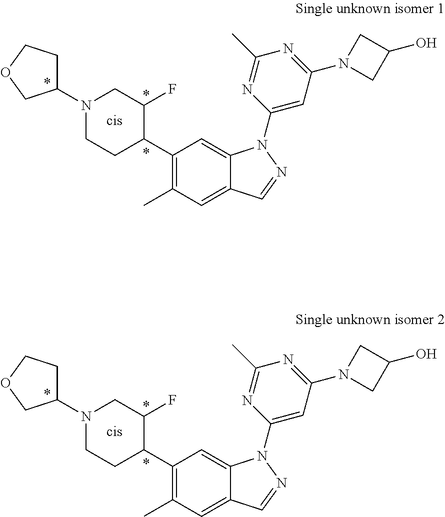 [SMILES: Cc1nc(N2CC(O)C2)cc(-n2ncc3cc(C)c(C4CCN(C5CCOC5)CC4F)cc32)n1 ; Cc1nc(N2CC(O)C2)cc(-n2ncc3cc(C)c(C4CCN(C5CCOC5)CC4F)cc32)n1]

The title compounds were prepared by a procedure similar to that described for E1 and E2 starting from a solution of cis-6-(3-fluoro-1-(tetrahydrofuran-3-yl)piperidin-4-yl)-5-methyl-1H-indazole (D33), 1-(6-iodo-2-methylpyrimidin-4-yl)azetidin-3-ol in toluene, CuI, K₃PO₄ and N,N′-dimethylethylenediamine at 100° C.

Chiral Separation:

Method: Column: AD-H; Column size: 0.46 cm I.D.×15 cm L; Mobile phase: Supercritical CO₂:EtOH (0.1% NH₃H₂O)=60:40; Flow rate: 0.5 mL/min; Wave length: UV 254 nm; Temperature: 25° C.; Sample in EtOH

Single Unknown Isomer 1

¹H NMR (400 MHz, CDCl₃) 8.89 (s, 1H), 8.06 (s, 1H), 7.52 (s, 1H), 6.59 (s, 1H), 4.94˜4.79 (m, 2H), 4.43˜4.39 (m, 2H), 4.02˜3.99 (m, 3H), 3.94˜3.91 (m, 1H), 3.84˜3.80 (m, 1H), 3.77˜3.73 (m, 1H), 3.28˜3.26 (m, 1H), 3.20˜3.17 (m, 1H), 3.10˜3.04 (m, 2H), 2.63 (s, 3H), 2.47 (s, 3H), 2.33˜2.29 (m, 1H), 2.21˜2.18 (m, 2H), 2.10˜2.09 (m, 1H), 1.99˜1.86 (m, 3H).

¹⁹F NMR (376 MHz, CDCl₃) δ 183.29 (s)

LC-MS [mobile phase: from 80% water (0.1% FA) and 20% MeCN (0.1% FA) to 5% water (0.1% FA) and 95% MeCN (0.1% FA) in 9 min]: Purity 100%; Rt=2.80 min; MS Calcd: 466.5, MS Found: 467.3 [M+H]⁺.

Chiral HPLC [Column: AD-H; Column size: 0.46 cm I.D.×15 cm L; Injection: 2 μl; Mobile phase: HEP:EtOH (0.1% DEA)=60:40; Flow rate: 0.5 ml; Wave length: UV 254 nm; Temperature: 25° C.]: Rt: 4.807 min, ee: 100%

Single Unknown Isomer 2

¹H NMR (400 MHz, CDCl₃) δ 8.89 (s, 1H), 8.06 (s, 1H), 7.52 (s, 1H), 6.59 (s, 1H), 4.93˜4.81 (m, 2H), 4.42˜4.40 (m, 2H), 4.01˜3.98 (m, 3H), 3.94˜3.91 (m, 1H), 3.83˜3.81 (m, 1H), 3.75˜3.71 (m, 1H), 3.48˜3.46 (m, 1H), 3.18˜3.15 (m, 2H), 2.87˜2.84 (m, 1H), 2.63 (s, 3H), 2.47 (s, 3H), 2.28˜2.27 (m, 1H), 2.26˜2.24 (m, 2H), 2.12˜2.10 (m, 1H), 1.97˜1.88 (m, 3H).

¹⁹F NMR (376 MHz, CDCl₃) δ 183.29 (s)

LC-MS [mobile phase: from 80% water (0.1% FA) and 20% MeCN (0.1% FA) to 5% water (0.1% FA) and 95% MeCN (0.1% FA) in 10 min]: Purity 100%; Rt=2.82 min; MS Calcd: 466.5, MS Found: 467.3 [M+H]⁺.

Chiral HPLC [Column: AD-H; Column size: 0.46 cm I.D.×15 cm L; Injection: 2 μl; Mobile phase: HEP:EtOH (0.1% DEA)=60:40; Flow rate: 0.5 ml; Wave length: UV 254 nm; Temperature: 25° C.]: Rt: 5.138 min, ee: 100%

### Examples 15 and 16

### cis-1-(6-(6-(3-Fluoro-1-(tetrahydrofuran-3-yl)piperidin-4-yl)-5-methyl-1H-indazol-1-yl)-2-methylpyrimidin-4-yl)azetidin-3-ol (Single Unknown Isomer 3, E15 and Single Unknown Isomer 4, E16)

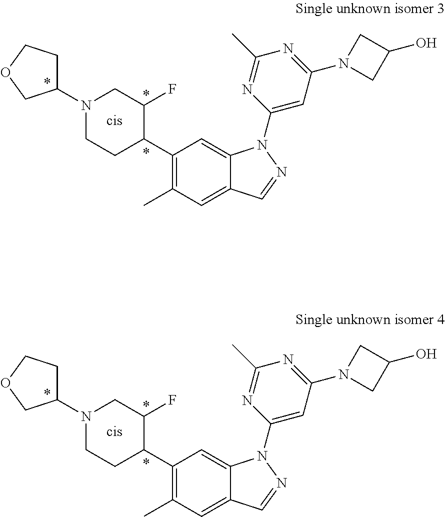 [SMILES: Cc1nc(N2CC(O)C2)cc(-n2ncc3cc(C)c(C4CCN(C5CCOC5)CC4F)cc32)n1 ; Cc1nc(N2CC(O)C2)cc(-n2ncc3cc(C)c(C4CCN(C5CCOC5)CC4F)cc32)n1]

The title compounds were prepared by a procedure similar to that described for E1 and E2 starting from a solution of cis-6-(3-fluoro-1-(tetrahydrofuran-3-yl)piperidin-4-yl)-5-methyl-1H-indazole (D34) and 1-(6-iodo-2-methylpyrimidin-4-yl)azetidin-3-ol in toluene, CuI, K₃PO₄ and N,N′-dimethylethylenediamine at 100° C.

Chiral Separation:

Method: Column: AD-H; Column size: 0.46 cm I.D.×15 cm L; Mobile phase: Supercritical CO₂:EtOH (0.1% NH₃H₂O)=60:40; Flow rate: 0.5 mL/min; Wave length: UV 254 nm; Temperature: 25° C.; Sample solution in EtOH

Single Unknown Isomer 3

¹H NMR (400 MHz, CDCl₃) δ 8.88 (s, 1H), 8.06 (s, 1H), 7.52 (s, 1H), 6.59 (s, 1H), 4.93˜4.76 (m, 2H), 4.42˜4.38 (m, 2H), 4.00˜3.98 (m, 3H), 3.92˜3.90 (m, 1H), 3.83˜3.81 (m, 1H), 3.75˜3.72 (m, 1H), 3.46 (m, 1H), 3.16˜3.07 (m, 2H), 2.84˜2.82 (m, 2H), 2.63 (s, 3H), 2.47 (s, 3H), 2.28˜2.24 (m, 2H), 2.19˜2.09 (m, 1H), 1.96˜1.85 (m, 3H).

¹⁹F NMR (376 MHz, CDCl₃) δ 183.18 (s)

LC-MS [mobile phase: from 80% water (0.1% FA) and 20% MeCN (0.1% FA) to 5% water (0.1% FA) and 95% MeCN (0.1% FA) in 9 min]: Purity 98%; Rt=2.89 min; MS Calcd: 466.5, MS Found: 467.3 [M+H]⁺.

Chiral HPLC [Column: AD-H; Column size: 0.46 cm I.D.×15 cm L; Injection: 2 μl; Mobile phase: HEP:EtOH (0.1% DEA)=60:40; Flow rate: 0.5 ml/min; Wave length: UV 254 nm; Temperature: 25° C.]: Rt: 2.261 min, ee: 100%

Single Unknown Isomer 4

¹H NMR (400 MHz, CDCl₃) δ 8.89 (s, 1H), 8.06 (s, 1H), 7.52 (s, 1H), 6.59 (s, 1H), 4.94˜4.79 (m, 2H), 4.43˜4.39 (m, 2H), 4.00˜3.97 (m, 3H), 3.94˜3.90 (m, 1H), 3.82˜3.80 (m, 1H), 3.75˜3.73 (m, 1H), 3.28˜3.26 (m, 1H), 3.20˜3.17 (m, 1H), 3.10˜3.06 (m, 2H), 2.63 (s, 3H), 2.47 (s, 3H), 2.34˜2.26 (m, 1H), 2.25˜2.18 (m, 2H), 2.12˜2.05 (m, 1H), 1.97˜1.89 (m, 3H).

¹⁹F NMR (376 MHz, CDCl₃) δ 183.18 (s)

LC-MS [mobile phase: from 80% water (0.1% FA) and 20% MeCN (0.1% FA) to 5% water (0.1% FA) and 95% MeCN (0.1% FA) in 9 min]: Purity 98%; Rt=2.94 min; MS Calcd: 466.5, MS Found: 467.3 [M+H]⁺.

Chiral HPLC [Column: AD-H; Column size: 0.46 cm I.D.×15 cm L; Injection: 2 μl; Mobile phase: HEP:EtOH (0.1% DEA)=60:40; Flow rate: 0.5 ml; Wave length: UV 254 nm; Temperature: 25° C.]: Rt: 3.538 min, ee: 100%

### Examples 17 and 18

### (3S)-1-(2-Methoxy-6-(5-methyl-6-(1-(tetrahydrofuran-3-yl)piperidin-4-yl)-1H-indazol-1-yl)pyrimidin-4-yl)pyrrolidin-3-ol (Single Unknown Isomer 1, E17 and Single Unknown Isomer 2, E18)

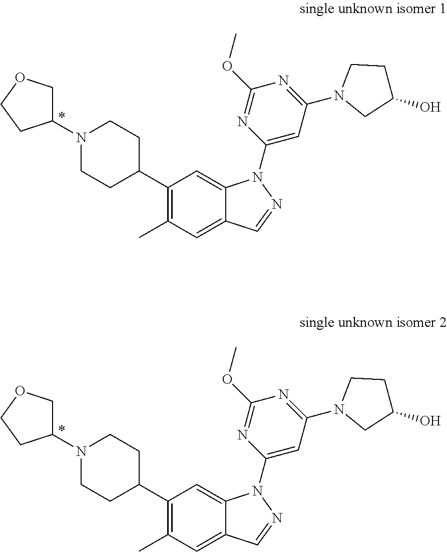 [SMILES: COc1nc(N2CC[C@H](O)C2)cc(-n2ncc3cc(C)c(C4CCN(C5CCOC5)CC4)cc32)n1 ; COc1nc(N2CC[C@H](O)C2)cc(-n2ncc3cc(C)c(C4CCN(C5CCOC5)CC4)cc32)n1]

The title compounds were prepared by a procedure similar to that described for E11 and E12 starting from a mixture of (S)-1-(2-methoxy-6-(5-methyl-6-(piperidin-4-yl)-1H-indazol-1-yl)pyrimidin-4-yl)pyrrolidin-3-ol, dihydrofuran-3(2H)-one, NaBH₃CN and AcOH in DCM at room temperature.

Chiral Separation:

Method: column: Chiralpak ID; 5 μm 250 mm×4.6 mm; Phase: Supercritical CO₂:EtOH (0.1% NH₃H₂O)=50:50; 10 mL/min, 214 nm.

Single Unknown Isomer 1:

¹H NMR (400 MHz, CDCl₃) δ 8.76 (s, 1H), 8.06 (s, 1H), 7.50 (s, 1H), 6.61 (s, 1H), 4.64 (s, 1H), 4.13 (s, 3H), 4.01-3.92 (m, 2H), 3.86-3.67 (m, 5H), 3.17-3.13 (m, 1H), 3.05-3.01 (m, 1H), 2.95-2.92 (m, 1H), 2.86-2.80 (m, 1H), 2.46 (s, 3H), 2.28-2.06 (m, 5H), 1.96-1.83 (m, 5H), 1.71 (s, 1H).

Chiral-HPLC [Chiralpak ID 5 um, 4.6×250 mm; Phase: Hex:EtOH:DEA=70:30:0.2; Flow rate: 1.0 mL/min; Wave length: 230 nm; Temperature: 30° C.]: Rt=9.793 min.

LC-MS [column: C₁₈; column size: 4.6×50 mm; mobile phase: B (MeCN) A (0.02% NH₄Ac); gradient (B %)]: Rt=3.891 min, MS Calcd.: 478, MS Found: 479 [M+H]⁺.

Single Unknown Isomer 2

¹H NMR (400 MHz, CDCl₃) δ 8.76 (s, 1H), 8.07 (s, 1H), 7.51 (s, 1H), 6.62 (s, 1H), 4.64 (s, 1H), 4.13 (s, 3H), 3.99-3.92 (m, 2H), 3.86-3.67 (m, 5H), 3.17-3.15 (m, 1H), 3.03-3.02 (m, 1H), 2.95-2.93 (m, 1H), 2.86-2.80 (m, 1H), 2.46 (s, 3H), 2.25-2.07 (m, 5H), 1.94-1.83 (m, 5H), 1.66 (s, 1H).

Chiral-HPLC (Chiralpak ID 5 m, 4.6×250 mm; Phase: Hex:EtOH:DEA=70:30:0.2; Flow rate: 1.0 mL/min; Wave length: 230 nm; Temperature: 30° C.): Rt=14.573 min.

LC-MS [column: C₁₈; column size: 4.6×50 mm; mobile phase: B (MeCN) A (0.02% NH₄Ac); gradient (B %)]: Rt=4.004 min, MS Calcd.: 478, MS Found: 479 [M+H]⁺.

### Examples 19 and 20

### (3R)-1-(2-Methoxy-6-(5-methyl-6-(1-(tetrahydrofuran-3-yl)piperidin-4-yl)-1H-indazol-1-yl)pyrimidin-4-yl)pyrrolidin-3-ol (Single Unknown Isomer 1, E19 and Single Unknown Isomer 2, E20)

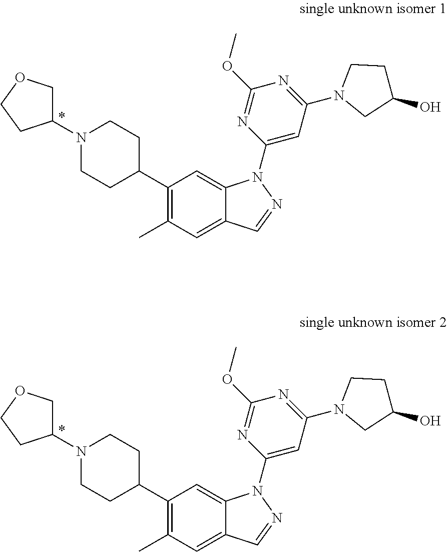 [SMILES: COc1nc(N2CC[C@@H](O)C2)cc(-n2ncc3cc(C)c(C4CCN(C5CCOC5)CC4)cc32)n1 ; COc1nc(N2CC[C@@H](O)C2)cc(-n2ncc3cc(C)c(C4CCN(C5CCOC5)CC4)cc32)n1]

The title compounds were prepared by a procedure similar to that described for E11 and E12 starting from (R)-1-(2-methoxy-6-(5-methyl-6-(piperidin-4-yl)-1H-indazol-1-yl)pyrimidin-4-yl)pyrrolidin-3-ol, dihydrofuran-3(2H)-one, NaBH₃CN in DCM and AcOH (2 drops) at room temperature.

LCMS [column: C₁₈; column size: 4.6×30 mm 5 μm; Dikwa Diamonsil plus; mobile phase: B(MeCN)A (0.02% NH₄Ac+5% MeCN); gradient (B %) in 4 min-5-95-POS; flow 1.5 mL/min, stop time 4 mins]: Rt=2.126 min; MS Calcd.: 478, MS Found: 479 [M+H]⁺.

Chiral Separation:

Method: column: Chiralpak ID; 5 μm 250 mm×4.6 mm; Phase: Supercritical CO₂:EtOH (0.1% NH₃H₂O)=50:50; 10 mL/min, 254 nm.

Single Unknown Isomer 1

¹H NMR (400 MHz, CDCl₃) δ 8.76 (s, 1H), 8.06 (s, 1H), 7.50 (s, 1H), 6.61 (s, 1H), 4.63 (s, 1H), 4.12 (s, 3H), 4.01-3.92 (m, 2H), 3.86-3.66 (m, 5H), 3.17-3.14 (m, 1H), 3.05-3.01 (m, 1H), 2.94-2.92 (m, 1H), 2.85-2.80 (m, 1H), 2.46 (s, 3H), 2.28-2.06 (m, 5H), 1.96-1.83 (m, 6H).

Chiral-HPLC [Chiralpak ID 5 μm 4.6×250 mm; Phase: Hex:EtOH:DEA=50:50:0.2; Flow rate: 1.0 mL/min; Wave Length: 230 nm; Temperature: 30° C.]: Rt=9.793 min.

LC-MS [column: C₁₈; column size: 4.6×50 mm; mobile phase: B (MeCN), A (0.02% NH₄Ac); gradient (B %)]: Rt=4.012 min, MS Calcd.: 478, MS Found: 479 [M+H]⁺.

Single Unknown Isomer 2

¹H NMR (400 MHz, CDCl₃) δ 8.76 (s, 1H), 8.06 (s, 1H), 7.50 (s, 1H), 6.61 (s, 1H), 4.64 (s, 1H), 4.13 (s, 3H), 4.13-3.92 (m, 2H), 3.86-3.67 (m, 5H), 3.17-3.14 (m, 1H), 3.05-3.02 (m, 1H), 2.95-2.92 (m, 1H), 2.84-2.81 (m, 1H), 2.46 (s, 3H), 2.28-2.08 (m, 5H), 2.06-2.86 (m, 5H), 1.72 (s, 1H).

Chiral-HPLC [Chiralpak ID 5 um 4.6×250 mm; Phase: Hex:EtOH:DEA=50:50:0.2; Flow rate: 1.0 mL/min; Wave Length: 230 nm; Temperature: 30° C.)]: Rt=14.573 min.

LC-MS [column: C₁₈; column size: 4.6×50 mm; mobile phase: B (MeCN), A (0.02% NH₄Ac); gradient (B %)]: Rt=2.349 min, MS Calcd.: 478, MS Found: 479 [M+H]⁺.

### Examples 21, 22, 23 and 24

### 1-(4-(2-Methoxy-6-(5-methyl-6-(1-(tetrahydrofuran-3-yl)piperidin-4-yl)-1H-indazol-1-yl)pyrimidin-4-yl)morpholin-2-yl)ethanol (Single Unknown Isomer 1, E21; Single Unknown Isomer 2, E22; Single Unknown Isomer 3, E23; Single Unknown Isomer 4, E24)

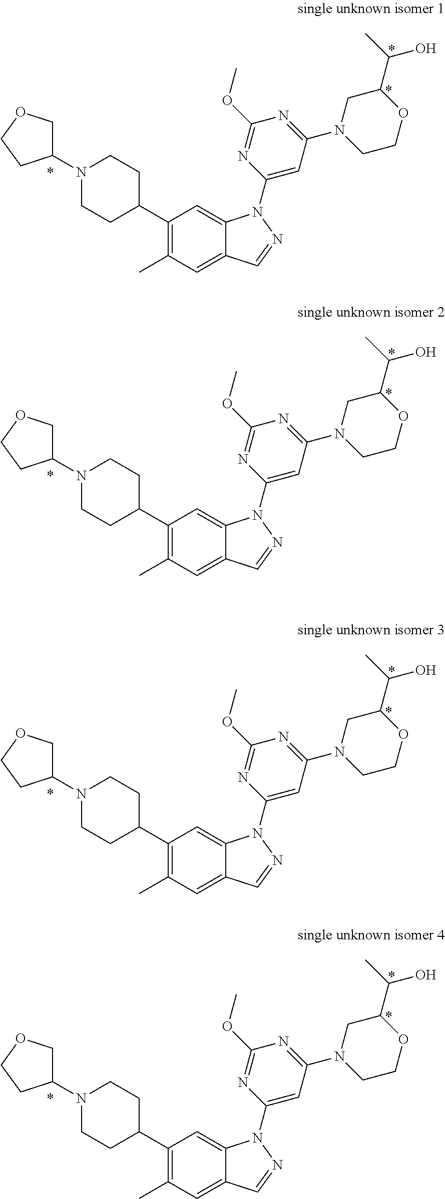 [SMILES: COc1nc(N2CCOC(C(C)O)C2)cc(-n2ncc3cc(C)c(C4CCN(C5CCOC5)CC4)cc32)n1 ; COc1nc(N2CCOC(C(C)O)C2)cc(-n2ncc3cc(C)c(C4CCN(C5CCOC5)CC4)cc32)n1 ; COc1nc(N2CCOC(C(C)O)C2)cc(-n2ncc3cc(C)c(C4CCN(C5CCOC5)CC4)cc32)n1 ; COc1nc(N2CCOC(C(C)O)C2)cc(-n2ncc3cc(C)c(C4CCN(C5CCOC5)CC4)cc32)n1]

The title compounds were prepared by a procedure similar to that described for E11 and E12 starting from a solution of 1-(4-(2-methoxy-6-(5-methyl-6-(piperidin-4-yl)-1H-indazol-1-yl)pyrimidin-4-yl)morpholin-2-yl)ethanol, dihydrofuran-3(2H)-one, NaBH₃CN in DCM and AcOH.

¹HNMR (400 MHz, CDCl₃) δ 8.73 (s, 1H), 8.06 (s, 1H), 7.51 (s, 1H), 6.85 (s, 1H), 4.40-4.24 (m, 2H), 4.13 (s, 3H), 4.06-3.67 (m, 7H), 3.46-3.41 (m, 1H), 3.27-2.83 (m, 6H), 2.46 (s, 3H), 2.34-1.93 (m, 8H), 1.29 (d, J=8.4 Hz, 3H).

Chiral Separation:

Method: CHIRALPAK IA-3 5 cm I.D.×25 cm L; Phase: EtOH/NH₃H₂O=100/0.1 (V/V); Flow rate: 60 mL/min; Wave Length: 254 nm; Temperature: 35° C. single unknown isomer 1

¹H NMR (400 MHz, CDCl₃) δ 8.73 (s, 1H), 8.06 (s, 1H), 7.51 (s, 1H), 6.85 (s, 1H), 4.38-4.23 (m, 2H), 4.13 (s, 3H), 4.05-3.83 (m, 4H), 3.71-3.65 (m, 3H), 3.46-3.42 (m, 1H), 3.18-2.80 (m, 6H), 2.46 (s, 3H), 2.33-2.27 (m, 2H), 2.05-1.96 (m, 2H), 1.90-1.79 (m, 5H), 1.28 (d, J=6.4 Hz, 3H).

Chiral-HPLC [CHIRALPAK IA-3 0.46 cm I.D.×25 cm L; Phase: EtOH/DEA=100/0.1 (V/V); Flow rate: 0.3 mL/min; Wave Length: 254 nm; Temperature: 35° C.]: Rt=17.973 min.

LC-MS [column: C₁₈; column size: 4.6×50 mm; mobile phase: B (MeCN), A (0.02% NH₄Ac); gradient (B %)]: Rt=4.237 min, MS Calcd.: 522, MS Found: 523 [M+H]⁺.

Single Unknown Isomer 2

¹H NMR (400 MHz, CDCl₃) δ 8.73 (s, 1H), 8.06 (s, 1H), 7.51 (s, 1H), 6.85 (s, 1H), 4.35-4.23 (m, 2H), 4.12 (s, 3H), 4.04-3.92 (m, 4H), 3.85-3.79 (m, 1H), 3.72-3.65 (m, 2H), 3.46-3.42 (m, 1H), 3.18-2.80 (m, 6H), 2.46 (s, 3H), 2.32-2.10 (m, 2H), 2.04-2.02 (m, 2H), 1.92-1.82 (m, 5H), 1.28 (d, J=6.8 Hz, 3H).

Chiral-HPLC [CHIRALPAK IA-3 0.46 cm I.D.×25 cm L; Phase: EtOH/DEA=100/0.1 (V/V); Flow rate: 0.3 mL/min; Wave Length: 254 nm; Temperature: 35° C.]: Rt=19.116 min.

LC-MS [column: C₁₈; column size: 4.6×50 mm; mobile phase: B (MeCN), A (0.02% NH₄Ac); gradient (B %)]: Rt=3.637 min, MS Calcd.: 522, MS Found: 523 [M+H]⁺.

Single Unknown Isomer 3

¹H NMR (400 MHz, CDCl₃) δ 8.73 (s, 1H), 8.06 (s, 1H), 7.51 (s, 1H), 6.85 (s, 1H), 4.38-4.23 (m, 2H), 4.12 (s, 3H), 4.04-3.92 (m, 4H), 3.85-3.79 (m, 1H), 3.72-3.65 (m, 2H), 3.46-3.42 (m, 1H), 3.16-2.80 (m, 6H), 2.46 (s, 3H), 2.29-2.03 (m, 4H), 1.94-1.80 (m, 5H), 1.28 (d, J=6.4 Hz, 3H).

Chiral-HPLC [CHIRALPAK IA-3 0.46 cm I.D.×25 cm L; Phase: EtOH/DEA=100/0.1 (V/V); Flow rate: 0.3 mL/min; Wave Length: 254 nm; Temperature: 35° C.]: Rt=23.611 min.

LC-MS [column: C₁₈; column size: 4.6×50 mm; mobile phase: B (MeCN), A (0.02% NH₄Ac); gradient (B %)]: Rt=3.648 min, MS Calcd.: 522, MS Found: 523 [M+H]⁺.

Single Unknown Isomer 4

¹H NMR (400 MHz, CDCl₃) δ 8.73 (s, 1H), 8.06 (s, 1H), 7.51 (s, 1H), 6.85 (s, 1H), 4.38-4.23 (m, 2H), 4.12 (s, 3H), 4.08-3.92 (m, 4H), 3.85-3.79 (m, 1H), 3.71-3.65 (m, 2H), 3.46-3.42 (m, 1H), 3.19-2.80 (m, 6H), 2.46 (s, 3H), 2.30-2.04 (m, 4H), 2.00-1.80 (m, 5H), 1.28 (d, J=6.4 Hz, 3H).

Chiral-HPLC [CHIRALPAK IA-3 0.46 cm I.D.×25 cm L; Phase: EtOH/DEA=100/0.1 (V/V); Flow rate: 0.3 mL/min; Wave Length: 254 nm; Temperature: 35° C.]: Rt=25.808 min.

LC-MS [column: C₁₈; column size: 4.6×50 mm; mobile phase: B (MeCN), A (0.02% NH₄Ac); gradient (B %)]: Rt=3.647 min, MS Calcd.: 522, MS Found: 523 [M+H]⁺.

### Examples 25 and 26

### ((2S)-4-(6-(5-Chloro-6-(1-(tetrahydrofuran-3-yl)piperidin-4-yl)-1H-indazol-1-yl)-2-methylpyrimidin-4-yl)morpholin-2-yl)methanol (Single Unknown Isomer 1, E25 and Single Unknown Isomer 2, E26)

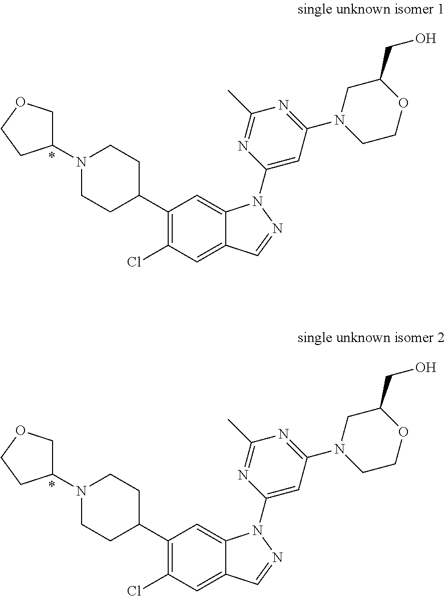 [SMILES: Cc1nc(N2CCO[C@H](CO)C2)cc(-n2ncc3cc(Cl)c(C4CCN(C5CCOC5)CC4)cc32)n1 ; Cc1nc(N2CCO[C@H](CO)C2)cc(-n2ncc3cc(Cl)c(C4CCN(C5CCOC5)CC4)cc32)n1]

The title compounds were prepared by a procedure similar to that described for E1 and E2 starting from a mixture of 5-chloro-6-(1-(tetrahydrofuran-3-yl)piperidin-4-yl)-1H-indazole, (S)-(4-(6-iodo-2-methylpyrimidin-4-yl)morpholin-2-yl)methanol, N,N-dimethylcyclohexane-1,2-diamine, CuI and K₃PO₄ in toluene.

¹H NMR (400 MHz, CDCl₃) δ 8.74 (s, 1H), 8.16 (s, 1H), 7.81 (s, 1H), 7.04 (s, 1H), 4.40-4.27 (m, 3H), 4.14-4.09 (m, 3H), 3.87-3.63 (m, 6H), 3.42-3.39 (m, 1H), 3.30-3.25 (m, 1H), 3.17-3.11 (m, 1H), 3.04-2.96 (m, 2H), 2.73 (s, 3H), 2.41-2.23 (m, 6H).

Chiral Separation:

Method: column: Chiralpak ID; 5 m 20×150 mm; Phase: Supercritical CO₂:EtOH=70:30, Flow rate: 12 mL/min; Wave length: 230 nm.

Single Unknown Isomer 1

¹H NMR (400 MHz, CDCl₃) δ 8.90 (s, 1H), 8.07 (s, 1H), 7.74 (s, 1H), 6.95 (s, 1H), 4.34-4.25 (m, 2H), 4.10-3.93 (m, 3H), 3.86-3.64 (m, 6H), 3.23-2.92 (m, 6H), 2.63 (s, 3H), 2.25-2.21 (m, 2H), 2.17-2.09 (m, 4H), 1.99-1.95 (m, 3H).

Chiral-HPLC [column: Chiralpak IF 5 μm 4.6×250 mm; Phase: Hex:EtOH=70:30; Flow rate: 1.0 mL/min; Wave length: 230 nm; Temperature: 30° C.]: Rt=11.319 min

LC-MS [column: C₁₈; column size: 4.6×50 mm; mobile phase: B (MeCN) A (0.02% NH₄Ac); gradient (B %)]: Rt=4.053 min, MS Calcd.: 512, MS Found: 513 [M+H]⁺.

Single Unknown Isomer 2

¹H NMR (400 MHz, CDCl₃) δ 8.90 (s, 1H), 8.07 (s, 1H), 7.75 (s, 1H), 6.96 (s, 1H), 4.33-4.27 (m, 2H), 4.11-3.95 (m, 3H), 3.87-3.68 (m, 6H), 3.21-2.92 (m, 6H), 2.63 (s, 3H), 2.33-2.24 (m, 2H), 2.15-2.08 (m, 2H), 1.98-1.85 (m, 5H).

Chiral-HPLC [column: Chiralpak IF 5 μm 4.6×250 mm; Phase:Hex:EtOH=70:30; Flow rate: 1.0 mL/min; Wave length: 230 nm; Temperature: 30° C.]: Rt=14.219 min

LC-MS [column: C₁₈; column size: 4.6×50 mm; mobile phase: B (MeCN) A (0.02% NH₄Ac); gradient (B %)]: Rt=4.068 min, MS Calcd.: 512, MS Found: 513 [M+H]⁺.

### Examples 27 and 28

### 1-(6-(5-Chloro-6-(1-(tetrahydrofuran-3-yl)piperidin-4-yl)-1H-indazol-1-yl)-2-methylpyrimidin-4-yl)azetidin-3-ol (Single Unknown Enantiomer 1, E27 and Single Unknown Enantiomer 2, E28)

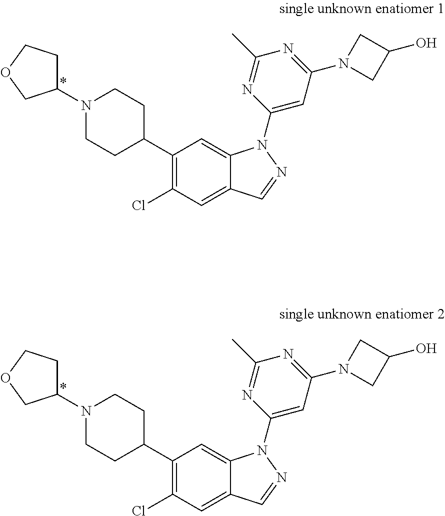 [SMILES: Cc1nc(N2CC(O)C2)cc(-n2ncc3cc(Cl)c(C4CCN(C5CCOC5)CC4)cc32)n1 ; Cc1nc(N2CC(O)C2)cc(-n2ncc3cc(Cl)c(C4CCN(C5CCOC5)CC4)cc32)n1]

The title compounds were prepared by a procedure similar to that described for E1 and E2 starting from a solution of 5-chloro-6-(1-(tetrahydrofuran-3-yl)piperidin-4-yl)-1H-indazole, 1-(6-iodo-2-methylpyrimidin-4-yl)azetidin-3-ol in toluene, CuI, K₃PO₄ and N,N′-dimethylethyle-ne-diamine.

Chiral Separation:

Method: Column: AD-H; Column size: 0.46 cm I.D.×15 cm L; Mobile phase: Supercritical CO₂:EtOH (0.1% NH₃H₂O)=60:40; Flow rate: 0.5 mL/min; Wave length: UV 254 nm; Temperature: 25° C.; Sample solution in EtOH

Single Unknown Enantiomer 1

¹H NMR (400 MHz, CDCl₃) δ 8.9 (s, 1H), 8.07 (s, 1H), 7.74 (s, 1H), 6.59 (s, 1H), 4.83 (s, 1H), 4.43˜4.39 (t, 2H), 4.02˜3.94 (m, 4H), 3.84˜3.82 (m, 1H), 3.73˜3.69 (m, 1H), 3.20 2.95 (m, 4H), 2.61 (s, 3H), 2.29˜2.26 (m, 2H), 2.13˜2.10 (m, 3H), 2.07˜1.85 (m, 3H).

LC-MS [mobile phase: from 80% water (0.1% FA) and 20% MeCN (0.1% FA) to 5% water (0.1% FA) and 95% MeCN (0.1% FA) in 9 min]: Rt=3.01 min; MS Calcd: 468.20, MS Found: 469.20 [M+H]⁺.

Chiral HPLC [Column: AD-H; Column size: 0.46 cm I.D.×15 cm L; Injection: 2 μl; Mobile phase: HEP:IPA (0.1% DEA)=60:40; Flow rate: 0.5 ml; Wave length: UV 254 nm; Temperature: 25° C.]: Rt: 5.435 min, ee 100%;

Single Unknown Enantiomer 2

¹H NMR (400 MHz, CDCl₃) 8.92 (s, 1H), 8.07 (s, 1H), 7.73 (s, 1H), 6.59 (s, 1H), 4.84 (s, H), 4.42˜4.38 (t, 2H), 4.02˜3.96 (m, 4H), 3.86˜3.82 (m, 1H), 3.73˜3.69 (m, 1H), 3.20 2.95 (m, 4H), 2.69 (s, 3H), 2.32˜2.23 (m, 2H), 2.13˜2.08 (m, 3H), 1.94˜1.84 (m, 3H).

LC-MS [mobile phase: from 80% water (0.1% FA) and 20% MeCN (0.1% FA) to 5% water (0.1% FA) and 95% MeCN (0.1% FA) in 9 min]: Rt=3.03 min; MS Calcd: 468.20, MS Found: 469.2 [M+H]⁺.

Chiral HPLC [Column: AD-H; Column size: 0.46 cm I.D.×15 cm L; Injection: 2 μl; Mobile phase: HEP:IPA (0.1% DEA)=60:40; Flow rate: 0.5 ml; Wave length: UV 254 nm; Temperature: 25° C.]: Rt: 6.459 min, ee 100%.

### Examples 29 and 30

### 1-(6-(5-Chloro-6-(1-(tetrahydrofuran-3-yl)piperidin-4-yl)-1H-indazol-1-yl)-2-methoxy-pyrimidin-4-yl)azetidin-3-ol (Single Unknown Enantiomer 1, E29 and Single Unknown Enantiomer 2, E30)

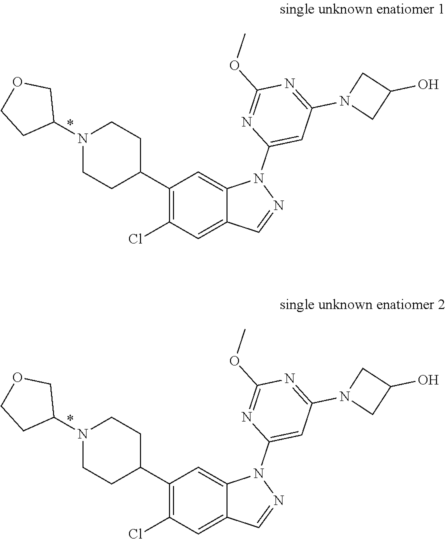 [SMILES: COc1nc(N2CC(O)C2)cc(-n2ncc3cc(Cl)c(C4CCN(C5CCOC5)CC4)cc32)n1 ; COc1nc(N2CC(O)C2)cc(-n2ncc3cc(Cl)c(C4CCN(C5CCOC5)CC4)cc32)n1]

The title compounds were prepared by a procedure similar to that described for E1 and E2 starting from a solution of 5-chloro-6-(1-(tetrahydrofuran-3-yl)piperidin-4-yl)-1H-indazole and 1-(6-iodo-2-methoxylpyrimidin-4-yl)azetidin-3-ol in toluene, CuI, K₃PO₄ and N,N′-dimethylethylenediamine at 100° C.

LC-MS [mobile phase: from 50% water (0.1% FA) and 50% MeCN (0.1% FA) to 5% water (0.1% FA) and 95% MeCN (0.1% FA) in 2.6 min]: Rt=0.76 min; MS Calcd: 484.20, MS Found: 485.2 [M+H]⁺.

Chiral Separation:

Method: Column: AD-H; Column size: 0.46 cm I.D.×15 cm L; Mobile phase: Supercritical CO₂:IPA (0.1% NH₃H₂O)=60:40; Flow rate: 0.5 mL/min; Wave length: UV 254 nm; Temperature: 25° C.; Sample solution in EtOH.

Single Unknown Enantiomer 1

¹H NMR (400 MHz, CDCl₃) δ 8.83 (s, 1H), 8.08 (s, 1H), 7.74 (s, 1H), 6.48 (s, 1H), 4.86˜4.84 (m, 1H), 4.43˜4.40 (m, 2H), 4.11 (s, 3H), 4.04˜3.91 (m, 4H), 3.84˜3.81 (m, 1H), 3.71˜3.66 (m, 1H), 3.15˜3.11 (m, 2H), 3.06˜3.01 (m, 1H), 2.94˜2.91 (m, 1H), 2.31˜2.21 (m, 3H), 2.11˜2.01 (m, 3H), 1.94˜1.89 (m, 1H), 1.83˜1.77 (m, 2H).

LC-MS [mobile phase: from 80% water (0.1% FA) and 20% MeCN (0.1% FA) to 5% water (0.1% FA) and 95% MeCN (0.1% FA) in 9.0 min]: Rt=3.56 min; MS Calcd: 484.20, MS Found: 485.3 [M+H]⁺.

Chiral HPLC [AD-H; Column size: 0.46 cm I.D.×15 cm L; Injection: 2 μl; Mobile phase: HEP:IPA (0.1% DEA)=60:40; Flow rate: 0.5 mL; Wave length: UV 254 nm; Temperature: 25° C.]: Rt: 3.429 min, ee: 100%.

Single Unknown Enantiomer 2

¹H NMR (400 MHz, CDCl₃) δ 8.83 (s, 1H), 8.08 (s, 1H), 7.74 (s, 1H), 6.48 (s, 1H), 4.85˜4.83 (m, 1H), 4.44˜4.40 (m, 2H), 4.11 (s, 3H), 4.04˜3.93 (m, 4H), 3.84˜3.81 (m, 1H), 3.71˜3.66 (m, 1H), 3.16˜3.12 (m, 2H), 3.04˜3.01 (m, 1H), 2.94˜2.91 (m, 1H), 2.32˜2.21 (m, 3H), 2.07˜2.00 (m, 3H), 1.94˜1.90 (m, 1H), 1.82˜1.78 (m, 2H).

LC-MS [mobile phase: from 80% water (0.1% FA) and 20% MeCN (0.1% FA) to 5% water (0.1% FA) and 95% MeCN (0.1% FA) in 9.0 min]: Purity: 100% @ 254 nm; Rt=3.57 min; MS Calcd: 484.20, MS Found: 485.3 [M+H]⁺.

Chiral HPLC [AD-H; Column size: 0.46 cm I.D.×15 cm L; Injection: 2 μl; Mobile phase: HEP:IPA (0.1% DEA)=60:40; Flow rate: 0.5 mL; Wave length: UV 254 nm; Temperature: 25° C.]: Rt: 3.677 min, ee: 100%.

### Examples 31 and 32

### 1-(2-Methoxy-6-(5-methyl-6-(1-(tetrahydrofuran-3-yl)piperidin-4-yl)-1H-indazol-1-yl)-pyrimidin-4-yl)azetidin-3-ol (Single Unknown Enantiomer 1, E31 and Single Unknown Enantiomer 2, E32)

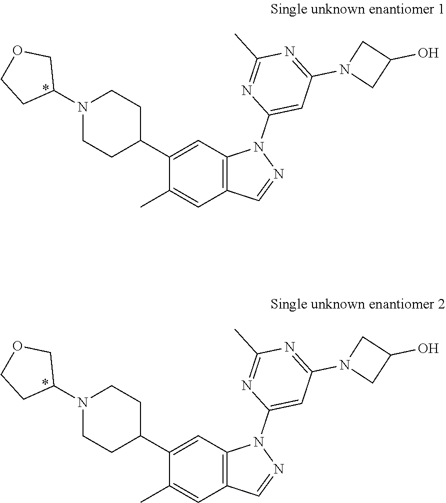 [SMILES: Cc1nc(N2CC(O)C2)cc(-n2ncc3cc(C)c(C4CCN(C5CCOC5)CC4)cc32)n1 ; Cc1nc(N2CC(O)C2)cc(-n2ncc3cc(C)c(C4CCN(C5CCOC5)CC4)cc32)n1]

The title compounds were prepared by a procedure similar to that described for E1 and E2 starting from a solution of 5-methyl-6-(1-(tetrahydrofuran-3-yl)piperidin-4-yl)-1H-indazole, 1-(6-iodo-2-methylpyrimidin-4-yl)azetidin-3-ol in toluene/THF, DMEDA, CuI and K₃PO₄ at 90° C.

Chiral Separation:

Method: AD-H, 0.46 cm I.D.×15 cm L, Mobile phase: Supercritical CO₂:IPA (0.1% NH₃H₂O)=60:40, Flow rate: 0.5 mL/min, Wave length: 254 nm, Temperature: 25° C.

Single Unknown Enantiomer 1

¹H NMR (400 MHz, CDCl₃) δ 8.80 (s, 1H), 68.05 (s, 1H), δ 7.49 (s, 1H), δ 6.58 (s, 1H), δ 4.83 (s, 1H), δ 4.42˜4.38 (m, 2H), δ 4.02˜3.94 (m, 4H), δ 3.84˜3.82 (m, 1H), δ 3.74˜3.70 (m, 1H), δ 3.49 (s, 1H), δ 3.21˜3.18 (d, J=10.4 Hz, 1H), δ 3.06˜3.04 (m, 1H), δ 2.98˜2.96 (d, J=9.6 Hz, 1H), δ 2.85˜2.82 (m, 1H), δ 2.61 (s, 3H), δ 2.45 (s, 3H), δ 2.26˜2.21 (m, 2H), δ 2.11˜2.09 (m, 1H), δ 1.96˜1.91 (m, 6H).

LC-MS [mobile phase: From 90% water (0.1% FA) and 10% MeCN (0.1% FA) to 5% water (0.1% FA) and 95% MeCN (0.1% FA) in 9 min]: Rt=3.99 min; MS Calcd.:448.5, MS Found: 449.4 [M+H]⁺.

Chiral HPLC [AD-H, 0.46 cm I.D.×15 cm L, Mobile phase: HEP:PA (0.1% DEA)=60:40, Flow rate: 0.5 mL/min, 254 nm, Temperature: 25° C.]: Rt=1.961 min, ee: 100%

Single Unknown Enantiomer 2

¹H NMR (400 MHz, CDCl₃) δ 8.80 (s, 1H), δ8.05 (s, 1H), 7.52 (s, 1H), δ 6.58 (s, 1H), δ 4.82 (s, 1H), δ 4.42˜4.38 (m, 2H), δ 4.01˜3.93 (m, 4H), δ 3.84˜3.81 (m, 1H), δ 3.74˜3.70 (m, 1H), δ 3.21˜3.18 (m, 2H), δ 3.05˜2.96 (m, 2H), δ 2.84˜2.82 (m, 1H), δ 2.61 (s, 3H), δ 2.45 (s, 3H), δ 2.26˜2.20 (m, 2H), δ 2.12˜2.09 (m, 1H), δ 1.97˜1.91 (m, 6H).

LC-MS [mobile phase: From 90% water (0.1% FA) and 10% MeCN (0.1% FA) to 5% water (0.1% FA) and 95% MeCN (0.1% FA) in 9 min]: Rt=3.98 min; MS Calcd.:448.5, MS Found: 449.4 [M+H]⁺.

Chiral HPLC [Chiral HPLC [AD-H, 0.46 cm I.D.×15 cm L, Mobile phase: HEP:IPA (0.1% DEA)=60:40, Flow rate: 0.5 mL/min, 254 nm, Temperature: 25° C.]: Rt=2.686 min, ee: 99.4%

### Examples 33 and 34

### ((2S)-4-(6-(6-(1-(3-Deuterium-tetrahydrofuran-3-yl)piperidin-4-yl)-5-methyl-1H-indazol 1-yl)-2-methylpyrimidin-4-yl)morpholin-2-yl)methanol (Single Unknown Isomer 1, E33 and Single Unknown Isomer 2, E34)

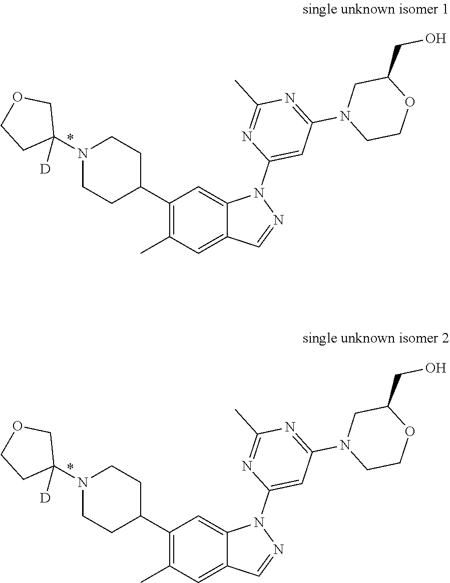 [SMILES: [2H]C1(N2CCC(c3cc4c(cnn4-c4cc(N5CCO[C@H](CO)C5)nc(C)n4)cc3C)CC2)CCOC1 ; [2H]C1(N2CCC(c3cc4c(cnn4-c4cc(N5CCO[C@H](CO)C5)nc(C)n4)cc3C)CC2)CCOC1]

A mixture of 6-(1-(3-deuteriumtetrahydrofuran-3-yl)piperidin-4-yl)-5-methyl-1H-indazole (120 mg, 0.420 mmol), (S)-(4-(6-iodo-2-methylpyrimidin-4-yl)morpholin-2-yl)methanol (167 mg, 0.500 mmol), N,N′-dimethylcyclohexane-1,2-diamine (119 mg, 0.840 mmol), CuI (80.0 mg, 0.420 mmol) and K₃PO₄ (178 mg, 0.840 mmol) in toluene (3 mL) was stirred at 100° C. for 2 hrs, then diluted with EtOAc (30 mL), washed with brine (30 mL), dried over Na₂SO₄, filtered and concentrated. The residue was purified by silica gel chromatography column (DCM/MeOH=15/1) to give the desired product (30 mg, 14%) as a yellow oil.

¹H NMR (400 MHz, CDCl₃) δ 8.78 (s, 1H), 8.05 (s, 1H), 7.49 (s, 1H), 6.94 (s, 1H), 4.32-4.27 (m, 2H), 4.07-3.94 (m, 3H), 3.86-3.65 (m, 6H), 3.21-2.82 (m, 5H), 2.63 (s, 3H), 2.45 (s, 3H), 2.30-2.21 (m, 3H), 2.14-2.08 (m, 1H), 1.98-1.91 (m, 5H).

Chiral Separation:

Method: column: Chiralpak ID; 5 μm 20×150 mm; Phase: Supercritical CO₂:IPA=50:50; Flow rate: 8 mL/min, Wave length: 254 nm.

Single Unknown Isomer 1

¹H NMR (400 MHz, CDCl₃) δ 8.78 (s, 1H), 8.05 (s, 1H), 7.50 (s, 1H), 6.94 (s, 1H), 4.32-4.29 (m, 2H), 4.07-3.94 (m, 3H), 3.86-3.65 (m, 6H), 3.22-3.08 (m, 2H), 3.00-2.83 (m, 3H), 2.63 (s, 3H), 2.46 (s, 3H), 2.31-2.22 (m, 2H), 2.15-2.09 (m, 1H), 1.94-1.93 (m, 5H), 1.27-1.20 (m, 1H).

Chiral-HPLC [column: chiral pak IE, 5 μm 250 mm×4.6 mm; mobile phase: Hex:IPA=50:50; flow rate: 1 mL/min; Wave length 230 nm; Temperature: 30° C.]: Rt=7.891 min.

LC-MS [column: C₁₈; column size: 4.6×50 mm; mobile phase: B (MeCN) A (0.1% FA); gradient (B %)]: Rt=2.954 min, MS Calcd.: 493, MS Found: 494 [M+H]⁺.

Single Unknown Isomer 2

¹H NMR (400 MHz, CDCl₃) δ 8.78 (s, 1H), 8.06 (s, 1H), 7.50 (s, 1H), 6.95 (s, 1H), 4.32-4.29 (m, 2H), 4.08-3.94 (m, 3H), 3.86-3.66 (m, 6H), 3.21-3.07 (m, 2H), 3.00-2.83 (m, 3H), 2.64 (s, 3H), 2.46 (s, 3H), 2.28-2.22 (m, 2H), 2.15-2.09 (m, 1H), 1.94 (br s, 5H), 1.69-1.62 (m, 1H).

Chiral-HPLC [column: chiral pak IE, 5 μm 250 mm×4.6 mm; mobile phase: Hex:IPA=50:50; flow rate: 1 mL/min; Wave length: 230 nm; Temperature: 30° C.]: Rt=10.583 min.

LC-MS [column: C₁₈; column size: 4.6×50 mm; mobile phase: B (MeCN) A (0.1% FA); gradient (B %)]: Rt=2.324 min, MS Calcd.: 493, MS Found: 494 [M+H]⁺.

### Examples 35 and 36

### ((2R)-4-(6-(6-(1-(3-Eeuterium-tetrahydrofuran-3-yl)piperidin-4-yl)-5-methyl-1H-indazol-1-yl)-2-methylpyrimidin-4-yl)morpholin-2-yl)methanol (Single Unknown Isomer 1, E35 and Single Unknown Isomer 2, E36)

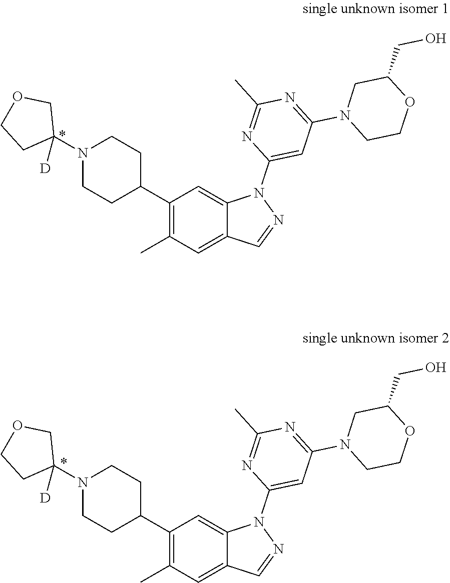 [SMILES: [2H]C1(N2CCC(c3cc4c(cnn4-c4cc(N5CCO[C@@H](CO)C5)nc(C)n4)cc3C)CC2)CCOC1 ; [2H]C1(N2CCC(c3cc4c(cnn4-c4cc(N5CCO[C@@H](CO)C5)nc(C)n4)cc3C)CC2)CCOC1]

The title compounds were prepared by a procedure similar to that described for E1 and E2 starting from a mixture of 6-(1-(3-deuteriumtetrahydrofuran-3-yl)piperidin-4-yl)-5-methyl-1H-indazole, (R)-(4-(6-iodo-2-methylpyrimidin-4-yl)morpholin-2-yl)methanol, CuI, K₃PO₄ and N,N′-dimethylcyclohexane-1,2-diamine in toluene and DMSO.

LCMS [column: C₁₈; column size: 4.6×30 mm 5 μm; Dikwa Diamonsil plus; mobile phase: B (MeCN) A (0.02% NH4Ac+5% MeCN); gradient (B %) in 4 min-10-95-POS; flow rate: 1.5 mL/min, stop time 4 mins]: Rt=2.057 min; MS Calcd.: 493, MS Found: 494 [M+H]⁺.

Chiral Separation:

Method: column: Chiralpak ID; 5 μm 20×150 mm; Phase: Supercritical CO₂:EtOH (0.1% NH₃H₂O)=50:50, Flow rate: 8 mL/min; Wave length: 214 nm

Single Unknown Isomer 1

¹HNMR (400 MHz, CDCl₃) δ 8.78 (s, 1H), 8.05 (s, 1H), 7.50 (s, 1H), 6.95 (s, 1H), 4.32-4.29 (m, 2H), 4.08-3.99 (m, 3H), 3.84-3.67 (m, 6H), 3.21-3.08 (m, 2H), 3.00-2.92 (m, 2H), 2.86-2.83 (m, 1H), 2.63 (s, 3H), 2.46 (s, 3H), 2.31-2.24 (m, 2H), 2.14-2.08 (m, 1H), 1.94-1.92 (m, 5H).

LCMS [column: C₁₈; column size: 4.6×50 mm; mobile phase: B (MeCN) A (0.02% NH₄Ac); gradient (B %) in 6 mins]: Rt=3.927 min; MS Calcd.:493, MS Found: 494 [M+H]⁺.

Chiral HPLC [Chiralpak ID 5 μm 4.6×250 mm; Phase: Hex:IPA:DEA=50:50:0.2; Flow rate: 1.0 mL/min; Wave length: 230 nm; Temperature: 30° C.]: Rt=9.239 min.

Single Unknown Isomer 2

¹HNMR (400 MHz, CDCl₃) δ 8.78 (s, 1H), 8.06 (s, 1H), 7.50 (s, 1H), 6.94 (s, 1H), 4.34-4.28 (m, 2H), 4.05-3.98 (m, 3H), 3.86-3.67 (m, 6H), 3.20-3.08 (m, 2H), 3.00-2.92 (m, 2H), 2.87-2.82 (m, 1H), 2.64 (s, 3H), 2.46 (s, 3H), 2.30-2.26 (m, 2H), 2.23-2.12 (m, 1H), 2.08-1.93 (m, 5H).

LCMS [column: C1₈; column size: 4.6×50 mm; mobile phase: B (MeCN) A (0.02% NH₄Ac); gradient (B %) in 6 mins]: Rt=3.947 min; MS Calcd.: 493, MS Found: 494 [M+H]⁺.

Chiral HPLC [Chiralpak ID 5 μm 4.6×250 mm; Phase: Hex:IPA:DEA=50:50:0.2; Flow rate: 1.0 mL/min; Wave length: 230 nm; Temperature: 30° C.]: Rt=14.337 min.

### Examples 37 and 38

### 3-Methyl-1-(2-methyl-6-(5-methyl-6-(1-(tetrahydrofuran-3-yl)piperidin-4-yl)-1H-indazol-1-yl)pyrimidin-4-yl)azetidin-3-ol (Single Unknown Enantiomer 1, E37 and Single Unknown Enantiomer 2, E38)

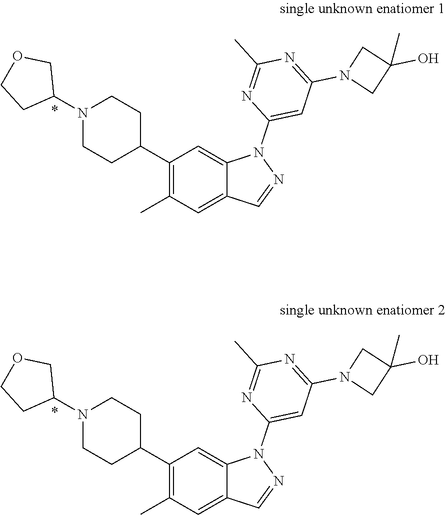 [SMILES: Cc1nc(N2CC(C)(O)C2)cc(-n2ncc3cc(C)c(C4CCN(C5CCOC5)CC4)cc32)n1 ; Cc1nc(N2CC(C)(O)C2)cc(-n2ncc3cc(C)c(C4CCN(C5CCOC5)CC4)cc32)n1]

The title compounds were prepared by a procedure similar to that described for E1 and E2 starting from a mixture of 5-methyl-6-(1-(tetrahydrofuran-3-yl)piperidin-4-yl)-1H-indazole, 1-(6-iodo-2-methylpyrimidin-4-yl)-3-methylazetidin-3-ol, N,N′-dimethylcyclohexane-1,2-diamine, CuI and K₃PO₄ in toluene at 100° C.

LCMS [column: C₁₈; column size: 4.6×30 mm 5 μm; Dikwa Diamonsil plus; mobile phase: B (MeCN) A1 (0.02% NH₄Ac+5% MeCN); gradient(B %) in 4 mins. 10-95-POS; flow rate: 1.5 mL/min]: Rt=2.325 min; MS Calcd.:462, MS Found: 463 [M+H]⁺.

Chiral Separation:

Method: column: Chiralpak ID 5 μm 20×150 mm; Phase: Supercritical CO₂:IPA (0.1% NH₃H₂O)=60:40, flow rate: 8 mL/min; Wave length: 214 nm.

Single Unknown Enantiomer 1

¹H NMR (400 MHz, CDCl₃) δ 8.80 (s, 1H), 8.05 (s, 1H), 7.49 (s, 1H), 6.59 (s, 1H), 4.10-3.93 (m, 6H), 3.86-3.74 (m, 2H), 3.23-3.22 (m, 1H), 3.08-2.98 (m, 2H), 2.86-2.83 (m, 1H), 2.63 (s, 3H), 2.45 (s, 3H), 2.29-2.09 (m, 4H), 1.94-1.88 (m, 5H), 1.78-1.62 (m, 2H).

Chiral-HPLC [Column: Chiralpak ID 250 mm×4.6 mm 5 um; Mobile phase: Hex:IPA:DEA=60:40:0.2; Flow rate:1 mL/min; Wave length: 230 nm; Temperature=ambient]: Rt=7.126 min.

LC-MS [column: C₁₈; column size: 4.6×50 mm; mobile phase: B (MeCN) A (0.1% FA); gradient (B %)]: Rt=2.741 min, MS Calcd.: 462, MS Found: 463 [M+H]⁺.

Single Unknown Enantiomer 2

¹H NMR (400 MHz, CDCl₃) δ 8.80 (s, 1H), 8.05 (s, 1H), 7.49 (s, 1H), 6.59 (s, 1H), 4.09-3.94 (m, 6H), 3.87-3.70 (m, 2H), 3.20 (d, J=10.8 Hz, 1H), 3.05-2.96 (m, 2H), 2.85-2.81 (m, 1H), 2.63 (s, 3H), 2.45 (s, 3H), 2.36-2.20 (m, 3H), 2.14-2.09 (m, 1H), 1.97-1.92 (m, 5H), 1.70 (br s, 2H).

Chiral-HPLC [Column: Chiralpak ID 250 mm×4.6 mm 5 um; Mobile phase: Hex:IPA:DEA=60:40:0.2; Flow rate:1 mL/min; Wave length: 230 nm; Temperature=ambient]: Rt=9.805 min.

LC-MS [column: C₁₈; column size: 4.6×50 mm; mobile phase: B (MeCN) A (0.1% FA); gradient (B %)]: Rt=3.866 min, MS Calcd.: 462, MS Found: 463 [M+H]⁺.

### Examples 39, 40, 41 and 42

### 1-(1-(6-(5-Chloro-6-(1-(tetrahydrofuran-3-yl)piperidin-4-yl)-1H-indazol-1-yl)-2-methylpyrimidin-4-yl)azetidin-3-yl)ethanol (Single Unknown Isomer 1, E39; Single Unknown Isomer 2, E40; Single Unknown Isomer 3, E41; Single Unknown Isomer 4, E42)

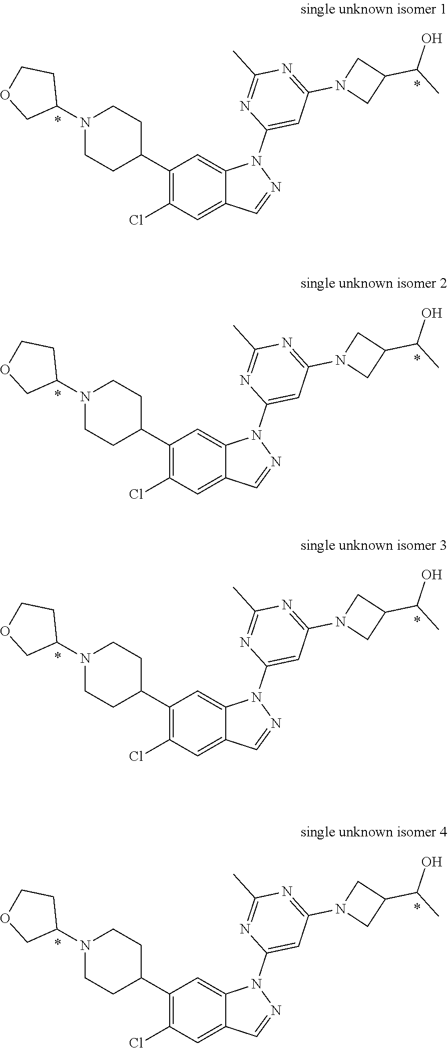 [SMILES: Cc1nc(N2CC(C(C)O)C2)cc(-n2ncc3cc(Cl)c(C4CCN(C5CCOC5)CC4)cc32)n1 ; Cc1nc(N2CC(C(C)O)C2)cc(-n2ncc3cc(Cl)c(C4CCN(C5CCOC5)CC4)cc32)n1 ; Cc1nc(N2CC(C(C)O)C2)cc(-n2ncc3cc(Cl)c(C4CCN(C5CCOC5)CC4)cc32)n1 ; Cc1nc(N2CC(C(C)O)C2)cc(-n2ncc3cc(Cl)c(C4CCN(C5CCOC5)CC4)cc32)n1]

The title compound was prepared by a procedure similar to that described for E1 and E2 starting from a solution of 1-(1-(6-iodo-2-methyl pyrimidin-4-yl)azetidin-3-yl)ethanol, 5-chloro-6-(1-(tetrahydrofuran-3-yl)piperid in-4-yl)-1H-indazole in toluene, CuI, K₃PO₄.3H₂O, N,N′dimethylethylenediamine.

LC-MS [mobile phase: from 70% water (0.1% FA) and 30% MeCN (0.1% FA) to 5% water (0.1% FA) and 95% MeCN (0.1% FA) in 2.6 min]: Rt=0.95 min; MS Calcd: 496.24, MS Found: 497.2 [M+H]⁺.

Chiral Separation

Method: Column: AD-H; Column size: 0.46 cm I.D.×15 cm L; mobile phase: Supercritical CO₂:EtOH (0.1% NH₃H₂O)=60:40; Flow rate: 0.5 mL/min; Wave length: UV 254 nm; Temperature: 25° C.; Sample solution in EtOH

Single Unknown Isomer 1

¹H NMR (400 MHz, CDCl₃) δ 8.91 (s, 1H), 8.06 (s, 1H), 7.73 (s, 1H), 6.58 (s, 1H), 4.20˜4.18 (m, 2H), 4.17˜3.96 (m, 5H), 3.91˜3.82 (m, 2H), 3.73˜3.71 (m, 1H), 3.17˜2.95 (m, 4H), 2.75 (br s, 1H), 2.62 (s, 3H), 2.29˜2.26 (m, 2H), 2.11˜2.03 (m, 3H), 1.93˜1.85 (m, 3H), 1.23˜1.22 (d, J=6 Hz, 3H).

LC-MS [mobile phase: from 90% water (0.1% FA) and 10% MeCN (0.1% FA) to 5% water (0.1% FA) and 95% MeCN (0.1% FA) in 9.0 min]: Purity: >97% @ 254 nm; Rt=4.19 min; MS Calcd: 496.24, MS Found: 497.3 [M+H]⁺.

Chiral HPLC [Column: AD-H; Column size: 0.46 cm I.D.×15 cm L; Injection: 2 μl; mobile phase: HEP:EtOH (0.05% DEA)=60:40; Flow rate: 0.5 ml/min; Wave length: UV 254 nm; Temperature: 25° C.]: Rt: 5.256 min, ee 100%

Single Unknown Isomer 2

¹H NMR (400 MHz, CDCl₃) δ 8.91 (s, 1H), 8.06 (s, 1H), 7.73 (s, 1H), 6.58 (s, 1H), 4.18 (br s, 2H), 4.07˜3.91 (m, 5H), 3.89˜3.82 (m, 2H), 3.73˜3.71 (m, 1H), 3.20˜2.95 (m, 4H), 2.77 2.75 (m, 1H), 2.62 (s, 3H), 2.29˜2.26 (m, 2H), 2.11˜2.02 (m, 3H), 1.94˜1.85 (m, 3H), 1.23˜1.22 (d, J=6 Hz, 3H).

LC-MS [mobile phase: from 90% water (0.1% FA) and 10% MeCN (0.1% FA) to 5% water (0.1% FA) and 95% MeCN (0.1% FA) in 9.0 min]: Purity: 100% @ 254 nm; Rt=4.20 min; MS Calcd: 496.24, MS Found: 497.3 [M+H]⁺.

Chiral HPLC [Column: AD-H; Column size: 0.46 cm I.D.×15 cm L; Injection: 2 μl; mobile phase: HEP:EtOH (0.05% DEA)=60:40; Flow rate: 0.5 ml/min; Wave length: UV 254 nm; Temperature: 25° C.]: Rt: 5.524 min, ee 97%;

Single Unknown Isomer 3

¹H NMR (400 MHz, CDCl₃) δ 8.91 (s, 1H), 8.06 (s, 1H), 7.73 (s, 1H), 6.58 (s, 1H), 4.20˜4.17 (m, 2H), 4.07˜3.96 (m, 5H), 3.89˜3.82 (m, 2H), 3.73˜3.71 (m, 1H), 3.20˜2.95 (m, 4H), 2.77˜2.75 (m, 1H), 2.62 (s, 3H), 2.29˜2.26 (m, 2H), 2.11˜2.03 (m, 3H), 1.93˜1.85 (m, 3H), 1.23˜1.22 (d, J=6.4 Hz, 3H).

LC-MS [mobile phase: from 90% water (0.1% FA) and 10% MeCN (0.1% FA) to 5% water (0.1% FA) and 95% MeCN (0.1% FA) in 9.0 min]:Purity: 100% @ 254 nm; Rt=4.19 min; MS Calcd: 496.24, MS Found: 497.3 [M+H]⁺.

Chiral HPLC [Column: AD-H; Column size: 0.46 cm I.D.×15 cm L; Injection: 2 μl; mobile phase: HEP:EtOH (0.05% DEA)=60:40; Flow rate: 0.5 ml/min; Wave length: UV 254 nm; Temperature: 25° C.]: Rt: 5.777 min, ee 97%;

Single Unknown Isomer 4

¹H NMR (400 MHz, CDCl₃) δ 8.91 (s, 1H), 8.06 (s, 1H), 7.73 (s, 1H), 6.58 (s, 1H), 4.21˜4.17 (m, 2H), 4.05˜3.94 (m, 5H), 3.88˜3.82 (m, 2H), 3.73˜3.69 (m, 1H), 3.20˜2.98 (m, 4H), 2.77˜2.75 (m, 1H), 2.61 (s, 3H), 2.29˜2.26 (m, 2H), 2.09˜2.03 (m, 3H), 1.93˜1.85 (m, 3H), 1.23˜1.22 (d, J=6 Hz, 3H).

LC-MS [mobile phase: from 90% water (0.1% FA) and 10% MeCN (0.1% FA) to 5% water (0.1% FA) and 95% MeCN (0.1% FA) in 9.0 min]: Purity: >86% @ 254 nm; Rt=4.16 min; MS Calcd: 496.24, MS Found: 497.3 [M+H]⁺.

Chiral HPLC [Column: AD-H; Column size: 0.46 cm I.D.×15 cm L; Injection: 2 μl; mobile phase: HEP:EtOH (0.05% DEA)=60:40; Flow rate: 0.5 ml/min; Wave length: UV 254 nm; Temperature: 25° C.]: Rt: 6.022 min, ee 98%;

### Examples 43, 44, 45 and 46

### 1-(1-(6-(5-Chloro-6-(1-(tetrahydrofuran-3-yl)piperidin-4-yl)-1H-indazol-1-yl)-2-methylpyr-imidin-4-yl)azetidin-3-yl)propan-2-ol (Single Unknown Isomer 1, E43; Single Unknown Isomer 2, E44; Single Unknown Isomer 3, E45; Single Unknown Isomer 4, E46)

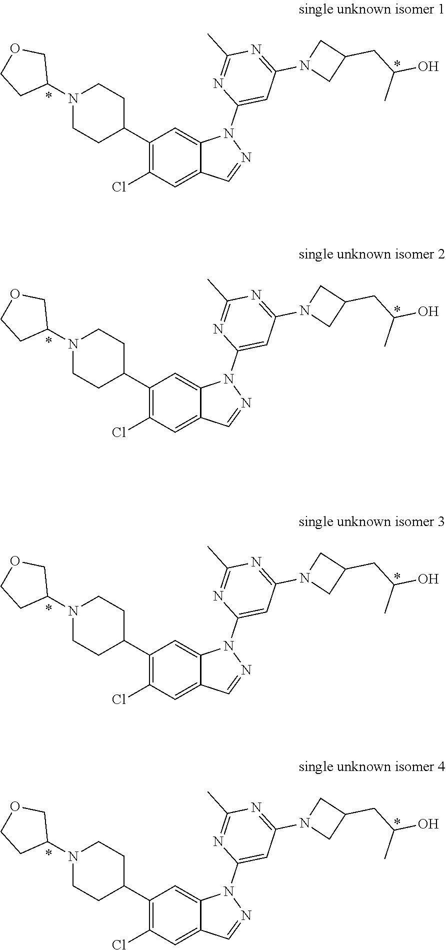 [SMILES: Cc1nc(N2CC(CC(C)O)C2)cc(-n2ncc3cc(Cl)c(C4CCN(C5CCOC5)CC4)cc32)n1 ; Cc1nc(N2CC(CC(C)O)C2)cc(-n2ncc3cc(Cl)c(C4CCN(C5CCOC5)CC4)cc32)n1 ; Cc1nc(N2CC(CC(C)O)C2)cc(-n2ncc3cc(Cl)c(C4CCN(C5CCOC5)CC4)cc32)n1 ; Cc1nc(N2CC(CC(C)O)C2)cc(-n2ncc3cc(Cl)c(C4CCN(C5CCOC5)CC4)cc32)n1]

The title compound was prepared by a procedure similar to that described for E1 and E2 starting from a mixture of 5-chloro-6-(1-(tetrahydrofuran-3-yl)piperidin-4-yl)-1H-indazole, 1-(1-(6-iodo-2-methylpyrimidi-n-4-yl)azetidin-3-yl)propan-2-ol, N,N′-dimethyl-ethane-1,2-diamine, CuI and K₃PO₄.3H₂O in toluene.

LC-MS [mobile phase: from 90% water (0.1% FA) and 10% MeCN (0.1% FA) to 50% water (0.1% FA) and 50% MeCN (0.1% FA) in 2.6 min]: Purity: 99% @ 254 nm; Rt=0.88 min; MS Calcd: 510.2, MS Found: 511.2 [M+H]⁺.

Chiral Separation:

Method: AD-H, 0.46 cm I.D×15 cm L, Phase: Supercritical CO₂: EtOH (0.1% NH₃H₂O)=60/40, Flow rate: 0.5 mL/min, Wave length: 254 nm, Temperature: 25° C.

Single Unknown Isomer 1

¹H NMR (400 MHz, CDCl₃) δ 8.91 (s, 1H), 8.06 (s, 1H), 7.73 (s, 1H), 6.55 (s, 1H), 4.30-4.26 (m, 2H), 4.01-3.96 (m, 2H), 3.89-3.80 (m, 4H), 3.74-3.70. (m, 1H), 3.21-2.96 (m, 5H), 2.61 (s, 3H), 2.33-2.24 (m, 2H), 2.14-2.08 (m, 3H), 1.98-1.64 (m, 5H), 1.25 (d, J=6.4 Hz, 3H).

LC-MS [mobile phase: from 90% water (0.1% FA) and 10% MeCN (0.1% FA) to 90% water (0.1% FA) and 10% MeCN (0.1% FA) in 9 min]: purity 100%, Rt=4.16 min; MS Calcd: 510.3, MS Found: 511.2 [M+H]⁺.

Chiral HPLC [AD-H, 0.46 cm I.D×15 cm L, Phase: HEP: EtOH (0.1% DEA)=60/40, Flow rate: 0.5 mL/min, Wave length: 254 nm, Temperature: 25° C.]: Rt: 5.103 min; ee: 100%

Single Unknown Isomer 2

¹H NMR (400 MHz, CDCl₃) δ 8.91 (s, 1H), 8.06 (s, 1H), 7.73 (s, 1H), 6.55 (s, 1H), 4.30-4.26 (m, 2H), 4.01-3.96 (m, 2H), 3.89-3.80 (m, 4H), 3.74-3.70. (m, 1H), 3.21-2.96 (m, 5H), 2.61 (s, 3H), 2.33-2.24 (m, 2H), 2.14-2.08 (m, 3H), 1.98-1.64 (m, 5H), 1.25 (d, J=6.4 Hz, 3H).

LC-MS [mobile phase: from 90% water (0.1% FA) and 10% MeCN (0.1% FA) to 90% water (0.1% FA) and 10% MeCN (0.1% FA) in 9 min]: purity 100%, Rt=4.15 min; MS Calcd: 510.2, MS Found: 511.2 [M+H]⁺.

Chiral HPLC [AD-H, 0.46 cm I.D×15 cm L, Phase: HEP: EtOH (0.1% DEA)=60/40, Flow rate: 0.5 mL/min, Wave length: 254 nm, Temperature: 25° C.]: Rt: 5.246 min; ee: 100%

Single Unknown Isomer 3

¹H NMR (400 MHz, CDCl₃) δ 8.91 (s, 1H), 8.06 (s, 1H), 7.73 (s, 1H), 6.55 (s, 1H), 4.30-4.26 (m, 2H), 4.01-3.96 (m, 2H), 3.89-3.80 (m, 4H), 3.74-3.70. (m, 1H), 3.21-2.96 (m, 5H), 2.61 (s, 3H), 2.33-2.24 (m, 2H), 2.14-2.08 (m, 3H), 1.98-1.64 (m, 5H), 1.25 (d, J=6.4 Hz, 3H).

LC-MS [mobile phase: from 90% water (0.1% FA) and 10% MeCN (0.1% FA) to 90% water (0.1% FA) and 10% MeCN (0.1% FA) in 9 min]: purity 94%, Rt=4.17 min; MS Calcd: 510.2, MS Found: 511.2 [M+H]⁺.

Chiral HPLC [AD-H, 0.46 cm I.D×15 cm L, Phase: HEP: EtOH (0.1% DEA)=60/40, Flow rate: 0.5 mL/min, Wave length: 254 nm, Temperature: 25° C.]: Rt: 5.596 min; ee: 99.9%

Single Unknown Isomer 4

¹H NMR (400 MHz, CDCl₃) δ 8.91 (s, 1H), 8.06 (s, 1H), 7.73 (s, 1H), 6.55 (s, 1H), 4.30-4.26 (m, 2H), 4.01-3.96 (m, 2H), 3.89-3.80 (m, 4H), 3.74-3.70. (m, 1H), 3.21-2.96 (m, 5H), 2.61 (s, 3H), 2.33-2.24 (m, 2H), 2.14-2.08 (m, 3H), 1.98-1.64 (m, 5H), 1.25 (d, J=6.4 Hz, 3H).

LC-MS [mobile phase: from 90% water (0.1% FA) and 10% MeCN (0.1% FA) to 90% water (0.1% FA) and 10% MeCN (0.1% FA) in 9 min]: purity 92%, Rt=4.14 min; MS Calcd: 510.2, MS Found: 511.2 [M+H]⁺.

Chiral HPLC [AD-H, 0.46 cm I.D×15 cm L, Phase: HEP: EtOH (0.1% DEA)=60/40, Flow rate: 0.5 mL/min, Wave length: 254 nm, Temperature: 25° C.]: Rt: 5.735 min; ee: 99.7%

### Examples 47 and 48

### 1-(6-(5-Chloro-6-(1-(tetrahydrofuran-3-yl)piperidin-4-yl)-1H-indazol-1-yl)-2-methoxypyri-midin-4-yl)-3-methylazetidin-3-ol (Single Unknown Enantiomer 1, E47 and Single Unknown Enantiomer 2, E48)

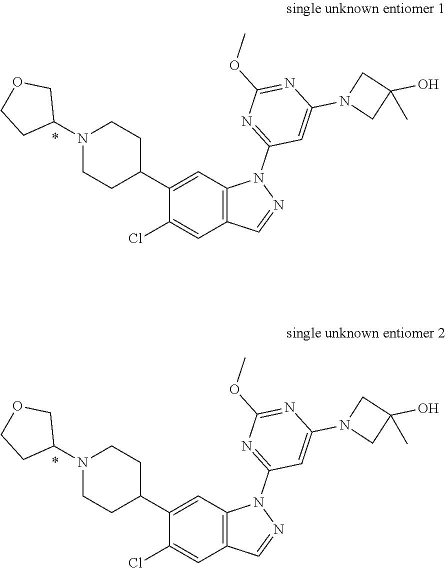 [SMILES: COc1nc(N2CC(C)(O)C2)cc(-n2ncc3cc(Cl)c(C4CCN(C5CCOC5)CC4)cc32)n1 ; COc1nc(N2CC(C)(O)C2)cc(-n2ncc3cc(Cl)c(C4CCN(C5CCOC5)CC4)cc32)n1]

The title compounds were prepared by a procedure similar to that described as E1 and E2 starting from a suspension of 5-chloro-6-(1-(tetrahydrofuran-3-yl)piperidin-4-yl)-1H-indazole, 1-(6-iodo-2-methoxypyrimidin-4-yl)-3-methylazetidin-3-ol, N,N′-dimethyl-cyclohexane-1,2-diamine, CuI, K₃PO₄ in toluene.

Chiral Separation:

Method: column: Chiralpak IA; 5 μm 20×150 mm; Phase: Supercritical CO₂:EtOH=70:30; flow rate:11 mL/min, Wave length: 254 nm

Single Unknown Enantiomer 1

¹HNMR (400 MHz, CDCl₃) δ 8.83 (s, 1H), 8.08 (s, 1H), 7.75 (s, 1H), 6.49 (s, 1H), 4.11 (s, 3H), 4.09-4.06 (m, 4H), 4.01-3.92 (m, 2H), 3.82 (q, J=8.0 Hz, 1H), 3.69 (t, J=7.6 Hz, 1H), 3.16-3.02 (m, 3H), 2.95-2.92 (m, 1H), 2.27 (q, J=12 Hz, 2H), 2.21-2.01 (m, 4H), 1.96-1.89 (m, 1H), 1.87-1.75 (m, 2H), 1.62 (s, 3H).

LCMS [column: Phenomenex Kinetex 5 μm EVO, C₁₈; column size: 4.6×50 mm; mobile phase: B (MeCN), A (0.02% NH₄Ac); gradient (B %) in 6 mins]: Rt=3.669 min, MS Calcd.:498, MS Found: 499 [M+H]⁺.

Chiral-HPLC [column: chiral pak IA, 5 μm 250 mm×4.6 mm; mobile phase: Hex:EtOH=70:30; flow rate: 1 mL/min; Wave length: 230 nm; Temperature: 30° C.]: Rt=7.142 min

Single Unknown Enantiomer 2

¹HNMR (400 MHz, CDCl₃) δ 8.83 (s, 1H), 8.08 (s, 1H), 7.74 (s, 1H), 6.48 (s, 1H), 4.11 (s, 3H), 4.10-4.06 (m, 4H), 4.01-3.92 (m, 2H), 3.82 (q, J=8.0 Hz, 1H), 3.70 (t, J=7.6 Hz, 1H), 3.17-3.03 (m, 3H), 2.95-2.92 (m, 1H), 2.27 (q, J=12 Hz, 3H), 2.21-2.00 (m, 3H), 1.95-1.90 (m, 1H), 1.82-1.79 (m, 2H), 1.62 (s, 3H).

LCMS [column: Phenomenex Kinetex 5 μm EVO, C₁₈; column size: 4.6×50 mm; mobile phase: B (MeCN) A (0.02% NH₄Ac); gradient (B %) in 6 mins]: Rt=3.672 min; MS Calcd.:498, MS Found: 499 [M+H]⁺.

Chiral-HPLC [column: chiral pak IA, 5 μm 250 mm×4.6 mm; mobile phase: Hex:EtOH=70:30; flow rate: 1 mL/min; Wave length: 230 nm; Temperature: 30° C.]: Rt=9.859 min.

### Examples 49 to 52

### 1-(1-(2-Methoxy-6-(5-methyl-6-(1-(tetrahydrofuran-3-yl)piperidin-4-yl)-1H-indazol-1-yl)pyrimidin-4-yl)azetidin-3-yl)propan-2-ol (Single Unknown Isomer 1, E49; Single Unknown Isomer 2, E50; Single Unknown Isomer 3, E51; Single Unknown Isomer 4, E52)

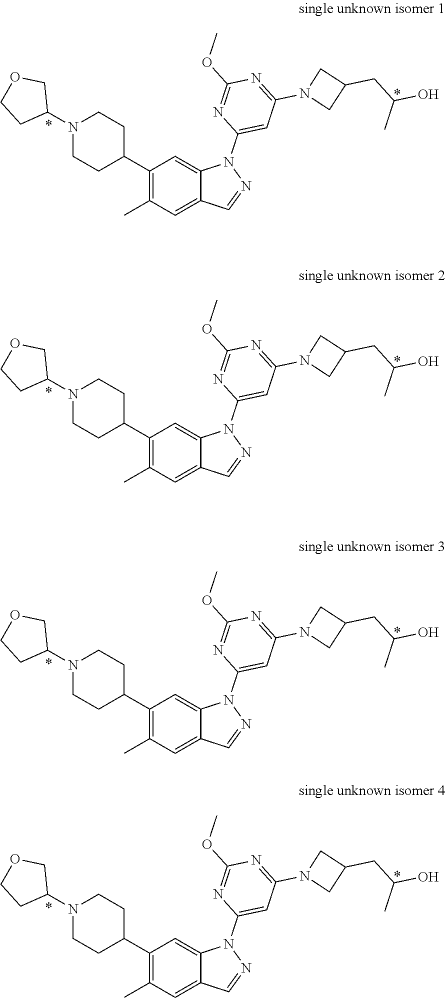 [SMILES: COc1nc(N2CC(CC(C)O)C2)cc(-n2ncc3cc(C)c(C4CCN(C5CCOC5)CC4)cc32)n1 ; COc1nc(N2CC(CC(C)O)C2)cc(-n2ncc3cc(C)c(C4CCN(C5CCOC5)CC4)cc32)n1 ; COc1nc(N2CC(CC(C)O)C2)cc(-n2ncc3cc(C)c(C4CCN(C5CCOC5)CC4)cc32)n1 ; COc1nc(N2CC(CC(C)O)C2)cc(-n2ncc3cc(C)c(C4CCN(C5CCOC5)CC4)cc32)n1]

The title compounds were prepared by a procedure similar to that described as E1 and E2 starting from a solution of 1-(1-(6-iodo-2-methoxypyrimidin-4-yl)azetidin-3-yl)propan-2-ol, 5-methyl-6-(tetrahydrofuran-3-yl)-1H-indazole in toluene, N¹,N²-dimethylethane-1,2-diamine, CuI and K₃PO₄.

Chiral Separation:

Method: AD-H, 0.46 cm I.D×15 cm L, Phase: Supercritical CO₂: ⁱPrOH (0.1% NH₃H₂O)=60/40, flow rate: 0.5 mL/min, Wave length: 254 nm, Temperature: 25° C.

Single Unknown Isomer 1

¹H NMR (400 MHz, CDCl₃) δ 8.74 (s, 1H), 8.05 (s, 1H), 7.49 (s, 1H), 6.43 (s, 1H), 4.29-4.28 (m, 2H), 4.26 (s, 3H), 3.98-3.68 (m, 7H), 3.16-2.82 (m, 5H), 2.45 (s, 3H), 2.24-1.63 (m, 11H), 1.24-1.22 (m, 3H).

LC-MS [mobile phase: from 80% water (0.1% FA) and 20% MeCN (0.1% FA) to 5% water (0.1% FA) and 95% MeCN (0.1% FA) in 9 min], purity: 98.55%; Rt=3.72 min; MS Calcd: 506, MS Found: 507 [M+H]⁺.

Chiral HPLC [method: AD-H, 0.46 cm I.D×15 cm L, Phase: HEP:ⁱPrOH (0.05% DEA)=60/40, flow rate: 0.5 mL/min, Wave length: 254 nm, Temperature: 25° C.]: Rt: 5.234 min; ee: 100%

Single Unknown Isomer 2

¹H NMR (400 MHz, CDCl₃) δ 8.74 (s, 1H), 8.05 (s, 1H), 7.49 (s, 1H), 6.43 (s, 1H), 4.29-4.28 (m, 2H), 4.26 (s, 3H), 3.98-3.68 (m, 7H), 3.16-2.82 (m, 5H), 2.45 (s, 3H), 2.24-1.63 (m, 11H), 1.24-1.22 (m, 3H).

LC-MS [mobile phase: from 80% water (0.1% FA) and 20% MeCN (0.1% FA) to 5% water (0.1% FA) and 95% MeCN (0.1% FA) in 9 min]: purity: 100%; Rt=3.71 min; MS Calcd: 506, MS Found: 507 [M+H]⁺.

Chiral HPLC [method: AD-H, 0.46 cm I.D×15 cm L, Phase: HEP:ⁱPrOH (0.05% DEA)=60/40, flow rate: 0.5 mL/min, Wave length: 254 nm, Temperature: 25° C.]: Rt: 5.420 min; ee: 90.7%

Single Unknown Isomer 3

¹H NMR (400 MHz, CDCl₃) δ 8.74 (s, 1H), 8.05 (s, 1H), 7.49 (s, 1H), 6.43 (s, 1H), 4.29-4.28 (m, 2H), 4.26 (s, 3H), 3.98-3.68 (m, 7H), 3.16-2.82 (m, 5H), 2.45 (s, 3H), 2.24-1.63 (m, 11H), 1.24-1.22 (m, 3H).

LC-MS [mobile phase: from 80% water (0.1% FA) and 20% MeCN (0.1% FA) to 5% water (0.1% FA) and 95% MeCN (0.1% FA) in 9 min]: purity: 100%; Rt=3.74 min; MS Calcd: 506, MS Found: 507 [M+H]⁺.

Chiral HPLC [method: AD-H, 0.46 cm I.D×15 cm L, Phase: HEP:ⁱPrOH (0.05% DEA)=60/40, flow rate: 0.5 mL/min, Wave length: 254 nm, Temperature: 25° C.]: Rt: 6.645 min; ee: 100%

Single Unknown Isomer 4

¹H NMR (400 MHz, CDCl₃) 8.74 (s, 1H), 8.05 (s, 1H), 7.49 (s, 1H), 6.43 (s, 1H), 4.29-4.28 (m, 2H), 4.26 (s, 3H), 3.98-3.68 (m, 7H), 3.16-2.82 (m, 5H), 2.45 (s, 3H), 2.24-1.63 (m, 11H), 1.24-1.22 (m, 3H).

LC-MS [mobile phase: from 80% water (0.1% FA) and 20% MeCN (0.1% FA) to 5% water (0.1% FA) and 95% MeCN (0.1% FA) in 9 min]: purity: 100%; Rt=3.71 min; MS Calcd: 506, MS Found: 507 [M+H]⁺.

Chiral HPLC [method: AD-H, 0.46 cm I.D×15 cm L, Phase: HEP:ⁱPrOH (0.05% DEA)=60/40, flow rate: 0.5 mL/min, Wave length: 254 nm, Temperature: 25° C.]: Rt: 7.015 min; ee: 97.7%

### Examples 53 and 54

### 4-(6-(6-(3-Fluoro-1-(tetrahydrofuran-3-yl)piperidin-4-yl)-5-methyl-1H-indazo-1-yl)-2-methoxypyrimidin-4-yl)piperazin-2-one (Single Unknown Isomer 1, E53; Single Unknown Isomer 2, E54)

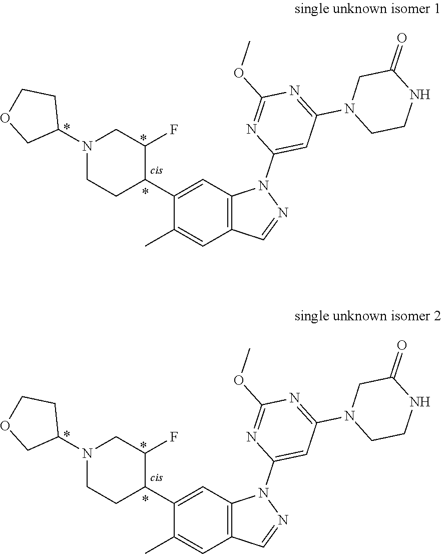 [SMILES: COc1nc(N2CCNC(=O)C2)cc(-n2ncc3cc(C)c(C4CCN(C5CCOC5)CC4F)cc32)n1 ; COc1nc(N2CCNC(=O)C2)cc(-n2ncc3cc(C)c(C4CCN(C5CCOC5)CC4F)cc32)n1]

A mixture of cis-1-(6-chloro-2-methoxypyrimidin-4-yl)-6-(3-fluoro-1-(tetrahydrofuran-3-yl)piperidin-4-yl)-5-methyl-1H-indazole (D70, 100 mg, 0.220 mmol), piperazin-2-one (24.0 mg, 0.240 mmol) and Et₃N (67.0 mg, 0.660 mmol) in DMF (3 mL) was stirred at 40° C. overnight. The reaction mixture was poured into water (50 mL) and extracted with EtOAc (30 mL×3). The combined organic layers were dried over Na₂SO₄, filtered and concentrated. The residue was purified by Prep-HPLC (A: water, B: MeCN, A:B=80:20 to A:B=5:95) to give the title product as a white solid. (35 mg, yield 30%). The chiral mixture was separated by chiral prep-HPLC.

Chiral Separation:

Method: AD-H, 0.46 cm I.D×15 cm L, Phase: Supercritical CO₂: EtOH (0.1% NH₃H₂O)=60/40, Flow rate: 0.5 mL/min, Wave length: 254 nm, Temperature: 25° C.

Single Unknown Isomer 1

¹H NMR (400 MHz, CDCl₃) δ 8.84 (s, 1H), 8.08 (s, 1H), 7.54 (s, 1H), 6.84 (s, 1H), 6.04 (s, 1H), 4.88˜4.77 (m, 1H), 4.33 (s, 2H), 4.11 (s, 3H), 4.01 (s, 3H), 3.99˜3.98 (m, 1H), 3.93˜3.91 (m, 1H), 3.74˜3.72 (m, 1H), 3.52 (s, 2H), 3.23˜3.20 (m, 1H), 3.18˜3.14 (m, 2H), 3.09˜3.02 (m, 1H), 2.48 (s, 3H), 2.34˜2.31 (m, 1H), 2.29˜2.22 (m, 1H), 2.20˜2.08 (m, 1H), 1.96˜1.88 (m, 3H).

¹⁹F NMR (376 MHz, CDCl₃) δ-183.33.

LC-MS [mobile phase: from 95% water (0.1% FA) and 5% MeCN (0.1% FA) to 5% water (0.1% FA) and 95% MeCN (0.1% FA) in 9 min]: purity 100%, Rt=4.37 min; MS Calcd: 509.5, MS Found: 510.4 [M+H]⁺.

Chiral HPLC [AD-H, 0.46 cm I.D.×15 cm L, Phase: HEP:EtOH (0.1% DEA)=60/40, Flow rate: 0.5 mL/min, Wave length: 254 nm, Temperature: 25° C.]: Rt: 3.207 min, ee: 100%.

Single Unknown Isomer 2

¹H NMR (400 MHz, CDCl₃) 8.84 (s, 1H), 8.08 (s, 1H), 7.54 (s, 1H), 6.84 (s, 1H), 6.03 (s, 1H), 4.88˜4.76 (m, 0.54H), 4.33 (s, 2H), 4.11 (s, 3H), 4.01 (s, 3H), 3.99˜3.98 (m, 1H), 3.93˜3.91 (m, 1H), 3.74˜3.72 (m, 1H), 3.52 (s, 2H), 3.44 (s, 1H), 3.16˜3.14 (m, 2H), 2.82˜2.81 (m, 1H), 2.48 (s, 3H), 2.26˜2.23 (m, 2H), 2.09 (s, 1H), 1.96˜1.88 (m, 3H).

¹⁹F NMR (376 MHz, CDCl₃) δ-183.22.

LC-MS [mobile phase: from 95% water (0.1% FA) and 5% MeCN (0.1% FA) to 5% water (0.1% FA) and 95% MeCN (0.1% FA) in 9 min]: purity 100%, Rt=4.36 min; MS Calcd: 509.5, MS Found: 510.4 [M+H]⁺.

Chiral HPLC [AD-H, 0.46 cm I.D.×15 cm L, Phase: HEP:EtOH (0.1% DEA)=60/40, Flow rate: 0.5 mL/min, Wave length: 254 nm, Temperature: 25° C.]: Rt: 5.775 min, ee: 100%.

### Example 55

### 4-(6-(6-(3-Fluoro-1-(tetrahydrofuran-3-yl)piperidin-4-yl)-5-methyl-1H-indazol-1-yl)-2-methoxypyrimidin-4-yl)piperazin-2-one (Single Unknown Isomer 3)

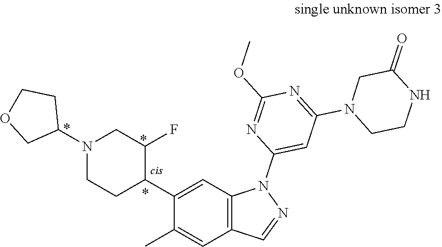 [SMILES: COc1nc(N2CCNC(=O)C2)cc(-n2ncc3cc(C)c(C4CCN(C5CCOC5)CC4F)cc32)n1]

The title compound was prepared by a procedure similar to that described as E53 and E54 starting from a mixture of cis-1-(6-chloro-2-methoxypyrimidin-4-yl)-6-(3-fluoro-1-(tetrahydrofuran-3-yl)piperidin-4-yl)-5-methyl-1H-indazole (D71), piperazin-2-one and NEt₃ in DMF at 40° C.

Chiral Separation:

Method: AD-H, 0.46 cm I.D×15 cm L, Phase: Supercritical CO₂:EtOH (0.1% NH₃H₂O)=60/40, Flow rate: 0.5 mL/min, Wave length: 254 nm, Temperature: 25° C.

¹H NMR (400 MHz, CDCl₃) δ 8.84 (s, 1H), 8.08 (s, 1H), 7.54 (s, 1H), 6.84 (s, 1H), 6.04 (s, 1H), 4.93˜4.88 (m, 0.57H), 4.33 (s, 2H), 4.11 (s, 3H), 4.01 (s, 3H), 3.99˜3.98 (m, 1H), 3.93˜3.91 (m, 1H), 3.74˜3.72 (m, 1H), 3.52 (s, 2H), 3.43˜3.42 (m, 1H), 3.16˜3.14 (m, 2H), 2.83˜2.79 (m, 1H), 2.48 (s, 3H), 2.26˜2.23 (m, 2H), 2.11˜2.09 (m, 1H), 1.96˜1.88 (m, 3H).

¹⁹F NMR (376 MHz, CDCl₃) δ-183.22.

LC-MS [mobile phase: from 95% water (0.1% FA) and 5% MeCN (0.1% FA) to 5% water (0.1% FA) and 95% MeCN (0.1% FA) in 9 min]: purity 100%, Rt=4.39 min; MS Calcd: 509.5, MS Found: 510.4 [M+H]⁺.

Chiral HPLC [AD-H, 0.46 cm I.D×15 cm L, Phase: HEP:EtOH (0.1% DEA)=60/40, Flow rate: 0.5 mL/min, Wave length: 254 nm, Temperature: 25° C.]: Rt: 1.732 min, ee: 100%.

### Examples 56 and 57

### 1-((1-(2-Methyl-6-(5-methyl-6-(1-(tetrahydrofuran-3-yl)piperidin-4-yl)-1H-indazol-1-yl)pyrimidin-4-yl)azetidin-3-yl)oxy)propan-2-ol (Single Unknown Isomer 1, E56 and Single Unknown Isomer 2, E57)

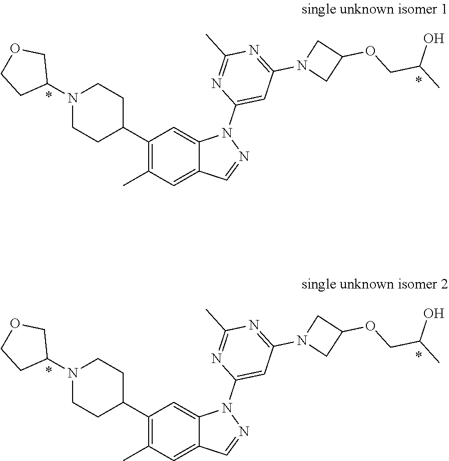 [SMILES: Cc1nc(N2CC(OCC(C)O)C2)cc(-n2ncc3cc(C)c(C4CCN(C5CCOC5)CC4)cc32)n1 ; Cc1nc(N2CC(OCC(C)O)C2)cc(-n2ncc3cc(C)c(C4CCN(C5CCOC5)CC4)cc32)n1]

To a solution of 1-((1-(2-methyl-6-(5-methyl-6-(piperidin-4-yl)-1H-indazol-1-yl)pyrimidin-4-yl)azetidin-3-yl)oxy)propan-2-ol (D77,110 mg, 0.250 mmol), dihydrofuran-3(2H)-one (108 mg, 1.26 mmol) and AcOH (1 drop) in DCE (6 mL) was added NaBH₃CN (32.0 mg, 0.500 mmol). The mixture was stirred at room temperature for 20 hrs, then quenched with a solution of sat. NaHCO₃ (3 drops) and concentrated. The purification via silica gel chromatography column (DCM/MeOH=15/1) afforded the title product (49 mg, 39%) as a colorless oil.

LCMS [column: C₁₈; column size: 4.6×30 mm 5 μm; Dikwa Diamonsil plus; mobile phase: B (MeCN) A₁ (0.02% NH₄Ac+5% MeCN); gradient (B %) in 4 mins. 10-95-POS; flow rate: 1.5 mL/min]: Rt=2.025 min; MS Calcd.: 506, MS Found: 507 [M+H]⁺.

Chiral Separation:

Method: Chiralpak IA 250 mm×4.6 mm 5 um; Mobile phase: Supercritical CO₂: IPA (0.1% NH₃H₂O)=70:30; Flow rate:1 mL/min; Wave length: 230 nm; Temperature=ambient

Single Unknown Isomer 1

¹H NMR (400 MHz, CDCl₃) δ 8.80 (s, 1H), 8.06 (s, 1H), 7.50 (s, 1H), 6.60 (s, 1H), 4.52-4.49 (m, 1H), 4.47-4.32 (m, 2H), 4.06-3.94 (m, 5H), 3.87-3.73 (m, 2H), 3.47-3.44 (m, 1H), 3.29-3.20 (m, 2H), 3.08-2.98 (m, 2H), 2.87-2.83 (m, 1H), 2.64 (s, 3H), 2.46 (s, 3H), 2.30-2.12 (m, 4H), 2.01-1.89 (m, 5H), 1.19 (d, J=8.8 Hz, 3H).

Chiral-HPLC [Chiralpak IA 250 mm×4.6 mm 5 um; Mobile phase: Hex:IPA:DEA=70:30:0.2; Flow rate:1 mL/min; Wave length: 230 nm; Temperature=ambient]: Rt=8.726 min.

LC-MS [column: C₁₈; column size: 4.6×50 mm; mobile phase: B (MeCN) A (0.1% FA); gradient (B %)]: Rt=2.784 min, MS Calcd.: 506, MS Found: 507 [M+H]⁺.

Single Unknown Isomer 2

¹H NMR (400 MHz, CDCl₃) δ 8.80 (s, 1H), 8.06 (s, 1H), 7.50 (s, 1H), 6.60 (s, 1H), 4.52-4.47 (m, 1H), 4.37-4.31 (m, 2H), 4.06-3.94 (m, 5H), 3.87-3.70 (m, 2H), 3.47-3.44 (m, 1H), 3.29-3.17 (m, 2H), 3.05-2.96 (m, 2H), 2.86-2.81 (m, 1H), 2.64 (s, 3H), 2.46 (s, 3H), 2.29-2.09 (m, 4H), 2.00-1.87 (m, 5H), 1.19 (d, J=6.0 Hz, 3H).

Chiral-HPLC [Chiralpak IA 250 mm×4.6 mm 5 um; Mobile phase: Hex:IPA:DEA=70:30:0.2; Flow rate:1 mL/min; Wave length: 230 nm; Temperature=ambient]: Rt=10.678 min.

LC-MS [column: C₁₈; column size: 4.6×50 mm; mobile phase: B (MeCN) A (0.1% FA); gradient (B %)]: Rt=3.012 min, MS Calcd.: 506, MS Found: 507 [M+H]⁺.

### Examples 58 and 59

### 1-((1-(2-Methyl-6-(5-methyl-6-(1-(tetrahydrofuran-3-yl)piperidin-4-yl)-1H-indazol-1-yl)pyrimidin-4-yl)azetidin-3-yl)oxy)propan-2-ol (Single Unknown Isomer 3, E58 and Single Unknown Isomer 4, E59)

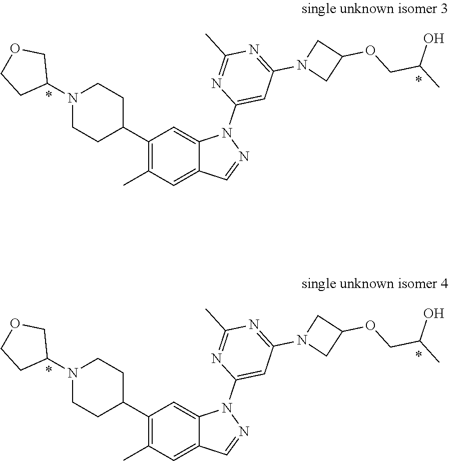 [SMILES: Cc1nc(N2CC(OCC(C)O)C2)cc(-n2ncc3cc(C)c(C4CCN(C5CCOC5)CC4)cc32)n1 ; Cc1nc(N2CC(OCC(C)O)C2)cc(-n2ncc3cc(C)c(C4CCN(C5CCOC5)CC4)cc32)n1]

The title compounds were prepared by a procedure similar to that described as E56 and E57 starting from a solution of 1-((1-(2-methyl-6-(5-methyl-6-(piperidin-4-yl)-1H-indazol-1-yl)pyrimidin-4-yl)azetidin-3-yl)oxy)propan-2-ol (D79, dihydrofuran-3(2H)-one and AcOH (cat.) in DCE and NaBH₃CN.

LCMS [column: C₁₈; column size: 4.6×30 mm 5 μm; Dikwa Diamonsil plus; mobile phase: B (MeCN) A1 (0.02% NH₄Ac+5% MeCN); gradient (B %) in 4 mins. 10-95-POS; flow rate: 1.5 mL/min]: Rt=2.036 min; MS Calcd.:506, MS Found: 507 [M+H]⁺.

Chiral Separation:

Method: column: Chiralpak IA 5 μm 20×150 mm; Phase: Supercritical CO₂:IPA (0.1% NH₃H₂O)=70:30, Flow rate:10 mL/min; Wave length: 254 nm.

Single Unknown Isomer 3

¹H NMR (400 MHz, CDCl₃) δ 8.80 (s, 1H), 8.06 (s, 1H), 7.50 (s, 1H), 6.60 (s, 1H), 4.53-4.47 (m, 1H), 4.36-4.32 (m, 2H), 4.06-3.94 (m, 5H), 3.87-3.70 (m, 2H), 3.49-3.44 (m, 1H), 3.29-3.18 (m, 2H), 3.06-2.97 (m, 2H), 2.86-2.81 (m, 1H), 2.64 (s, 3H), 2.46 (s, 3H), 2.26-1.91 (m, 4H), 1.66-1.57 (m, 5H), 1.19 (d, J=6.4 Hz, 3H).

Chiral-HPLC [Column: Chiralpak IA 250 mm×4.6 mm 5 um; Mobile phase: Hex:EtOH:DEA=70:30:0.2; F:1 mL/min; WL: 230 nm; T=30° C.]: Rt=9.520 min.

LC-MS [column: C₁₈; column size: 4.6×50 mm; mobile phase: B (MeCN) A (0.02% NH₄Ac); gradient (B %)]: Rt=4.045 min, MS Calcd.: 506, MS Found: 507 [M+H]⁺.

Single Unknown Isomer 4

¹H NMR (400 MHz, CDCl₃) δ 8.80 (s, 1H), 8.06 (s, 1H), 7.50 (s, 1H), 6.60 (s, 1H), 4.51-4.47 (m, 1H), 4.36-4.32 (m, 2H), 4.06-3.94 (m, 5H), 3.87-3.71 (m, 2H), 3.47-3.44 (m, 1H), 3.29-3.20 (m, 2H), 3.06-2.98 (m, 2H), 2.86-2.82 (m, 1H), 2.64 (s, 3H), 2.46 (s, 3H), 2.16-2.02 (m, 4H), 1.94-1.88 (m, 5H), 1.19 (d, J=6.4 Hz, 3H).

Chiral-HPLC [Column: Chiralpak IA 250 mm×4.6 mm 5 um; Mobile phase: Hex:EtOH:DEA=70:30:0.2; Flow rate:1 mL/min; Wave length: 230 nm; Temperature=30° C.]: Rt=11.039 min.

LC-MS [column: C₁₈; column size: 4.6×50 mm; mobile phase: B (MeCN) A (0.02% NH₄Ac); gradient (B %)]: Rt=4.041 min, MS Calcd.: 506, MS Found: 507 [M+H]⁺.

### Examples 60 to 63

### (6-Methyl-4-(2-methyl-6-(5-methyl-6-(1-(tetrahydrofuran-3-yl)piperidin-4-yl)-1H-indazol-1-yl)pyrimidin-4-yl)morpholin-2-yl)methanol (Single Unknown Isomer 1, E60; Single Unknown Isomer 2, E61; Single Unknown Isomer 3, E62; Single Unknown Isomer 4, E63)

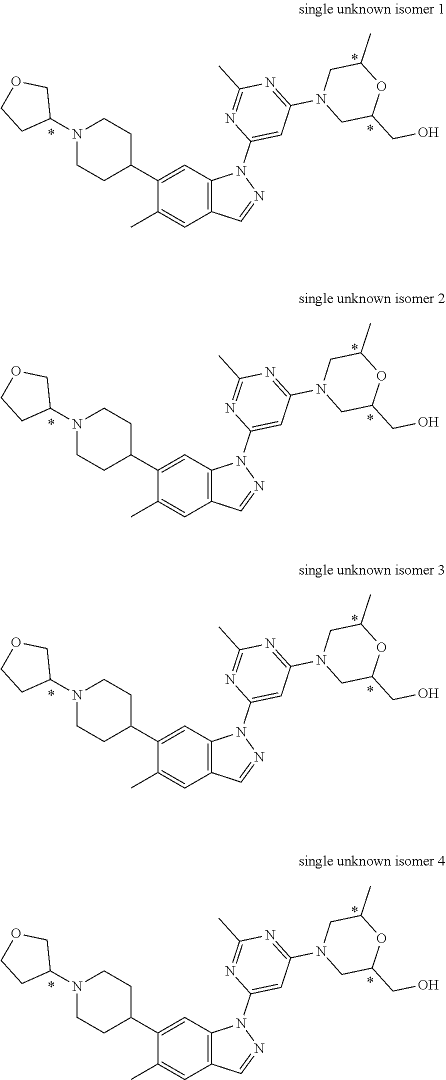 [SMILES: Cc1nc(N2CC(C)OC(CO)C2)cc(-n2ncc3cc(C)c(C4CCN(C5CCOC5)CC4)cc32)n1 ; Cc1nc(N2CC(C)OC(CO)C2)cc(-n2ncc3cc(C)c(C4CCN(C5CCOC5)CC4)cc32)n1 ; Cc1nc(N2CC(C)OC(CO)C2)cc(-n2ncc3cc(C)c(C4CCN(C5CCOC5)CC4)cc32)n1 ; Cc1nc(N2CC(C)OC(CO)C2)cc(-n2ncc3cc(C)c(C4CCN(C5CCOC5)CC4)cc32)n1]

The title compounds were prepared by a procedure similar to that described as E1 and E2 starting from a solution of 5-methyl-6-(1-(tetrahydrofuran-3-yl)piperidin-4-yl)-1H-indazole, (4-(6-iodo-2-methylpyrimidin-4-yl)-6-methylmorpholin-2-yl)methanol (isomer 1, D80) in toluene, CuI, K₃PO₄ and N,N′-dimethylethylenediamine at 100° C.

Chiral Separation:

Method: Column: AD-H; Column size: 0.46 cm I.D.×15 cm L; Mobile phase: Supercritical CO₂:EtOH (0.1% NH₃H₂O)=60:40; Flow rate: 0.5 mL/min; Wave length: UV 254 nm; Temperature: 25° C.; Sample solution in EtOH

Single Unknown Isomer 1

¹H NMR (400 MHz, CDCl₃) δ 8.78 (s, 1H), 8.05 (s, 1H), 7.49 (s, 1H), 6.93 (s, 1H), 4.36 4.32 (m, 2H), 3.99˜3.94 (m, 2H), 3.84˜3.72 (m, 6H), 3.20˜3.18 (m, 1H), 3.05˜2.96 (m, 2H), 2.84˜2.81 (m, 2H), 2.68˜2.65 (m, 1H), 2.63 (s, 3H), 2.45 (s, 3H), 2.26˜2.23 (m, 2H), 2.13˜2.09 (m, 2H), 1.96˜1.93 (m, 5H), 1.30˜1.29 (d, J=6 Hz, 3H).

LC-MS [mobile phase: from 80% water (0.1% FA) and 20% MeCN (0.1% FA) to 5% water (0.1% FA) and 95% MeCN (0.1% FA) in 2.0 min]: Purity: 100% @ 254 nm; Rt=1.12 min; MS Calcd: 506.64, MS Found: 507.4 [M+H]⁺.

Chiral HPLC [Column: AD-H; Column size: 0.46 cm I.D.×15 cm L; Injection: 2 μl; Mobile phase: HEP:EtOH (0.05% DEA)=60:40; Flow rate: 0.5 mL; Wave length: UV 254 nm; Temperature: 25° C.]: Rt: 2.187 min, ee 100%;

Single Unknown Isomer 2

¹H NMR (400 MHz, CDCl₃) δ 8.78 (s, 1H), 8.05 (s, 1H), 7.50 (s, 1H), 6.93 (s, 1H), 4.35˜4.32 (m, 2H), 3.99˜3.94 (m, 2H), 3.84˜3.70 (m, 6H), 3.21˜3.18 (m, 1H), 3.05˜2.96 (m, 2H), 2.84˜2.80 (m, 2H), 2.68˜2.65 (m, 1H), 2.63 (s, 3H), 2.46 (s, 3H), 2.26˜2.25 (m, 2H), 2.13˜2.08 (m, 2H), 1.93˜1.92 (m, 5H), 1.30˜1.29 (d, J=6.4 Hz, 3H).

LC-MS [mobile phase: from 80% water (0.1% FA) and 20% MeCN (0.1% FA) to 5% water (0.1% FA) and 95% MeCN (0.1% FA) in 2.0 min]: Purity: 100% @ 254 nm; Rt=1.12 min; MS Calcd: 506.64, MS Found: 507.4 [M+H]⁺.

Chiral HPLC [Column: AD-H; Column size: 0.46 cm I.D.×15 cm L; Injection: 2 μl; Mobile phase: HEP:EtOH (0.05% DEA)=60:40; Flow rate: 0.5 mL; Wave length: UV 254 nm; Temperature: 25° C.]: Rt: 2.730 min, ee 99%;

Single Unknown Isomer 3

¹H NMR (400 MHz, CDCl₃) δ 8.78 (s, 1H), 8.05 (s, 1H), 7.50 (s, 1H), 6.93 (s, 1H), 4.35 4.32 (m, 2H), 3.99˜3.94 (m, 2H), 3.84˜3.70 (m, 6H), 3.21˜3.18 (m, 1H), 3.05˜2.96 (m, 2H), 2.84˜2.81 (m, 2H), 2.68˜2.64 (m, 1H), 2.63 (s, 3H), 2.45 (s, 3H), 2.28˜2.23 (m, 2H), 2.13˜2.07 (m, 2H), 1.96˜1.91 (m, 5H), 1.30˜1.29 (d, J=6.4 Hz, 3H).

LC-MS [mobile phase: from 80% water (0.1% FA) and 20% MeCN (0.1% FA) to 5% water (0.1% FA) and 95% MeCN (0.1% FA) in 2.0 min]: Purity: 100% @ 254 nm; Rt=1.13 min; MS Calcd: 506.64, MS Found: 507.4 [M+H]⁺.

Chiral HPLC [Column: AD-H; Column size: 0.46 cm I.D.×15 cm L; Injection: 2 μl; Mobile phase: HEP:EtOH (0.05% DEA)=60:40; Flow rate: 0.5 mL/min; Wave length: UV 254 nm; Temperature: 25° C.]: Rt: 5.122 min, ee 100%;

Single Unknown Isomer 4

¹H NMR (400 MHz, CDCl₃) δ 8.78 (s, 1H), 8.05 (s, 1H), 7.50 (s, 1H), 6.93 (s, 1H), 4.35˜4.32 (m, 2H), 3.99 3.94 (m, 2H), 3.84˜3.70 (m, 6H), 3.21˜3.18 (m, 1H), 3.04˜2.96 (m, 2H), 2.84˜2.80 (m, 2H), 2.68˜2.65 (m, 1H), 2.63 (s, 3H), 2.45 (s, 3H), 2.26˜2.21 (m, 2H), 2.15˜2.10 (m, 2H), 1.93˜1.92 (m, 5H), 1.30˜1.29 (d, J=6 Hz, 3H).

LC-MS [mobile phase: from 80% water (0.1% FA) and 20% MeCN (0.1% FA) to 5% water (0.1% FA) and 95% MeCN (0.1% FA) in 2.0 min]: Purity: 100% @ 254 nm; Rt=1.12 min; MS Calcd: 506.64, MS Found: 507.4 [M+H]⁺.

Chiral HPLC [Column: AD-H; Column size: 0.46 cm I.D.×15 cm L; Injection: 2 μl; Mobile phase: HEP:EtOH (0.05% DEA)=60:40; Flow rate: 0.5 mL/min; Wave length: UV 254 nm; Temperature: 25° C.]: Rt: 5.848 min, ee 100%;

### Examples 64 to 67

### (6-Methyl-4-(2-methyl-6-(5-methyl-6-(1-(tetrahydrofuran-3-yl)piperidin-4-yl)-1H-indazol-1-yl)pyrimidin-4-yl)morpholin-2-yl)methanol (single unknown isomer 5, E64; Single Unknown Isomer 6, E65; Single Unknown Isomer 7, E66; Single Unknown Isomer 8, E67)

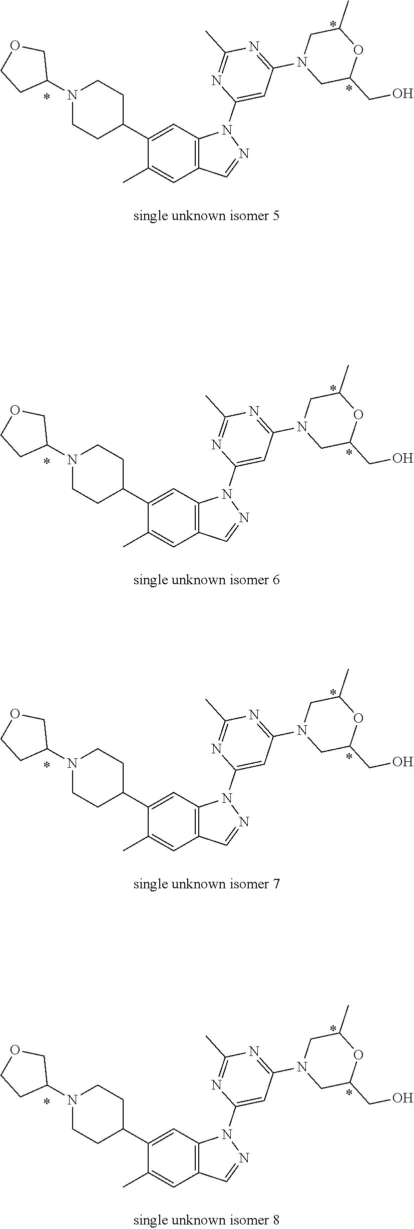 [SMILES: Cc1nc(N2CC(C)OC(CO)C2)cc(-n2ncc3cc(C)c(C4CCN(C5CCOC5)CC4)cc32)n1 ; Cc1nc(N2CC(C)OC(CO)C2)cc(-n2ncc3cc(C)c(C4CCN(C5CCOC5)CC4)cc32)n1 ; Cc1nc(N2CC(C)OC(CO)C2)cc(-n2ncc3cc(C)c(C4CCN(C5CCOC5)CC4)cc32)n1 ; Cc1nc(N2CC(C)OC(CO)C2)cc(-n2ncc3cc(C)c(C4CCN(C5CCOC5)CC4)cc32)n1]

The title compound was prepared by a procedure similar to that described as E1 and E2 starting from a solution of 5-methyl-6-(1-(tetrahydrofuran-3-yl)piperidin-4-yl)-1H-indazole, (4-(6-iodo-2-methylpyrimidin-4-yl)-6-methylmorpholin-2-yl)methanol (isomer 2, D81) in toluene, CuI, K₃PO₄ and N,N′-dimethylethylenediamine at 100° C.

Chiral Separation:

Method: Column: AD-H; Column size: 0.46 cm I.D.×15 cm L; Mobile phase: Supercritical CO₂:EtOH (0.1% NH₃H₂O)=60:40; Flow rate: 0.5 mL/min; Wave length: UV 254 nm; Temperature: 25° C.; Sample solution in EtOH

Single Unknown Isomer 5

¹H NMR (400 MHz, CDCl₃) 8.77 (s, 1H), 8.05 (s, 1H), 7.50 (s, 1H), 6.92 (s, 1H), 4.07˜4.00 (m, 2H), 3.98˜3.90 (m, 3H), 3.86 3.82 (m, 2H), 3.74˜3.68 (m, 4H), 3.34˜3.29 (m, 1H), 3.21˜3.17 (m, 1H), 3.05˜2.97 (m, 2H), 2.84˜2.80 (m, 1H), 2.62 (s, 3H), 2.46 (s, 3H), 2.28˜2.22 (m, 2H), 2.12˜2.10 (m, 1H), 1.96˜1.93 (m, 5H), 1.26˜1.25 (d, J=6 Hz, 3H).

LC-MS [mobile phase: from 80% water (0.1% FA) and 20% MeCN (0.1% FA) to 5% water (0.1% FA) and 95% MeCN (0.1% FA) in 2.0 min]: Rt=1.08 min; MS Calcd: 506.64, MS Found: 507.3 [M+H].

Chiral HPLC [Column: AD-H; Column size: 0.46 cm I.D.×15 cm L; Injection: 2 μl; Mobile phase: HEP:EtOH (0.05% DEA)=60:40; Flow rate: 0.5 mL; Wave length: UV 254 nm; Temperature: 25° C.]: Rt: 2.286 min, ee 100%;

Single Unknown Isomer 6

¹H NMR (400 MHz, CDCl₃) δ 8.77 (s, 1H), 8.05 (s, 1H), 7.50 (s, 1H), 6.92 (s, 1H), 4.05˜4.00 (m, 2H), 3.97˜3.94 (m, 3H), 3.89˜3.82 (m, 2H), 3.74˜3.66 (m, 4H), 3.34˜3.31 (m, 1H), 3.21˜3.19 (m, 1H), 3.05˜2.96 (m, 2H), 2.85˜2.82 (m, 1H), 2.62 (s, 3H), 2.46 (s, 3H), 2.27˜2.23 (m, 2H), 2.13˜2.08 (m, 1H), 1.96˜1.91 (m, 5H), 1.26˜1.25 (d, J=6 Hz, 3H).

LC-MS [mobile phase: from 80% water (0.1% FA) and 20% MeCN (0.1% FA) to 5% water (0.1% FA) and 95% MeCN (0.1% FA) in 2.0 min]: Rt=1.08 min; MS Calcd: 506.64, MS Found: 507.3 [M+H]⁺.

Chiral HPLC [Column: AD-H; Column size: 0.46 cm I.D.×15 cm L; Injection: 2 μl; Mobile phase: HEP:EtOH (0.05% DEA)=60:40; Flow rate: 0.5 mL; Wave length: UV 254 nm; Temperature: 25° C.]: Rt: 3.222 min, ee 99%;

Single Unknown Isomer 7

¹H NMR (400 MHz, CDCl₃) 8.78 (s, 1H), 8.06 (s, 1H), 7.50 (s, 1H), 6.92 (s, 1H), 4.05˜4.00 (m, 2H), 3.97˜3.93 (m, 3H), 3.87˜3.82 (m, 2H), 3.73 3.65 (m, 4H), 3.34˜3.32 (m, 1H), 3.22˜3.20 (m, 1H), 3.06˜2.95 (m, 2H), 2.88˜2.82 (m, 1H), 2.63 (s, 3H), 2.46 (s, 3H), 2.26˜2.21 (m, 2H), 2.14˜2.10 (m, 1H), 1.97˜1.92 (m, 5H), 1.26˜1.25 (d, J=6 Hz, 3H).

LC-MS [mobile phase: from 80% water (0.1% FA) and 20% MeCN (0.1% FA) to 5% water (0.1% FA) and 95% MeCN (0.1% FA) in 2.0 min]: Rt=1.07 min; MS Calcd: 506.64, MS Found: 507.3 [M+H]⁺.

Chiral HPLC [Column: AD-H; Column size: 0.46 cm I.D.×15 cm L; Injection: 2 μl; Mobile phase: HEP:EtOH (0.05% DEA)=60:40; Flow rate: 0.5 mL; Wave length: UV 254 nm; Temperature: 25° C.]: Rt: 3.316 min, ee 100%;

Single Unknown Isomer 8

¹H NMR (400 MHz, CDCl₃) δ 8.77 (s, 1H), 8.06 (s, 1H), 7.50 (s, 1H), 6.92 (s, 1H), 4.06-4.04 (m, 2H), 3.99˜3.94 (m, 3H), 3.86˜3.82 (m, 2H), 3.73˜3.65 (m, 4H), 3.34˜3.31 (m, 1H), 3.20˜3.18 (m, 1H), 3.04˜2.96 (m, 2H), 2.84˜2.81 (m, 1H), 2.62 (s, 3H), 2.46 (s, 3H), 2.26˜2.22 (m, 2H), 2.11˜2.10 (m, 1H), 1.94˜1.92 (m, 5H), 1.26˜1.25 (d, J=6.4 Hz, 3H).

LC-MS [mobile phase: from 80% water (0.1% FA) and 20% MeCN (0.1% FA) to 5% water (0.1% FA) and 95% MeCN (0.1% FA) in 2.0 min]: Rt=1.07 min; MS Calcd: 506.64, MS Found: 507.3 [M+H]⁺.

Chiral HPLC [Column: AD-H; Column size: 0.46 cm I.D.×15 cm L; Injection: 2 μl; Mobile phase: HEP:EtOH (0.05% DEA)=60:40; Flow rate: 0.5 mL; Wave length: UV 254 nm; Temperature: 25° C.]: Rt: 3.929 min, ee 95.7%

### Examples 68 and 69

### (3R)-3-Methyl-4-(2-methyl-6-(5-methyl-6-(1-(tetrahydrofuran-3-yl)piperidin-4-yl)-1H-indazol-1-yl)pyrimidin-4-yl)morpholine (Single Unknown Isomer 1, E68 and Single Unknown Isomer 2, E69)

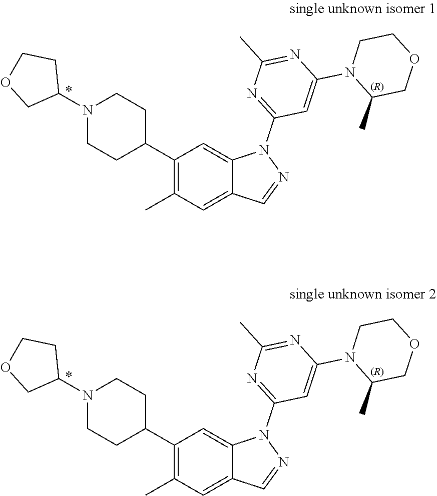

To a solution of 5-methyl-6-(1-(tetrahydrofuran-3-yl)piperidin-4-yl)-1H-indazole (87 mg, 0.26 mmol) and (R)-4-(6-iodo-2-methylpyrimidin-4-yl)-3-methylmorpholine (74 mg, 0.26 mmol) in toluene (20 mL) were added CuI (74 mg, 0.39 mmol), K₃PO₄ (138 mg, 0.520 mmol) and N,N′-dimethylethylenediamine (46 mg, 0.52 mmol). The reaction mixture was stirred at 100° C. for 4 h. LC-MS showed the reaction was completed. The reaction mixture was concentrated to remove solvent, dissolved in CH₂Cl₂ (20 mL) and water (20 mL) and treated with a solution of sat. NH₄OH (50 mL). The organic layer was separated and the aqueous layer was extracted with CH₂Cl₂ (2×20 mL). The combined organic layers were washed with brine (2×50 mL), dried over anhydrous Na₂SO₄, filtered and concentrated. The residue was purified by silica gel chromatography eluted with EtOAc to give the desired product as a white solid (100 mg, yield: 78%).

LC-MS [mobile phase: from 40% water (0.1% NH₄OH) and 60% MeCN (0.1% NH₄OH) to 5% water (0.1% NH₄OH) and 95% MeCN (0.1% NH₄OH) in 2.6 min]: purity 98%, Rt=1.27 min; MS Calcd: 492.61, MS Found: 493.3 [M+H]⁺.

The racemic (3R)-3-methyl-4-(2-methyl-6-(5-methyl-6-(1-(tetrahydrofuran-3-yl)piperi-din-4-yl)-1H-indazol-1-yl)pyrimidin-4-yl)morpholine (100 mg) was separated by chiral prep-HPLC [Method: Column: AD-H; Column size: 0.46 cm I.D×15 cm L; Mobile phase: Supercritical CO2:IPA (0.1% NH3-H₂O)=70:30; Flow rate: 0.5 mL/min; Wave length: UV 254 nm; Temperature: 25° C.; Sample solution in EtOH] to afford below two white solids

Single Unknown Isomer 1

¹H NMR (400 MHz, CDCl₃) δ 8.73 (s, 1H), 8.05 (s, 1H), 7.50 (s, 1H), 6.79 (s, 1H), 4.45 (s, 1H), 4.11˜4.00 (m, 4H), 3.98˜3.92 (m, 3H), 3.83˜3.73 (m, 4H), 3.69˜3.57 (m, 1H), 3.35 3.29 (m, 1H), 3.14˜3.05 (m, 1H), 3.03˜3.00 (m, 1H), 2.94˜2.92 (m, 1H), 2.84˜2.80 (m, 1H), 2.45 (s, 3H), 2.24˜2.21 (m, 2H), 2.10˜2.06 (m, 1H), 2.01˜1.89 (m, 5H), 1.21 1.20 (d, J=4.4 Hz, 3H).

LC-MS [mobile phase: from 80% water (0.1% NH₄OH) and 20% MeCN (0.1% NH₄OH) to 5% water (0.1% NH₄OH) and 95% MeCN (0.1% NH₄OH) in 2.6 min]: Rt=2.27 min; MS Calcd: 492.61, MS Found: 493.3 [M+H]⁺.

Chiral HPLC [Column: AD-H; Column size: 0.46 cm I.D.×15 cm L; Injection: 2 μl; Mobile phase: HEP:IPA (0.1% DEA)=70:30; Flow rate: 0.5 mL; Wave length: UV 254 nm; Temperature: 25° C.]: Rt: 4.320 min, ee 100%; Single unknown isomer 2

¹H NMR (400 MHz, CDCl₃) δ 8.75 (s, 1H), 8.05 (s, 1H), 7.50 (s, 1H), 6.79 (s, 1H), 4.68 (s, 1H), 4.11˜4.02 (m, 4H), 3.99˜3.92 (m, 3H), 3.83˜3.71 (m, 4H), 3.69˜3.55 (m, 1H), 3.36˜3.29 (m, 1H), 3.17˜3.05 (m, 1H), 3.03˜3.01 (m, 1H), 2.95˜2.93 (m, 1H), 2.84˜2.81 (m, 1H), 2.46 (s, 3H), 2.27˜2.19 (m, 2H), 2.11˜2.06 (m, 1H), 2.01˜1.89 (m, 5H), 1.21 1.20 (d, J=4.4 Hz, 3H).

LC-MS [mobile phase: from 80% water (0.1% NH₄OH) and 20% MeCN (0.1% NH₄OH) to 5% water (0.1% NH₄OH) and 95% MeCN (0.1% NH₄OH) in 2.6 min]: Rt=2.29 min; MS Calcd: 492.61, MS Found: 493.3 [M+H]⁺.

Chiral HPLC [Column: AD-H; Column size: 0.46 cm I.D.×15 cm L; Injection: 2 μl; Mobile phase: HEP:IPA (0.1% DEA)=70:30; Flow rate: 0.5 mL; Wave length: UV 254 nm; Temperature: 25° C.]: Rt: 4.960 min, ee 100%;

### Examples 70 and 71

### (3S)-3-Methyl-4-(2-methyl-6-(5-methyl-6-(1-(tetrahydrofuran-3-yl)piperidin-4-yl)-1H-indazol-1-yl)pyrimidin-4-yl)morpholine (Single Unknown Isomer 1, E70 and Single Unknown Isomer 2, E71)

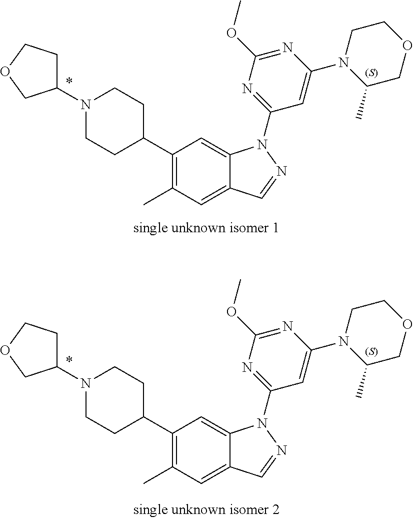

The title compounds were prepared by a procedure similar to that described as E₁ and E₂ starting from a solution of 5-methyl-6-(1-(tetrahydrofuran-3-yl)piperidin-4-yl)-1H-indazole and (S)-4-(6-iodo-2-methoxypyrimidin-4-yl)-3-methylmorpholine in toluene, CuI, K₃PO₄.3H₂O and N,N′-dimethylethylenediamine at 100° C.

LC-MS [mobile phase: from 60% water (0.1% FA) and 40% MeCN (0.1% FA) to 5% water (0.1% FA) and 95% MeCN (0.1% FA) in 2.6 min]: purity 98%, Rt=0.87 min; MS Calcd: 492.61, MS Found: 493.4 [M+H]⁺.

Chiral Separation:

Method: Column: AD-H; Column size: 0.46 cm I.D.×15 cm L; Mobile phase: Supercritical CO₂:IPA (0.1% NH₃H₂O)=70:30; Flow rate: 0.5 mL; Wave length: UV 254 nm; Temperature: 25° C.; Sample solution in EtOH.

Single Unknown Isomer 1

¹H NMR (400 MHz, CDCl₃) δ 8.75 (s, 1H), 8.05 (s, 1H), 7.50 (s, 1H), 6.79 (s, 1H), 4.45 (s, 1H), 4.11˜4.01 (m, 4H), 3.99˜3.92 (m, 3H), 3.85˜3.71 (m, 4H), 3.69˜3.55 (m, 1H), 3.37 3.30 (m, 1H), 3.16˜3.05 (m, 1H), 3.03˜3.01 (m, 1H), 2.95˜2.92 (m, 1H), 2.83˜2.81 (m, 1H), 2.46 (s, 3H), 2.24˜2.19 (m, 2H), 2.09˜2.08 (m, 1H), 1.98˜1.89 (m, 5H), 1.350 1.333 (d, J=6.8 Hz, 3H).

LC-MS [mobile phase: from 80% water (0.1% NH₄OH) and 20% MeCN (0.1% NH₄OH) to 5% water (0.1% NH₄OH) and 95% MeCN (0.1% NH₄OH) in 2.6 min]: Rt=2.29 min; MS Calcd: 492.61, MS Found: 493.3 [M+H]⁺.

Chiral HPLC [Column: AD-H; Column size: 0.46 cm I.D.×15 cm L; Injection: 2 μl; Mobile phase: HEP:IPA (0.1% DEA)=70:30; Flow rate: 0.5 mL; Wave length: UV 254 nm; Temperature: 25° C.]: Rt: 4.311 min, ee 100%;

Single Unknown Isomer 2

¹H NMR (400 MHz, CDCl₃) δ 8.75 (s, 1H), 8.05 (s, 1H), 7.50 (s, 1H), 6.79 (s, 1H), 4.45 (s, 1H), 4.11˜4.08 (m, 4H), 3.99˜3.93 (m, 3H), 3.83˜3.71 (m, 4H), 3.69˜3.55 (m, 1H), 3.35˜3.29 (m, 1H), 3.17˜3.05 (m, 1H), 3.03˜3.01 (m, 1H), 2.95˜2.93 (m, 1H), 2.84˜2.83 (m, 1H), 2.45 (s, 3H), 2.27˜2.19 (m, 2H), 2.10˜2.07 (m, 1H), 1.91˜1.87 (m, 5H), 1.34˜1.33 (d, J=6.8 Hz, 3H).

LC-MS [mobile phase: from 80% water (0.1% NH₄OH) and 20% MeCN (0.1% NH₄OH) to 5% water (0.1% NH₄OH) and 95% MeCN (0.1% NH₄OH) in 2.6 min]: Purity: 70%, Rt=2.29 & 2.31 min; MS Calcd: 492.61, MS Found: 493.3 [M+H]⁺.

Chiral HPLC [Column: AD-H; Column size: 0.46 cm I.D.×15 cm L; Injection: 2 μl; Mobile phase: HEP:IPA (0.1% DEA)=70:30; Flow rate: 0.5 mL; Wave length: UV 254 nm; Temperature: 25° C.]: Rt: 4.814 min, ee 99%.

### Example 72

### (3R)-3-Methyl-4-(2-methyl-6-(5-methyl-6-(1-(tetrahydrofuran-3-yl)piperidin-4-yl)-1H-indazol-1-yl)pyrimidin-4-yl)morpholine

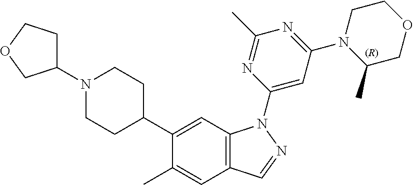

The title compound was prepared by a procedure similar to that described as E1 and E2 starting from a mixture of 5-methyl-6-(1-(tetrahydrofuran-3-yl)piperidin-4-yl)-1H-indazole and (R)-4-(6-iodo-2-methylpyrimidin-4-yl)-3-methylmorpholine in toluene, DMEDA, CuI and K₃PO₄.

LC-MS [mobile phase: 80% water (0.1% FA) and 20% MeCN (0.1% FA) in 2.6 min]: Rt=1.17 min; MS Calcd.:476.3, MS Found: 477.3 [M+H]⁺.

### Example 73

### (3S)-3-Methyl-4-(2-methyl-6-(5-methyl-6-(1-(tetrahydrofuran-3-yl)piperidin-4-yl)-1H-indazol-1-yl)pyrimidin-4-yl)morpholine

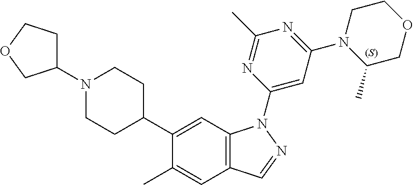

The title compound was prepared by a procedure similar to that described as E1 and E2 starting from 5-methyl-6-(1-(tetrahydrofuran-3-yl)piperidin-4-yl)-1H-indazole and (S)-4-(6-iodo-2-methylpyrimidin-4-yl)-3-methylmorpholine in toluene, DMEDA, CuI and K₃PO₄.

LC-MS [mobile phase: 80% water (0.1% FA) and 20% MeCN (0.1% FA) in 2.6 min]: Rt=1.19 min; MS Calcd.:476.3, MS Found: 477.3 [M+H]⁺.

### Examples 74 and 75

### 4-(2-Methoxy-6-(5-methyl-6-(1-(tetrahydrofuran-3-yl)piperidin-4-yl)-1H-indazol-1-yl)pyrimidin-4-yl)morpholine (Single Unknown Isomer 1, E74 and Single Unknown Isomer 2, E75)

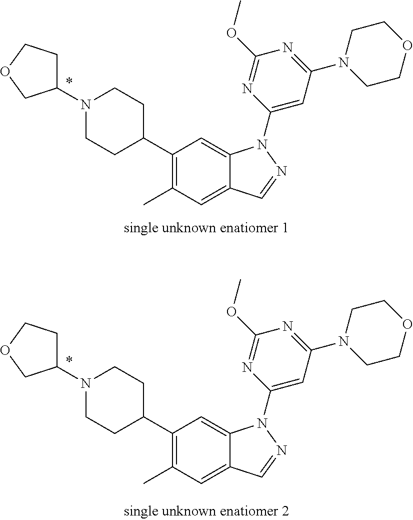 [SMILES: COc1nc(N2CCOCC2)cc(-n2ncc3cc(C)c(C4CCN(C5CCOC5)CC4)cc32)n1 ; COc1nc(N2CCOCC2)cc(-n2ncc3cc(C)c(C4CCN(C5CCOC5)CC4)cc32)n1]

The title compound was prepared by a procedure similar to that described as E1 and E2 starting from a solution of 4-(6-iodo-2-methoxypyrimidin-4-yl)morpholine and 5-methyl-6-(tetrahydrofuran-3-yl)-1H-indazole in toluene, N¹,N²-dimethylethane-1,2-diamine, CuI and K₃PO₄.3H₂O.

LC-MS [mobile phase: mobile phase: from 50% water (0.1% FA) and 50% MeCN (0.1% FA) to 5% water (0.1% FA) and 95% MeCN (0.1% FA) in 2.6 min]: Rt=0.81 min; MS Calcd.:478.5, MS Found: 479.4 [M+H]⁺.

Chiral Separation:

Method: Column: AD-H; Column size: 0.46 cm I.D.×15 cm L; Mobile phase: Supercritical CO₂:IPA (0.1% NH₃H₂O)=60:40; Flow rate: 0.5 mL/min; Wave length: UV 254 nm; Temperature: 25° C.; Sample solution in EtOH

Single Unknown Isomer 1

¹H NMR (400 MHz, CDCl₃) δ 8.74 (s, 1H), 8.05 (s, 1H), 7.50 (s, 1H), 6.83 (s, 1H), 4.12 (s, 3H), 4.00˜3.93 (m, 2H), 3.91˜3.83 (m, 5H), 3.79˜3.67 (m, 5H), 3.17˜3.14 (m, 1H), 3.05˜3.01 (m, 1H), 2.95˜2.92 (m, 1H), 2.86˜2.80 (m, 1H), 2.46 (s, 3H), 2.27˜2.19 (m, 2H), 2.11˜2.07 (m, 1H), 1.94˜1.81 (m, 5H).

LC-MS [mobile phase: from 80% water (0.1% FA) and 20% MeCN (0.1% FA) to 5% water (0.1% FA) and 95% MeCN (0.1% FA) in 9 min], purity: 100%; Rt=3.67 min; MS Calcd: 478.5, MS Found: 479.3 [M+H]⁺.

Chiral HPLC [Column: AD-H; Column size: 0.46 cm I.D×15 cm L; Injection: 2 μl; Mobile phase: HEP:IPA (0.1% DEA)=60:40; Flow rate: 0.5 mL/min; Wave length: UV 254 nm; Temperature: 25° C.]: Rt: 4.590 min, ee: 100%

Single Unknown Isomer 2

¹H NMR (400 MHz, CDCl₃) δ 8.74 (s, 1H), 8.05 (s, 1H), 7.50 (s, 1H), 6.83 (s, 1H), 4.12 (s, 3H), 4.00˜3.93 (m, 2H), 3.91˜3.83 (m, 5H), 3.79˜3.67 (m, 5H), 3.17˜3.14 (m, 1H), 3.05˜3.01 (m, 1H), 2.95˜2.92 (m, 1H), 2.86˜2.80 (m, 1H), 2.46 (s, 3H), 2.28˜2.19 (m, 2H), 2.11˜2.07 (m, 1H), 1.96˜1.89 (m, 5H).

LC-MS [mobile phase: from 80% water (0.1% FA) and 20% MeCN (0.1% FA) to 5% water (0.1% FA) and 95% MeCN (0.1% FA) in 10 min]: purity: 98.6%; Rt=3.70 min; MS Calcd: 478.5, MS Found: 479.3 [M+H]⁺.

Chiral HPLC [Column: AD-H; Column size: 0.46 cm I.D×15 cm L; Injection: 2 μl; Mobile phase: HEP:IPA (0.1% DEA)=60:40; Flow rate: 0.5 mL/min; Wave length: UV 254 nm; Temperature: 25° C.]: Rt: 5.611 min, ee: 100%

### Examples 76 and 77

### 4-(2-Methyl-6-(5-methyl-6-(1-(tetrahydrofuran-3-yl)piperidin-4-yl)-1H-indazol-1-yl)pyrimidin-4-yl)morpholine (Single Unknown Isomer 1, E76 and Single Unknown Isomer 2, E77)

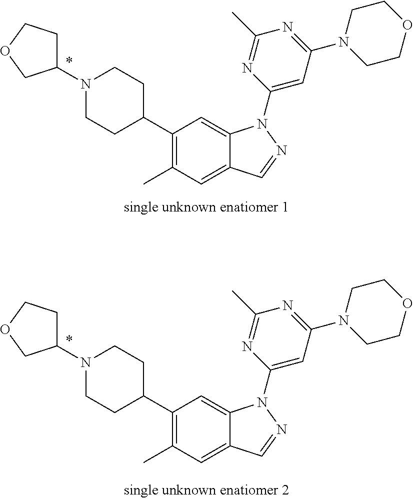 [SMILES: Cc1nc(N2CCOCC2)cc(-n2ncc3cc(C)c(C4CCN(C5CCOC5)CC4)cc32)n1 ; Cc1nc(N2CCOCC2)cc(-n2ncc3cc(C)c(C4CCN(C5CCOC5)CC4)cc32)n1]

The title compound was prepared by a procedure similar to that described as E1 and E2 starting from a solution of 4-(6-iodo-2-methylpyrimidin-4-yl)morpholine and 5-methyl-6-(1-(tetrahydrofuran-3-yl)piperidin-4-yl)-1H-indazole in toluene, N¹,N²-dimethylethane-1,2-diamine, CuI and K₃PO₄.3H₂O at 100° C.

LC-MS [mobile phase: from 50% water (0.1% FA) and 50% MeCN (0.1% FA) to 5% water (0.1% FA) and 95% MeCN (0.1% FA) in 2.6 min]: Rt=0.77 min; MS Calcd.:462.5, MS Found: 463.3 [M+H]⁺.

Chiral Separation:

Method: Column: AD-H; Column size: 0.46 cm I.D.×15 cm L; Mobile phase: Supercritical CO₂:EtOH (0.1% NH₃H₂O)=60:40; Flow rate: 0.5 mL/min; Wave length: UV 254 nm; Temperature: 25° C.; Sample solution in EtOH

Single Unknown Isomer 1

¹H NMR (400 MHz, CDCl₃) δ 8.80 (s, 1H), 8.04 (s, 1H), 7.49 (s, 1H), 6.94 (s, 1H), 3.99˜3.94 (m, 2H), 3.86˜3.84 (m, 5H), 3.80˜3.70 (m, 5H), 3.21˜3.18 (m, 1H), 3.05˜2.96 (m, 2H), 2.85˜2.82 (m, 1H), 2.63 (s, 3H), 2.45 (s, 3H), 2.29˜2.25 (m, 2H), 2.24˜2.20 (m, 1H), 2.12˜1.93 (m, 5H).

LC-MS [mobile phase: from 80% water (0.1% FA) and 20% MeCN (0.1% FA) to 5% water (0.1% FA) and 95% MeCN (0.1% FA) in 9 min]: purity: 100%; Rt=3.51 min; MS Calcd: 462.5, MS Found: 463.3 [M+H]⁺.

Chiral HPLC [Column: AD-H; Column size: 0.46 cm I.D.×15 cm L; Injection: 2 μl; Mobile phase: HEP:EtOH (0.1% DEA)=60:40; Flow rate: 0.5 mL/min; Wave length: UV 254 nm; Temperature: 25° C.]: Rt: 2.771 min, ee: 100%

Single Unknown Isomer 2

¹H NMR (400 MHz, CDCl₃) δ 8.79 (s, 1H), 8.04 (s, 1H), 7.49 (s, 1H), 6.94 (s, 1H), 4.01˜3.94 (m, 2H), 3.86˜3.84 (m, 5H), 3.80˜3.70 (m, 5H), 3.21˜3.18 (m, 1H), 3.05˜2.96 (m, 2H), 2.85˜2.82 (m, 1H), 2.63 (s, 3H), 2.45 (s, 3H), 2.29˜2.24 (m, 2H), 2.23˜2.20 (m, 1H), 2.13˜1.92 (m, 5H).

LC-MS [mobile phase: from 80% water (0.1% FA) and 20% MeCN (0.1% FA) to 5% water (0.1% FA) and 95% MeCN (0.1% FA) in 9 min]: Rt=3.53 min; MS Calcd: 462.5, MS Found: 463.3 [M+H]⁺.

Chiral HPLC [Column: AD-H; Column size: 0.46 cm I.D.×15 cm L; Injection: 2 μl; Mobile phase: HEP:EtOH (0.1% DEA)=60:40; Flow rate: 0.5 mL/min; Wave length: UV 254 nm; Temperature: 25° C.]: Rt: 3.332 min, ee: 99%

### Examples 78 to 81

### cis-(3-Methyl-4-(2-methyl-6-(5-methyl-6-(1-(tetrahydrofuran-3-yl)piperidin-4-yl)-1H-indazol-1-yl)pyrimidin-4-yl)morpholin-2-yl)methanol (Single Unknown Isomer 1, E78; Single Unknown Isomer 2, E79; Single Unknown Isomer 3, E80; Single Unknown Isomer 4, E81)

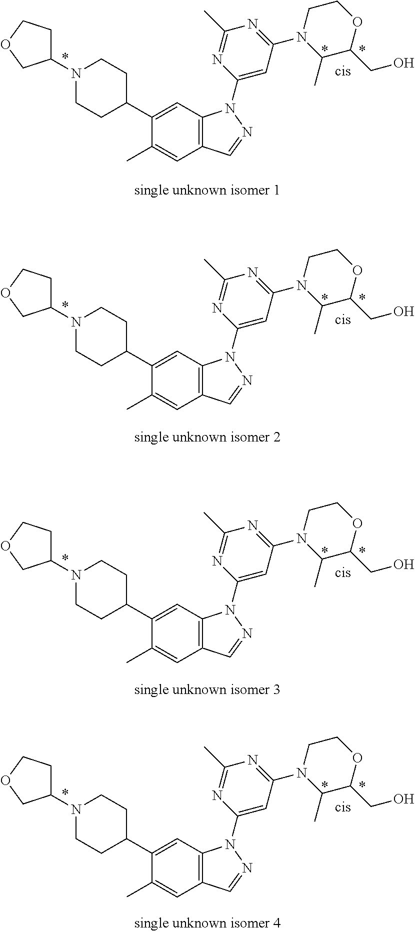 [SMILES: Cc1nc(N2CCOC(CO)C2C)cc(-n2ncc3cc(C)c(C4CCN(C5CCOC5)CC4)cc32)n1 ; Cc1nc(N2CCOC(CO)C2C)cc(-n2ncc3cc(C)c(C4CCN(C5CCOC5)CC4)cc32)n1 ; Cc1nc(N2CCOC(CO)C2C)cc(-n2ncc3cc(C)c(C4CCN(C5CCOC5)CC4)cc32)n1 ; Cc1nc(N2CCOC(CO)C2C)cc(-n2ncc3cc(C)c(C4CCN(C5CCOC5)CC4)cc32)n1]

To a solution of cis-(3-methylmorpholin-2-yl)methanol (84.0 mg, 0.500 mmol)(D94) and 1-(6-chloro-2-methylpyrimidin-4-yl)-5-methyl-6-(1-(tetrahydrofuran-3-yl)piperidin-4-yl)-1H-indazole (206 mg, 0.500 mmol) in DMF (25 mL) was added DIPEA (260 mg, 2.00 mmol). The reaction mixture was stirred at 80° C. overnight, then at 100° C. for one day, diluted with water (50 mL) and extracted with EtOAc (3×100 mL). The combined organic layers were washed with water (3×150 mL) and brine (200 mL), dried over anhydrous Na₂SO₄, filtered and concentrated to give a residue. The residue was purified by silica gel column chromatography (CH₂Cl₂:MeOH=40:1) to give the title compound (160 mg, yield: 63%) as a white solid.

LC-MS [mobile phase: from 90% water (0.1% FA) and 10% MeCN (0.1% FA) to 5% water (0.1% FA) and 95% MeCN (0.1% FA) in 9 min]: Rt=4.12 min; MS Calcd: 506.30, MS Found: 507.3 [M+H]⁺.

Chiral Separation:

Method: Column: AD-H; Column size: 0.46 cm I.D.×15 cm L; Mobile phase: Supercritical CO₂:EtOH (0.1% NH₃H₂O)=60:40; Flow rate: 0.5 mL; Wave length: UV 254 nm; Temperature: 25° C.; Sample solution in EtOH

Single Unknown Isomer 1

¹H NMR (400 MHz, CDCl₃) δ 8.78 (s, 1H), 8.05 (s, 1H), 7.50 (s, 1H), 6.91 (s, 1H), 4.10˜4.06 (m, 1H), 4.00˜3.94 (m, 2H), 3.85˜3.61 (m, 7H), 3.25˜3.19 (m, 2H), 3.06˜2.97 (m, 2H), 2.84˜2.82 (m, 1H), 2.63 (s, 3H), 2.46 (s, 3H), 2.28˜2.23 (m, 2H), 2.13˜2.10 (m, 1H), 1.93˜1.92 (m, 6H), 1.15˜1.14 (d, J=6.4 Hz, 3H).

LC-MS [mobile phase: from 90% water (0.1% FA) and 10% MeCN (0.1% FA) to 5% water (0.1% FA) and 95% MeCN (0.1% FA) in 9 min]: Rt=4.30 min; MS Calcd: 506.30, MS Found: 507.3 [M+H]⁺.

Chiral HPLC [Column: AD-H; Column size: 0.46 cm I.D.×15 cm L; Injection: 2 μl; Mobile phase: HEP:EtOH (0.05% DEA)=60:40; Flow rate: 0.5 mL; Wave length: UV 254 nm; Temperature: 25° C.]: Rt: 1.882 min, ee: 100%

Single Unknown Isomer 2

¹H NMR (400 MHz, CDCl₃) δ 8.78 (s, 1H), 8.05 (s, 1H), 7.50 (s, 1H), 6.91 (s, 1H), 4.10˜4.06 (m, 1H), 4.00˜3.94 (m, 2H), 3.85˜3.60 (m, 7H), 3.21˜3.19 (m, 2H), 3.06˜2.97 (m, 2H), 2.84˜2.62 (m, 1H), 2.63 (s, 3H), 2.46 (s, 3H), 2.28˜2.22 (m, 2H), 2.13˜2.10 (m, 1H), 1.94˜1.92 (m, 6H), 1.15˜1.14 (d, J=6.8 Hz, 3H).

LC-MS [mobile phase: from 90% water (0.1% FA) and 10% MeCN (0.1% FA) to 5% water (0.1% FA) and 95% MeCN (0.1% FA) in 9 min]: Rt=4.22 min; MS Calcd: 506.30, MS Found: 507.3 [M+H]⁺.

Chiral HPLC [Column: AD-H; Column size: 0.46 cm I.D.×15 cm L; Injection: 2 μl; Mobile phase: HEP:EtOH (0.05% DEA)=60:40; Flow rate: 0.5 mL; Wave length: UV 254 nm; Temperature: 25° C.]: Rt: 2.248 min, ee: 100% single unknown isomer 3

¹H NMR (400 MHz, CDCl₃) δ 8.78 (s, 1H), 8.05 (s, 1H), 7.50 (s, 1H), 6.91 (s, 1H), 4.10˜4.06 (m, 1H), 4.00˜3.94 (m, 2H), 3.85˜3.63 (m, 7H), 3.21˜3.19 (m, 2H), 3.06˜2.97 (m, 2H), 2.84 (m, 1H), 2.63 (s, 3H), 2.46 (s, 3H), 2.28˜2.22 (m, 2H), 2.13˜2.11 (m, 1H), 1.94˜1.92 (m, 6H), 1.15˜1.14 (d, J=6.4 Hz, 3H).

LC-MS [mobile phase: from 90% water (0.1% FA) and 10% MeCN (0.1% FA) to 5% water (0.1% FA) and 95% MeCN (0.1% FA) in 9 min]: Rt=4.26 min; MS Calcd: 506.30, MS Found: 507.3 [M+H]⁺.

Chiral HPLC [Column: AD-H; Column size: 0.46 cm I.D.×15 cm L; Injection: 2 μl; Mobile phase: HEP:EtOH (0.05% DEA)=60:40; Flow rate: 0.5 mL; Wave length: UV 254 nm; Temperature: 25° C.]: Rt: 3.221 min, ee: 100%

Single Unknown Isomer 4

¹H NMR (400 MHz, CDCl₃) δ 8.78 (s, 1H), 8.05 (s, 1H), 7.50 (s, 1H), 6.91 (s, 1H), 4.10˜4.06 (m, 1H), 4.01˜3.94 (m, 2H), 3.87˜3.61 (m, 7H), 3.26˜3.19 (m, 2H), 3.06˜2.97 (m, 2H), 2.84˜2.82 (m, 1H), 2.63 (s, 3H), 2.46 (s, 3H), 2.27˜2.23 (m, 2H), 2.13˜2.11 (m, 1H), 1.94˜1.92 (m, 6H), 1.15˜1.14 (d, J=6.8 Hz, 3H).

LC-MS [mobile phase: from 90% water (0.1% FA) and 10% MeCN (0.1% FA) to 5% water (0.1% FA) and 95% MeCN (0.1% FA) in 9 min]: Rt=4.28 min; MS Calcd: 506.30, MS Found: 507.3 [M+H]⁺.

Chiral HPLC [Column: AD-H; Column size: 0.46 cm I.D.×15 cm L; Injection: 2 μl; Mobile phase: HEP:EtOH (0.05% DEA)=60:40; Flow rate: 0.5 mL; Wave length: UV 254 nm; Temperature: 25° C.]: Rt: 3.709 min, ee: 100%

### Examples 82 and 83

### ((2R)-4-(2-Methoxy-6-(5-methyl-6-(1-(tetrahydrofuran-3-yl)piperidin-4-yl)-1H-indazol-1-yl)pyrimidin-4-yl)morpholin-2-yl)methanol (Single Unknown Isomer 1, E82 and Single Unknown Isomer 2, E83)

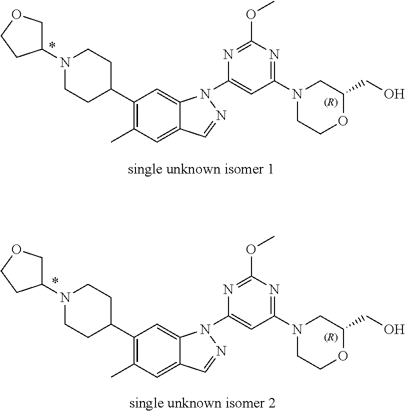

The title compound was prepared by a procedure similar to that described as E1 and E2 starting from a solution of (R)-(4-(6-iodo-2-methoxypyrimidin-4-yl)morpholin-2-yl)methanol and 5-methyl-6-(1-(tetrahydrofuran-3-yl)piperidin-4-yl)-1H-indazole in toluene, N¹,N²-dimethylethane-1,2-diamine, CuI and K₃PO₄.3H₂O at 100° C.

LC-MS [mobile phase: from 50% water (0.1% FA) and 50% MeCN (0.1% FA) to 5% water (0.1% FA) and 95% MeCN (0.1% FA) in 2.6 min]: Rt=0.76 min; MS Calcd.:508.6, MS Found: 509.3 [M+H]⁺.

Chiral Separation:

Method: Column: AD-H; Column size: 0.46 cm I.D.×15 cm L; Mobile phase: Supercritical CO₂:IPA (0.1% NH₃H₂O)=60:40; Flow rate: 0.5 mL/min; Wave length: UV 254 nm; Temperature: 25° C.; Sample solution in EtOH

Single Unknown Isomer 1

¹H NMR (400 MHz, CDCl₃) δ 8.73 (s, 1H), 8.06 (s, 1H), 7.50 (s, 1H), 6.84 (s, 1H), 4.27 (s, 2H), 4.24 (s, 3H), 4.12˜4.04 (m, 1H), 3.98˜3.95 (m, 2H), 3.93˜3.91 (m, 2H), 3.83˜3.69 (m, 4H), 3.16˜3.14 (m, 2H), 3.05˜2.96 (m, 3H), 2.83 (m, 1H), 2.46 (s, 3H), 2.24˜2.22 (m, 2H), 2.10˜2.08 (m, 1H), 1.96˜1.89 (m, 6H).

LC-MS [mobile phase: from 50% water (0.1% FA) and 50% MeCN (0.1% FA) to 5% water (0.1% FA) and 95% MeCN (0.1% FA) in 2.6 min]: Rt=0.77 min; MS Calcd: 508.6, MS Found: 509.3 [M+H]⁺.

Chiral HPLC [Column: AD-H; Column size: 0.46 cm I.D.×15 cm L; Injection: 2 μl; Mobile phase: HEP:IPA (0.1% DEA)=60:40; Flow rate: 0.5 mL/min; Wave length: UV 254 nm; Temperature: 25° C.]: Rt: 5.060 min, ee: 100%

Single Unknown Isomer 2

¹H NMR (400 MHz, CDCl₃) δ 8.73 (s, 1H), 8.06 (s, 1H), 7.50 (s, 1H), 6.84 (s, 1H), 4.30 (s, 2H), 4.28 (s, 3H), 4.12˜4.04 (m, 1H), 3.98˜3.95 (m, 2H), 3.93˜3.91 (m, 2H), 3.83˜3.67 (m, 4H), 3.16˜3.14 (m, 2H), 3.05˜2.93 (m, 3H), 2.83 (m, 1H), 2.46 (s, 3H), 2.24˜2.21 (m, 2H), 2.08 (s, 1H), 1.96˜1.89 (m, 6H).

LC-MS [mobile phase: from 50% water (0.1% FA) and 50% MeCN (0.1% FA) to 5% water (0.1% FA) and 95% MeCN (0.1% FA) in 2.6 min], purity: 99%; Rt=0.77 min; MS Calcd: 508.6, MS Found: 509.3 [M+H]⁺.

Chiral HPLC [Column: AD-H; Column size: 0.46 cm I.D.×15 cm L; Injection: 2 μl; Mobile phase: HEP:IPA (0.1% DEA)=60:40; Flow rate: 0.5 mL/min; Wave length: UV 254 nm; Temperature: 25° C.]: Rt: 5.621 min, ee: 99%

### Examples 84 and 85

### ((2S)-4-(2-Methoxy-6-(5-methyl-6-(1-(tetrahydrofuran-3-yl)piperidin-4-yl)-1H-indazol-1-yl)pyrimidin-4-yl)morpholin-2-yl)methanol (Single Unknown Isomer 1, E84 and Single Unknown Isomer 2, E85)

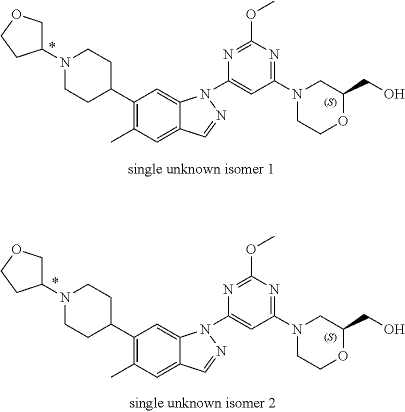

The title compound was prepared by a procedure similar to that described as E1 and E2 starting from a solution of (S)-(4-(6-iodo-2-methoxypyrimidin-4-yl)morpholin-2-yl)methanol and 5-methyl-6-(1-(tetrahydrofuran-3-yl)piperidin-4-yl)-1H-indazole in toluene, N¹,N²-dimethylethane-1,2-diamine, CuI and K₃PO₄.3H₂O at 100° C.

LC-MS [mobile phase: from 50% water (0.1% FA) and 50% MeCN (0.1% FA) to 5% water (0.1% FA) and 95% MeCN (0.1% FA) in 2.6 min]: Rt=0.77 min; MS Calcd.:508.6, MS Found: 509.3 [M+H]⁺.

Chiral Separation:

Method: Column: AD-H; Column size: 0.46 cm I.D.×15 cm L; Mobile phase: Supercritical CO₂:IPA (0.1% NH₃H₂O)=60:40; Flow rate: 0.5 mL/min; Wave length: UV 254 nm; Temperature: 25° C.; Sample solution in EtOH

Single Unknown Isomer 1

¹H NMR (400 MHz, CDCl₃) δ 8.73 (s, 1H), 8.05 (s, 1H), 7.50 (s, 1H), 6.84 (s, 1H), 4.27 (s, 2H), 4.12 (s, 3H), 4.04 (m, 1H), 3.98˜3.95 (m, 2H), 3.93˜3.91 (m, 2H), 3.83˜3.67 (m, 4H), 3.16˜3.14 (m, 2H), 3.05˜2.93 (m, 3H), 2.83 (m, 1H), 2.45 (s, 3H), 2.24˜2.21 (m, 2H), 2.10-2.07 (m, 1H), 1.90-1.89 (m, 6H).

LC-MS [mobile phase: from 50% water (0.1% FA) and 50% MeCN (0.1% FA) to 5% water (0.1% FA) and 95% MeCN (0.1% FA) in 2.6 min]: Rt=0.77 min; MS Calcd: 508.6, MS Found: 509.2 [M+H]⁺.

Chiral HPLC [Column: AD-H; Column size: 0.46 cm I.D.×15 cm L; Injection: 2 μl; Mobile phase: HEP:IPA (0.1% DEA)=60:40; Flow rate: 0.5 mL/min; Wave length: UV 254 nm; Temperature: 25° C.]: Rt: 4.877 min, ee: 100%

Single Unknown Isomer 2

¹H NMR (400 MHz, CDCl₃) δ 8.73 (s, 1H), 8.06 (s, 1H), 7.50 (s, 1H), 6.84 (s, 1H), 4.30 (s, 2H), 4.13 (s, 3H), 4.09˜4.04 (m, 1H), 3.98˜3.95 (m, 2H), 3.93˜3.91 (m, 2H), 3.83˜3.66 (m, 4H), 3.16˜3.14 (m, 2H), 3.99˜2.93 (m, 3H), 2.84 (m, 1H), 2.46 (s, 3H), 2.25˜2.23 (m, 2H), 2.10˜2.06 (m, 1H), 1.91˜1.76 (m, 6H).

LC-MS [mobile phase: from 50% water (0.1% FA) and 50% MeCN (0.1% FA) to 5% water (0.1% FA) and 95% MeCN (0.1% FA) in 2.6 min]: Rt=0.77 min; MS Calcd: 508.6, MS Found: 509.2 [M+H]⁺.

Chiral HPLC [Column: AD-H; Column size: 0.46 cm I.D.×15 cm L; Injection: 2 μl; Mobile phase: HEP:APA (0.1% DEA)=60:40; Flow rate: 0.5 mL/min; Wave length: UV 254 nm; Temperature: 25° C.]: Rt: 5.646 min, ee: 99%

### Examples 86 and 87

### ((3R)-4-(2-Methoxy-6-(5-methyl-6-(1-(tetrahydrofuran-3-yl)piperidin-4-yl)-1H-indazol-1-yl)pyrimidin-4-yl)morpholin-3-yl)methanol (Single Unknown Isomer 1, E86 and Single Unknown Isomer 2, E87)

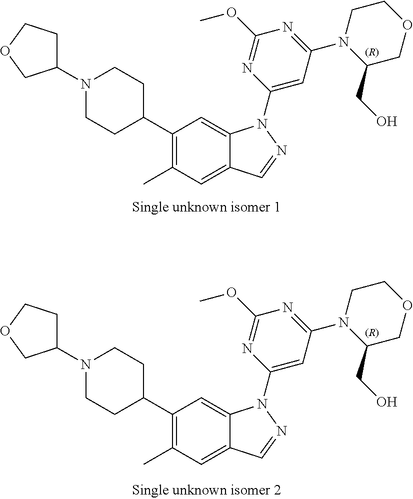

The title compound was prepared by a procedure similar to that described as E1 and E2 starting from a solution of 5-methyl-6-(1-(tetrahydrofuran-3-yl)piperidin-4-yl)-1H-indazole and (R)-(4-(6-iodo-2-methoxypyrimidin-4-yl)morpholin-3-yl)methanol in toluene, CuI, K₃PO₄ and N,N′-dimethylethylenediamine at 100° C.

LC-MS [mobile phase: from 90% water (0.1% FA) and 10% MeCN (0.1% FA) to 5% water (0.1% FA) and 95% MeCN (0.1% FA) in 9 min]: Rt=4.08 min; MS Calcd: 508.61, MS Found: 509.3 [M+H]⁺.

Chiral Separation:

Method: Column: AD-H; Column size: 0.46 cm I.D.×15 cm L; Mobile phase: Supercritical CO₂:IPA (0.1% NH₃H2)=70:30; Flow rate: 0.5 mL/min; Wave length: UV 254 nm; Temperature: 25° C.; Sample solution in EtOH

Single Unknown Isomer 1

¹H NMR (400 MHz, CDCl₃) δ 8.72 (s, 1H), 8.05 (s, 1H), 7.50 (s, 1H), 6.86 (s, 1H), 4.55 (m, 1H), 4.11 (s, 3H), 4.04 4.03 (m, 1H), 3.98 3.91 (m, 6H), 3.83˜3.81 (m, 1H), 3.70˜2.61 (m, 3H), 3.40 (m, 1H), 3.17˜3.14 (m, 1H), 3.04 (m, 1H), 2.96˜2.93 (m, 1H), 2.84 (m, 1H), 2.46 (m, 3H), 2.22˜2.19 (m, 2H), 2.10˜2.08 (m, 1H), 1.91 (m, 5H).

LC-MS [mobile phase: from 90% water (0.1% FA) and 10% MeCN (0.1% FA) to 5% water (0.1% FA) and 95% MeCN (0.1% FA) in 9 min]: Purity: 100% @ 254 nm; Rt=4.29 min; MS Calcd: 508.61, MS Found: 509.3 [M+H]⁺.

Chiral HPLC [Column: AD-H; Column size: 0.46 cm I.D.×15 cm L; Injection: 2 μl; Mobile phase: HEP:IPA (0.1% DEA)=70:30; Flow rate: 0.5 mL; Wave length: UV 254 nm; Temperature: 25° C.]: Rt: 1.428 min, ee 100%;

Single Unknown Isomer 2

¹H NMR (400 MHz, CDCl₃) δ 8.72 (s, 1H), 8.05 (s, 1H), 7.50 (s, 1H), 6.86 (s, 1H), 4.55 (m, 1H), 4.11 (s, 3H), 4.04˜4.03 (m, 1H), 3.98˜3.91 (m, 6H), 3.83˜3.81 (m, 1H), 3.70˜2.61 (m, 3H), 3.40 (m, 1H), 3.17˜3.14 (m, 1H), 3.04 (m, 1H), 2.96˜2.93 (m, 1H), 2.84 (m, 1H), 2.46 (m, 3H), 2.22˜2.19 (m, 2H), 2.10˜2.08 (m, 1H), 1.91 (m, 5H).

LC-MS [mobile phase: from 90% water (0.1% FA) and 10% MeCN (0.1% FA) to 5% water (0.1% FA) and 95% MeCN (0.1% FA) in 9 min]: Rt=4.30 min; MS Calcd: 508.61, MS Found: 509.3 [M+H]⁺.

Chiral HPLC [Column: AD-H; Column size: 0.46 cm I.D.×15 cm L; Injection: 2 μl; Mobile phase: HEP:IPA (0.1% DEA)=70:30; Flow rate: 0.5 mL; Wave length: UV 254 nm; Temperature: 25° C.]: Rt: 1.726 min, ee 99.7%;

### Examples 88 and 89

### ((3S)-4-(2-Methoxy-6-(5-methyl-6-(1-(tetrahydrofuran-3-yl)piperidin-4-yl)-1H-indazol-1-yl)pyrimidin-4-yl)morpholin-3-yl)methanol (Single Unknown Isomer 1, E88 and Single Unknown Isomer 2, E89)

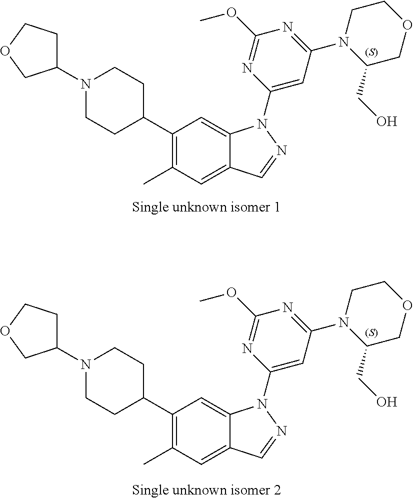

The title compound was prepared by a procedure similar to that described as E1 and E2 starting from a solution of 5-methyl-6-(1-(tetrahydrofuran-3-yl)piperidin-4-yl)-1H-indazole and (S)-(4-(6-iodo-2-methoxypyrimidin-4-yl)morpholin-3-yl)methanol in toluene, CuI, K₃PO₄ and N,N′-dimethylethylenediamine at 100° C.

LC-MS [mobile phase: from 90% water (0.1% FA) and 10% MeCN (0.1% FA) to 5% water (0.1% FA) and 95% MeCN (0.1% FA) in 2.6 min]: Rt=1.210 min; MS Calcd: 508.61, MS Found: 509.3 [M+H]⁺.

Chiral Separation:

Method: Column: AS-H; Column size: 0.46 cm I.D.×15 cm L; Mobile phase: Supercritical CO₂:IPA (0.1% NH₃H₂O)=70:30; Flow rate: 0.5 mL/min; Wave length: UV 254 nm; Temperature: 25° C.; Sample solution in EtOH

Single Unknown Isomer 1

¹H NMR (400 MHz, CDCl₃) δ 8.72 (s, 1H), 8.05 (s, 1H), 7.50 (s, 1H), 6.86 (s, 1H), 4.58˜4.54 (m, 1H), 4.11 (s, 3H), 4.04˜4.03 (m, 1H), 3.98˜3.91 (m, 5H), 3.83˜3.81 (m, 1H), 3.79 2.67 (m, 2H), 3.62 3.58 (m, 1H), 3.42 3.39 (m, 1H), 3.17˜3.14 (m, 1H), 3.05 3.01 (m, 1H), 2.95˜2.93 (m, 1H), 2.83˜2.82 (m, 1H), 2.45 (m, 3H), 2.26˜2.22 (m, 2H), 2.12˜2.07 (m, 2H), 1.91˜1.86 (m, 6H).

LC-MS [mobile phase: from 50% water (0.1% NH₄OH) and 50% MeCN (0.1% NH₄OH) to 5% water (0.1% NH₄OH) and 95% MeCN (0.1% NH₄OH) in 2.6 min]: Purity: 94% @ 254 nm; Rt=0.95 min; MS Calcd: 508.61, MS Found: 509.3 [M+H]⁺.

Chiral HPLC [Column: AS-H; Column size: 0.46 cm I.D.×15 cm L; Injection: 2 μl; Mobile phase: HEP:IPA (0.1% DEA)=70:30; Flow rate: 0.5 mL; Wave length: UV 254 nm; Temperature: 25° C.]: Rt: 3.689 min, ee 100%;

Single Unknown Isomer 2

¹H NMR (400 MHz, CDCl₃) δ 8.72 (s, 1H), 8.05 (s, 1H), 7.50 (s, 1H), 6.86 (s, 1H), 4.55 4.52 (m, 1H), 4.11 (s, 3H), 4.03˜4.02 (m, 1H), 3.98˜3.90 (m, 5H), 3.85˜3.81 (m, 1H), 3.79˜2.67 (m, 2H), 2.62˜3.59 (m, 1H), 3.43˜3.40 (m, 1H), 3.17˜3.14 (m, 1H), 3.05˜3.02 (m, 1H), 2.95˜2.93 (m, 1H), 2.85˜2.80 (m, 1H), 2.46 (s, 3H), 2.28˜2.22 (m, 2H), 2.19˜2.07 (m, 2H), 1.96˜1.84 (m, 6H).

LC-MS [mobile phase: from 50% water (0.1% NH₄OH) and 50% MeCN (0.1% NH₄OH) to 5% water (0.1% NH₄OH) and 95% MeCN (0.1% NH₄OH) in 2.6 min]: Rt=0.92 min; MS Calcd: 508.61, MS Found: 509.3 [M+H]⁺.

Chiral HPLC [Column: AS-H; Column size: 0.46 cm I.D.×15 cm L; Injection: 2 μl; Mobile phase: HEP:IPA (0.1% DEA)=70:30; Flow rate: 0.5 mL; Wave length: UV 254 nm; Temperature: 25° C.]: Rt: 3.806 min, ee 99%;

### Examples 90 and 91

### (2R)-2-Methyl-4-(2-methyl-6-(5-methyl-6-(1-(tetrahydrofuran-3-yl)piperidin-4-yl)-1H-indazol-1-yl)pyrimidin-4-yl)morpholine (Single Unknown Isomer 1, E90 and Single Unknown Isomer 2, E91)

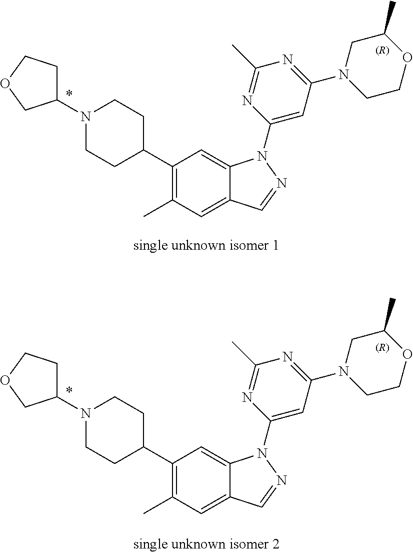

To a solution of 1-(6-chloro-2-methylpyrimidin-4-yl)-5-methyl-6-(1-(tetrahydrofuran-3-yl)piperidin-4-yl)-1H-indazole (100 mg, 0.240 mmol) and (R)-2-methylmorpholine hydrochloride (33.0 mg, 0.240 mmol) in DMF (30 mL) was added DIEA (155 mg, 1.20 mmol) at rt. The reaction mixture was stirred at 80° C. overnight. LC-MS showed reaction was completed. The reaction mixture was cooled to room temperature, diluted with water (50 mL) and extracted with EtOAc (3×50 mL). The combined organic layers were washed with water (3×50 mL), brine (50 mL), dried and filtered and concentrated to give a residue. The residue was purified by silica gel chromatography eluted with EtOAc:MeOH (20:1) to give the desired product as a yellow solid (100 mg, yield: 86%).

LC-MS [mobile phase: from 50% water (0.1% FA) and 50% MeCN (0.1% FA) to 5% water (0.1% FA) and 95% MeCN (0.1% FA) in 2.6 min]: Rt=0.81 min; MS Calcd.:476.6, MS Found: 477.3 [M+H]⁺.

Chiral Separation:

Method: Column: AD-H; Column size: 0.46 cm I.D.×15 cm L; Mobile phase: Supercritical CO₂:EtOH (0.1% NH₃H₂O)=60:40; Flow rate: 0.5 mL; Wave length: UV 254 nm; Temperature: 25° C.; Sample solution in EtOH

Single Unknown Isomer 1

¹H NMR (400 MHz, CDCl₃) δ 8.80 (s, 1H), 8.05 (s, 1H), 7.49 (s, 1H), 6.93 (s, 1H), 4.28 (s, 2H) 4.02˜3.94 (m, 3H), 3.84˜3.82 (m, 1H), 3.74˜3.64 (m, 3H), 3.21˜3.17 (m, 1H), 3.05˜2.96 (m, 3H), 2.84 (s, 1H), 2.74˜2.68 (m, 1H), 2.63 (s, 3H), 2.45 (s, 3H), 2.26 (s, 2H), 2.25˜2.23 (m, 1H), 2.13˜1.91 (m, 5H), 1.28˜1.26 (d, J=8.0 Hz, 3H)

LC-MS [mobile phase: from 50% water (0.1% FA) and 50% MeCN (0.1% FA) to 5% water (0.1% FA) and 95% MeCN (0.1% FA) in 2.6 min]: purity: 99%; Rt=0.8 min; MS Calcd: 476.6, MS Found: 477.3 [M+H]⁺.

Chiral HPLC [Column: AD-H; Column size: 0.46 cm I.D.×15 cm L; Injection: 2 μl; Mobile phase: HEP:EtOH (0.1% DEA)=60:40; Flow rate: 0.5 mL; Wave length: UV 254 nm; Temperature: 25° C.]: Rt: 2.027 min, ee: 100%

Single Unknown Isomer 2

¹H NMR (400 MHz, CDCl₃) δ 8.80 (s, 1H), 8.05 (s, 1H), 7.49 (s, 1H), 6.93 (s, 1H), 4.29 (s, 2H) 4.01˜3.94 (m, 3H), 3.84˜3.82 (m, 1H), 3.74˜3.64 (m, 3H), 3.21˜3.17 (m, 1H), 3.05˜2.96 (m, 3H), 2.84 (s, 1H), 2.74˜2.68 (m, 1H), 2.63 (s, 3H), 2.45 (s, 3H), 2.26 (s, 2H), 2.24˜2.23 (m, 1H), 2.13˜1.92 (m, 5H), 1.28˜1.26 (d, J=8.0 Hz, 3H)

LC-MS [mobile phase: from 50% water (0.1% FA) and 50% MeCN (0.1% FA) to 5% water (0.1% FA) and 95% MeCN (0.1% FA) in 2.0 min]: purity: 99%; Rt=0.8 min; MS Calcd: 476.6, MS Found: 477.3 [M+H]⁺.

Chiral HPLC [Column: AD-H; Column size: 0.46 cm I.D.×15 cm L; Injection: 2 μl; Mobile phase: HEP:EtOH (0.1% DEA)=60:40; Flow rate: 0.5 mL; Wave length: UV 254 nm; Temperature: 25° C.]: Rt: 2.460 min, ee: 98%

### Examples 92 and 93

### (2S)-2-Methyl-4-(2-methyl-6-(5-methyl-6-(1-(tetrahydrofuran-3-yl)piperidin-4-yl)-1H-indazol-1-yl)pyrimidin-4-yl)morpholine (Single Unknown Isomer 1, E92; Single Unknown Isomer 2, E93)

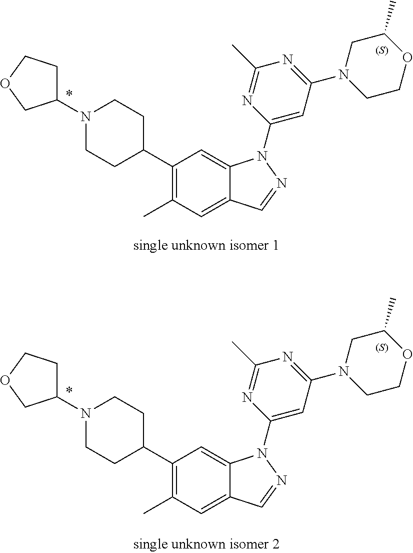

The title compound was prepared by a procedure similar to that described for E90 and E91 starting from a solution of 1-(6-chloro-2-methylpyrimidin-4-yl)-5-methyl-6-(1-(tetrahydrofuran-3-yl)piperidin-4-yl)-1H-indazole and (S)-2-methylmorpholine hydrochl-oride in DMF and DIEA at 80° C.

LC-MS [mobile phase: from 80% water (0.1% FA) and 20% MeCN (0.1% FA) to 5% water (0.1% FA) and 95% MeCN (0.1% FA) in 2.6 min]: Rt=0.97 min; MS Calcd.:476.6, MS Found: 477.3 [M+H]⁺.

Chiral Separation:

Method: Column: AD-H; Column size: 0.46 cm I.D.×15 cm L; Mobile phase: Supercritical CO₂:EtOH (0.1% NH₃H₂O)=60:40; Flow rate: 0.5 mL/min; Wave length: UV 254 nm; Temperature: 25° C.; Sample solution in EtOH

Single Unknown Isomer 1

¹H NMR (400 MHz, CDCl₃) δ 8.80 (s, 1H), 8.05 (s, 1H), 7.49 (s, 1H), 6.93 (s, 1H), 4.28 (s, 2H) 4.02˜3.94 (m, 3H), 3.84˜3.82 (m, 1H), 3.72˜3.64 (m, 3H), 3.19 (s, 1H), 3.05˜3.02 (m, 3H), 2.82 (s, 1H), 2.74˜2.68 (m, 1H), 2.63 (s, 3H), 2.45 (s, 3H), 2.25 (s, 2H), 2.24 (s, 1H), 2.09˜1.94 (m, 5H), 1.28˜1.26 (d, J=8.0 Hz, 3H)

LC-MS [mobile phase: from 50% water (0.1% FA) and 50% MeCN (0.1% FA) to 5% water (0.1% FA) and 95% MeCN (0.1% FA) in 2.6 min], purity: 99%; Rt=0.8 min; MS Calcd: 476.6, MS Found: 477.3 [M+H]⁺.

Chiral HPLC [Column: AD-H; Column size: 0.46 cm I.D.×15 cm L; Injection: 2 μl; Mobile phase: HEP:EtOH (0.1% DEA)=60:40; Flow rate: 0.5 mL/min; Wave length: UV 254 nm; Temperature: 25° C.]: Rt: 1.919 min, ee: 100%

Single Unknown Isomer 2

¹H NMR (400 MHz, CDCl₃) δ 8.80 (s, 1H), 8.05 (s, 1H), 7.49 (s, 1H), 6.93 (s, 1H), 4.28 (s, 2H) 4.01˜3.94 (m, 3H), 3.86˜3.82 (m, 1H), 3.74˜3.62 (m, 3H), 3.21˜3.18 (m, 1H), 3.09˜2.97 (m, 3H), 2.84 (s, 1H), 2.74˜2.68 (m, 1H), 2.63 (s, 3H), 2.45 (s, 3H), 2.25 (s, 2H), 2.13 (s, 1H), 2.12˜1.92 (m, 5H), 1.28˜1.26 (d, J=8.0 Hz, 3H)

LC-MS [mobile phase: from 50% water (0.1% FA) and 50% MeCN (0.1% FA) to 5% water (0.1% FA) and 95% MeCN (0.1% FA) in 2.6 min]: purity: 99%; Rt=0.8 min; MS Calcd: 476.6, MS Found: 477.3 [M+H]⁺.

Chiral HPLC [Column: AD-H; Column size: 0.46 cm I.D.×15 cm L; Injection: 2 μl; Mobile phase: HEP:EtOH (0.1% DEA)=60:40; Flow rate: 0.5 mL/min; Wave length: UV 254 nm; Temperature: 25° C.]: Rt: 2.465 min, ee: 100%

### Example 94

### ((R)-4-(6-(3-deuterium-5-methyl-6-(1-((R)-tetrahydrofuran-3-yl)piperidin-4-yl)-1H-indazol-1-yl)-2-methylpyrimidin-4-yl)morpholin-2-yl)methanol

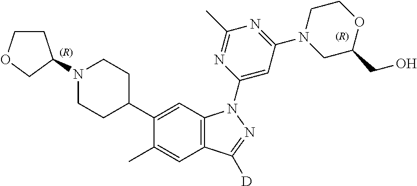

The title compound was prepared by a procedure similar to that described for E1 and E2 starting from a suspension of (R)-3-deuterium-5-methyl-6-(1-(tetrahydrofuran-3-yl)piperidin-4-yl)-1H-indazole (D105), (R)-(4-(6-iodo-2-methylpyrimidin-4-yl) morpholin-2-yl)methanol (D12), CuI and K₃PO₄ in toluene and DMEDA.

¹H NMR (400 MHz, CDCl₃): δ 8.78 (s, 1H), 8.05 (s, 0.01H), 7.50 (s, 1H), 6.95 (s, 1H), 4.32˜4.29 (m, 2H), 4.08˜3.94 (m, 3H), 3.86˜3.68 (m, 6H), 3.21˜2.92 (m, 5H), 2.88˜2.80 (m, 1H), 2.64 (s, 3H), 2.46 (s, 3H), 2.30˜2.18 (m, 2H), 2.16˜2.08 (m, 1H), 2.00˜1.92 (m, 5H).

LC-MS [mobile phase: from 80% water (0.1% FA) and 20% MeCN (0.1% FA) to 5% water (0.1% FA) and 95% MeCN (0.1% FA) in 12 min]: Rt=4.88 min; MS Calcd: 493.3, MS Found: 494.4 [M+H]⁺.

Chiral HPLC [Column: AD, Column size: 4.6×250 mm, 5 μm. Injection: 10 μl, Mobile phase: CO₂/EtOH/MeCN/DEA 60/34/6/0.08, Flow rate: 2.8 mL/min, Wave length: UV 254 nm, Temperature: 35° C.]: Rt=13.383 min, ee: 100%

### Example 95

### ((R)-4-(6-(3-deuterium-5-methyl-6-(1-((S)-tetrahydrofuran-3-yl)piperidin-4-yl)-1H-indazol-1-yl)-2-methylpyrimidin-4-yl)morpholin-2-yl)methanol

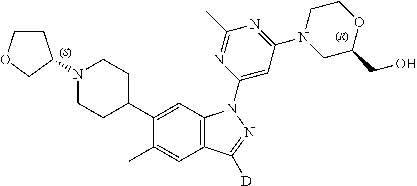

The title compound was prepared by a procedure similar to that described for E1 and E2 starting from a suspension of (S)-3-deuterium-5-methyl-6-(1-(tetrahydrofuran-3-yl)piperidin-4-yl)-1H-indazole (D104), (R)-(4-(6-iodo-2-methylpyrimidin-4-yl) morpholin-2-yl)methanol (D12), CuI and K₃PO₄ in toluene and DMEDA.

¹H NMR (400 MHz, CDCl₃): δ 8.78 (s, 1H), 8.05 (s, 0.01H), 7.50 (s, 1H), 6.95 (s, 1H), 4.32˜4.29 (m, 2H), 4.08˜3.94 (m, 3H), 3.86˜3.66 (m, 6H), 3.24˜3.20 (m, 1H), 3.15˜2.92 (m, 4H), 2.89˜2.79 (m, 1H), 2.64 (s, 3H), 2.46 (s, 3H), 2.30˜2.23 (m, 2H), 2.15˜2.08 (m, 1H), 2.04˜1.93 (m, 5H).

LC-MS [mobile phase: from 80% water (0.1% FA) and 20% MeCN (0.1% FA) to 5% water (0.1% FA) and 95% MeCN (0.1% FA) in 12 min]: Rt=4.75 min; MS Calcd: 493.3, MS Found: 494.5[M+H]⁺.

Chiral HPLC [method: Column: AD, Column size: 4.6×250 mm, 5 μm (UPC). Injection: 10 μl, Mobile phase: CO₂/EtOH/MeCN/DEA 60/34/6/0.08, Flow rate: 2.8 mL/min, Wave length: UV 254 nm, Temperature: 35° C.]: Rt=19.055 min, ee: 99.42%

### Example 96

### ((R)-4-(2-methyl-6-(5-methyl-6-(1-((S)-tetrahydrofuran-3-yl)piperidin-4-yl-2,2,6,6-d4)-1H-indazol-1-yl)pyrimidin-4-yl)morpholin-2-yl)methanol

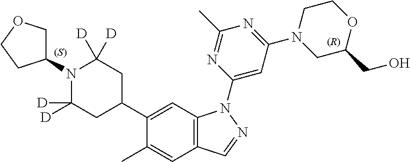

A mixture of (R)-(4-(2-methyl-6-(5-methyl-6-(piperidin-4-yl-2,2,6,6-d4)-1H-indazol-1-yl)pyrimidin-4-yl)morpholin-2-yl)methanol (D116, 55 mg, 0.13 mmol), (R)-tetrahydro-furan-3-yl-4-methylbenzenesulfonate (D12, 63 mg, 0.26 mmol), K₂CO₃ (36 mg, 0.26 mmol) in MeCN (10 mL) was stirred at 100° C. overnight and then filtered. The filtrate was purified by pre-HPLC (Waters2767/Qda, Waters XBridge Prep C₁₈ 10 μm OBD™ 19×250 nm, flow rate: 30 mL/min, 254 nm, mobile phase: MeCN/H₂O (0.1% NH₃.H₂O), from 5:95 to 95:5) to give the title product as a white solid (5.0 mg, yield: 13%).

¹H NMR (400 MHz, CDCl₃): δ 8.78 (s, 1H), 8.05 (s, 1H), 7.49 (s, 1H), 6.95 (s, 1H), 4.31˜4.27 (m, 2H), 4.08˜4.04 (m, 1H), 3.99˜3.93 (m, 2H), 3.84˜3.67 (m, 7H), 3.12˜3.04 (m, 2H), 2.97˜2.84 (m, 2H), 2.63 (s, 3H), 2.45 (s, 3H), 2.17˜2.10 (br, 1H), 1.96˜1.89 (m, 4H).

LC-MS [mobile phase: from 90% water (0.1% FA) and 10% MeCN (0.1% FA) to 5% water (0.1% FA) and 95% MeCN (0.1% FA) in 12 min]: Rt=4.941 min; MS Calcd.:496.3, MS Found: 497.4 [M+H]⁺.

Chiral HPLC [method: Column: AD, 5 μm, 4.6×250 cm (Daicel). Injection: 10 μl, Mobile phase: CO₂/EtOH/MeCN/DEA 60/34/6/0.08, Flow rate: 2.8 mL/min, Wave length: UV 254 nm, Temperature: 35° C.]: Rt=19.195 min, ee: 99.28%

### Examples 97, 98, 99 and 100

### 1-(1-(2-methoxy-6-(5-methyl-6-(1-(tetrahydrofuran-3-yl)piperidin-4-yl)-1H-indazol-1-yl)pyrimidin-4-yl)azetidin-3-yl)ethanol (Single Unknown Isomer 1, Rt=1.821 Min; Single Unknown Isomer 2, Rt=1.997 Min; Single Unknown Isomer 3, Rt=4.235 Min; Single unknown isomer 4, Rt=4.530 min)

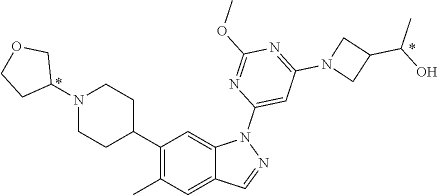 [SMILES: COc1nc(N2CC(C(C)O)C2)cc(-n2ncc3cc(C)c(C4CCN(C5CCOC5)CC4)cc32)n1]

The title compounds were prepared by a procedure similar to that described for E1 and E2 starting from a solution of 1-(1-(6-iodo-2-methoxypyrimidin-4-yl)azetidin-3-yl)ethanol (D117) and 5-methyl-6-(tetrahydrofuran-3-yl)-1H-indazole (D10) in toluene and N¹,N²-dimethylethane-1,2-diamine, CuI and K₃PO₄ at reflux for 4 hours.

Chiral Separation:

method: AD-H, 0.46 cm×15 cm, Phase: Supercritical CO₂: EtOH (0.05% NH3.H₂O)=60/40, Flow rate: 0.5 mL/min, Wave length:: 254 nm, Temperature: 25° C.

Peak 1 (E97): Single Unknown Isomer 1, Rt=1.821 Min

¹H NMR (400 MHz, CDCl₃): δ 8.74 (s, 1H), 8.05 (s, 1H), 7.50 (s, 1H), 6.45 (s, 1H), 4.21-3.68 (m, 11H), 3.17-2.74 (m, 5H), 2.45 (s, 3H), 2.29-1.85 (m, 10H), 1.21 (t, J=6.0 Hz, 3H).

LC-MS [mobile phase: from 80% water (0.1% FA) and 20% MeCN (0.1% FA) to 5% water (0.1% FA) and 95% MeCN (0.1% FA) in 10 min]: Rt=3.31 min; MS Calcd: 492, MS Found: 493 [M+H]⁺.

Chiral HPLC [method: AD-H, 0.46 cm×15 cm, Phase: HEP: EtOH (0.05% DEA)=60/40, Flow rate: 0.5 mL/min, Wave length: 254 nm, Temperature: 25° C.]: Rt=1.821 min; ee: 96.5%

Peak 2 (E98): Single Unknown Isomer 2, Rt=1.997 Min

¹H NMR (400 MHz, CDCl₃): δ 8.74 (s, 1H), 8.05 (s, 1H), 7.50 (s, 1H), 6.45 (s, 1H), 4.21-3.68 (m, 11H), 3.17-2.74 (m, 5H), 2.45 (s, 3H), 2.29-1.85 (m, 10H), 1.21 (t, J=6.0 Hz, 3H).

LC-MS [mobile phase: from 80% water (0.1% FA) and 20% MeCN (0.1% FA) to 5% water (0.1% FA) and 95% MeCN (0.1% FA) in 10 min]: Rt=3.35 min; MS Calcd: 492, MS Found: 493 [M+H]⁺.

Chiral HPLC [method: AD-H, 0.46 cm×15 cm, Phase: HEP: EtOH (0.05% DEA)=60/40, Flow rate: 0.5 mL/min, Wave length: 254 nm, Temperature: 25° C.]: Rt=1.997 min; ee: 98.5%.

Peak 3 (E99): Single Unknown Isomer 3, Rt=4.235 Min

¹H NMR (400 MHz, CDCl₃): δ 8.74 (s, 1H), 8.05 (s, 1H), 7.50 (s, 1H), 6.45 (s, 1H), 4.21-3.68 (m, 11H), 3.17-2.74 (m, 5H), 2.45 (s, 3H), 2.29-1.85 (m, 10H), 1.21 (t, J=6.0 Hz, 3H).

LC-MS [mobile phase: from 80% water (0.1% FA) and 20% MeCN (0.1% FA) to 5% water (0.1% FA) and 95% MeCN (0.1% FA) in 10 min]: Rt=3.42 min; MS Calcd: 492, MS Found: 493 [M+H]⁺.

Chiral HPLC [method: AD-H, 0.46 cm×15 cm, Phase: HEP: EtOH (0.05% DEA)=60/40, Flow rate: 0.5 mL/min, W: 254 nm, T: 25° C.]: Rt=4.235 min; ee: 100%.

Peak 4 (E100): Single Unknown Isomer 4, Rt=4.530 Min

¹H NMR (400 MHz, CDCl₃): δ 8.74 (s, 1H), 8.05 (s, 1H), 7.50 (s, 1H), 6.45 (s, 1H), 4.21-3.68 (m, 11H), 3.17-2.74 (m, 5H), 2.45 (s, 3H), 2.29-1.85 (m, 10H), 1.21 (t, J=6.0 Hz, 3H).

LC-MS [mobile phase: from 80% water (0.1% FA) and 20% MeCN (0.1% FA) to 5% water (0.1% FA) and 95% MeCN (0.1% FA) in 10 min]: Rt=3.39 min; MS Calcd: 492, MS Found: 493 [M+H]⁺.

Chiral HPLC [method: AD-H, 0.46 cm×15 cm, Phase: HEP: EtOH (0.05% DEA)=60/40, Flow rate: 0.5 mL/min, Wave length: 254 nm, Temperature: 25° C.]: Rt=4. 530 min, ee: 97.5%.

### Examples 101 and 102

### cis-((2R)-4-(6-(6-(3-fluoro-1-(tetrahydrofuran-3-yl)piperidin-4-yl)-5-methyl-1H-indazol-1-yl)-2-methylpyrimidin-4-yl)morpholin-2-yl)methanol (from Peak 2, D34) (Single Unknown Isomer 1, Rt=2.984 Min; Single Unknown Isomer 2, Rt=3.948 Min)

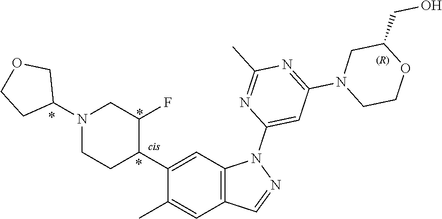

The title compounds were prepared by a procedure similar to that described for E1 and E2 starting from a solution of cis-6-(3-fluoro-1-(tetrahydrofuran-3-yl)piperidin-4-yl)-5-methyl-1H-indazole (peak 2, D34), (R)-(4-(6-iodo-2-methylpyrimidin-4-yl)morpholin-2-yl)met-hanol (D12), CuI, K₃PO₄ and N¹,N²-dimethylethane-1,2-diamine in toluene at 90° C. under N₂ protection.

Chiral Separation:

method: AD-H, 0.46 cm×15 cm, Phase: Supercritical CO₂:EtOH (0.1% NH₃.H₂O)=60/40, Flow rate: 0.5 mL/min, Wave length: 254 nm, Temperature: 25° C.

Peak 1 (E101): Single Unknown Isomer 1, Rt=2.984 Min

¹H NMR (400 MHz, MeOD): δ 8.85 (s, 1H), 8.16 (s, 1H), 7.62 (s, 1H), 7.04 (s, 1H), 4.94˜4.92 (m, 1H), 4.43˜4.42 (m, 1H), 4.39˜4.30 (m, 1H), 4.05˜3.98 (m, 3H), 3.90˜3.74 (m, 1H), 3.69˜3.65 (m, 1H), 3.64˜3.59 (m, 6H), 3.47˜3.45 (m, 1H), 3.25˜3.19 (m, 2H), 3.10˜3.07 (m, 1H), 2.88˜2.82 (m, 2H), 2.60 (s, 3H), 2.47 (s, 3H), 2.35˜2.29 (m, 1H), 2.18˜2.15 (m, 2H), 2.03˜1.95 (m, 1H).

¹⁹F NMR (376 MHz, MeOD): δ-184.80.

LC-MS [mobile phase: from 70% water (0.1% FA) and 30% MeCN (0.1% FA) to 5% water (0.1% FA) and 95% MeCN (0.1% FA) in 12 min]: Rt=7.78 min; MS Calcd: 510.6, MS Found: 511.3 [M+H]⁺.

Chiral HPLC [method: AD-H, 0.46 cm×15 cm, Phase: HEP: EtOH (0.1% DEA)=60/40, Flow rate: 0.5 mL/min, Wave length: 254 nm, Temperature: 25° C.]: Rt=2.984 min, ee: 100%.

Peak 2 (E102): Single Unknown Isomer 2, Rt=3.948 Min

¹H NMR (400 MHz, MeOD): δ 8.85 (s, 1H), 8.15 (s, 1H), 7.61 (s, 1H), 7.04 (s, 1H), 4.88˜4.74 (m, 1H), 4.43˜4.42 (m, 1H), 4.39˜4.30 (m, 1H), 4.05˜3.98 (m, 2H), 3.90˜3.87 (m, 1H), 3.78˜3.69 (m, 2H), 3.66˜3.60 (m, 5H), 3.30˜3.28 (m, 2H), 3.27˜3.19 (m, 2H), 2.88˜2.82 (m, 1H), 2.60 (s, 3H), 2.47 (s, 3H), 2.25˜2.29 (m, 2H), 2.18˜2.15 (m, 1H), 2.03˜1.95 (m, 3H).

¹⁹F NMR (376 MHz, MeOD): δ-184.80.

LC-MS [mobile phase: from 70% water (0.1% FA) and 30% MeCN (0.1% FA) to 5% water (0.1% FA) and 95% MeCN (0.1% FA) in 12 min]: Rt=7.80 min; MS Calcd: 510.6, MS Found: 511.3 [M+H]⁺.

Chiral HPLC [method: AD-H, 0.46 cm×15 cm, Phase: HEP: EtOH (0.1% DEA)=60/40, Flow rate: 0.5 mL/min, Wave length: 254 nm, Temperature: 25° C.]: Rt=3.948 min, ee: 98.1%.

### Examples 103 and 104

### Cis-((2S)-4-(6-(6-(3-fluoro-1-(tetrahydrofuran-3-yl)piperidin-4-yl)-5-methyl-1H-indazol-1-yl)-2-methylpyrimidin-4-yl)morpholin-2-yl)methanol (from Peak 1, D33) (Single Unknown Isomer 1, Rt=5.134 Min; Single Unknown Isomer 2, Rt=5.400 Min)

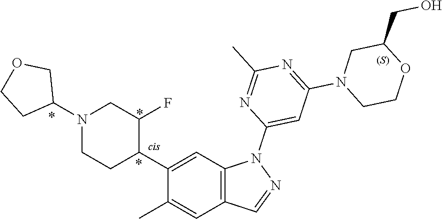

The title compounds were prepared by a procedure similar to that described for E1 and E2 starting from a solution of cis-6-(3-fluoro-1-(tetrahydrofuran-3-yl)piperidin-4-yl)-5-methyl-1H-indazole (peak 1, D33), (S)-(4-(6-iodo-2-methylpyrimidin-4-yl)morpholin-2-yl)methanol (D3), CuI, K₃PO₄ and N¹,N²-dimethylethane-1,2-diamine in toluene at 90° C. under N₂.

Chiral Separation:

AD-H, 0.46 cm×15 cm, Mobile phase: Supercritical C₂:EtOH(0.1% NH₃.H₂O)=60/40, Flow rate: 0.5 mL/min, Wave length: 254 nm, Temperature: 25° C.

Peak 1 (E103): Single Unknown Isomer 1, Rt=5.134 Min

¹H NMR (400 MHz, MeOD): δ 8.85 (s, 1H), 8.15 (s, 1H), 7.59 (s, 1H), 7.04 (s, 1H), 4.78˜4.71 (m, 1H), 4.43˜4.42 (m, 1H), 4.39˜4.30 (m, 1H), 4.05˜3.98 (m, 2H), 3.90˜3.79 (m, 1H), 3.77˜3.75 (m, 2H), 3.66˜3.61 (m, 4H), 3.25˜3.19 (m, 3H), 3.10˜3.07 (m, 2H), 2.88˜2.82 (m, 1H), 2.60 (s, 3H), 2.47 (s, 3H), 2.35˜2.29 (m, 2H), 2.18˜2.15 (m, 1H), 2.03˜1.95 (m, 3H).

¹⁹F NMR (376 MHz, MeOD): δ-184.82.

LC-MS [mobile phase: from 70% water (0.1% FA) and 30% MeCN (0.1% FA) to 5% water (0.1% FA) and 95% MeCN (0.1% FA) in 12 min]: Rt=7.92 min; MS Calcd: 510.6, MS Found: 511.3 [M+H]⁺.

Chiral HPLC [AD-H, 0.46 cm×15 cm, Phase: HEP:EtOH (0.1% DEA)=60/40, Flow rate: 0.5 mL/min, Wave length: 254 nm, Temperature: 25° C.]: Rt=5.134 min, ee: 100%.

Peak 2 (E104): Single Unknown Isomer 2, Rt=5.400 Min

¹H NMR (400 MHz, MeOD): δ 8.85 (s, 1H), 8.15 (s, 1H), 7.59 (s, 1H), 7.04 (s, 1H), 4.78˜4.71 (m, 1H), 4.43˜4.42 (m, 1H), 4.39˜4.30 (m, 1H), 4.05˜3.98 (m, 2H), 3.90˜3.79 (m, 1H), 3.77˜3.75 (m, 2H), 3.66˜3.61 (m, 4H), 3.47˜3.45 (m, 1H), 3.25˜3.19 (m, 2H), 3.10˜3.07 (m, 1H), 2.88˜2.82 (m, 2H), 2.60 (s, 3H), 2.47 (s, 3H), 2.35˜2.29 (m, 2H), 2.18˜2.15 (m, 1H), 2.03˜1.95 (m, 3H).

¹⁹F NMR (376 MHz, MeOD): δ-184.82.

LC-MS [mobile phase: from 70% water (0.1% FA) and 30% MeCN (0.1% FA) to 5% water (0.1% FA) and 95% MeCN (0.1% FA) in 12 min]: Rt=7.90 min; MS Calcd: 510.6, MS Found: 511.3 [M+H]⁺.

Chiral HPLC [AD-H, 0.46 cm×15 cm, Phase: HEP:EtOH (0.1% DEA)=60/40, Flow rate: 0.5 mL/min, Wave length: 254 nm, Temperature: 25° C.]: Rt=5.400 min, ee: 99.1%.

### Examples 105 and 106

### Cis-((2S)-4-(6-(6-(3-fluoro-1-(tetrahydrofuran-3-yl)piperidin-4-yl)-5-methyl-1H-indazol-1-yl)-2-methylpyrimidin-4-yl)morpholin-2-yl)methanol (From Peak 2, D34) (Single Unknown Isomer 1, Rt=5.082 Min; Single Unknown Isomer 2, Rt=5.826 Min)

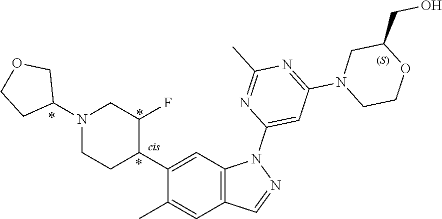

The title compounds were prepared by a procedure similar to that described for E1 and E2 starting from a solution of cis-6-(3-fluoro-1-(tetrahydrofuran-3-yl)piperidin-4-yl)-5-methyl-1H-indazole (from Peak 2, D34), (S)-(4-(6-iodo-2-methylpyrimidin-4-yl)morpholin-2-yl)methanol (D3), CuI and K₃PO₄ in toluene and N¹,N²-dimethylethane-1,2-diamine at 90° C. for 2 hrs under N₂.

Chiral Separation:

method: AD-H, 0.46 cm×15 cm, Mobile phase: Supercritical CO₂:EtOH (0.1% NH₃H₂O)=60/40, Flow rate: 0.5 mL/min, Wave length: 254 nm, Temperature: 25° C.

Peak 1 (E105): Single Unknown Isomer 1, Rt=5.082 Min

¹H NMR (400 MHz, MeOD): δ 8.85 (s, 1H), 8.15 (s, 1H), 7.59 (s, 1H), 7.04 (s, 1H), 4.79˜4.71 (m, 1H), 4.43˜4.42 (m, 1H), 4.39˜4.30 (m, 1H), 4.05˜3.98 (m, 2H), 3.90˜3.79 (m, 1H), 3.77˜3.75 (m, 2H), 3.66˜3.61 (m, 4H), 3.47˜3.45 (m, 1H), 3.25˜3.19 (m, 2H), 3.10˜3.07 (m, 1H), 2.88˜2.82 (m, 2H), 2.60 (s, 3H), 2.47 (s, 3H), 2.35˜2.29 (m, 2H), 2.18˜2.15 (m, 1H), 2.03˜1.95 (m, 3H).

¹⁹F NMR (376 MHz, MeOD): δ-184.74.

LC-MS [mobile phase: from 70% water (0.1% FA) and 30% MeCN (0.1% FA) to 5% water (0.1% FA) and 95% MeCN (0.1% FA) in 12 min]: Rt=7.89 min; MS Calcd: 510.6, MS Found: 511.3 [M+H]⁺.

Chiral HPLC [method: AD-H, 0.46 cm×15 cm, Phase: HEP: EtOH (0.1% DEA)=60/40, Flow rate: 0.5 mL/min, Wave length: 254 nm, Temperature: 25° C.]: Rt=5.134 min, ee: 100%.

Peak 2 (E106): Single Unknown Isomer 2, Rt=5.826 Min

¹H NMR (400 MHz, MeOD): δ 8.85 (s, 1H), 8.15 (s, 1H), 7.61 (s, 1H), 7.04 (s, 1H), 4.98˜4.94 (m, 1H), 4.43˜4.42 (m, 1H), 4.39˜4.30 (m, 1H), 4.05˜3.98 (m, 2H), 3.90˜3.87 (m, 2H), 3.78˜3.69 (m, 1H), 3.66˜3.60 (m, 5H), 3.58˜3.47 (m, 1H), 3.25˜3.19 (m, 2H), 3.10˜3.07 (m, 1H), 2.88˜2.82 (m, 1H), 2.60 (m, 5H), 2.47 (s, 3H), 2.25˜2.29 (m, 1H), 2.18˜2.15 (m, 2H), 2.03˜1.95 (m, 1H).

¹⁹F NMR (376 MHz, MeOD): δ-184.74.

LC-MS [mobile phase: from 70% water (0.1% FA) and 30% MeCN (0.1% FA) to 5% water (0.1% FA) and 95% MeCN (0.1% FA) in 12 min]: Rt=7.80 min; MS Calcd: 510.6, MS Found: 511.3[M+H]⁺.

Chiral HPLC [method: AD-H, 0.46 cm×15 cm, Phase: HEP: EtOH (0.1% DEA)=60/40, Flow rate: 0.5 mL/min, Wave length: 254 nm, Temperature: 25° C.]: Rt=5.400 min, ee: 100%.

### Examples 107 and 108

### Cis-4-(6-(6-(3-fluoro-1-(tetrahydrofuran-3-yl)-piperidin-4-yl)-5-methyl-1H-indazol-1-yl)-2-methoxypyrimidin-4-yl)morpholine (from Peak 1, D33) (Single Unknown Isomer 1, Rt=2.541 Min; Single Unknown Isomer 2, Rt=2.985 Min)

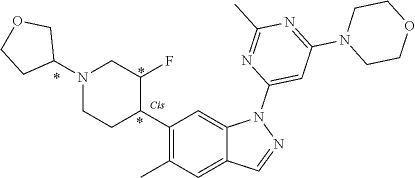 [SMILES: Cc1nc(N2CCOCC2)cc(-n2ncc3cc(C)c(C4CCN(C5CCOC5)CC4F)cc32)n1]

The title compounds were prepared by a procedure similar to that described for E1 and E2 starting from a mixture of cis-4-(6-(6-(3-fluoro-1-(tetrahydrofuran-3-yl)piperidin-4-yl)-5-methyl-1H-indazol-1-yl)-2-methylpyrimidin-4-yl)morpholine (from Peak 1, D33), 4-(6-iodo-2-methylpyrimidin-4-yl)morpholine (D118), CuI, K₃PO₄ in toluene/THF and DMEDA at 80° C. for 2 hour.

LC-MS [mobile phase: from 80% water (0.1% FA) and 20% MeCN (0.1% FA) to 5% water (0.1% FA) and 95% MeCN (0.1% FA) in 2.0 min]: Rt=1.24 min; MS Calcd.: 480.6, MS Found: 481.4 [M+H]⁺.

Chiral Separation:

Method: Column: AD, Column size: 0.46 cm×15 cm. Mobile phase: Supercritical CO₂:EtOH (0.1% NH₃.H₂O)=60:40, Flow rate: 0.5 mL/min, Wave length: UV 254 nm, Temperature: 25° C.

Peak 1 (E107): Single Unknown Isomer 1, Rt=2.541 Min

¹H NMR (400 MHz, CDCl₃): δ 8.88 (s, 1H), 8.06 (s, 1H), 7.52 (s, 1H), 6.95 (s, 1H), 4.93˜4.79 (m, 1H), 4.00˜3.99 (m, 1H), 3.92˜3.90 (m, 1H), 3.80˜3.79 (m, 5H), 3.72˜3.71 (m, 5H), 3.29˜3.26 (m, 1H), 3.20˜3.17 (m, 1H), 3.07˜3.04 (m, 2H), 2.63 (s, 3H), 2.48 (s, 3H), 2.22˜2.20 (m, 1H), 2.11˜2.09 (m, 1H), 1.99˜1.96 (m, 1H), 1.90˜1.86 (m, 2H), 1.58˜1.56 (m, 1H).

¹⁹F NMR (376.5 MHz, CDCl₃): δ 183.28 (s)

LC-MS [mobile phase: 95% water (0.1% FA) and 5% MeCN (0.1% FA) to 5% water (0.1% FA) and 95% MeCN (0.1% FA) in 10.0 min]: Rt=4.88 min; MS Calcd.:480.6, MS Found: 481.3 [M+H]⁺.

Chiral HPLC [method: Column: AD, Column size: 0.46 cm×15 cm. Injection: 2 μl, Mobile phase: HEP:EtOH (0.1% DEA)=60:40, Flow rate: 0.5 mL/min, Wave length: UV 254 nm, Temperature: 25° C.]: Rt=2.541 min, ee: 100%

Peak 2 (E108): Single Unknown Isomer 2, Rt=2.985 Min

¹H NMR (400 MHz, CDCl₃): δ 8.88 (s, 1H), 8.06 (s, 1H), 7.52 (s, 1H), 6.95 (s, 1H), 4.95˜4.82 (m, 1H), 4.00˜3.99 (m, 1H), 3.92˜3.90 (m, 1H), 3.80˜3.79 (m, 5H), 3.72˜3.71 (m, 5H), 3.46˜3.49 (m, 1H), 3.19˜3.09 (m, 2H), 2.87˜2.85 (m, 1H), 2.63 (s, 3H), 2.48 (s, 3H), 2.27˜2.25 (m, 2H), 2.13˜2.10 (m, 1H), 1.96˜1.93 (m, 2H), 1.89˜1.86 (m, 1H).

¹⁹F NMR (376.5 MHz, CDCl₃): δ 183.18 (s)

LC-MS [mobile phase: 95% water (0.1% FA) and 5% MeCN (0.1% FA) to 5% water (0.1% FA) and 95% MeCN (0.1% FA) in 10.0 min]: Rt=4.91 min; MS Calcd.:480.6, MS Found: 481.3 [M+H]⁺.

Chiral HPLC [method: Column: AD, Column size: 0.46 cm×15 cm. Injection: 2 μl, Mobile phase: HEP:EtOH (0.1% DEA)=60:40, Flow rate: 0.5 mL/min, Wave length: UV 254 nm, Temperature: 25° C.]: Rt=2.985 min, ee: 100%

### Examples 109 and 110

### Cis-4-(6-(6-(3-fluoro-1-(tetrahydrofuran-3-yl)-piperidin-4-yl)-5-methyl-1H-indazol-1-yl)-2-methoxypyrimidin-4-yl)morpholine (from Peak 2, D34) (Single Unknown Isomer 1, Rt=2.174 Min, Single Unknown Isomer 2, Rt=3.041 Min)

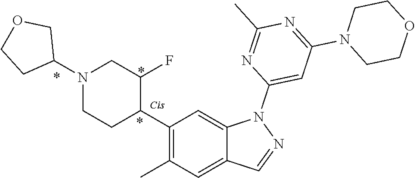 [SMILES: Cc1nc(N2CCOCC2)cc(-n2ncc3cc(C)c(C4CCN(C5CCOC5)CC4F)cc32)n1]

The title compounds were prepared by a procedure similar to that described for E1 and E2 starting from a mixture of 4-(6-iodo-2-methylpyrimidin-4-yl)morpholine, CuI, K₃PO₄ in toluene/THF and DMEDA at 80° C. for 2 hrs.

LC-MS [mobile phase: from 50% water (0.1% NH₄OH) and 50% CH₃CN (0.1% NH₄OH) to 5% water (0.1% NH₄OH) and 95% CH₃CN (0.1% NH4OH) in 2.0 min]: Rt=1.48 min; MS Calcd.: 480.6, MS Found: 481.4 [M+H]⁺.

Chiral prep-HPLC

Method: AD-H, 0.46 cm I.D.×15 cm L, Mobile phase: Supercritical CO₂:EtOH (0.1% NH₃.H₂O)=60:40, Flow rate: 0.5 mL/min, 254 nm, Temperature: 25° C.

Peak 1 (E109): Single Unknown Isomer 1

¹H NMR (400 MHz, CDCl₃): δ 8.88 (s, 1H), 8.06 (s, 1H), 7.53 (s, 1H), 6.95 (s, 1H), 4.94˜4.83 (m, 1H), 4.00˜3.99 (m, 1H), 3.94˜3.92 (m, 1H), 3.90˜3.71 (m, 10H), 3.49˜3.46 (m, 1H), 3.17˜3.12 (m, 2H), 2.86˜2.84 (m, 1H), 2.63 (s, 3H), 2.48 (s, 3H), 2.29˜2.24 (m, 2H), 2.11˜2.10 (m, 1H), 1.99˜1.89 (m, 3H).

¹⁹F NMR (376.5 MHz, CDCl₃): δ 183.17 (s)

LC-MS [mobile phase: 95% water (0.1% FA) and 5% CH₃CN (0.1% FA) to 5% water (0.1% FA) and 95% CH₃CN (0.1% FA) in 9.0 min]: Rt=4.85 min; MS Calcd.:480.6, MS Found: 481.3 [M+H]⁺.

Chiral HPLC [AD Column size: 0.46 cm I.D.×15 cm L. Injection: 2 μl, Mobile phase: HEP:EtOH (0.1% DEA)=60:40, Flow rate: 0.5 mL/min, Wave length: UV 254 nm, Temperature: 25° C.]: Rt=2.174 min, ee: 100%

Peak 2 (E110): Single Unknown Isomer 2

¹H NMR (400 MHz, CDCl₃): δ 8.88 (s, 1H), 8.06 (s, 1H), 7.53 (s, 1H), 6.95 (s, 1H), 4.94˜4.82 (m, 1H), 4.00˜3.99 (m, 1H), 3.94˜3.92 (m, 1H), 3.90˜3.71 (m, 10H), 3.29˜3.05 (m, 4H), 2.63 (s, 3H), 2.48 (s, 3H), 2.34˜2.31 (m, 1H), 2.21˜2.18 (m, 1H), 2.11˜2.09 (m, 1H), 1.99˜1.09 (m, 3H).

¹⁹F NMR (376.5 MHz, CDCl₃): δ 183.28 (s)

LC-MS [mobile phase: 95% water (0.1% FA) and 5% CH₃CN (0.1% FA) to 5% water (0.1% FA) and 95% CH₃CN (0.1% FA) in 9.0 min]: Rt=4.84 min; MS Calcd.:480.6, MS Found: 481.3 [M+H]⁺.

Chiral HPLC [Column: AD Column size: 0.46 cm I.D.×15 cm L. Injection: 2 μl Mobile phase: HEP:EtOH (0.1% DEA)=60:40, Flow rate: 0.5 mL/min, Wave length: UV 254 nm, Temperature: 25° C.]: Rt=3.041 min, ee: 100%

### Examples 111 and 112

### ((3R)-4-(2-methyl-6-(5-methyl-6-(1-(tetrahydrofuran-3-yl)piperidin-4-yl)-1H-indaz-ol-1-yl)pyrimidin-4-yl)morpholin-3-yl)methanol (Single Unknown Isomer 1, Rt=6.040 Min; Single Unknown Isomer 2, Rt=6.445 Min)

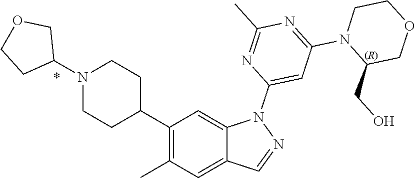

The title compounds were prepared by a procedure similar to that described for E1 and E2 starting from a solution of (R)-(4-(6-iodo-2-methylpyrimidin-4-yl)morpholin-3-yl)me-thanol (D119) and 5-methyl-6-(tetrahydrofuran-3-yl)-1H-indazole (D10) in toluene and N¹,N²-dimethylethane-1,2-diamine, CuI and K₃PO4.3H₂O at 100° C. for 4 hours.

LC-MS [mobile phase: from 60% water (0.1% FA) and 40% MeCN (0.1% FA) to 5% water (0.1% FA) and 95% MeCN (0.1% FA) in 2.0 min]: Rt=0.80 min; MS Calcd.:492.3, MS Found: 493.2 [M+H]⁺.

Chiral Separation:

Method: Column: AD-H; Column size: 0.46 cm×15 cm; Mobile phase: Supercritical CO₂:IPA (0.1% NH₃.H₂O)=60:40; Flow rate: 0.5 ml; Wave length: UV 254 nm; Temperature: 25° C.; Sample solution in EtOH

Peak 1 (E111): Single Unknown Isomer 1, Rt=6.040 Min

¹H NMR (400 MHz, CDCl₃): δ 8.77 (s, 1H), 8.05 (s, 1H), 7.50 (s, 1H), 6.97 (s, 1H), 4.65˜4.63 (m, 1H), 4.11˜3.96 (m, 7H), 3.87˜3.84 (m, 1H), 3.86˜3.61 (m, 3H), 3.43˜3.67 (m, 1H), 3.22˜3.18 (m, 1H), 3.07˜3.00 (m, 2H), 2.84˜2.81 (m, 1H), 2.62 (s, 3H), 2.49 (s, 3H), 2.33˜2.31 (m, 2H), 2.25˜2.17 (m, 1H), 1.97˜1.93 (m, 5H).

LC-MS [mobile phase: from 70% water (0.1% FA) and 30% MeCN (0.1% FA) to 5% water (0.1% FA) and 95% MeCN (0.1% FA) in 2.6 min]: Rt=0.88 min; MS Calcd: 492.3, MS Found: 493.3 [M+H]⁺.

Chiral HPLC [AD-H; Column size: 0.46 cm×15 cm; Injection: 2 μl; Mobile phase: HEP:IPA (0.1% DEA)=60:40; Flow rate: 0.5 ml; Wave length: UV 254 nm; Temperature: 25° C.]: Rt=6.040 min, ee: 100%

Peak 2 (E112): Single Unknown Isomer 2, Rt=6.445 Min

¹H NMR (400 MHz, CDCl₃): δ 8.77 (s, 1H), 8.05 (s, 1H), 7.50 (s, 1H), 6.97 (s, 1H), 4.65˜4.63 (m, 1H), 4.11˜3.96 (m, 7H), 3.87˜3.84 (m, 1H), 3.86˜3.61 (m, 3H), 3.43˜3.67 (m, 1H), 3.22˜3.18 (m, 1H), 3.07˜3.00 (m, 2H), 2.84˜2.81 (m, 1H), 2.62 (s, 3H), 2.49 (s, 3H), 2.33˜2.31 (m, 2H), 2.25˜2.17 (m, 1H), 1.97˜1.93 (m, 5H).

LC-MS [mobile phase: from 70% water (0.1% FA) and 30% MeCN (0.1% FA) to 5% water (0.1% FA) and 95% MeCN (0.1% FA) in 2.6 min]: Rt=0.88 min; MS Calcd: 492.3, MS Found: 493.3 [M+H]⁺.

Chiral HPLC [AD-H; Column size: 0.46 cm×15 cm; Injection: 2 μl; Mobile phase: HEP:PA (0.1% DEA)=60:40; Flow rate: 0.5 ml; Wave length: UV 254 nm; Temperature: 25° C.]: Rt=6.445 min, ee: 99%

### Examples 113 and 114

### ((3S)-4-(2-methyl-6-(5-methyl-6-(1-(tetrahydrofuran-3-yl)piperidin-4-yl)-1H-indaz-ol-1-yl)pyrimidin-4-yl)morpholin-3-yl)methanol (Single Unknown Isomer 1, Rt=5.102 Min; Single Unknown Isomer 2, Rt=5.288 Min))

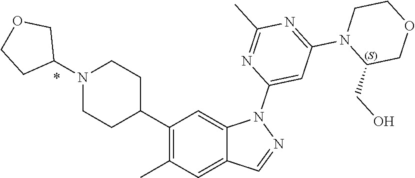

The title compounds were prepared by a procedure similar to that described for E1 and E2 starting from a solution of (S)-(4-(6-iodo-2-methylpyrimidin-4-yl)morpholin-3-yl)methanol (D120) and 5-methyl-6-(1-(tetrahydrofuran-3-yl)piperidin-4-yl)-1H-indazole (D10) in toluene, CuI, K₃PO₄ and N,N′-dimethylethylenediamine at 100° C. for 5 h.

LC-MS [mobile phase: from 80% water (0.1% FA) and 20% MeCN (0.1% FA) to 5% water (0.1% FA) and 95% MeCN (0.1% FA) in 2.6 min]: Rt=0.86 min; MS Calcd: 492.3, MS Found: 493.4 [M+H]⁺.

Chiral Separation:

Method: Column: AD-H; Column size: 0.46 cm×15 cm; Mobile phase: Supercritical CO₂:EtOH (0.1% NH₃.H₂O)=60:40; Flow rate: 0.5 ml; Wave length: UV 254 nm; Temperature: 25° C.; Sample solution in EtOH

Peak 1 (E113): Single Unknown Isomer 1, Rt=5.102 Min

¹H NMR (400 MHz, CDCl₃) δ 8.77 (s, 1H), 8.04 (s, 1H), 7.49 (s, 1H), 6.97 (s, 1H), 4.63 (br, 1H), 4.11˜3.94 (m, 7H), 3.86˜3.83 (m, 1H), 3.74˜3.61 (m, 3H), 3.43˜3.37 (m, 1H), 3.21˜3.19 (d, J=8.0 Hz, 1H), 3.06˜2.96 (m, 2H), 2.86˜2.82 (m, 1H), 2.62 (s, 3H), 2.46 (s, 3H), 2.26˜2.23 (m, 2H), 2.13˜2.10 (m, 1H), 1.94˜1.92 (m, 5H).

LC-MS [mobile phase: from 60% water (0.1% FA) and 40% MeCN (0.1% FA) to 5% water (0.1% FA) and 95% MeCN (0.1% FA) in 2.6 min]: Rt=0.84 min; MS Calcd: 492.3, MS Found: 493.2 [M+H]⁺.

Chiral HPLC [AD-H; Column size: 0.46 cm×15 cm; Injection: 2 μl; Mobile phase: HEP:EtOH (0.05% DEA)=60:40; Flow rate: 0.5 ml; Wave length: UV 254 nm; Temperature: 25° C.]: Rt=5.102 min, ee: 100%.

Peak 2 (E114): Single Unknown Isomer 2, Rt=5.288 Min

¹H NMR (400 MHz, CDCl₃) δ 8.77 (s, 1H), 8.05 (s, 1H), 7.50 (s, 1H), 6.97 (s, 1H), 4.64 (br, 1H), 4.10˜3.94 (m, 7H), 3.86˜3.83 (m, 1H), 3.74˜3.61 (m, 3H), 3.43˜3.37 (m, 1H), 3.21˜3.19 (d, J=8.0 Hz, 1H), 3.06˜2.97 (m, 2H), 2.86˜2.82 (m, 1H), 2.62 (s, 3H), 2.46 (s, 3H), 2.26˜2.24 (m, 2H), 2.13˜2.11 (m, 1H), 1.93 (s, 5H).

LC-MS [mobile phase: from 60% water (0.1% FA) and 40% MeCN (0.1% FA) to 5% water (0.1% FA) and 95% MeCN (0.1% FA) in 2.6 min]: Rt=0.84 min; MS Calcd: 492.3, MS Found: 493.2 [M+H]⁺.

Chiral HPLC [AD-H; Column size: 0.46 cm×15 cm; Injection: 2 μl; Mobile phase: HEP:EtOH (0.05% DEA)=60:40; Flow rate: 0.5 ml; Wave length: UV 254 nm; Temperature: 25° C.]: Rt=5.288 min, ee: 99.3%;

### Examples 115 and 116

### Cis-4-(6-(6-(3-fluoro-1-(tetrahydrofuran-3-yl)-piperidin-4-yl)-5-methyl-1H-indazol-1-yl)-2-methoxypyrimidin-4-yl)morpholine (from Peak 2, D34) (Single Unknown Isomer 1, Rt=1.588 Min; Single Unknown Isomer 2, Rt=2.669 Min)

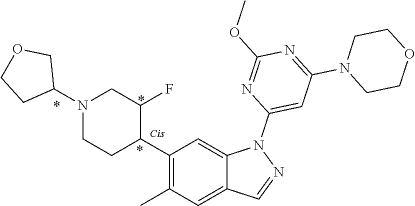 [SMILES: COc1nc(N2CCOCC2)cc(-n2ncc3cc(C)c(C4CCN(C5CCOC5)CC4F)cc32)n1]

The title compounds were prepared by a procedure similar to that described for E1 and E2 starting from a mixture of cis-4-(6-(6-(3-fluoro-1-(tetrahydrofuran-3-yl)piperidin-4-yl)-5-methyl-1H-indazol-1-yl)-2-methoxypyrimidin-4-yl)morpholine (from Peak 2, D34), 4-(6-iodo-2-methoxypyrimidin-4-yl)morpholine (D87), CuI, K₃PO₄ in toluene/THF and DMEDA at 80° C. for 2 hour under N₂.

LC-MS [mobile phase: from 50% water (0.1% NH₄OH) and 50% MeCN (0.1% NH₄OH) to 5% water (0.1% NH₄OH) and 95% MeCN (0.1% NH₄OH) in 2.0 min]: Rt=1.51 min; MS Calcd.: 496, MS Found: 497 [M+H]⁺.

Chiral Separation:

Method: AD-H, 0.46 cm×15 cm, Mobile phase: Supercritical CO₂:EtOH (0.1% NH₃.H₂O)=60:40, Flow rate: 0.5 mL/min, 254 nm, Temperature: 25° C.

Peak 1 (E115): Single Unknown Isomer 1, Rt=1.588 Min

¹H NMR (400 MHz, CDCl₃): δ 8.85 (s, 1H), 8.06 (s, 1H), 7.53 (s, 1H), 6.83 (s, 1H), 4.92˜4.76 (m, 1H), 4.11 (s, 3H), 4.00˜3.96 (m, 1H), 3.92˜3.90 (m, 1H), 3.83˜3.71 (m, 10H), 3.46˜3.44 (m, 1H), 3.17˜3.12 (m, 2H), 2.84˜2.82 (m, 1H), 2.48 (s, 3H), 2.26˜2.22 (m, 2H), 2.10˜2.09 (m, 1H), 1.94˜1.92 (m, 2H), 1.86˜1.81 (m, 1H).

¹⁹F NMR (376.5 MHz, CDCl₃): δ 183.21 (s)

LC-MS [mobile phase: 95% water (0.1% FA) and 5% MeCN (0.1% FA) to 5% water (0.1% FA) and 95% MeCN (0.1% FA) in 9.0 min]: Rt=5.16 min; MS Calcd.:496.6, MS Found: 497.3 [M+H]⁺.

Chiral HPLC [method: Column: AD, Column size: 0.46 cm×15 cm. Injection: 2 μl, Mobile phase: HEP:EtOH (0.1% DEA)=60:40, Flow rate: 0.5 mL/min, Wave length: UV 254 nm, Temperature: 25° C.]: Rt=1.588 min, ee: 100%

Peak 2 (E116): Single Unknown Isomer 2, Rt=2.669 Min

¹H NMR (400 MHz, CDCl₃): δ 8.85 (s, 1H), 8.06 (s, 1H), 7.53 (s, 1H), 6.83 (s, 1H), 4.89˜4.76 (m, 1H), 4.11 (s, 3H), 4.00˜3.97 (m, 1H), 3.92˜3.90 (m, 1H), 3.85˜3.72 (m, 10H), 3.20˜3.04 (m, 4H), 2.48 (s, 3H), 2.32˜2.31 (m, 1H), 2.21˜2.19 (m, 1H), 2.08˜2.07 (m, 1H), 1.96˜1.83 (m, 3H).

¹⁹F NMR (376.5 MHz, CDCl₃): δ 183.32 (s)

LC-MS [mobile phase: 95% water (0.1% FA) and 5% MeCN (0.1% FA) to 5% water (0.1% FA) and 95% MeCN (0.1% FA) in 9.0 min]: Rt=4.98 min; MS Calcd.:496.6, MS Found: 497.3 [M+H]⁺.

Chiral HPLC [method: Column: AD, Column size: 0.46 cm×15 cm. Injection: 2 μl, Mobile phase: HEP:EtOH (0.1% DEA)=60:40, Flow rate: 0.5 mL/min, Wave length: UV 254 nm, Temperature: 25° C.]: Rt=2.669 min, ee: 100%

### Examples 117 and 118

### ((2R)-4-(6-(6-(3-fluoro-1-(tetrahydrofuran-3-yl)piperidin-4-yl)-5-methyl-1H-indazol-1-yl)-2-methoxypyrimidin-4-yl)morpholin-2-yl)methanol (from Peak 2, D34) (Single Unknown Isomer 1, Rt=1.249 Min; Single Unknown Isomer 2, Rt=1.410 Min)

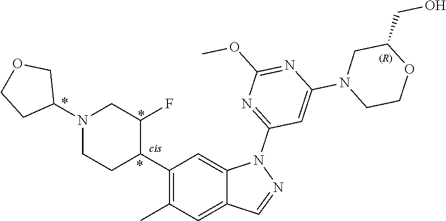

The title compounds were prepared by a procedure similar to that described for E1 and E2 starting from a solution of cis-6-(3-fluoro-1-(tetrahydrofuran-3-yl)piperidin-4-yl)-5-methyl-1H-indazole (from Peak 2, D34) and (R)-(4-(6-iodo-2-methoxy pyrimidin-4-yl)morpholin-2-yl)methanol (D25) in toluene and N¹,N²-dimethylethane-1,2-diamine, CuI and K₃PO₄.3H₂O at 100° C. for 4 h.

LC-MS [mobile phase: from 50% water (0.1% FA) and 50% MeCN (0.1% FA) to 5% water (0.1% FA) and 95% MeCN (0.1% FA) in 2.0 min]: Rt=0.80 min; MS Calcd.:526.3, MS Found: 527.3 [M+H]⁺.

Chiral Separation:

Method: Column: AD-H, Column size: 0.46 cm×15 cm; Mobile phase: Supercritical CO₂:IPA (0.1% NH₃.H₂O)=60:40; Flow rate: 0.5 ml; Wave length: UV 254 nm; Temperature: 25° C.; Sample solution in EtOH

Peak 1 (E117): Single Unknown Isomer 1, Rt=1.249 Min

¹H NMR (400 MHz, CDCl₃): δ 8.85 (s, 1H), 8.07 (s, 1H), 7.53 (s, 1H), 6.91 (s, 1H), 4.90˜4.89 (m, 0.5H), 4.79˜4.77 (m, 0.5H), 4.28˜4.25 (m, 2H), 4.11 (s, 3H), 4.07˜4.05 (m, 1H), 3.99˜3.97 (m, 1H), 3.92˜3.88 (m, 1H), 3.87˜3.74 (m, 2H), 3.71˜3.62 (m, 4H), 3.45˜3.43 (m, 1H), 3.17˜3.07 (m, 3H), 3.00˜2.94 (m, 1H), 2.83˜2.81 (m, 1H), 2.48 (s, 3H), 2.29˜2.20 (m, 2H), 2.12˜2.06 (m, 1H), 2.00˜1.89 (m, 3H), 1.87-1.80 (m, 1H).

¹⁹F NMR (376 MHz, CDCl₃): δ 183.222 (s)

LC-MS [mobile phase: from 70% water (0.1% FA) and 30% MeCN (0.1% FA) to 5% water (0.1% FA) and 95% MeCN (0.1% FA) in 2.6 min]: Rt=0.96 min; MS Calcd: 526.3, MS Found: 527.2 [M+H]⁺.

Chiral HPLC[Column: AD-H, Column size: 0.46 cm×15 cm; Injection: 2 μl; Mobile phase: HEP:IPA (0.1% DEA)=60:40; Flow rate: 0.5 ml; Wave length: UV 254 nm; Temperature: 25° C.]: Rt=1.249 min, ee: 100%

Peak 2 (E118): Single Unknown Isomer 2, Rt=1.410 Min

¹H NMR (400 MHz, CDCl₃): δ 8.85 (s, 1H), 8.07 (s, 1H), 7.53 (s, 1H), 6.91 (s, 1H), 4.90˜4.89 (m, 0.5H), 4.79˜4.77 (m, 0.5H), 4.28˜4.25 (m, 2H), 4.11 (s, 3H), 4.07˜4.05 (m, 1H), 3.99˜3.97 (m, 1H), 3.92˜3.88 (m, 1H), 3.87˜3.74 (m, 2H), 3.71˜3.62 (m, 4H), 3.45˜3.43 (m, 1H), 3.17˜3.07 (m, 3H), 3.00˜2.94 (m, 1H), 2.83˜2.81 (m, 1H), 2.48 (s, 3H), 2.29˜2.20 (m, 2H), 2.12˜2.06 (m, 1H), 2.00˜1.89 (m, 3H), 1.87˜1.80 (m, 1H).

¹⁹F NMR (376 MHz, CDCl₃): δ 183.222 (s)

LC-MS [mobile phase: from 70% water (0.1% FA) and 30% MeCN (0.1% FA) to 5% water (0.1% FA) and 95% MeCN (0.1% FA) in 2.6 min]: Rt=0.96 min; MS Calcd: 526.3, MS Found: 527.2 [M+H]⁺.

Chiral HPL [Column: AD-H, Column size: 0.46 cm×15 cm; Injection: 2 μl; Mobile phase: HEP:IPA (0.1% DEA)=60:40; Flow rate: 0.5 ml; Wave length: UV 254 nm; Temperature: 25° C.]: Rt=1.410 min, ee: 100%

### Examples 119 and 120

### ((2R)-4-(6-(6-(3-fluoro-1-(tetrahydrofuran-3-yl)piperidin-4-yl)-5-methyl-1H-indazol-1-yl)-2-methoxypyrimidin-4-yl)morpholin-2-yl)methanol (from Peak 1, D33) (Single Unknown Isomer 1, Rt=3.879 Min; Single Unknown Isomer 2, Rt=6.171 Min)

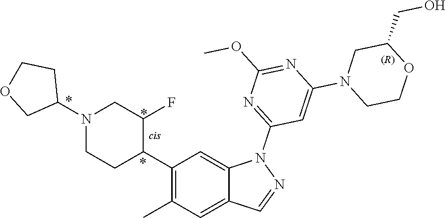

The title compounds were prepared by a procedure similar to that described for E1 and E2 starting from a solution of cis-6-(3-fluoro-1-(tetrahydrofuran-3-yl)piperidin-4-yl)-5-methyl-1H-indazole (from Peak 1, D33) and (R)-(4-(6-iodo-2-methoxy pyrimidin-4-yl)morpho-lin-2-yl)methanol (D25) in toluene and N¹,N²-dimethylethane-1,2-diamine, CuI and K₃PO₄.3H₂O at 100° C. for 4 h.

LC-MS [mobile phase: from 60% water (0.1% FA) and 40% MeCN (0.1% FA) to 5% water (0.1% FA) and 95% MeCN (0.1% FA) in 2.6 min]: Rt=0.88 min; MS Calcd.:526.3, MS Found: 527.3 [M+H]⁺.

Chiral Separation:

Method: Column: AD-H; Column size: 0.46 cm×15 cm; Mobile phase: Supercritical CO₂:IPA (0.1% NH₃H₂O)=60:40; Flow rate: 0.5 ml; Wave length: UV 254 nm; Temperature: 25° C.; Sample solution in EtOH

Peak 1 (E119): Single Unknown Isomer 1, Rt=3.879 Min

¹H NMR (400 MHz, CDCl₃): b 8.85 (s, 1H), 8.08 (s, 1H), 7.54 (s, 1H), 6.85 (s, 1H), 4.89˜4.76 (m, 1H), 4.31˜4.24 (m, 2H), 4.12 (s, 3H), 4.07˜4.04 (m, 1H), 3.99˜3.98 (m, 1H), 3.92˜3.89 (m, 1H), 3.82˜3.68 (m, 6H), 3.25˜2.94 (m, 6H), 2.48 (s, 3H), 2.33˜2.28 (m, 1H), 2.23˜2.17 (m, 1H), 2.09˜2.17 (m, 1H), 1.96˜1.81 (m, 4H).

¹⁹F NMR (376 MHz, CDCl₃): δ 183.331 (s)

LC-MS [mobile phase: from 60% water (0.1% FA) and 40% MeCN (0.1% FA) to 5% water (0.1% FA) and 95% MeCN (0.1% FA) in 2.6 min]: Rt=0.88 min; MS Calcd: 526.3, MS Found: 527.2 [M+H]⁺.

Chiral HPLC [AD-H; Column size: 0.46 cm×15 cm; Injection: 2 μl; Mobile phase: HEP:APA (0.1% DEA)=60:40; Flow rate: 0.5 ml; Wave length: UV 254 nm; Temperature: 25° C.]: Rt=3.879 min, ee: 100%

Peak 2 (E120): Single Unknown Isomer 2, Rt=6.171 Min

¹H NMR (400 MHz, CDCl₃): δ 8.85 (s, 1H), 8.07 (s, 1H), 7.54 (s, 1H), 6.85 (s, 1H), 4.91˜4.78 (m, 1H), 4.31˜4.24 (m, 2H), 4.12 (s, 3H), 4.07˜4.05 (m, 1H), 3.99˜3.98 (m, 1H), 3.92˜3.89 (m, 1H), 3.82˜3.68 (m, 6H), 3.46˜3.43 (m, 1H), 3.17˜3.12 (m, 3H), 3.00˜2.94 (m, 1H), 2.84˜2.82 (m, 1H), 2.48 (s, 3H), 2.27˜2.21 (m, 2H), 2.11˜2.08 (m, 1H), 1.95˜1.80 (m, 4H).

¹⁹F NMR (376 MHz, CDCl₃): δ 183.236 (s).

LC-MS [mobile phase: from 60% water (0.1% FA) and 40% MeCN (0.1% FA) to 5% water (0.1% FA) and 95% MeCN (0.1% FA) in 2.6 min]: Rt=0.87 min; MS Calcd: 526.3, MS Found: 527.2 [M+H]⁺.

Chiral HPLC [AD-H; Column size: 0.46 cm×15 cm; Injection: 2 μl; Mobile phase: HEP:PA (0.1% DEA)=60:40; Flow rate: 0.5 ml; Wave length: UV 254 nm; Temperature: 25° C.]: Rt=6.171 min, ee: 100%.

### Examples 121 and 122

### ((2S)-4-(6-(6-(3-fluoro-1-(tetrahydrofuran-3-yl)piperidin-4-yl)-5-methyl-1H-indazol-1-yl)-2-methoxypyrimidin-4-yl)morpholin-2-yl)methanol (from Peak 1, D33) (Single Unknown Isomer 1, Rt=2.412 Min; Single Unknown Isomer 2, Rt=4.104 Min)

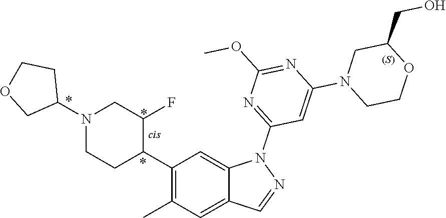

The title compounds were prepared by a procedure similar to that described for E1 and E2 starting from a solution of 6-(3-fluoro-1-(tetrahydrofuran-3-yl)piperidin-4-yl)-5-methyl-1H-indazole (from Peak 1, D33) and (S)-(4-(6-iodo-2-methoxypyrimidin-4-yl)morpholin-2-yl)methanol in toluene (D96), CuI, K₃PO₄ and N,N′-dimethylethylenediamine at 100° C. for 4 h.

LC-MS [mobile phase: from 60% water (0.1% FA) and 40% MeCN (0.1% FA) to 5% water (0.1% FA) and 95% MeCN (0.1% FA) in 2.6 min]: Rt=0.849 min; MS Calcd: 526.60, MS Found: 527.2 [M+H]⁺.

Chiral Separation:

Method: Column: AD-H; Column size: 0.46×15 cm; Mobile phase: Supercritical CO₂:EtOH (0.1% NH₃.H₂O)=60:40; Flow rate: 0.5 ml; Wave length: UV 254 nm; Temperature: 25° C.; Sample solution in EtOH

Peak 1 (E121): Single Unknown Isomer 1, Rt=2.412 Min

¹H NMR (400 MHz, CDCl₃) 8.72 (s, 1H), 8.05 (s, 1H), 7.50 (s, 1H), 6.86 (s, 1H), 4.58 4.54 (m, 1H), 4.31˜4.21 (m, 2H), 4.11 (s, 3H), 4.11˜3.66 (m, 9H), 3.25˜2.94 (m, 6H), 2.48 (m, 3H), 2.33˜1.86 (m, 7H).

¹⁹F NMR (376 MHz, CDCl₃) δ-183.33 (s)

LC-MS [mobile phase: from 70% water (0.1% FA) and 30% MeCN (0.1% FA) to 5% water (0.1% FA) and 95% MeCN (0.1% FA) in 2.6 min]: Rt=0.975 min; MS Calcd: 526, MS Found: 527.3 [M+H]⁺.

Chiral HPLC [Column: AD-H; Column size: 0.46×15 cm; Injection: 2 μl; Mobile phase: HEP:EtOH (0.1% DEA)=60:40; Flow rate: 0.5 ml; Wave length: UV 254 nm; Temperature: 25° C.]: Rt=2.367 min, ee 100%;

Peak 2 (E122): Single Unknown Isomer 2, Rt=4.104 Min

¹H NMR (400 MHz, CDCl₃) δ 8.85 (s, 1H), 8.07 (s, 1H), 7.53 (s, 1H), 6.84 (s, 1H), 4.90-4.89 (m, 1H), 4.77-4.76 (m, 2H), 4.28 (s, 3H), 4.24-4.11 (m, 1H), 4.07-4.05 (m, 1H), 3.99-3.98 (m, 1H), 3.88-3.80 (m, 2H), 3.78-2.68 (m, 4H), 3.45-3.42 (m, 1H), 3.17-3.09 (m, 3H), 3.00-2.94 (m, 1H), 2.83-2.81 (m, 1H), 2.48 (s, 3H), 2.28-2.20 (m, 2H), 2.11-2.05 (m, 1H), 1.96-1.83 (m, 4H).

¹⁹F NMR (376 MHz, CDCl₃) δ-183.33 (s)

LC-MS [mobile phase: from 50% water (0.1% NH₄OH) and 50% MeCN (0.1% NH₄OH) to 5% water (0.1% NH₄OH) and 95% MeCN (0.1% NH₄OH) in 2.6 min]: Rt=0.92 min; MS Calcd: 508.61, MS Found: 509.3 [M+H]⁺.

Chiral HPLC [Column: AD-H; Column size: 0.46×15 cm; Injection: 2 μl; Mobile phase: HEP:EtOH (0.1% DEA)=60:40; Flow rate: 0.5 ml; Wave length: UV 254 nm; Temperature: 25° C.]: Rt=3.806 min, ee 99%

### Examples 123 and 124

### ((2S)-4-(6-(6-(3-fluoro-1-(tetrahydrofuran-3-yl)piperidin-4-yl)-5-methyl-1H-indazol-1-yl)-2-methoxypyrimidin-4-yl)morpholin-2-yl)methanol (from Peak 2, D34) (Single Unknown Isomer 1, Rt=2.740 Min; Single Unknown Isomer 2, Rt=10.595 Min)

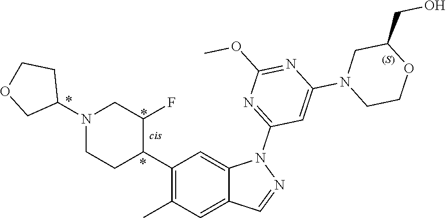

The title compounds were prepared by a procedure similar to that described for E1 and E2 starting from a solution of 6-(3-fluoro-1-(tetrahydrofuran-3-yl)piperidin-4-yl)-5-methyl-1H-indazole (Peak 2, D34) and (S)-(4-(6-iodo-2-methoxypyrimidin-4-yl)morpholin-2-yl)methanol in toluene (D96), CuI, K₃PO₄ and N,N′-dimethylethylenediamine at 100° C. for 4 h.

LC-MS [mobile phase: from 60% water (0.1% FA) and 40% MeCN (0.1% FA) to 5% water (0.1% FA) and 95% MeCN (0.1% FA) in 2.6 min]: Rt=0.88 min; MS Calcd: 526.3, MS Found: 527.2 [M+H]⁺.

Chiral Separation:

Method: Column: AD-H; Column size: 0.46 cm×15 cm; Mobile phase: Supercritical CO₂:IPA (0.1% NH₃.H₂O)=60:40; Flow rate: 0.5 ml; Wave length: UV 254 nm; Temperature: 25° C.; Sample solution in EtOH

Peak 1 (E123): Single Unknown Isomer 1, Rt=2.740 Min

¹H NMR (400 MHz, CDCl₃) 8.85 (s, 1H), 8.08 (s, 1H), 7.54 (s, 1H), 6.85 (s, 1H), 4.90˜4.77 (m, 1H), 4.31˜4.24 (m, 2H), 4.12 (s, 3H), 4.08˜4.05 (m, 1H), 4.00˜3.98 (m, 1H), 3.93˜3.89 (m, 1H), 3.83˜3.68 (m, 6H), 3.45˜3.43 (m, 1H), 3.18˜3.10 (m, 3H), 3.01˜2.94 (m, 1H), 2.84˜2.81 (m, 1H), 2.48 (s, 3H), 2.26˜2.20 (m, 2H), 2.11˜2.09 (m, 1H), 1.96˜1.80 (m, 4H).

¹⁹F NMR (376 MHz, CDCl₃): δ 183.222 (s)

LC-MS [mobile phase: from 60% water (0.1% NH₄OH) and 40% MeCN (0.1% NH₄OH) to 5% water (0.1% NH₄OH) and 95% MeCN (0.1% NH₄OH) in 2.6 min]: Rt=0.88 min; MS Calcd: 526.3, MS Found: 527.2 [M+H]⁺.

Chiral HPLC [Column: AD-H; Column size: 0.46 cm×15 cm; Injection: 2 μl; Mobile phase: HEP:IPA (0.1% DEA)=60:40; Flow rate: 0.5 ml; Wave length: UV 254 nm; Temperature: 25° C.]: Rt=2.740 min, ee 100%

Peak 2 (E124): Single Unknown Isomer 2, Rt=10.595 Min

5 ¹H NMR (400 MHz, CDCl₃) 8.85 (s, 1H), 8.07 (s, 1H), 7.54 (s, 1H), 6.85 (s, 1H), 4.89 4.77 (m, 1H), 4.31˜4.24 (m, 2H), 4.12 (s, 3H), 4.08˜4.05 (m, 1H), 3.99˜3.98 (m, 1H), 3.91˜3.88 (m, 1H), 3.82˜3.68 (m, 6H), 3.25˜2.94 (m, 6H), 2.48 (s, 3H), 2.33˜2.29 (m, 1H), 2.23˜2.18 (m, 1H), 2.11˜2.08 (m, 1H), 1.96˜1.82 (m, 4H).

¹⁹F NMR (376 MHz, CDCl₃): δ 183.338 (s)

LC-MS [mobile phase: from 60% water (0.1% NH₄OH) and 40% MeCN (0.1% NH₄OH) to 5% water (0.1% NH₄OH) and 95% MeCN (0.1% NH₄OH) in 2.6 min]: Rt=0.87 min; MS Calcd: 526.3, MS Found: 527.2 [M+H]⁺.

Chiral HPLC [Column: AD-H; Column size: 0.46 cm×15 cm; Injection: 2 μl; Mobile phase: HEP:IPA (0.1% DEA)=60:40; Flow rate: 0.5 ml; Wave length: UV 254 nm; Temperature: 25° C.]: Rt=10.595 min, ee 100%;

### Examples 125 and 126

### 4-(6-(6-(3-fluoro-1-(tetrahydrofuran-3-yl)-piperidin-4-yl)-5-methyl-1H-indazol-1-yl)-2-methoxypyrimidin-4-yl)morpholine (From peak 1, D33) (Single Unknown Isomer 1, Rt=2.237 Min; Single Unknown Isomer 2, Rt=3.319 Min)

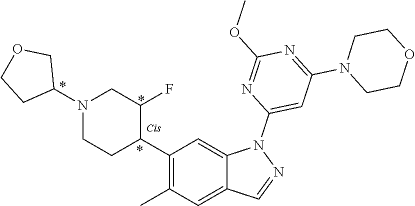 [SMILES: COc1nc(N2CCOCC2)cc(-n2ncc3cc(C)c(C4CCN(C5CCOC5)CC4F)cc32)n1]

The title compounds were prepared by a procedure similar to that described for E1 and E2 starting from a mixture of cis-4-(6-(6-(3-fluoro-1-(tetrahydrofuran-3-yl)piperidin-4-yl)-5-methyl-1H-indazol-1-yl)-2-methoxypyrimidin-4-yl)morpholine (from Peak 1, D33), 4-(6-iodo-2-methoxypyrimidin-4-yl)morpholine (087), CuI, K₃PO₄ in toluene/THF and DMEDA at 80° C. for 2 hrs under N₂.

LC-MS [mobile phase: from 80% water (0.1% FA) and 20% MeCN (0.1% FA) to 5% water (0.1% FA) and 95% MeCN (0.1% FA) in 2.0 min]: Rt=1.23 min; MS Calcd.: 480.6, MS Found: 481.4 [M+H]⁺.

Chiral Separation:

Method: AD-H, 0.46 cm×15 cm, Mobile phase: Supercritical CO₂:EtOH (0.1% NH₃.H₂O)=60:40, Flow rate: 0.5 mL/min, 254 nm, Temperature: 25° C.

Peak 1 (E125): Single Unknown Isomer 1, Rt=2.237 Min

¹H NMR (400 MHz, CDCl₃): δ 8.85 (s, 1H), 8.06 (s, 1H), 7.53 (s, 1H), 6.83 (s, 1H), 4.88˜4.75 (m, 1H), 4.11 (s, 3H), 3.99˜3.96 (m, 1H), 3.92˜3.88 (m, 1H), 3.79˜3.76 (m, 5H), 3.72˜3.71 (m, 5H), 3.25˜3.22 (m, 1H), 3.19˜3.16 (m, 1H), 3.09˜3.11 (m, 1H), 3.04˜3.01 (m, 1H), 2.48 (s, 3H), 2.32˜2.28 (m, 1H), 2.23˜2.17 (m, 1H), 2.09˜2.08 (m, 1H), 1.94˜1.92 (m, 2H), 1.83˜1.81 (m, 1H).

¹⁹F NMR (376.5 MHz, CDCl₃): δ 183.33 (s)

LC-MS [mobile phase: 95% water (0.1% FA) and 5% MeCN (0.1% FA) to 5% water (0.1% FA) and 95% MeCN (0.1% FA) in 10.0 min]: Rt=5.14 min; MS Calcd.:496.6, MS Found: 497.3 [M+H]⁺.

Chiral HPLC [method: Column: AD Column size: 0.46 cm×15 cm. Injection: 2 μl, Mobile phase: HEP:EtOH (0.1% DEA)=60:40, Flow rate: 0.5 mL/min, Wave length: UV 254 nm, Temperature: 25° C.]: Rt=2.237 min, ee: 100%

Peak 2 (E126): Single Unknown Isomer 2, Rt=3.319 Min

¹H NMR (400 MHz, CDCl₃): δ 8.85 (s, 1H), 8.06 (s, 1H), 7.53 (s, 1H), 6.83 (s, 1H), 4.92˜4.76 (m, 1H), 4.11 (s, 3H), 4.00˜3.96 (m, 1H), 3.92˜3.90 (m, 1H), 3.79˜3.76 (m, 5H), 3.72˜3.71 (m, 5H), 3.44˜3.43 (m, 1H), 3.17˜3.12 (m, 2H), 2.84˜2.82 (m, 1H), 2.48 (s, 3H), 2.26˜2.22 (m, 2H), 2.10˜2.09 (m, 1H), 1.94˜1.92 (m, 2H), 1.86˜1.81 (m, 1H).

¹⁹F NMR (376.5 MHz, CDCl₃): δ 183.22 (s)

LC-MS [mobile phase: 95% water (0.1% FA) and 5% MeCN (0.1% FA) to 5% water (0.1% FA) and 95% MeCN (0.1% FA) in 10.0 min]: Rt=5.09 min; MS Calcd.:496.6, MS Found: 497.3 [M+H]⁺.

Chiral HPLC [method: Column: AD, Column size: 0.46 cm×15 cm. Injection: 2 μl, Mobile phase: HEP:EtOH (0.1% DEA)=60:40, Flow rate: 0.5 mL/min, Wave length: UV 254 nm, Temperature: 25° C.]: Rt=3.319 min, ee: 100%

### Examples 127 and 128

### 1-(6-(5-chloro-6-(1-(tetrahydrofuran-3-yl)piperidin-4-yl)-1H-indazol-1-yl)-2-methyl-pyrimidin-4-yl)-3-methylazetidin-3-ol (Single Unknown Isomer 1 and Single Unknown Isomer 2)

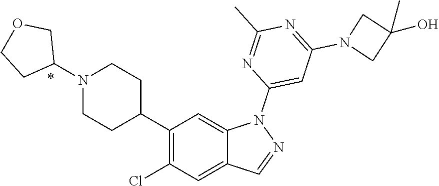 [SMILES: Cc1nc(N2CC(C)(O)C2)cc(-n2ncc3cc(Cl)c(C4CCN(C5CCOC5)CC4)cc32)n1]

The title compounds were prepared by a procedure similar to that described for E1 and E2 starting from a mixture of 5-chloro-6-(1-(tetrahydrofuran-3-yl)piperidin-4-yl)-1H-indazole (D32), 1-(6-iodo-2-methylpyrimidin-4-yl)-3-methylazetidin-3-ol (D54), N,N′-dimethylcy-clo-hexane-1,2-diamine, CuI and K₃PO₄ in toluene at 100° C. for 3 hours.

LCMS [column: C₁₈; column size: 2.1 mm×50 mm; Waters ACQUITY UPLC BEH; mobile phase: B (MeCN); A (0.02% NH₄Ac+5% MeCN in water); flow rate: 0.5 ml/min; gradient (B %) in 2.5 mins. 2.5 min-5-95-POS]: Rt=1.620 min; MS Calcd.:483, MS Found: 484 [M+H]*.

Chiral Separation:

Method: column: CHIRALPAK IA; 5.0 cm×25 cm; Mobile phase: EtOH/MeCN (0.1% NH₃.H₂O)=90/10; Flow rate: 60 ml/min, Wave length: 254 nm.

Peak 1 (E127): Single Unknown Isomer 1, Rt=5.529 Min

¹H NMR (400 MHz, MeOD): δ 8.92 (s, 1H), 8.20 (s, 1H), 7.85 (s, 1H), 6.65 (s, 1H), 4.06-3.91 (m, 6H), 3.78 (q, J=8.4 Hz, 1H), 3.70 (q, J=6.8 Hz, 1H), 3.30-3.20 (m, 2H), 3.09-2.99 (m, 2H), 2.58 (s, 3H), 2.35-2.30 (m, 2H), 2.17-2.15 (m, 1H), 2.00-1.85 (m, 5H), 1.55 (s, 3H).

Chiral-HPLC [column: CHIRALPAK IA 0.46 cm×15 cm; mobile phase: EtOH/DEA=100/0.1; flow rate: 1 mL/min; Wave length: 254 nm; Temperature: 35° C.]: Rt=5.529 min.

LC-MS [column: C₁₈; column size: 4.6×50 mm; mobile phase: B (MeCN), A (0.02% NH₄AC in water); gradient (B %)]: Rt=3.830 min, MS Calcd.: 482, MS Found: 483 [M+H]⁺.

Peak 2 (E128): Single Unknown Isomer 2, Rt=6.048 Min

¹H NMR (400 MHz, MeOD): δ 8.92 (s, 1H), 8.20 (s, 1H), 7.85 (s, 1H), 6.65 (s, 1H), 4.06˜3.91 (m, 6H), 3.78 (q, J=8.4 Hz, 1H), 3.70 (q, J=6.8 Hz, 1H), 3.30˜3.20 (m, 2H), 3.09˜2.99 (m, 2H), 2.58 (s, 3H), 2.35˜2.30 (m, 2H), 2.17˜2.15 (m, 1H), 2.00˜1.85 (m, 5H), 1.55 (s, 3H).

Chiral-HPLC [column: CHIRALPAK IA 0.46 cm×15 cm; mobile phase: EtOH/DEA=100/0.1; flow rate: 1 mL/min; Wave length: 254 nm; Temperature: 35° C.]: Rt=6.048 min.

LC-MS [column: C₁₈; column size: 4.6×50 mm; mobile phase: B (MeCN), A (0.02% NH₄AC in water); gradient (B %)]: Rt=3.818 min, MS Calcd.: 482, MS Found: 483 [M+H]⁺.

### Examples 129 and 130

### ((2R)-4-(6-(5-chloro-6-(1-(tetrahydrofuran-3-yl)piperidin-4-yl)-1H-indazol-1-yl)-2-methylpyrimidin-4-yl)morpholin-2-yl)methanol (Single Unknown Isomer 1 and Single Unknown Isomer 2)

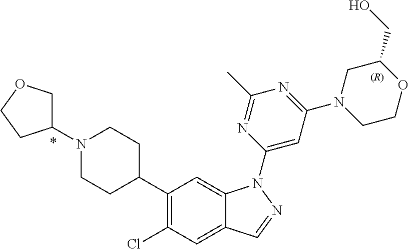

The title compounds were prepared by a procedure similar to that described for E1 and E2 starting from a mixture of 5-chloro-6-(1-(tetrahydrofuran-3-yl)piperidin-4-yl)-1H-indazole (D32), (R)-(4-(6-iodo-2-methylpyrimidin-4-yl)morpholin-2-yl)methanol (D12), N,N′-dimeth-ylcyclohexane-1,2-diamine, CuI and K₃PO₄ in toluene at 100° C. for 4.5 hours.

¹H NMR (400 MHz, CDCl₃) δ 8.90 (s, 1H), 8.07 (s, 1H), 7.74 (s, 1H), 6.95 (s, 1H), 4.32-4.29 (m, 2H), 4.09-3.95 (m, 3H), 3.85-3.65 (m, 6H), 3.22-2.93 (m, 5H), 2.63 (s, 3H), 2.33-1.71 (m, 9H).

Chiral Separation:

Method: column: CHIRALPAK AD-H; 0.46 cm×15 cm; mobile phase: EtOH/MeCN (0.1% NH₃.H₂O)=80/20; flow rate: 1 mL/min; Wave length: 254 nm; Temperature: 35° C. Peak 1 (E129): Single Unknown Isomer 1, Rt=6.253 Min

¹H NMR (400 MHz, CDCl₃): δ 8.90 (s, 1H), 8.07 (s, 1H), 7.75 (s, 1H), 6.95 (s, 1H), 4.32-4.29 (m, 2H), 4.09-3.95 (m, 3H), 3.85-3.65 (m, 6H), 3.22-2.92 (m, 6H), 2.63 (s, 3H), 2.33-1.71 (m, 9H).

Chiral-HPLC [column: CHIRALPAK AD-H; 0.46 cm×15 cm; mobile phase: EtOH/ACN/DEA=80/20/0.1; flow rate: 1 mL/min; Wave length: 254 nm; Temperature: 35° C.]: Rt=6.253 min.

LC-MS [column: C₁₈; column size: 4.6×50 mm; mobile phase: B (MeCN), A (0.1% FA in water); gradient (B %)]: Rt=3.191 min, MS Calcd.: 512, MS Found: 513 [M+H]⁺.

Peak 2 (E130): Single Unknown Isomer 2, Rt=8.943 Min

¹H NMR (400 MHz, CDCl₃): δ 8.90 (s, 1H), 8.07 (s, 1H), 7.75 (s, 1H), 6.95 (s, 1H), 4.32-4.29 (m, 2H), 4.09-3.95 (m, 3H), 3.85-3.65 (m, 6H), 3.22-2.92 (m, 6H), 2.63 (s, 3H), 2.33-1.71 (m, 9H).

Chiral-HPLC [column: CHIRALPAK AD-H; 0.46 cm×15 cm; mobile phase: EtOH/MeCN/DEA=80/20/0.1; flow rate: 1 mL/min; Wave Length: 254 nm; Temperature: 35° C.]: Rt=8.943 min.

LC-MS [column: C₁₈; column size: 4.6×50 mm; mobile phase: B (MeCN), A (0.1% FA in water); gradient (B %)]: Rt=3.186 min, MS Calcd.: 512, MS Found: 513 [M+H]⁺.

### Examples 131 and 132

### trans-3-((2-methyl-6-(5-methyl-6-(1-(tetrahydrofuran-3-yl)piperidin-4-yl)-1H-indaz-ol-1-yl)pyrimidin-4-yl)amino)cyclobutanol (Single Unknown Isomer 1, Rt=12.140 Min and Single Unknown Isomer 2, Rt=15.228 Min)

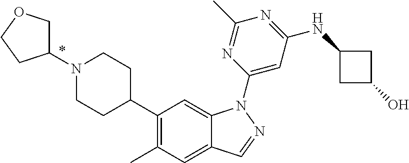 [SMILES: Cc1nc(N[C@H]2C[C@H](O)C2)cc(-n2ncc3cc(C)c(C4CCN(C5CCOC5)CC4)cc32)n1]

To a mixture of trans-3-((2-methyl-6-(5-methyl-6-(piperidin-4-yl)-1H-indazol-1-yl)pyrimidin-4-yl)amino)cyclobutanol (D123, 120 mg, 0.306 mmol), dihydrofuran-3(2H)-one (132 mg, 1.53 mmol) and catalyst HOAc in 4 mL of DCE was added NaBH₃CN (39.0 mg, 0.612 mmol). The reaction mixture was stirred at room temperature for 6 h and then concentrated in vacuo. The residue was purified by column chromatography on silica gel (DCM/MeOH=40/1 to 20/1) to give the title compound (120 mg, 85.0%) as a colorless oil.

¹HNMR (400 MHz, DMSO-d₆): δ 8.70 (s, 1H), 8.32 (s, 1H), 7.77 (s, 1H), 7.65 (s, 1H), 5.15 (br s, 1H), 4.31-4.29 (m, 1H), 4.17-4.16 (m, 1H), 4.03-3.98 (m, 3H), 3.80-3.77 (m, 1H), 3.66-3.64 (m, 1H), 3.56-3.52 (m, 2H), 3.29-3.23 (m, 4H), 2.52 (s, 3H), 2.43 (s, 3H), 2.33-2.27 (m, 2H), 2.25-2.19 (m, 4H), 2.10-2.05 (m, 2H), 1.93-1.87 (m, 2H).

Chiral Separation:

Method: column: 250 mm×4.6 mm 5 μm; mobile phase: Supercritical CO₂:EtOH (0.1% NH₃.H₂O)=80:20; flow rate: 1 mL/min; Wave Length: 254 nm; Temperature: 30° C.

Peak 1 (E131): Single Unknown Isomer 1, Rt=12.140 Min

¹H NMR (400 MHz, CDCl₃): δ 8.78 (s, 1H), 8.07 (s, 1H), 7.50 (s, 1H), 6.64 (s, 1H), 5.19 (br s, 1H), 4.62-4.59 (m, 1H), 4.30-4.28 (m, 1H), 4.01-3.94 (m, 2H), 3.86-3.80 (m, 1H), 3.74-3.71 (m, 1H), 3.22-3.18 (m, 1H), 3.04-2.97 (m, 2H), 2.86-2.82 (m, 1H), 2.61 (s, 3H), 2.51-2.44 (m, 5H), 2.35-2.21 (m, 4H), 2.13-2.11 (m, 1H), 1.94-1.87 (m, 5H).

Chiral-HPLC [column: 250 mm×4.6 mm 5 μm; mobile phase: Hex:EtOH:DEA=80:20:0.2; flow rate: 1 mL/min; Wave Length: 254 nm; Temperature: 30° C.]: Rt=12.140 min.

LC-MS [column: C₁₈; column size: 4.6×50 mm; mobile phase: B (MeCN), A (0.1% TFA in water); gradient (B %)]: Rt=2.450 min, MS Calcd.: 462, MS Found: 463 [M+H]⁺.

Peak 2 (E132): Single unknown isomer 2, Rt=15.228 min

¹H NMR (400 MHz, CDCl₃): δ 8.78 (s, 1H), 8.07 (s, 1H), 7.50 (s, 1H), 6.64 (s, 1H), 5.19 (br s, 1H), 4.62-4.59 (m, 1H), 4.30-4.28 (m, 1H), 4.01-3.94 (m, 2H), 3.86-3.80 (m, 1H), 3.75-3.71 (m, 1H), 3.22-3.18 (m, 1H), 3.06-2.98 (m, 2H), 2.86-2.82 (m, 1H), 2.61 (s, 3H), 2.51-2.44 (m, 5H), 2.35-2.21 (m, 4H), 2.15-2.10 (m, 1H), 1.94-1.87 (m, 5H).

Chiral-HPLC [column: 250×4.6 mm, 5 μm; mobile phase: Hex:EtOH:DEA=80:20:0.2; flow rate: 1 mL/min; Wave Length: 254 nm; Temperature: 30° C.]: Rt=15.228 min.

LC-MS [column: C₁₈; column size: 4.6×50 mm; mobile phase: B (MeCN), A (0.1% FA in water); gradient (B %)]: Rt=2.439 min, MS Calcd.: 462, MS Found: 463 [M+H]⁺.

### Examples 133 and 134

### Cis-3-((2-methyl-6-(5-methyl-6-(1-(tetrahydrofuran-3-yl)piperidin-4-yl)-1H-indazol-1-yl)pyrimidin-4-yl)amino)cyclobutanol (Single Unknown Isomer 1, Rt=10.500 Min and Single Unknown Isomer 2, Rt=14.311 Min)

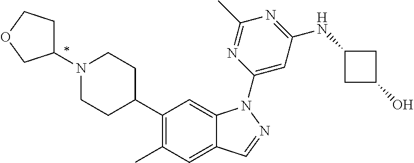 [SMILES: Cc1nc(N[C@H]2C[C@@H](O)C2)cc(-n2ncc3cc(C)c(C4CCN(C5CCOC5)CC4)cc32)n1]

The title compounds were prepared by a procedure similar to that described for E131 and E132 starting from a solution of cis-3-((2-methyl-6-(5-methyl-6-(piperidin-4-yl)-1H-indazol-1-yl)pyrimidin-4-yl)amino)cyclobutanol (D126), dihydrofuran-3(2H)-one and catalyst AcOH in DCM and NaBH₃CN at room temperature overnight.

LCMS [column: C₁₈; column size: 4.6×30 mm 5 μm, Dikwa Diamonsil plus; mobile phase: B (MeCN), A (0.02% NH₄Ac+5% MeCN in water); gradient (B %) in 4 mins. 10-95-POS; flow rate: 1.5 ml/min]: Rt=1.979 min; MS Calcd.:462, MS Found: 463 [M+H]⁺.

Chiral Separation:

Method: column: 250×4.6 mm 5 μm; mobile phase: Supercritical CO₂:EtOH (0.1% NH₃.H₂O)=70:30; flow rate: 1 mL/min, Wave length: 254 nm; Temperature: 30° C.

Peak 1 (E133): Single Unknown Isomer 1, Rt=10.500 Min

¹H NMR (400 MHz, CDCl₃): δ 8.78 (s, 1H), 8.04 (s, 1H), 7.49 (s, 1H), 6.66 (s, 1H), 5.30 (br s, 1H), 4.17-4.12 (m, 1H), 4.02-3.94 (m, 2H), 3.86-3.80 (m, 3H), 3.71 (d, J=11.2 Hz, 1H), 3.05-2.80 (m, 5H), 2.60 (s, 3H), 2.45 (s, 3H), 2.30-2.08 (m, 3H), 1.96-1.86 (m, 7H).

Chiral-HPLC [column: 250×4.6 mm 5 μm; mobile phase: Hex:EtOH:DEA=70:30:0.2; flow rate: 1 mL/min, Wave length: 254 nm; Temperature: 30° C.]: Rt=10.500 min.

LC-MS [column: C₁₈; column size: 4.6×50 mm; mobile phase: B (MeCN), A (0.02% NH₄Ac in water); gradient (B %)]: Rt=3.391 min, MS Calcd.: 462, MS Found: 463 [M+H]⁺.

Peak 2 (E134): Single Unknown Isomer 2, Rt=14.311 Min

¹H NMR (400 MHz, CDCl₃): δ 8.78 (s, 1H), 8.05 (s, 1H), 7.49 (s, 1H), 6.66 (s, 1H), 5.21 (br, 1H), 4.18-4.13 (m, 1H), 4.02-3.70 (m, 5H), 3.20 (d, J=10.8 Hz, 1H), 3.06-2.82 (m, 5H), 2.60 (s, 3H), 2.45 (s, 3H), 2.30-2.09 (m, 3H), 1.99-1.86 (m, 7H).

Chiral-HPLC [column: 250×4.6 mm 5 μm; mobile phase: Hex:EtOH:DEA=70:30:0.2; flow rate: 1 mL/min; Wave length: 254 nm; Temperature: 30° C.]: Rt=14.311 min.

LC-MS [column: C₁₈; column size: 4.6×50 mm; mobile phase: B (MeCN), A (0.02% NH₄Ac in water); gradient (B %)]: Rt=3.284 min, MS Calcd.: 462, MS Found: 463 [M+H]⁺.

### Examples 135, 136, 137, 138, 139, 140, 141 and 142

### 1-(4-(2-methyl-6-(5-methyl-6-(1-(tetrahydrofuran-3-yl)piperidin-4-yl)-1H-indazol-1-yl)pyrimidin-4-yl)morpholin-2-yl)ethanol (Single Unknown Isomer 1; Single Unknown Isomer 2; Single Unknown Isomer 3; Single Unknown Isomer 4; Single Unknown Isomer 5; Single Unknown Isomer 6; Single Unknown Isomer 7; Single Unknown Isomer 8)

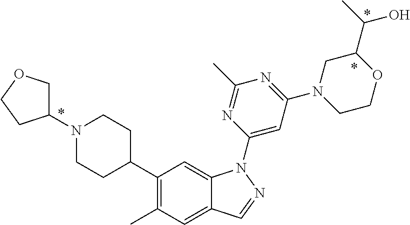 [SMILES: Cc1nc(N2CCOC(C(C)O)C2)cc(-n2ncc3cc(C)c(C4CCN(C5CCOC5)CC4)cc32)n1]

To a solution of 1-(4-(2-methyl-6-(5-methyl-6-(1-(tetrahydrofuran-3-yl)piperidin-4-yl)-1H-indazol-1-yl)pyrimidin-4-yl)morpholin-2-yl)ethanone (D128, 570 mg, 1.13 mmol) in MeOH (50 mL) was added NaBH₄ (107 mg, 2.83 mmol). When LC-MS showed the reaction was completed, the reaction mixture was quenched with water (20 mL) and extracted with CH₂Cl₂ (50 mL×2). The combined organic layers were washed with water (10 mL) and brine (10 mL), dried over Na₂SO₄ and filtered. The filtrate was concentrated and purified by column chromatography (PE:EtOAc=1:3) to give the desired product as a pale yellow solid (560 mg, yield: 97.0%).

LC-MS [mobile phase: from 70% water (0.1% FA) and 30% MeCN (0.1% FA) to 5% water (0.1% FA) and 95% MeCN (0.1% FA) in 2.6 min]: Rt=0.94 min, MS Calcd: 506.3, MS Found: 507.3 [M+H]⁺.

Chiral Separation:

Method: Column: AD-H; Column size: 0.46 cm×15 cm; Mobile phase: Supercritical CO₂:EtOH (0.1% NH₃.H₂O)=60:40; Flow rate: 0.5 ml/min; Wave length: UV 254 nm; Temperature: 25° C.; Sample solution in EtOH

Peak 1 (E135): Single Unknown Isomer 1, Rt=4.984 Min

¹H NMR (400 MHz, CDCl₃) δ 8.78 (s, 1H), 8.05 (s, 1H), 7.50 (s, 1H), 6.96 (s, 1H), 4.36˜4.27 (m, 2H), 4.05˜3.93 (m, 4H), 3.86˜3.81 (m, 1H), 3.75˜3.67 (m, 2H), 3.47˜3.42 (m, 1H), 3.22˜2.96 (m, 5H), 2.86˜2.82 (m, 1H), 2.63 (s, 3H), 2.46 (s, 3H), 2.27˜2.08 (m, 4H), 1.97˜1.92 (m, 5H), 1.30 (d, J=6.4 Hz, 3H).

LC-MS [mobile phase: from 80% water (0.1% FA) and 20% MeCN (0.1% FA) to 5% water (0.1% FA) and 95% MeCN (0.1% FA) in 2.6 min]: Rt=1.12 min; MS Calcd: 506.3, MS Found: 507.3 [M+H]⁺.

Chiral HPLC [Column: AD-H; Column size: 0.46 cm×15 cm; Mobile phase: Hex:EtOH (0.1% DEA)=60:40; Flow rate: 0.5 ml; Wave length: UV 254 nm; Temperature: 25° C.]: Rt=4.984 min, ee: 100%.

Peak 2 (E136): Single Unknown Isomer 2, Rt=5.123 Min

¹H NMR (400 MHz, CDCl₃) δ 8.78 (s, 1H), 8.05 (s, 1H), 7.50 (s, 1H), 6.96 (s, 1H), 4.36˜4.26 (m, 2H), 4.04˜3.93 (m, 4H), 3.86˜3.80 (m, 1H), 3.75˜3.66 (m, 2H), 3.47˜3.42 (m, 1H), 3.21˜2.97 (m, 5H), 2.86˜2.82 (m, 1H), 2.63 (s, 3H), 2.46 (s, 3H), 2.26˜2.10 (m, 4H), 1.97˜1.93 (m, 5H), 1.30 (d, J=6.4 Hz, 3H).

LC-MS [mobile phase: from 80% water (0.1% FA) and 20% MeCN (0.1% FA) to 5% water (0.1% FA) and 95% MeCN (0.1% FA) in 2.6 min]: Rt=1.12 min; MS Calcd: 506.3, MS Found: 507.3 [M+H]⁺.

Chiral HPLC [Column: AD-H; Column size: 0.46 cm×15 cm; Mobile phase: Hex:EtOH (0.1% DEA)=60:40; Flow rate: 0.5 ml; Wave length: UV 254 nm; Temperature: 25° C.]: Rt=5.123 min, ee: 98.2%.

Peak 3 (E137): Single Unknown Isomer 3, Rt=5.284 Min

¹H NMR (400 MHz, CDCl₃) δ 8.77 (s, 1H), 8.05 (s, 1H), 7.50 (s, 1H), 6.96 (s, 1H), 4.36˜4.27 (m, 2H), 4.05˜3.93 (m, 4H), 3.86˜3.80 (m, 1H), 3.75˜3.67 (m, 2H), 3.47˜3.42 (m, 1H), 3.22˜2.97 (m, 5H), 2.86˜2.82 (m, 1H), 2.63 (s, 3H), 2.46 (s, 3H), 2.26˜2.09 (m, 4H), 1.97˜1.92 (m, 5H), 1.30 (d, J=6.4 Hz, 3H).

LC-MS [mobile phase: from 80% water (0.1% FA) and 20% MeCN (0.1% FA) to 5% water (0.1% FA) and 95% MeCN (0.1% FA) in 2.6 min]: Rt=1.12 min; MS Calcd: 506.3, MS Found: 507.3 [M+H]f.

Chiral HPLC[Column: AD-H; Column size: 0.46 cm×15 cm; Mobile phase: Hex:EtOH (0.1% DEA)=60:40; Flow rate: 0.5 ml; Wave length: UV 254 nm; Temperature: 25° C.]: Rt=5.284 min, ee: 97.7%.

Peak 4 (E138): Single Unknown Isomer 4, Rt=5.439 Min

¹H NMR (400 MHz, CDCl₃) δ 8.79 (s, 1H), 8.05 (s, 1H), 7.50 (s, 1H), 6.95 (s, 1H), 4.37˜4.25 (m, 2H), 4.10˜4.07 (m, 1H), 4.00˜3.94 (m, 2H), 3.85˜3.68 (m, 4H), 3.37˜3.33 (m, 1H), 3.21˜3.19 (m, 1H), 3.12˜2.97 (m, 3H), 2.90˜2.84 (m, 2H), 2.64 (s, 3H), 2.46 (s, 3H), 2.27˜2.10 (m, 4H), 1.94˜1.92 (m, 5H), 1.30 (d, J=6.4 Hz, 3H).

LC-MS [mobile phase: from 80% water (0.1% FA) and 20% MeCN (0.1% FA) to 5% water (0.1% FA) and 95% MeCN (0.1% FA) in 2.6 min]: Rt=1.10 min; MS Calcd: 506.3, MS Found: 507.3 [M+H]⁺.

Chiral HPLC [Column: AD-H; Column size: 0.46 cm×15 cm; Mobile phase: Hex:EtOH (0.1% DEA)=60:40; Flow rate: 0.5 ml; Wave length: UV 254 nm; Temperature: 25° C.]: Rt=5.439 min, ee: 97.1%.

Peak 5 (E139): Single Unknown Isomer 5, Rt=5.546 Min

¹H NMR (400 MHz, CDCl₃) δ 8.77 (s, 1H), 8.05 (s, 1H), 7.50 (s, 1H), 6.96 (s, 1H), 4.36˜4.27 (m, 2H), 4.05˜3.93 (m, 4H), 3.86˜3.80 (m, 1H), 3.75˜3.67 (m, 2H), 3.47˜3.42 (m, 1H), 3.21˜2.97 (m, 5H), 2.86˜2.82 (m, 1H), 2.63 (s, 3H), 2.46 (s, 3H), 2.30˜2.10 (m, 4H), 1.94˜1.92 (m, 5H), 1.30 (d, J=6.4 Hz, 3H).

LC-MS [mobile phase: from 80% water (0.1% FA) and 20% MeCN (0.1% FA) to 5% water (0.1% FA) and 95% MeCN (0.1% FA) in 2.6 min]: Rt=1.12 min; MS Calcd: 506.3, MS Found: 507.3 [M+H]⁺.

Chiral HPLC[Column: AD-H; Column size: 0.46 cm×15 cm; Mobile phase: Hex:EtOH (0.1% DEA)=60:40; Flow rate: 0.5 ml; Wave length: UV 254 nm; Temperature: 25° C.]: Rt=5.546 min, ee: 100%.

Peak 6 (E140): Single Unknown Isomer 6, Rt=6.033 Min

¹H NMR (400 MHz, CDCl₃) δ 8.79 (s, 1H), 8.05 (s, 1H), 7.50 (s, 1H), 6.95 (s, 1H), 4.37˜4.25 (m, 2H), 4.10˜4.07 (m, 1H), 4.02˜3.94 (m, 2H), 3.85˜3.66 (m, 4H), 3.36˜3.33 (m, 1H), 3.22˜3.19 (m, 1H), 3.11˜2.97 (m, 3H), 2.90˜2.83 (m, 2H), 2.64 (s, 3H), 2.46 (s, 3H), 2.27˜2.10 (m, 4H), 1.94˜1.92 (m, 5H), 1.30 (d, J=6.4 Hz, 3H).

LC-MS [mobile phase: from 80% water (0.1% FA) and 20% MeCN (0.1% FA) to 5% water (0.1% FA) and 95% MeCN (0.1% FA) in 2.6 min]: Rt=1.11 min; MS Calcd: 506.3, MS Found: 507.3 [M+H]⁺.

Chiral HPLC [Column: AD-H; Column size: 0.46 cm×15 cm; Mobile phase: Hex:EtOH (0.1% DEA)=60:40; Flow rate: 0.5 ml; Wave length: UV 254 nm; Temperature: 25° C.]: Rt=6.033 min, ee: 99.3%.

Peak 7 (E141): Single Unknown Isomer 7, Rt=6.335 Min

¹H NMR (400 MHz, CDCl₃) 8.79 (s, 1H), 8.05 (s, 1H), 7.50 (s, 1H), 6.95 (s, 1H), 4.37˜4.25 (m, 2H), 4.10˜4.06 (m, 1H), 4.02˜3.94 (m, 2H), 3.87˜3.66 (m, 4H), 3.37˜3.32 (m, 1H), 3.21˜3.18 (m, 1H), 3.12˜2.97 (m, 3H), 2.90˜2.82 (m, 2H), 2.64 (s, 3H), 2.46 (s, 3H), 2.26˜2.10 (m, 4H), 1.94˜1.92 (m, 5H), 1.30 (d, J=6.4 Hz, 3H).

LC-MS [mobile phase: from 80% water (0.1% FA) and 20% MeCN (0.1% FA) to 5% water (0.1% FA) and 95% MeCN (0.1% FA) in 2.6 min]: Rt=1.11 min; MS Calcd: 506.3, MS Found: 507.3 [M+H]⁺.

Chiral HPLC [Column: AD-H; Column size: 0.46 cm×15 cm; Mobile phase: Hex:EtOH (0.1% DEA)=60:40; Flow rate: 0.5 ml; Wave length: UV 254 nm; Temperature: 25° C.]: Rt=6.335 min, ee: 100%.

Peak 8 (E142): Single Unknown Isomer 8, Rt=6.937 Min

¹H NMR (400 MHz, CDCl₃) δ 8.79 (s, 1H), 8.05 (s, 1H), 7.50 (s, 1H), 6.95 (s, 1H), 4.37˜4.25 (m, 2H), 4.09˜4.06 (m, 1H), 4.01˜3.94 (m, 2H), 3.86˜3.65 (m, 4H), 3.36˜3.33 (m, 1H), 3.22˜3.19 (m, 1H), 3.11˜2.97 (m, 3H), 2.90˜2.83 (m, 2H), 2.64 (s, 3H), 2.46 (s, 3H), 2.25˜2.10 (m, 4H), 1.94˜1.92 (m, 5H), 1.30 (d, J=6.4 Hz, 3H).

LC-MS [mobile phase: from 80% water (0.1% FA) and 20% MeCN (0.1% FA) to 5% water (0.1% FA) and 95% MeCN (0.1% FA) in 2.6 min]: Rt=1.10 min; MS Calcd: 506.3, MS Found: 507.3 [M+H]⁺.

Chiral HPLC [Column: AD-H; Column size: 0.46 cm×15 cm; Mobile phase: Hex:EtOH (0.1% DEA)=60:40; Flow rate: 0.5 ml; Wave length: UV 254 nm; Temperature: 25° C.]: Rt=6.937 min, ee: 98.5%.

### Example 143

### 2-methyl-6-(5-methyl-6-(1-(tetrahydrofuran-3-yl)piperidin-4-yl)-1H-indazol-1-yl)-N—((R)-tetrahydrofuran-3-yl)pyrimidin-4-amine

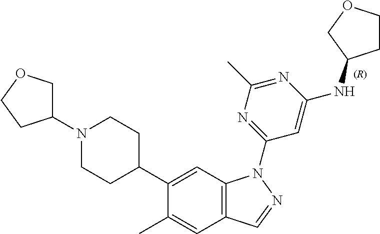

A mixture of (R)-2-methyl-6-(5-methyl-6-(piperidin-4-yl)-1H-indazol-1-yl)-N-(tetrahy-drofuran-3-yl)pyrimidin-4-amine (D131, 235 mg, 0.600 mmol), Dihydro-furan-3-one (258 mg, 3.00 mmol) and NaBH₃CN (76.0 mg, 1.20 mmol) in DCM (4.00 mL) was added AcOH (catalyst).

The reaction mixture was stirred at 40° C. overnight, then quenched with sat.NaHCO₃ (4 drops) and concentrated. The residue was purified by prep-HPLC (x-bridge C₁₈, 5 μm, 21.2×150 mm, 25-80% MeCN—H₂O (0.1% NH₄HCO₃), flow rate: 15 ml/min, GT12 mins.) to give the title product (102 mg, 37.0%) as a white solid.

¹H NMR (400 MHz, CDCl₃): δ 8.79 (s, 1H), 8.06 (s, 1H), 7.50 (s, 1H), 6.77 (s, 1H), 5.11-5.09 (m, 1H), 4.45 (s, 1H), 4.03-3.90 (m, 4H), 3.83-3.70 (m, 4H), 3.21-3.19 (m, 1H), 3.06-2.97 (m, 2H), 2.87-2.82 (m, 1H), 2.61 (s, 3H), 2.46 (s, 3H), 2.40-1.92 (m, 4H), 1.61 (s, 6H).

LCMS [column: C₁₈; column size: 4.6×50 mm; mobile phase: B(MeCN), A (0.02% NH₄Ac in water); gradient (B %) in 6 mins]: Rt=3.661 min; MS Calcd.:462, MS Found: 463 [M+H]⁺.

### Example 144

### 2-methyl-6-(5-methyl-6-(1-(tetrahydrofuran-3-yl)piperidin-4-yl)-1H-indazol-1-yl)-N—((S)-tetrahydrofuran-3-yl)pyrimidin-4-amine

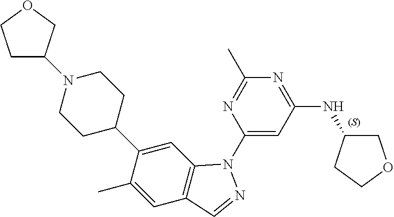

The title compound was prepared by a procedure similar to that described for E143 starting from a solution of (S)-2-methyl-6-(5-methyl-6-(piperidin-4-yl)-1H-indazol-1-yl)-N-(tetrahydrofuran-3-yl)pyrimidin-4-amine (D133), dihydrofuran-3(2H)-one and catalyst AcOH in DCM and NaBH₃CN at room temperature overnight.

¹HNMR (400 MHz, CDCl₃): δ 8.79 (s, 1H), 8.06 (s, 1H), 7.50 (s, 1H), 6.77 (s, 1H), 5.17 (br s, 1H), 4.44 (br, 1H), 4.03-3.71 (m, 8H), 3.27-2.82 (m, 4H), 2.61 (s, 3H), 2.46 (s, 3H), 2.40-2.19 (m, 4H), 2.04-1.86 (m, 6H).

LCMS [column: C₁₈; column size: 4.6 mm×50 mm; mobile phase: B (MeCN): A (0.1% FA in water); gradient (B %) in 6 mins]: Rt=2.585 min; MS Calcd.:462, MS Found: 463 [M+H]⁺.

### Examples 145, 146, 147 and 148

### 3-((2-methyl-6-(5-methyl-6-(1-(tetrahydrofuran-3-yl)piperidin-4-yl)-1H-indazol-1-yl)pyrimidin-4-yl)oxy)cyclobutanol (Single Unknown Isomer 1, Single Unknown Isomer 2, Single Unknown Isomer 3 and Single Unknown Isomer 4)

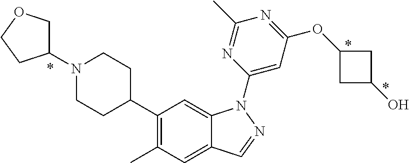 [SMILES: Cc1nc(OC2CC(O)C2)cc(-n2ncc3cc(C)c(C4CCN(C5CCOC5)CC4)cc32)n1]

To a solution of 3-((2-methyl-6-(5-methyl-6-(piperidin-4-yl)-1H-indazol-1-yl)pyrimidin-4-yl)oxy)cyclobutanol (D139, 500 mg, 1.27 mmol), dihydrofuran-3(2H)-one (546 mg, 6.35 mmol) and NaBH₃CN (160 mg, 2.54 mmol) in DCM (10.0 mL) was added catalyst AcOH. The reaction mixture was stirred at 40° C. for 3 hrs, treated with sat.NaHCO₃ (4 drops) and concentrated. The residue was purified by silica gel chromatography column (DCM/MeOH=20:1) to give the title product (330 mg, 56.0%) as a white solid.

¹H NMR (400 MHz, CDCl₃): δ 8.77 (s, 1H), 8.07 (s, 1H), 7.51 (s, 1H), 7.05 (s, 1H), 4.87-4.83 (m, 1H), 4.12-4.09 (m, 1H), 4.05-3.79 (m, 6H), 3.49 (s, 3H), 3.29-2.87 (m, 6H), 2.70 (s, 3H), 2.46 (s, 3H), 2.41-2.36 (m, 3H), 2.20-2.13 (m, 3H).

The product was separated by chiral-HPLC to afford isomer 1 (1 mg, 0.3%), isomer 4 (29 mg, 9%) and the mixture of isomer 2 and 3 (100 mg, 30%).

Chiral Separation:

Method: column: Superchiral S-AD, column size: 250 mm×4.6 mm, 5 μm; Phase: Supercritical CO₂/IPA/NH₃.H₂O=70/30/0.3; Flow rate: 12 ml/min; Wave length: 214 nm.

Peak 1 (E145): Single Unknown Isomer 1

Chiral HPLC [column: Superchiral S-AD, column size: 250 mm×4.6 mm, 5 μm; Phase: Hex/IPA/DEA=70/30/0.3; Flow rate: 12 ml/min; Wave length: 214 nm]: Rt=7.307 min.

¹H NMR (400 MHz, CDCl₃): δ 8.77 (s, 1H), 8.08 (s, 1H), 7.51 (s, 1H), 7.04 (s, 1H), 5.44-5.38 (m, 1H), 4.71-4.65 (m, 1H), 4.03-3.70 (m, 4H), 3.22-2.81 (m, 4H), 2.70 (s, 3H), 2.59-2.49 (m, 4H), 2.46 (s, 3H), 2.26-2.11 (m, 3H), 1.94 (s, 6H).

LC-MS [column: C₁₈; column size: 4.6×50 mm; mobile phase: B (MeCN), A (0.02% NH₄Ac in water); gradient (B %)]: Rt=3.853 min, MS Calcd.: 463, MS Found: 464 [M+H]⁺.

Peak 4 (E148): Single Unknown Isomer 4

Chiral HPLC [column: Superchiral S-AD, column size: 250 mm×4.6 mm, 5 μm; Phase: Hex/IPA/DEA=70/30/0.3; Flow rate: 12 ml/min; Wave length: 214 nm]: Rt=11.055 min.

¹H NMR (400 MHz, CDCl₃): δ 8.76 (s, 1H), 8.07 (s, 1H), 7.51 (s, 1H), 7.06 (s, 1H), 4.89-4.81 (m, 1H), 4.13-4.08 (m, 1H), 4.02-3.70 (m, 4H), 3.22-3.19 (m, 1H), 3.06-2.98 (m, 4H), 2.88-2.81 (m, 1H), 2.69 (s, 3H), 2.46 (s, 3H), 2.30-2.10 (m, 5H), 1.97-1.83 (m, 6H).

LC-MS [column: C₁₈; column size: 4.6×50 mm; mobile phase: B (MeCN), A (0.02% NH₄Ac in water); gradient (B %)]: Rt=2.876 min, MS Calcd.: 463, MS Found: 464 [M+H]⁺.

The mixture of isomer 2 and isomer 3 (100 mg) was further separated by chiral-HPLC to afford the chirally pure isomer 2 (30 mg, 30%) and isomer 3 (1 mg, 1%).

Chiral prep-HPLC: column: Superchiral S-AD, column size: 250×4.6 mm, 5 μm; Phase: Supercritical CO₂/EtOH/NH₃.H₂O=80/20/0.3; Flow rate: 14 ml/min; Wave Length: 214 nm.

Peak 2 (E146): Single Unknown Isomer 2

Chiral HPLC [column: Superchiral S-AD, column size: 250×4.6 mm, 5 μm; Phase: Hex/EtOH/DEA=80/20/0.3; Flow rate: 14 ml/min; Wave Length: 214 nm.]: Rt=20.253 min.

¹H NMR (400 MHz, CDCl₃): δ 8.77 (s, 1H), 8.07 (s, 1H), 7.51 (s, 1H), 7.06 (s, 1H), 4.89-4.81 (m, 1H), 4.13-4.08 (m, 1H), 4.02-3.70 (m, 4H), 3.22-3.19 (m, 1H), 3.06-2.98 (m, 4H), 2.88-2.81 (m, 1H), 2.69 (s, 3H), 2.46 (s, 3H), 2.30-2.10 (m, 5H), 1.97-1.83 (m, 6H).

LC-MS [column: C₁₈; column size: 4.6 mm×50 mm; mobile phase: B (MeCN), A (0.02% NH₄Ac in water); gradient (B %)]: Rt=3.494 min, MS Calcd.: 463, MS Found: 464 [M+H]⁺.

Peak 3 (E147): Single Unknown Isomer 3

Chiral HPLC [column: Superchiral S-AD, column size: 250×4.6 mm, 5 μm; Phase: Hex/EtOH/DEA=80/20/0.3; Flow rate: 14 ml/min; Wave Length: 214 nm]: Rt=16.535 min.

¹H NMR (400 MHz, CDCl₃): δ 8.77 (s, 1H), 8.08 (s, 1H), 7.51 (s, 1H), 7.04 (s, 1H), 5.44-5.38 (m, 1H), 4.71-4.65 (m, 1H), 4.03-3.70 (m, 4H), 3.22-2.81 (m, 4H), 2.70 (s, 3H), 2.59-2.49 (m, 4H), 2.46 (s, 3H), 2.26-2.11 (m, 3H), 1.94 (s, 6H).

LC-MS [column: C₁₈; column size: 4.6 mm×50 mm; mobile phase: B (MeCN), A (0.02% NH₄Ac in water); gradient (B %)]: Rt=3.612 min, MS Calcd.: 463, MS Found: 464 [M+H]⁺.

### Examples 149 and 150

### ((2S)-4-(6-(6-(1-(4-fluorotetrahydrofuran-3-yl)piperidin-4-yl)-5-methyl-1H-indazol-1-yl)-2-methylpyrimidin-4-yl)morpholin-2-yl)methanol (Single Unknown Isomer 1, Rt=5.761 Min; Single Unknown Isomer 2, Rt=6.008 Min)

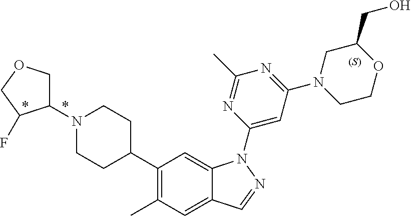

The title compound were prepared by a procedure similar to that described for E1 and E2 starting from a mixture of 6-(1-(4-fluorotetrahydrofuran-3-yl)piperidin-4-yl)-5-methyl-1H-indazole (D143) in toluene, (S)-(4-(6-iodo-2-methylpyrimidin-4-yl)morpholin-2-yl)methanol, CuI, K₃PO₄ and DMEDA at 90° C. for 2 hrs under N₂. According to the synthetic route and biological data the product could be cis configuration.

LC-MS [mobile phase: from 50% water (0.1% FA) and 50% MeCN (0.1% FA) to 5% water (0.1% FA) and 95% MeCN (0.1% FA) in 2.0 min]: Rt=0.36 min; MS Calcd.:510.28, MS Found: 511.3 [M+H]⁺.

Chiral Separation:

Method: Column: AD-H; Column size: 0.46 cm×15 cm; Mobile phase: Supercritical CO₂:IPA (0.1% NH₃.H₂O)=60:40; Flow rate: 0.5 mL/min; Wave length: UV 254 nm; Temperature: 25° C.; Sample solution in EtOH

Peak 1 (E149): Single Unknown Isomer 1, Rt=5.761 Min

¹H NMR (400 MHz, CDCl₃) δ 8.79 (s, 1H), 8.06 (s, 1H), 7.51 (s, 1H), 6.96 (s, 1H), 5.30˜5.14 (m, 1H), 4.33˜3.93 (m, 6H), 3.78˜3.65 (m, 5H), 3.36˜3.32 (m, 1H), 3.11˜3.08 (m, 2H), 2.98˜2.87 (m, 3H), 2.64 (s, 3H), 2.46 (s, 3H), 2.35˜2.29 (m, 2H), 2.00˜1.80 (m, 5H).

¹⁹F NMR (376 MHz, CDCl₃) δ 176.32 (s, 1F).

LC-MS [mobile phase: from 80% water (0.1% FA) and 20% MeCN (0.1% FA) to 5% water (0.1% FA) and 95% MeCN (0.1% FA) in 2.0 min]: Rt=1.07 min; MS Calcd: 510.28, MS Found: 511.4 [M+H]⁺.

Chiral HPLC[Column: AD-H; Column size: 0.46 cm×15 cm; Injection: 2 μl; Mobile phase: HEP:IPA (0.1% DEA)=60:40; Flow rate: 0.5 mL/min; Wave length: UV 254 nm; Temperature: 25° C.]: Rt=5.761 min, ee 99.53%;

Peak 2 (E150): Single Unknown Isomer 2, Rt=6.008 Min

¹H NMR (400 MHz, CDCl₃) δ 8.72 (s, 1H), 7.99 (s, 1H), 7.44 (s, 1H), 6.89 (s, 1H), 5.24˜5.08 (m, 1H), 4.23˜3.83 (m, 6H), 3.74˜3.58 (m, 5H), 3.29˜3.25 (m, 1H), 3.13˜3.04 (m, 2H), 2.89˜2.76 (m, 3H), 2.57 (s, 3H), 2.39 (s, 3H), 2.28˜2.22 (m, 2H), 1.94˜1.75 (m, 5H).

¹⁹F NMR (376 MHz, CDCl₃) δ 176.32 (s, 1F).

LC-MS [mobile phase: from 80% water (0.1% FA) and 20% MeCN (0.1% FA) to 5% water (0.1% FA) and 95% MeCN (0.1% FA) in 2.0 min]: Rt=1.07 min; MS Calcd: 510.28, MS Found: 511.4 [M+H]⁺.

Chiral HPLC [Column: AD-H; Column size: 0.46 cm×15 cm; Injection: 2 μl; Mobile phase: HEP:IPA (0.1% DEA)=60:40; Flow rate: 0.5 mL/min; Wave length: UV 254 nm; Temperature: 25° C.]: Rt=6.008 min, ee: 99.09%;

### Examples 151, 152, 130 and 154

### 4-(4-(1-(6-((S)-2-(hydroxymethyl)morpholino)-2-methylpyrimidin-4-yl)-5-methyl-1H-indazol-6-yl)piperidin-1-yl)tetrahydrofuran-3-ol (Single Unknown Isomer 1, Single Unknown Isomer 2, Single Unknown Isomer 3 and Single Unknown Isomer 4)

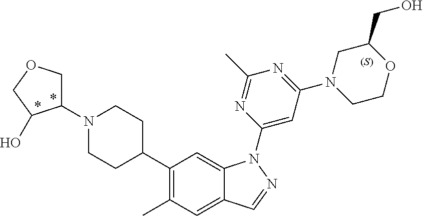

The title compounds were prepared by a procedure similar to that described for E1 and E2 starting from a suspension of 4-(4-(5-methyl-1H-indazol-6-yl)piperidin-1-yl)tetrah-ydrofuran-3-ol (D141), (S)-(4-(6-iodo-2-methylpyrimidin-4-yl)morpholin-2-yl)methanol (D3), CuI and K₃PO₄ in toluene and THE and N¹,N²-dimethylethane-1,2-diamine at 80° C. for 3 h under N₂.

LC-MS [mobile phase: from 70% water (0.1% FA) and 30% MeCN (0.1% FA) to 5% water (0.1% FA) and 95% MeCN (0.1% FA) in 2.0 min]: Rt=1.09 min; MS Calcd: 508.3, MS Found: 509.4 [M+H]⁺.

Chiral Separation:

AD-H 4.6×250 mm, 5 um (Daicel), Mobile phase: Supercritical CO₂/EtOH (0.2% NH₃.H₂O)=60/40, flow rate: 0.5 mL/min, temperature: 35° C.

Peak 1 (E151): Single Unknown Isomer 1, Rt=3.380 Min

¹H NMR (400 MHz, CDCl₃): δ 8.79 (s, 1H), 8.06 (s, 1H), 7.50 (s, 1H), 6.95 (s, 1H), 4.49˜4.45 (m, 1H), 4.33˜4.27 (m, 2H), 4.17˜4.13 (m, 1H), 4.08˜3.99 (m, 2H), 3.79˜3.66 (m, 6H), 3.38˜3.33 (m, 1H), 3.15˜3.07 (m, 1H), 2.98˜2.83 (m, 4H), 2.64 (s, 3H), 2.46 (s, 3H), 2.37˜2.28 (m, 2H), 1.96˜1.88 (m, 4H).

LC-MS [mobile phase: from 90% water (0.1% FA) and 10% MeCN (0.1% FA) to 5% water (0.1% FA) and 95% MeCN (0.1% FA) in 9 min]: Rt=4.24 min; MS Calcd: 508.3, MS Found: 509.4 [M+H]⁺.

Chiral HPLC [AD-H 4.6×250 mm, 5 um (Daicel), Mobile phase: Hexane/EtOH (0.2% DEA)=60/40, flow rate: 0.5 mL/min, temperature: 35° C.]: Rt=3.380 min, ee: 98.44%

Peak 2 (E152): Single Unknown Isomer 2, Rt=4.123 Min

¹H NMR (400 MHz, CDCl₃): δ 8.79 (s, 1H), 8.06 (s, 1H), 7.50 (s, 1H), 6.95 (s, 1H), 4.49˜4.45 (m, 1H), 4.32˜4.28 (m, 2H), 4.18˜4.14 (m, 1H), 4.08˜4.00 (m, 2H), 3.79˜3.67 (m, 6H), 3.37˜3.34 (m, 1H), 3.15˜3.08 (m, 1H), 2.98˜2.83 (m, 4H), 2.64 (s, 3H), 2.46 (s, 3H), 2.36˜2.28 (m, 2H), 1.96˜1.86 (m, 4H).

LC-MS [mobile phase: from 90% water (0.1% FA) and 10% MeCN (0.1% FA) to 5% water (0.1% FA) and 95% MeCN (0.1% FA) in 9 min]: Rt=4.22 min; MS Calcd: 508.3, MS Found: 509.4 [M+H]⁺.

Chiral HPLC [AD-H 4.6 mm×250 mm, 5 μm (Daicel), Phase: Hexane/EtOH (0.2% DEA)=60/40, flow rate: 0.5 mL/min, temperature: 35° C.]: Rt=4.123 min, ee: 97.32%

Peak 3 (E153): Single Unknown Isomer 2, Rt=4.881 Min

¹H NMR (400 MHz, CDCl₃): δ 8.80 (s, 1H), 8.06 (s, 1H), 7.51 (s, 1H), 6.96 (s, 1H), 4.33˜4.26 (m, 3H), 4.09˜3.96 (m, 4H), 3.83˜3.66 (m, 5H), 3.27˜3.24 (m, 1H), 3.15˜3.07 (m, 1H), 2.98˜2.83 (m, 4H), 2.64 (s, 3H), 2.51˜2.46 (m, 1H), 2.46 (s, 3H), 2.37˜2.30 (m, 1H), 1.94˜1.86 (m, 4H).

LC-MS [mobile phase: from 90% water (0.1% FA) and 10% MeCN (0.1% FA) to 5% water (0.1% FA) and 95% MeCN (0.1% FA) in 9 min]: Rt=4.21 min; MS Calcd: 508.3, MS Found: 509.4 [M+H]⁺.

Chiral HPLC [AD-H 4.6 mm×250 mm, 5 μm, Phase: Hexane/EtOH (0.2% DEA)=60/40, flow rate: 0.5 mL/min, Temperature: 35° C.]: Rt=4.881 min, ee: 99.38%

Peak 4 (E154): Single Unknown Isomer 2, Rt=6.712 Min

¹H NMR (400 MHz, CDCl₃): δ 8.88 (s, 1H), 8.07 (s, 1H), 7.54 (s, 1H), 6.95 (s, 1H), 4.66˜4.62 (m, 1H), 4.32˜4.29 (m, 2H), 4.21˜4.16 (m, 2H), 4.08˜4.01 (m, 3H), 3.98˜3.94 (m, 1H), 3.80˜3.68 (m, 5H), 3.45˜3.41 (m, 1H), 3.15˜2.92 (m, 5H), 2.62 (s, 3H), 2.47 (s, 3H), 2.47˜2.36 (m, 2H), 2.17˜2.11 (m, 2H).

LC-MS [mobile phase: from 90% water (0.1% FA) and 10% MeCN (0.1% FA) to 5% water (0.1% FA) and 95% MeCN (0.1% FA) in 9 min]: Rt=4.20 min; MS Calcd: 508.3, MS Found: 509.4 [M+H]⁺.

Chiral HPLC [AD-H 4.6×250 mm, 5 um (Daicel), Mobile phase: Hexane/EtOH (0.2% DEA)=60/40, flow rate: 0.5 mL/min, temperature: 35° C.]: Rt=6.712 min, ee: 98.66%

### Example 155

### ((2S)-4-(2-methyl-6-(5-methyl-6-(1-(4-methyltetrahydrofuran-3-yl)piperidin-4-yl)-1H-indazol-1-yl)pyrimidin-4-yl)morpholin-2-yl)methanol

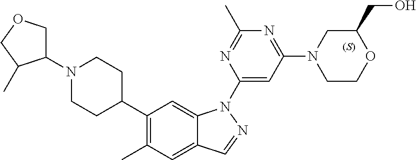

The title compound was prepared by a procedure similar to that described for E1 and E2 starting from a suspension of 5-methyl-6-(1-(4-methyltetrahydrofuran-3-yl)piperidin-4-yl)-1H-indazole (D149) and (S)-(4-(6-iodo-2-methylpyrimidin-4-yl)morpholin-2-yl)metha-nol (D3) in toluene, CuI, K₃PO₄.3H₂O and DMEDA at 100° C. for 4 h.

LC-MS [mobile phase: from 90% water (0.1% FA) and 10% MeCN (0.1% FA) to 5% water (0.1% FA) and 95% MeCN (0.1% FA) in 10.0 min]: Rt=4.38 min; MS Calcd.:506.3, MS Found: 507.4 [M+H]⁺.

¹H NMR (400 MHz, CDCl₃): δ 8.66 (s, 1H), 8.10 (s, 1H), 7.54 (s, 1H), 6.97 (s, 1H), 4.37˜4.29 (m, 3H), 4.09˜4.07 (m, 3H), 3.90˜3.87 (m, 1H), 3.78˜3.70 (m, 7H), 3.22˜3.17 (m, 3H), 3.07˜3.02 (m, 4H), 2.70 (s, 3H), 2.70˜2.62 (m, 2H), 2.46 (s, 3H), 2.12˜2.09 (m, 2H), 1.37˜1.35 (d, J=7.2 Hz, 3H).

### Example 156

### (2-methyl-4-(2-methyl-6-(5-methyl-6-(1-((S)-tetrahydrofuran-3-yl)piperidin-4-yl)-1H-indazol-1-yl)pyrimidin-4-yl)morpholin-2-yl)methanol

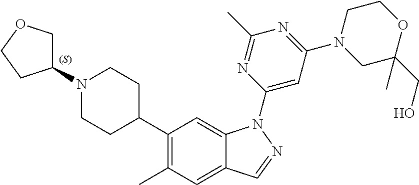

A mixture of (2-methyl-4-(2-methyl-6-(5-methyl-6-(piperidin-4-yl)-1H-indazol-1-yl)pyrimidin-4-yl)morpholin-2-yl)methanol (D157, 350 mg, 0.800 mmol), (R)-tetrahydrofuran-3-yl methanesulfonate (400 mg, 2.41 mmol) and K₂CO₃ (443 mg, 3.21 mmol) in MeCN (7.00 mL) was stirred at 100° C. for 40 hrs in a sealed tube. The reaction mixture was diluted with H₂O (30 mL), extracted with EtOAc (30 mL×3). The combined organic layers were concentrated and purified by silica gel chromatography column (DCM/MeOH=20/1) to give the product (115 mg, 28.0%) as a yellow solid. The product was further purified by prep-HPLC (Kinete EVO C₁₈ SN H16-100024 phenomenex, Waters-2 Kinete EVO C₁₈, 5 μm, 21.2 mm×150 mm Gradient: B % (20-80%), A (0.1% NH₄HCO₃ in water), B (MeCN), UV: 214 nm, flowrate 15 ml/min, 12 min-GT 7.5 mins) to afford the title product (60.0 mg, 15.0%) as a yellow solid.

¹HNMR (400 MHz, CD₃OD): δ 8.77 (s, 1H), 8.14 (s, 1H), 7.58 (s, 1H), 7.00 (s, 1H), 4.58 (br s, 1H), 4.05-4.00 (m, 1H), 3.95-3.71 (m, 7H), 3.63-3.50 (m, 4H), 3.46-3.38 (m, 2H), 3.25-3.24 (m, 1H), 3.08-3.07 (m, 1H), 2.73-2.66 (m, 2H), 2.59 (s, 3H), 2.48 (s, 3H), 2.30-2.26 (m, 1H), 2.08-1.96 (m, 4H), 1.23 (s, 3H).

LC-MS [column: C₁₈; column size: 4.6 mm×50 mm; mobile phase: B (MeCN), A (0.02% NH₄Ac in water); gradient (B %)]: Rt=3.453 min, MS Calcd.: 506, MS Found: 507 [M+H]⁺.

### F. Assays and Data

As stated above, the compounds of present invention are LRRK2 kinase inhibitors, and may be useful in the treatment of diseases mediated by LRRK2. The biological activities and/or properties of the compounds of present invention can be determined using any suitable assay, including assays for determining the activity of a candidate compound as a LRRK2 kinase inhibitor, as well as tissue and in vivo models.

### 1. Assays

a. Full Length G2019 Human LRRK2 Inhibition Mass Spectrometry Assay

This assay for Leucine Rich Repeat Kinase 2 (LRRK2) inhibition is based on the direct measurement of the peptide ‘LRRKtide’ (LRRKtide: RLGRDKYKT*LRQIRQ and “*” refers to the site of phosphorylation.) and phosphorylated ‘LRRKtide’ using a high throughput RapidFire mass spectrometry assay. Inhibitors are compounds that reduce the conversion of LRRKtide to phospho-LRRKtide.

### Human G2019 LRRK2 Plasmid Preparation

### Primers Used for PCR Cloning:

pHTBV-F:SEQ ID No: 1
LRRK2 wt-F1:SEQ ID No: 2
LRRK2 wt-R1: SEQ ID No: 3
LRRK2 wt-F2: SEQ ID No: 4
LRRK2 wt-R2: SEQ ID No: 5
LRRK2 wt-F3:SEQ ID No: 6
pHTBV-R: SEQ ID No: 7
   pHTBV1-N-Flag-hu LRRK2 was generated by PCR amplifying the full length LRRK2 sequence with N terminal Flag tag from pcDNA3.1(+)_Human_LRRK2 (NCBI Reference Sequence: NP_940980.3) with the primers described above, and cloned into pHTBV1mcs3 vector between BamHI and KpnI sites.

The G2019 full length Flag-LRRK2 coding sequence is SEQ ID No: 8.

The translated amino acid sequence for human G2019 full length N terminal flag tagged LRRK2 protein is SEQ ID No: 9.

### Insect Cell Cultures

Sf9 insect cells (Invitrogen Life Technologies, Carlsbad, Calif.) were maintained at 27° C. in SF 900 II SFM in 500-ml shaker flasks (Erlenmeyer, Corning). The cells were maintained in exponential growth phase and subcultured twice per week. For larger volumes, cells were grown in 2-liter shaker flasks (Erlenmeyer, Corning) while being agitated with 120 rpm at 27° C. incubator shaker.

### Generation of the BacMam Virus

To generate the recombinant BacMam virus, DH10Bac competent cells (10361-012, Invitrogen) were transformed by the genotypically normal human LRRK2 BacMam plasmid to generate the recombinant baculovirus DNA. The Sf9 insect cells were co-transfected with the mixture of recombinant baculovirus DNA and cellfectin (10362-100, Invitrogen). After 4 h of incubation at 27° C., the transfection media was replaced with Sf-900 μl SFM medium containing 5% HI FBS (Ser. No. 10/100,147, Invitrogen). The cells were further incubated for 4 days. The infected cell culture medium containing the baculovirus (P0 virus stock) was collected and amplified by further infecting the 200 ml Sf9 cells via 200-300 ul P0.

### Quantification of BacMam Viral Titre by BacPAKRapid Titer

The viral titre, measured as plaque forming unit (pfu)/ml was determined using BacPAK Papid Titer Kit (631406, Clontech) according to the manufacturer's protocol. The Sf9 cells seeded in 96-well plate with 3×10⁵ cells per well were incubated with serial dilution of the viral stocks for 1 h at 27° C., 50 μl methyl cellulose overlay was added to each well followed by 43-47 h incubation. The cells were then fixed in 4% paraformaldehyde (PFA). After blocking the cells with diluted normal goat serum, Mouse anti-gp64 antibody was added to the cells. After 30 min incubation, the cells were washed with phosphate buffered saline containing 0.2% Triton-X100 (PBST) and incubated for another 30 min with goat anti-mouse antibody/HRP conjugate. This was followed by blue peroxidase substrate which detects the single infected cells and foci of infected cells by their dark blue color.

### Protein Expression & Purification

a) Expression of Flag Tagged Full Length G2019 Human LRRK2

HEK293 6E cells were incubated in a 37° C. incubator with a humidified atmosphere of 5% CO₂ on an orbital shaker rotating at 110 rpm. On the day of transduction, the cell viability was higher than 98% and the cell density was in the range of 1×10⁶˜1.5×10⁶ cells/ml. HEK293 6E cells were centrifuged at 1,000 rpm for 10 min, and then the cells were resuspended in fresh Freestyle 293 expression medium (Invitrogen:12338) with 0.1% F-68 (Invitrogen:24040-032) but without antibiotics (G418) at density of 1×10⁶ cells/ml. BacMam virus with Flag-hu LRRK2 (genotypically normal) gene was centrifuged at 40,000 g for 2 hours, then resuspended in fresh Freestyle 293 expression medium. The resuspended virus was added into the cells in at MOI of 10. The cells were incubated in a 37° C. incubator with a humidified atmosphere of 5% CO₂ in air on an orbital shaker rotating at 110 rpm. Cultures were harvested at approximately 48 hours post-transduction by centrifugation at 4,000 rpm for 20 min and pellets were frozen for purification.

b) Purification of Flag Tagged Full Length G2019 Human LRRK2

The cell pellet was resuspended in (20 mL/liter cell culture) lysis buffer (50 mM TrisHCl pH7.5 at 4° C., 500 mM NaCl, 0.5 mM EDTA, 0.1% TritonX-100, 10% glycerol, freshly add 2 mM DTT), with protease inhibitors (Roche: 04693132001) and benzonase (Merck Millipore: 70746-3CN) at recommended concentration suggested by suppliers. The suspended cells were lysed by sonication on ice for 30 min (2 secs on/4 sec off, 20% amplitude), and centrifuged at 10,000 rpm for 30 minutes at 4° C. The supernatant was incubated with 1 mL per litre of cell culture of anti-Flag magnetic beads (Sigma-Aldrich: M8823) at 4° C. for 3 hours, then the beads were washed by 5 mL (5 column volume) binding buffer (50 mM Tris pH7.5@ 4C, 500 mM NaCl, 0.5 mM EDTA, 0.1% TritonX-100, 10% glycerol, freshly add 2 mM DTT) for three times. The Flag tagged LRRK2 proteins were eluted by Elution buffer (50 mM Tris pH7.5@ 4C, 500 mM NaCl, 0.5 mM EDTA, 0.1% TritonX-100, 10% glycerol, freshly add 2 mM DTT, 250 ug/ml Flag peptide (Sigma-Aldrich:F3290)) at 4° C. for 2 hours. Flag peptide was removed by Zeba Spin Desalting Columns, 7K MWCO (Thermo-Fisher: 89893) and the buffer of eluted LRRK2 proteins was exchanged into Storage Buffer (50 mM Tris pH7.5@4C, 150 mM NaCl, 0.5 mM EDTA, 0.02% Triton X-100, 2 mM DTT and 50% Glycerol) using Amicon Ultra Centrifugal Filter Units (100 kD) (Merck: UFC910096). Fractions containing LRRK2 proteins were pooled, aliquoted and stored at −80° C. Protein concentration was determined by Bradford protein assay, and protein purity was analyzed by NuPAG Novex 4-12% Bis-Tris Protein Gels (Invitrogen: NP0322BOX).

### Assay Protocol

1) A 10 mM test compound was dissolved in 100% DMSO and serially diluted 1 in 4. 100 nL of this dilution series was then added to a 384 well, v bottom polypropylene plate, excluding columns 6 and 18. 100 nL of DMSO was added to columns 6 and 18 as controls wells. Assay dilution gave a top final assay concentration of test compound of 100 μM
2) 50 μl of 1% formic acid in laboratory grade water was added to column 18 using a multidrop combi dispenser to act as a pre stopped assay control.
3) 5 μl of ‘enzyme solution’ containing 50 nM of purified recombinant Full length Flag-LRRK2 in assay buffer (50 mM Hepes (pH 7.2), 10 mM MgCl2, 150 mM NaCl, 5% glycerol, 0.0025% triton X-100 and 1 mM DTT) was added to all wells using a multidrop combi dispenser, giving a final assay concentration of 25 nM LRRK2 enzyme. This resulted in column 6 (enzyme plus DMSO) giving 0% inhibition and column 18 giving 100% inhibition (pre stopped control). Test plates were then incubated for 30 minutes at room temperature.
4) 5 μl ‘substrate solution’ containing 50 uM LRRKtide peptide substrate and 4 mM ATP was added to all wells of the plate using a multidrop combi dispenser giving a final assay concentration of 25 uM LRRKtide and 2 mM ATP. Test plates were then incubated for 1 hour at room temperature. (Incubation may vary depending on rate and linearity of reaction with different enzyme batches).
5) 50 μl of 1% formic acid in laboratory grade water was added to all wells (minus column 18) to quench the reaction, and plates were centrifuged at 3000 rpm for 10 minutes. Test plates were then analysed on an Agilent RapidFire High Throughput solid phase extraction system coupled to AB Sciex API 4000 triple quadropole mass spectrometer with the following setting:
   RapidFire settings:
   Sip Height=2 mm, Aspirate=500 ms, Load time=3000 ms, Elution time=3000 ms, Requilibration=500 ms.
   Flow rates: pump 1=1.5 mL/min, pump 2 1.25 mL/min pump 3=0.8 mL/min Mass Spectrometer Settings:
   LRRKtide Detection settings: Q1 mass 644.8 Da, Q3 mass 638.8, delustering potential 76 volts, collision energy 37 volts, CXP 34 volts.
   Phospho-LRRKtide Detection settings: Q1 mass 671.4 Da, Q3 mass 638.8, Declustering potential 76 volts, Collision energy 37 volts, CXP 34 volts.
   A C4 cartridge was used and running buffers were: A (aqueous) 0.1% formic acid in water B (organic) 0.1% formic acid, 80% acetonitrile, 20% water.
   Collision gas: 12, Curtain gas: 25, Ion Source gas (1): 60, Ion Source gas (2): 60, Ion Spray Voltage: 5500, Temperature: 600, Interface Heater: ON.
   Resolution Q1: low, Resolution Q3: low.
6) Data was analysed using ActivityBase software (IDBS). A percent conversion from LRRKtide to Phospho-LRRKtide was calculated using the following formula:

% conversion=(Phospho-LRRKtide product peak area/(Phospho-LRRKtide product peak area+LRRKtide substrate peak area))*100

b. Recombinant Cellular LRRK2 AlphaScreen Assay

To determine the activity of compounds against LRRK2 kinase activity in cells, the observed LRRK2 kinase-dependent modulation of LRRK2 Ser 935 phosphorylation (Dzamko et al., 2010, Biochem. J. 430: 405-413) was utilized to develop a quantitative 384 well plate-based immunoassay of LRRK2 Ser935 phosphorylation in the human neuroblastoma cell line SH-SY5Y, engineered to over-express recombinant full length LRRK2 protein.
A BacMam virus expressing full length recombinant LRRK2 was purchased from Invitrogen and amplified by inoculation of SF-9 cells at MOI 0.3 for 4-5 days in Sf-900 III SFM medium supplemented with 3% fetal bovine serum. Infected cell cultures were then centrifuged at 2000 g for 20 minutes, viral supernatant titer determined by anti-gp64 plaque assay and stored at 4° C.
Affinity-purified anti-phospho LRRK2 Ser935 sheep polyclonal antibody (Dzamko et al., 2010, Biochem. J. 430: 405-413) was biotinylated by standard methods (PerkinElmer). Anti-LRRK2 rabbit polyclonal antibody was purchased from Novus Biologicals. AlphaScreen Protein A IgG Kit (including acceptor and donor beads) was purchased from Perkin Elmer.
SH-SY5Y cells were grown in DMEM/F12 medium with 10% dialysed fetal bovine serum and harvested by treatment with 0.5% trypsin-EDTA for 5 minutes at 37° C. followed by centrifugation at 1000 rpm for 4 minutes. The cell pellet was resuspended in Opti-MEM reduced serum media (Invitrogen) at 200,000 cells/ml and mixed with the BacMam LRRK2 virus at MOI=50. 50 μl cell solutions were then dispensed to each well of a 384-well plate and incubated at 37° C., 5% CO₂ for 24 hours.
Serial dilutions of test compounds were prepared in Opti-MEM reduced serum media (Invitrogen) and 5.6 ul transferred from compound plate to cell assay plate to achieve a top final assay concentration of 10 uM. DMSO was used in certain wells as controls. Cells were incubated at 37° C., 5% CO₂ for 60 minutes. The medium was then removed and cells lysed by addition of 20 ul cell lysis buffer (Cell Signaling Technology) and incubation at 4° C. for 20 minutes. 10 ul of antibody/acceptor bead mix [(1/1000 biotinylated-pS935 LRRK2 antibody, 1/1000 total-LRRK2 antibody, 1/100 Acceptor beads in AlphaScreen detection buffer (25 mM Hepes (pH 7.4), 0.5% Triton X-100, 1 mg/ml Dextran 500 and 0.1% BSA)] was then added to each well and plates incubated for 2 hours at ambient temperature in the dark. 10 μl of donor beads solution (1/33.3 donor beads in AlphaScreen detection buffer) was then added to each well. Following incubation for a further 2 hours at ambient temperature in the dark, plates were read on an EnVision™ plate reader at emission 520-620 nm with excitation 680 nm. Dose response curve data was based on sigmoidal dose-response model.

c. FASSIF Solubility Assay

Compound solubility may be evaluated in the fasted state simulated intestinal media (FaSSIF) at pH 6.5. Certain amount of test compound was admixed with certain volume of FaSSIF to prepare a suspension of about 1 mg/ml. The suspension was incubated at 37° C. in the water bath shaker for 24 hours. At the 4^{th} and 24^{th} hour, the suspension was centrifugated at 14K rpm for 15 minutes. 100 μl of the supernatant was withdrawn and diluted with the same volume of 50% acetonitrile water solution and analysed with UPLC (Ultra performance Liquid Chromatography). FaSSIF solubility was calculated based on the peak area of the test compound.
The FaSSIF (170 ml) preparation 100 mg of lecithin and 274 mg (anhyd equiv) of NaTaurocholate were dissolved in about 150 ml of pH 6.5 buffer. The solution was made to the volume of 170 ml with the pH 6.5 buffer.
The pH 6.5 buffer solution (1 L) preparation 4.083 g KH₂PO₄ and 7.456 g KCl were dissolved in 800 ml of water, with 100 ml 0.1 M NaOH subsequently added. The solution was made to the volume of 1 L with water. The pH value of the buffer solution was measured and adjusted to be 6.50±0.1.
Standard solutions for UPLC calibration and solubility calculation 2 μM, 20 μM and 200 μM DMSO (50% ACN water) solutions.

### UPLC Method and Parameter

Instrument: Waters ACQUITY UPLC System
Column: Waters ACQUITY UPLC BEH C₁₈ (1.7 μm, 2.1×50 mm)
Mobile phase: A: 0.1% TFA in water/B: 0.1% TFA in CAN
Gradient: 0 min (A 95%/B 5%), 2 min (A 5%/B 95%), 2.5 min (A 5%/B 95%), 2.6 min (A 95%/B 5%), 3 min (A 95%/B 5%)
Flow rate: 0.8 mL/min; column temperature: 40° C.; injection volume: 1.0 μL; UV detection: 280 nm

d. CLND Solubility Assay

Kinetic solubility of a compound may be evaluated by the CLND (ChemiLuminescent Nitrogen Detection) solubility assay, based on known protocols (see, e.g., Bhattachar S. N.; Wesley J. A.; Seadeek C., Evaluation of the Chemiluminescent Nitrogen Detector for Solubility Determinations to Support Drug Discovery, J. Pharm. Biomed. Anal. 2006 (41):152-157; Kestranek A, Chervenek A, Logenberger J, Placko S. Chemiluminescent Nitrogen Detection (CLND) to Measure Kinetic Aqueous Solubility, Curr Protoc Chem Biol., 2013, 5(4):269-80). Typically, 5 μl of 10 mM DMSO stock solution of the test compound was diluted to 100 μl with pH7.4 phosphate buffered saline, equilibrated for 1 hour at room temperature, filtered through Millipore MultiscreenHTS-PCF filter plates (MSSL BPC). The filtrate is quantified by suitably calibrated flow injection Chemi-Luminescent Nitrogen Detection.

### 2. Assay Data

Compounds of Examples E1-E67, E74-E77, E94, E95, E100, E110, E127, E129, E136, E149, E99, E141, E102, E143, E155, E132, E140, E156, E106, E113, E128, E137, E142, E131, E144, E133, E139, E114, E145, E147 and E148, were tested in the recombinant cellular LRRK2 AlphaScreen assay and exhibited a pIC50 of ≥6.
Compounds of Examples E1, E4-E9, E11, E13, E14, E17, E19, E21, E22, E24, E25, E29, E30, E34, E44, E47-E50, E53-E55, E60, E63, E64, E74-E76, E126, E103, E119, E124, E97, E98, E130, E104, E116, E150, E153, E105, E107, E138, E152, E96, E101, E134, E135 and E151 were tested in the recombinant cellular LRRK2 AlphaScreen assay and exhibited a pIC50 of ≥7.
Compounds of Examples E5, E29, E53, E55, E117, E121, E120, E118, E123, E115, E122 and E125 were tested in the recombinant cellular LRRK2 AlphaScreen assay and exhibited a pIC50 of ≥8.
Example 5 exhibited an pIC50 of 8.3 in the recombinant cellular LRRK2 AlphaScreen assay. In addition, Examples E1, E4, E34, E35, E36, E96, E138 exhibited a pIC50 of 6.9, 7.0, 7.0, 6.7, 6.8, 7.0 and 7.2, respectively.
Compounds of Examples E1-E6, E13, E14, E16, E30, E33, E34, E39-E46, E117, E125, E150, E113, E128, E142, E133, E139, E111 and E146 were tested in the Full length G2019 human LRRK2 Inhibition Mass Spectrometry assay and exhibited a pIC50 of ≥6.0.
Compounds of Examples E121, E123, E122, E126, E103, E119, E124, E130, E104, E116, E153, E105, E107, E138, E101, E134, E135, E94, E95, E127, E129, E136, E149, E141, E102, E155, E140, E156, E106 and E137 were tested in the Full length G2019 human LRRK2 Inhibition Mass Spectrometry assay and exhibited a pIC50 of z 7.0.
Compounds of Examples E120, E118 and E115 were tested in the Full length G2019 human LRRK2 Inhibition Mass Spectrometry assay and exhibited a pIC50 of ≥8.0.
Compounds of Examples E1, E4, E34, E 95 and E138 were tested in the Full length G2019 human LRRK2 Inhibition Mass Spectrometry assay and exhibited an pIC50 of 7.1, 7.6, 7.2, 7.4 and 7.6, respectively.
Certain compounds of the invention were tested in the FaSSIF solubility assay and/or the CLIND solubility assay. The tested compounds exhibited an unexpected trend of increased solubility when compared with compounds disclosed in WO 2017/012576. Solubility increase was particularly pronounced for some compounds of the invention in at least one solubility assay. Illustratively, the FaSSIF (at 24 hr.) solubility of Examples E1, E4, E103, E135, E136 and E137 of the invention was about 2.5 to 15 times as much as that of Examples E183, E182, E177, E267, E268 and E269 of WO 2017/012576, respectively. The CLIND solubility of Examples E4, E102, E121, E134, E135 and E136 of the invention was about 3 to 16 times as much as that of Examples E182, E176, E171, E266, E267 and E268 of WO 2017/012576, respectively.

### 3. Sequence Listing

SEQ ID NO: 1 Primers used for PCR cloning of Human G2019 LRRK2
plasmids preparation: pHTBV-F
5′-GATCTCGACGGGCGCGGATCCACCATGGATTACAAGGATGACGACGAT-3′
SEQ ID NO: 2 Primers used for PCR cloning of Human G2019 LRRK2
plasmids preparation: LRRK2 wt-F1
5′-CATGGATTACAAGGATGACGACGATAAGATGGCTAGTGGCAGCTGTCAG-3′
SEQ ID NO: 3 Primers used for PCR cloning of Human G2019 LRRK2
plasmids preparation: LRRK2 wt-R1
5′-GTTCACGAGATCCACTATTCAGTAAGAGTTCCACCAATTTGGGACTG-3′
SEQ ID NO: 4 Primers used for PCR cloning of Human G2019 LRRK2
plasmids preparation: LRRK2 wt-F2
5′- GAATAGTGGATCTCGTGAACAAG-3′
SEQ ID NO: 5 Primers used for PCR cloning of Human G2019 LRRK2
plasmids preparation: LRRK2 wt-R2
5′- GTCAGACAAACTGCTTGGAACCAGC-3′
SEQ ID NO: 6 Primers used for PCR cloning of Human G2019 LRRK2
plasmids preparation: LRRK2 wt-F3
5′-CTGGTTCCAAGCAGTTTGTCTGACCACAGGCCTGTGATAG-3′
SEQ ID NO: 7 Primers used for PCR cloning of Human G2019
LRRK2 plasmids preparation: pHTBV-R
5′-GTTCTAGCCAAGCTTGGTACCCTATTACTCAACAGATGTTCGTCTC-3′
SEQ ID NO: 8 G2019 Full length Flag-LRRK2 coding sequence
atggattacaaggatgacgacgataagATGGCTAGTGGCAGCTGTCAGGGGTGCGAAGAGGACGA
GGAAACTCTGAAGAAGTTGATAGTCAGGCTGAACAATGTCCAGGAAGGAAAACAGATAGAAACGC
TGGTCCAAATCCTGGAGGATCTGCTGGTGTTCACGTACTCCGAGCACGCCTCCAAGTTATTTCAA
GGCAAAAATATCCATGTGCCTCTGTTGATCGTCTTGGACTCCTATATGAGAGTCGCGAGTGTGCA
GCAGGTGGGTTGGTCACTTCTGTGCAAATTAATAGAAGTCTGTCCAGGTACAATGCAAAGCTTAA
TGGGACCCCAGGATGTTGGAAATGATTGGGAAGTCCTTGGTGTTCACCAATTGATTCTTAAAATG
CTAACAGTTCATAATGCCAGTGTAAACTTGTCAGTGATTGGACTGAAGACCTTAGATCTCCTCCT
AACTTCAGGTAAAATCACCTTGCTGATACTGGATGAAGAAAGTGATATTTTCATGTTAATTTTTG
ATGCCATGCACTCATTTCCAGCCAATGATGAAGTCCAGAAACTTGGATGCAAAGCTTTACATGTG
CTGTTTGAGAGAGTCTCAGAGGAGCAACTGACTGAATTTGTTGAGAACAAAGATTATATGATATT
GTTAAGTGCGTTAACAAATTTTAAAGATGAAGAGGAAATTGTGCTTCATGTGCTGCATTGTTTAC
ATTCCCTAGCGATTCCTTGCAATAATGTGGAAGTCCTCATGAGTGGCAATGTCAGGTGTTATAAT
ATTGTGGTGGAAGCTATGAAAGCATTCCCTATGAGTGAAAGAATTCAAGAAGTGAGTTGCTGTTT
GCTCCATAGGCTTACATTAGGTAATTTTTTCAATATCCTGGTATTAAACGAAGTCCATGAGTTTG
TGGTGAAAGCTGTGCAGCAGTACCCAGAGAATGCAGCATTGCAGATCTCAGCGCTCAGCTGTTTG
GCCCTCCTCACTGAGACTATTTTCTTAAATCAAGATTTAGAGGAAAAGAATGAGAATCAAGAGAA
TGATGATGAGGGGGAAGAAGATAAATTGTTTTGGCTGGAAGCCTGTTACAAAGCATTAACGTGGC
ATAGAAAGAACAAGCACGTGCAGGAGGCCGCATGCTGGGCACTAAATAATCTCCTTATGTACCAA
AACAGTTTACATGAGAAGATTGGAGATGAAGATGGCCATTTCCCAGCTCATAGGGAAGTGATGCT
CTCCATGCTGATGCATTCTTCATCAAAGGAAGTTTTCCAGGCATCTGCGAATGCATTGTCAACTC
TCTTAGAACAAAATGTTAATTTCAGAAAAATACTGTTATCAAAAGGAATACACCTGAATGTTTTG
GAGTTAATGCAGAAGCATATACATTCTCCTGAAGTGGCTGAAAGTGGCTGTAAAATGCTAAATCA
TCTTTTTGAAGGAAGCAACACTTCCCTGGATATAATGGCAGCAGTGGTCCCCAAAATACTAACAG
TTATGAAACGTCATGAGACATCATTACCAGTGCAGCTGGAGGCGCTTCGAGCTATTTTACATTTT
ATAGTGCCTGGCATGCCAGAAGAATCCAGGGAGGATACAGAATTTCATCATAAGCTAAATATGGT
TAAAAAACAGTGTTTCAAGAATGATATTCACAAACTGGTCCTAGCAGCTTTGAACAGGTTCATTG
GAAATCCTGGGATTCAGAAATGTGGATTAAAAGTAATTTCTTCTATTGTACATTTTCCTGATGCA
TTAGAGATGTTATCCCTGGAAGGTGCTATGGATTCAGTGCTTCACACACTGCAGATGTATCCAGA
TGACCAAGAAATTCAGTGTCTGGGTTTAAGTCTTATAGGATACTTGATTACAAAGAAGAATGTGT
TCATAGGAACTGGACATCTGCTGGCAAAAATTCTGGTTTCCAGCTTATACCGATTTAAGGATGTT
GCTGAAATACAGACTAAAGGATTTCAGACAATCTTAGCAATCCTCAAATTGTCAGCATCTTTTTC
TAAGCTGCTGGTGCATCATTCATTTGACTTAGTAATATTCCATCAAATGTCTTCCAATATCATGG
AACAAAAGGATCAACAGTTTCTAAACCTCTGTTGCAAGTGTTTTGCAAAAGTAGCTATGGATGAT
TACTTAAAAAATGTGATGCTAGAGAGAGCGTGTGATCAGAATAACAGCATCATGGTTGAATGCTT
GCTTCTATTGGGAGCAGATGCCAATCAAGCAAAGGAGGGATCTTCTTTAATTTGTCAGGTATGTG
AGAAAGAGAGCAGTCCCAAATTGGTGGAACTCTTACTGAATAGTGGATCTCGTGAACAAGATGTA
CGAAAAGCGTTGACGATAAGCATTGGGAAAGGTGACAGCCAGATCATCAGCTTGCTCTTAAGGAG
GCTGGCCCTGGATGTGGCCAACAATAGCATTTGCCTTGGAGGATTTTGTATAGGAAAAGTTGAAC
CTTCTTGGCTTGGTCCTTTATTTCCAGATAAGACTTCTAATTTAAGGAAACAAACAAATATAGCA
TCTACACTAGCAAGAATGGTGATCAGATATCAGATGAAAAGTGCTGTGGAAGAAGGAACAGCCTC
AGGCAGCGATGGAAATTTTTCTGAAGATGTGCTGTCTAAATTTGATGAATGGACCTTTATTCCTG
ACTCTTCTATGGACAGTGTGTTTGCTCAAAGTGATGACCTGGATAGTGAAGGAAGTGAAGGCTCA
TTTCTTGTGAAAAAGAAATCTAATTCAATTAGTGTAGGAGAATTTTACCGAGATGCCGTATTACA
GCGTTGCTCACCAAATTTGCAAAGACATTCCAATTCCTTGGGGCCCATTTTTGATCATGAAGATT
TACTGAAGCGAAAAAGAAAAATATTATCTTCAGATGATTCACTCAGGTCATCAAAACTTCAATCC
CATATGAGGCATTCAGACAGCATTTCTTCTCTGGCTTCTGAGAGAGAATATATTACATCACTAGA
CCTTTCAGCAAATGAACTAAGAGATATTGATGCCCTAAGCCAGAAATGCTGTATAAGTGTTCATT
TGGAGCATCTTGAAAAGCTGGAGCTTCACCAGAATGCACTCACGAGCTTTCCACAACAGCTATGT
GAAACTCTGAAGAGTTTGACACATTTGGACTTGCACAGTAATAAATTTACATCATTTCCTTCTTA
TTTGTTGAAAATGAGTTGTATTGCTAATCTTGATGTCTCTCGAAATGACATTGGACCCTCAGTGG
TTTTAGATCCTACAGTGAAATGTCCAACTCTGAAACAGTTTAACCTGTCATATAACCAGCTGTCT
TTTGTACCTGAGAACCTCACTGATGTGGTAGAGAAACTGGAGCAGCTCATTTTAGAAGGAAATAA
AATATCAGGGATATGCTCCCCCTTGAGACTGAAGGAACTGAAGATTTTAAACCTTAGTAAGAACC
ACATTTCATCCCTATCAGAGAACTTTCTTGAGGCTTGTCCTAAAGTGGAGAGTTTCAGTGCCAGA
ATGAATTTTCTTGCTGCTATGCCTTTCTTGCCTCCTTCTATGACAATCCTAAAATTATCTCAGAA
CAAATTTTCCTGTATTCCAGAAGCAATTTTAAATCTTCCACACTTGCGGTCTTTAGATATGAGCA
GCAATGATATTCAGTACCTACCAGGTCCCGCACACTGGAAATCTTTGAACTTAAGGGAACTCTTA
TTTAGCCATAATCAGATCAGCATCTTGGACTTGAGTGAAAAAGCATATTTATGGTCTAGAGTAGA
GAAACTGCATCTTTCTCACAATAAACTGAAAGAGATTCCTCCTGAGATTGGCTGTCTTGAAAATC
TGACATCTCTGGATGTCAGTTACAACTTGGAACTAAGATCCTTTCCCAATGAAATGGGGAAATTA
AGCAAAATATGGGATCTTCCTTTGGATGAACTGCATCTTAACTTTGATTTTAAACATATAGGATG
TAAAGCCAAAGACATCATAAGGTTTCTTCAACAGCGATTAAAAAAGGCTGTGCCTTATAACCGAA
TGAAACTTATGATTGTGGGAAATACTGGGAGTGGTAAAACCACCTTATTGCAGCAATTAATGAAA
ACCAAGAAATCAGATCTTGGAATGCAAAGTGCCACAGTTGGCATAGATGTGAAAGACTGGCCTAT
CCAAATAAGAGACAAAAGAAAGAGAGATCTCGTCCTAAATGTGTGGGATTTTGCAGGTCGTGAGG
AATTCTATAGTACTCATCCCCATTTTATGACGCAGCGAGCATTGTACCTTGCTGTCTATGACCTC
AGCAAGGGACAGGCTGAAGTTGATGCCATGAAGCCTTGGCTCTTCAATATAAAGGCTCGCGCTTC
TTCTTCCCCTGTGATTCTCGTTGGCACACATTTGGATGTTTCTGATGAGAAGCAACGCAAAGCCT
GCATGAGTAAAATCACCAAGGAACTCCTGAATAAGCGAGGGTTCCCTGCCATACGAGATTACCAC
TTTGTGAATGCCACCGAGGAATCTGATGCTTTGGCAAAACTTCGGAAAACCATCATAAACGAGAG
CCTTAATTTCAAGATCCGAGATCAGCTTGTTGTTGGACAGCTGATTCCAGACTGCTATGTAGAAC
TTGAAAAAATCATTTTATCGGAGCGTAAAAATGTGCCAATTGAATTTCCCGTAATTGACCGGAAA
CGATTATTACAACTAGTGAGAGAAAATCAGCTGCAGTTAGATGAAAATGAGCTTCCTCACGCAGT
TCACTTTCTAAATGAATCAGGAGTCCTTCTTCATTTTCAAGACCCAGCACTGCAGTTAAGTGACT
TGTACTTTGTGGAACCCAAGTGGCTTTGTAAAATCATGGCACAGATTTTGACAGTGAAAGTGGAA
GGTTGTCCAAAACACCCTAAGGGAATTATTTCGCGTAGAGATGTGGAAAAATTTCTTTCAAAGAA
AAGGAAATTTCCAAAGAACTACATGTCACAGTATTTTAAGCTCCTAGAAAAATTCCAGATTGCTT
TGCCAATAGGAGAAGAATATTTGCTGGTTCCAAGCAGTTTGTCTGACCACAGGCCTGTGATAGAG
CTTCCCCATTGTGAGAACTCTGAAATTATCATCCGACTATATGAAATGCCTTATTTTCCAATGGG
ATTTTGGTCAAGATTAATCAATCGATTACTTGAGATTTCACCTTACATGCTTTCAGGGAGAGAAC
GAGCACTTCGCCCAAACAGAATGTATTGGCGACAAGGCATTTACTTAAATTGGTCTCCTGAAGCT
TATTGTCTGGTAGGATCTGAAGTCTTAGACAATCATCCAGAGAGTTTCTTAAAAATTACAGTTCC
TTCTTGTAGAAAAGGCTGTATTCTTTTGGGCCAAGTTGTGGACCACATTGATTCTCTCATGGAAG
AATGGTTTCCTGGGTTGCTGGAGATTGATATTTGTGGTGAAGGAGAAACTCTGTTGAAGAAATGG
GCATTATATAGTTTTAATGATGGTGAAGAACATCAAAAAATCTTACTTGATGACTTGATGAAGAA
AGCAGAGGAAGGAGATCTCTTAGTAAATCCAGATCAACCAAGGCTCACCATTCCAATATCTCAGA
TTGCCCCTGACTTGATTTTGGCTGACCTGCCTAGAAATATTATGTTGAATAATGATGAGTTGGAA
TTTGAACAAGCTCCAGAGTTTCTCCTAGGTGATGGCAGTTTTGGATCAGTTTACCGAGCAGCCTA
TGAAGGAGAAGAAGTGGCTGTGAAGATTTTTAATAAACATACATCACTCAGGCTGTTAAGACAAG
AGCTTGTGGTGCTTTGCCACCTCCACCACCCCAGTTTGATATCTTTGCTGGCAGCTGGGATTCGT
CCCCGGATGTTGGTGATGGAGTTAGCCTCCAAGGGTTCCTTGGATCGCCTGCTTCAGCAGGACAA
AGCCAGCCTCACTAGAACCCTACAGCACAGGATTGCACTCCACGTAGCTGATGGTTTGAGATACC
TCCACTCAGCCATGATTATATACCGAGACCTGAAACCCCACAATGTGCTGCTTTTCACACTGTAT
CCCAATGCTGCCATCATTGCAAAGATTGCTGACTACGGCATTGCTCAGTACTGCTGTAGAATGGG
GATAAAAACATCAGAGGGCACACCAGGGTTTCGTGCACCTGAAGTTGCCAGAGGAAATGTCATTT
ATAACCAACAGGCTGATGTTTATTCATTTGGTTTACTACTCTATGACATTTTGACAACTGGAGGT
AGAATAGTAGAGGGTTTGAAGTTTCCAAATGAGTTTGATGAATTAGAAATACAAGGAAAATTACC
TGATCCAGTTAAAGAATATGGTTGTGCCCCATGGCCTATGGTTGAGAAATTAATTAAACAGTGTT
TGAAAGAAAATCCTCAAGAAAGGCCTACTTCTGCCCAGGTCTTTGACATTTTGAATTCAGCTGAA
TTAGTCTGTCTGACGAGACGCATTTTATTACCTAAAAACGTAATTGTTGAATGCATGGTTGCTAC
ACATCACAACAGCAGGAATGCAAGCATTTGGCTGGGCTGTGGGCACACCGACAGAGGACAGCTCT
CATTTCTTGACTTAAATACTGAAGGATACACTTCTGAGGAAGTTGCTGATAGTAGAATATTGTGC
TTAGCCTTGGTGCATCTTCCTGTTGAAAAGGAAAGCTGGATTGTGTCTGGGACACAGTCTGGTAC
TCTCCTGGTCATCAATACCGAAGATGGGAAAAAGAGACATACCCTAGAAAAGATGACTGATTCTG
TCACTTGTTTGTATTGCAATTCCTTTTCCAAGCAAAGCAAACAAAAAAATTTTCTTTTGGTTGGA
ACCGCTGATGGCAAGTTAGCAATTTTTGAAGATAAGACTGTTAAGCTTAAAGGAGCTGCTCCTTT
GAAGATACTAAATATAGGAAATGTCAGTACTCCATTGATGTGTTTGAGTGAATCCACAAATTCAA
CGGAAAGAAATGTAATGTGGGGAGGATGTGGCACAAAGATTTTCTCCTTTTCTAATGATTTCACC
ATTCAGAAACTCATTGAGACAAGAACAAGCCAACTGTTTTCTTATGCAGCTTTCAGTGATTCCAA
CATCATAACAGTGGTGGTAGACACTGCTCTCTATATTGCTAAGCAAAATAGCCCTGTTGTGGAAG
TGTGGGATAAGAAAACTGAAAAACTCTGTGGACTAATAGACTGCGTGCACTTTTTAAGGGAGGTA
ATGGTAAAAGAAAACAAGGAATCAAAACACAAAATGTCTTATTCTGGGAGAGTGAAAACCCTCTG
CCTTCAGAAGAACACTGCTCTTTGGATAGGAACTGGAGGAGGCCATATTTTACTCCTGGATCTTT
CAACTCGTCGACTTATACGTGTAATTTACAACTTTTGTAATTCGGTCAGAGTCATGATGACAGCA
CAGCTAGGAAGCCTTAAAAATGTCATGCTGGTATTGGGCTACAACCGGAAAAATACTGAAGGTAC
ACAAAAGCAGAAAGAGATACAATCTTGCTTGACCGTTTGGGACATCAATCTTCCACATGAAGTGC
AAAATTTAGAAAAACACATTGAAGTGAGAAAAGAATTAGCTGAAAAAATGAGACGAACATCTGTT
GAGTAA
SEQ ID NO: 9 Translated protein sequence for human G2019
Full length LRRK2 flag tagged protein
MDYKDDDDKMASGSCQGCEEDEETLKKLIVRLNNVQEGKQIETLVQILEDLLVFTYSEHASKLFQ
GKNIHVPLLIVLDSYMRVASVQQVGWSLLCKLIEVCPGTMQSLMGPQDVGNDWEVLGVHQLILKM
LTVHNASVNLSVIGLKILDLLLTSGKITLLILDEESDIFMLIFDAMHSFPANDEVQKLGCKALHV
LFERVSEEQLTEFVENKDYMILLSALTNFKDEEEIVLHVLHCLHSLAIPCNNVEVLMSGNVRCYN
IVVEAMKAFPMSERIQEVSCCLLHRLTLGNFFNILVLNEVHEFVVKAVQQYPENAALQISALSCL
ALLTETIFLNQDLEEKNENQENDDEGEEDKLFWLEACYKALTWHRKNKHVQEAACWALNNLLMYQ
NSLHEKIGDEDGHFPAHREVMLSMLMHSSSKEVFQASANALSTLLEQNVNFRKILLSKGIHLNVL
ELMQKHIHSPEVAESGCKMLNHLFEGSNTSLDIMAAVVPKILTVMKRHETSLPVQLEALRAILHF
IVPGMPEESREDTEFHHKLNMVKKQCFKNDIHKLVLAALNRFIGNPGIQKCGLKVISSIVHFPDA
LEMLSLEGAMDSVLHTLQMYPDDQEIQCLGLSLIGYLITKKNVFIGTGHLLAKILVSSLYRFKDV
AEIQTKGFQTILAILKLSASFSKLLVHHSFDLVIFHQMSSNIMEQKDQQFLNLCCKCFAKVAMDD
YLKNVMLERACDQNNSIMVECLLLLGADANQAKEGSSLICQVCEKESSPKLVELLLNSGSREQDV
RKALTISIGKGDSQIISLLLRRLALDVANNSICLGGFCIGKVEPSWLGPLFPDKTSNLRKQTNIA
STLARMVIRYQMKSAVEEGTASGSDGNFSEDVLSKFDEWTFIPDSSMDSVFAQSDDLDSEGSEGS
FLVKKKSNSISVGEFYRDAVLQRCSPNLQRHSNSLGPIFDHEDLLKRKRKILSSDDSLRSSKLQS
HMRHSDSISSLASEREYITSLDLSANELRDIDALSQKCCISVHLEHLEKLELHQNALTSFPQQLC
ETLKSLTHLDLHSNKFTSFPSYLLKMSCIANLDVSRNDIGPSVVLDPTVKCPTLKQFNLSYNQLS
FVPENLTDVVEKLEQLILEGNKISGICSPLRLKELKILNLSKNHISSLSENFLEACPKVESFSAR
MNFLAAMPFLPPSMTILKLSQNKFSCIPEAILNLPHLRSLDMSSNDIQYLPGPAHWKSLNLRELL
FSHNQISILDLSEKAYLWSRVEKLHLSHNKLKEIPPEIGCLENLTSLDVSYNLELRSFPNEMGKL
SKIWDLPLDELHLNFDFKHIGCKAKDIIRFLQQRLKKAVPYNRMKLMIVGNTGSGKTTLLQQLMK
TKKSDLGMQSATVGIDVKDWPIQIRDKRKRDLVLNVVVDFAGREEFYSTHPHFMTQRALYLAVYD
LSKGQAEVDAMKPWLFNIKARASSSPVILVGTHLDVSDEKQRKACMSKITKELLNKRGFPAIRDY
HFVNATEESDALAKLRKTIINESLNFKIRDQLVVGQLIPDCYVELEKIILSERKNVPIEFPVIDR
KRLLQLVRENQLQLDENELPHAVHFLNESGVLLHFQDPALQLSDLYFVEPKWLCKIMAQILTVKV
EGCPKHPKGIISRRDVEKFLSKKRKFPKNYMSQYFKLLEKFQIALPIGEEYLLVPSSLSDHRPVI
ELPHCENSEIIIRLYEMPYFPMGFWSRLINRLLEISPYMLSGRERALRPNRMYWRQGIYLNWSPE
AYCLVGSEVLDNHPESFLKITVPSCRKGCILLGQVVDHIDSLMEEWFPGLLEIDICGEGETLLKK
WALYSFNDGEEHQKILLDDLMKKAEEGDLLVNPDQPRLTIPISQIAPDLILADLPRNIMLNNDEL
EFEQAPEFLLGDGSFGSVYRAAYEGEEVAVKIFNKHTSLRLLRQELVVLCHLHHPSLISLLAAGI
RPRMLVMELASKGSLDRLLQQDKASLTRTLQHRIALHVADGLRYLHSAMIIYRDLKPHNVLLFTL
YPNAAIIAKIADYGIAQYCCRMGIKTSEGTPGFRAPEVARGNVIYNQQADVYSFGLLLYDILTTG
GRIVEGLKFPNEFDELEIQGKLPDPVKEYGCAPWPMVEKLIKQCLKENPQERPTSAQVFDILNSA
ELVCLTRRILLPKNVIVECMVATHHNSRNASIWLGCGHTDRGQLSFLDLNTEGYTSEEVADSRIL
CLALVHLPVEKESWIVSGTQSGTLLVINTEDGKKRHTLEKMTDSVTCLYCNSFSKQSKQKNFLLV
GTADGKLAIFEDKTVKLKGAAPLKILNIGNVSTPLMCLSESTNSTERNVMWGGCGTKIFSFSNDF
TIQKLIETRTSQLFSYAAFSDSNIITVVVDTALYIAKQNSPVVEVWDKKTEKLCGLIDCVHFLRE
VMVKENKESKHKMSYSGRVKTLCLQKNTALWIGTGGGHILLLDLSTRRLIRVIYNFCNSVRVMMT
AQLGSLKNVMLVLGYNRKNTEGTQKQKEIQSCLTVVVDINLPHEVQNLEKHIEVRKELAEKMRRT
SVE
SEQ ID NO: 10: ‘LRRKtide’ peptide
H-RLGRDKYKTLRQIRQ-OH

1-24. (canceled)

25. A compound of Formula (I): 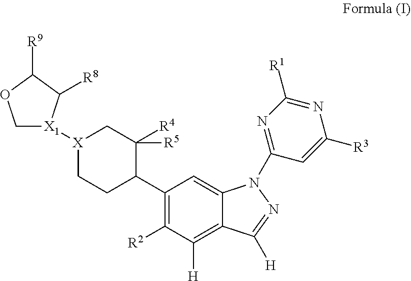
wherein:
X₁ is CR⁶;
wherein:
R⁶ is H or C₁₋₃alkyl;
wherein:
the C₁₋₃alkyl group of R⁶ optionally is substituted with one or more substituents independently selected from the group consisting of hydroxyl, halo and C₁₋₃alkoxyl;
R¹ is selected from the group consisting of CN, C₁₋₃ alkyl, C₁₋₃ alkoxy, C₁₋₃haloalkyl and C₃ cycloalkyl;
R² is selected from the group consisting of H, halo, CN, C₁₋₃alkyl and C₁₋₃haloalkyl;
R³ is selected from the group consisting of:
a) an N-linked 4-6 membered heterocyclyl ring optionally substituted with one or two substituents independently selected from the group consisting of oxo, halo, hydroxyl, C₁₋₆alkyl and C₁₋₆ alkoxyl;
wherein:
the C₁₋₆alkyl group is optionally substituted with one or two substituents independently selected from the group consisting of: halo, hydroxyl, C₁₋₃alkoxy and cyclopropyl;
the C₁₋₆ alkoxyl group is optionally substituted with one or two substitutents independently selected from halo, hydroxyl and C₁₋₃ alkoxyl;
when the N-linked 4-6 membered heterocyclyl ring contains a substitutable nitrogen atom, the N-linked 4-6 membered heterocyclyl ring optionally is substituted with a 4-6 membered heterocyclyl ring;
wherein:
the 4-6 membered heterocyclyl ring optionally is substituted with one, two or three substitutents independently selected from halo, hydroxyl, and C₁₋₃ alkoxyl; and
provided that:
the 4-6 membered heterocyclyl ring is attached to the substitutable nitrogen atom of the 4-6 membered heterocyclyl ring;
b) NHR⁷; and
c) OR⁷
R⁴ and R⁵ are independently selected from the group consisting of H, hydroxyl and halo;
R⁷ is independently selected from the group consisting of C₄₋₆ cycloalkyl and a nitrogen or oxygen containing 4-6 membered heterocyclyl;
wherein:
the C₄₋₆ cycloalkyl optionally is substituted with one, two or three substituents independently selected from halo, hydroxyl, C₁₋₃ alkoxyl and C₁₋₃ alkyl,
the nitrogen or oxygen containing 4-6 membered heterocyclyl optionally is substituted with one or more substitutents independently selected from halo, hydroxyl, C₁₋₃ alkoxyl and C₁₋₃ alkyl,
wherein:
the C₁₋₃ alky group defined for the C₄₋₆ cycloalkyl group and the nitrogen or oxygen containing 4-6 membered heterocyclyl is optionally substituted with one two or three halo or hydroxyl groups, and
R⁸ and R⁹ are independently selected from the group consisting of H, halo, methyl, ethyl, methoxyl and hydroxyl; or
a pharmaceutically acceptable salt thereof.

26. The compound of Formula (I) or a pharmaceutically acceptable salt thereof according to claim 25, wherein R¹ is selected from the group consisting of C₁₋₃ alkyl and C₁₋₃ alkoxyl.

27. The compound of Formula (I) or a pharmaceutically acceptable salt thereof according to claim 25, wherein R² is selected from the group consisting of H, halo and C₁₋₃alkyl.

28. The compound of Formula (I) or a pharmaceutically acceptable salt thereof according to claim 25, wherein R⁴ and R⁵ are independently selected from the group consisting of H and fluoro.

29. The compound of Formula (I) or a pharmaceutically acceptable salt thereof according to claim 28, wherein R⁴ and R⁵ are both H.

30. The compound of Formula (I) or a pharmaceutically acceptable salt thereof according to claim 25, wherein:
R³ is an N-linked 4-6 membered heterocyclyl ring optionally substituted with one or two substituents independently selected from the group consisting of halo, hydroxyl, C₁₋₃alkyl and C₁₋₃ alkoxyl;
wherein:
the C₁₋₃alkyl group optionally is substituted with one or two substituents independently selected from the group consisting of: halo, hydroxyl and C₁₋₃alkoxy; and
the C₁₋₃ alkoxyl group is optionally substituted with one or two substitutents independently selected from halo, hydroxyl and C₁₋₃ alkoxyl.

31. The compound or a pharmaceutically acceptable salt thereof according to claim 25, wherein R⁶ is H or unsubstituted C₁₋₃alkyl.

32. The compound or a pharmaceutically acceptable salt thereof according to claim 25, wherein R⁸ and R⁹, respectively, are both H.

33. A compound which is: 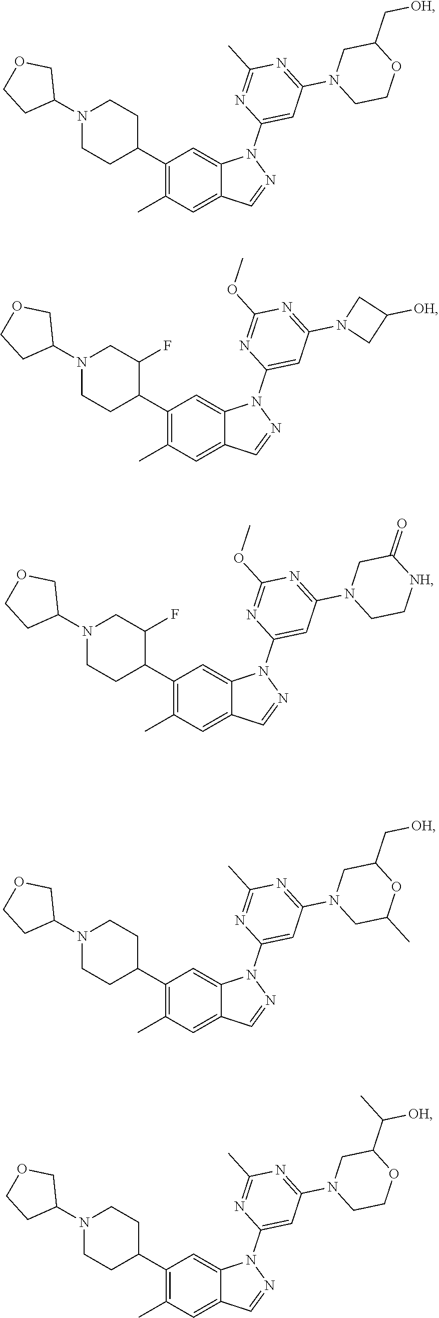 [SMILES: Cc1nc(N2CCOC(CO)C2)cc(-n2ncc3cc(C)c(C4CCN(C5CCOC5)CC4)cc32)n1 ; COc1nc(N2CC(O)C2)cc(-n2ncc3cc(C)c(C4CCN(C5CCOC5)CC4F)cc32)n1 ; COc1nc(N2CCNC(=O)C2)cc(-n2ncc3cc(C)c(C4CCN(C5CCOC5)CC4F)cc32)n1 ; Cc1nc(N2CC(C)OC(CO)C2)cc(-n2ncc3cc(C)c(C4CCN(C5CCOC5)CC4)cc32)n1 ; Cc1nc(N2CCOC(C(C)O)C2)cc(-n2ncc3cc(C)c(C4CCN(C5CCOC5)CC4)cc32)n1] 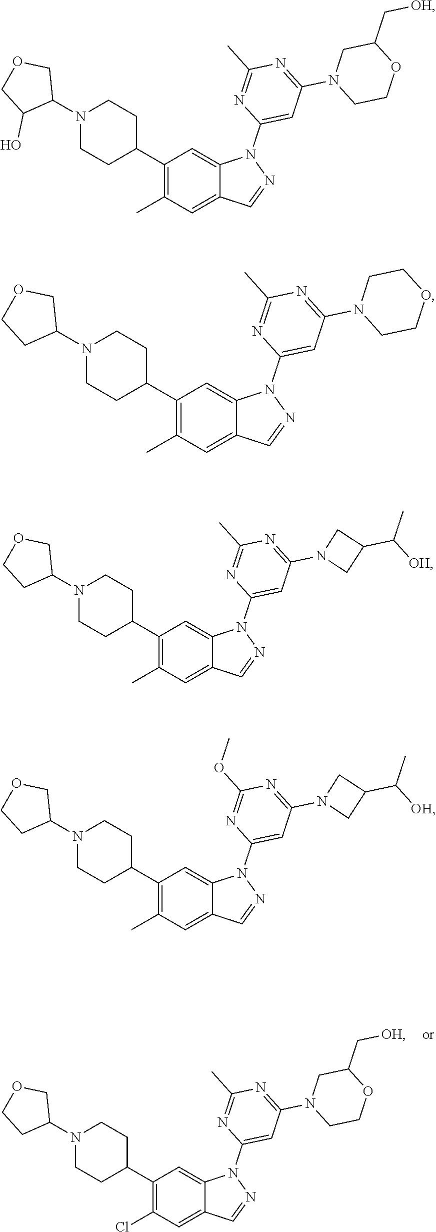 [SMILES: Cc1nc(N2CCOC(CO)C2)cc(-n2ncc3cc(C)c(C4CCN(C5COCC5O)CC4)cc32)n1 ; Cc1nc(N2CCOCC2)cc(-n2ncc3cc(C)c(C4CCN(C5CCOC5)CC4)cc32)n1 ; Cc1nc(N2CC(C(C)O)C2)cc(-n2ncc3cc(C)c(C4CCN(C5CCOC5)CC4)cc32)n1 ; COc1nc(N2CC(C(C)O)C2)cc(-n2ncc3cc(C)c(C4CCN(C5CCOC5)CC4)cc32)n1 ; Cc1nc(N2CCOC(CO)C2)cc(-n2ncc3cc(Cl)c(C4CCN(C5CCOC5)CC4)cc32)n1] 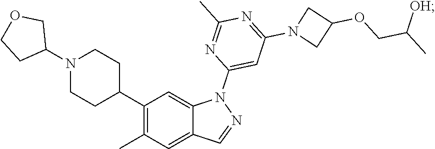 [SMILES: Cc1nc(N2CC(OCC(C)O)C2)cc(-n2ncc3cc(C)c(C4CCN(C5CCOC5)CC4)cc32)n1]
or
a pharmaceutically acceptable salt thereof.

34. A compound which is ((R)-4-(2-methyl-6-(5-methyl-6-(1-((S)-tetrahydrofuran-3-yl)piperidin-4-yl)-1H-indazol-1-yl)pyrimidin-4-yl)morpholin-2-yl)methanol 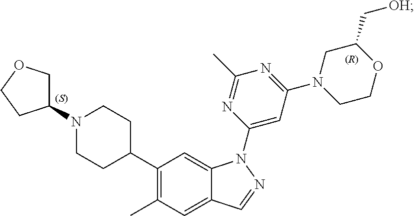
or
a pharmaceutically acceptable salt thereof.

35. The compound according to claim 10, which is ((R)-4-(2-methyl-6-(5-methyl-6-(1-((S)-tetrahydrofuran-3-yl)piperidin-4-yl)-1H-indazol-1-yl)pyrimidin-4-yl)morpholin-2-yl)methanol

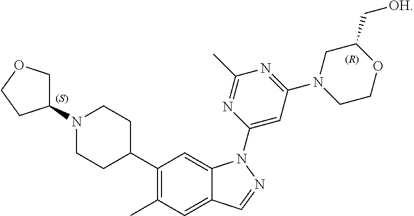

36. A pharmaceutical composition comprising a compound of Formula (I) or a pharmaceutically acceptable salt thereof according to claim 25 and a pharmaceutically acceptable excipient.

37. A method for treating a neurodegenerative disease, which comprises administering to a subject in need thereof a therapeutically effective amount of a compound of Formula (I) according to claim 25 or a pharmaceutically acceptable salt thereof.

38. The method for treating a neurodegenerative disease according to claim 37, wherein the neurodegenerative disease is Parkinson's disease, Alzheimer's disease or amyotrophic lateral sclerosis (ALS).

39. The method for treating a neurodegenerative disease according to claim 38, wherein the neurodegenerative disease is Parkinson's disease.

40. The method for treating a neurodegenerative disease according to claim 39, wherein the subject is a human.

41. The method for treating a neurodegenerative disease according to claim 39, wherein the subject is a human expressing the G2019S mutation in the LRRK2 kinase.

42. The method for treating a neurodegenerative disease according to claim 41, wherein the compound of Formula (I) is ((R)-4-(2-methyl-6-(5-methyl-6-(1-((S)-tetrahydrofuran-3-yl)piperidin-4-yl)-1H-indazol-1-yl)pyrimidin-4-yl)morpholin-2-yl)methanol 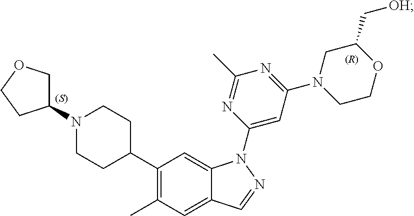
or
a pharmaceutically acceptable salt thereof.


SEQUENCE LISTING (10 sequences)

SEQ ID NO: 1 - DNA, 48 bp, Artificial
  Primers used for PCR cloning of Human G2019 LRRK2 plasmids preparation pHTBV-F
gatctcgacgggcgcggatccaccatggattacaaggatgacgacgat

SEQ ID NO: 2 - DNA, 49 bp, Artificial
  Primers used for PCR cloning of Human G2019 LRRK2 plasmids preparation LRRK2 wt-F1
catggattacaaggatgacgacgataagatggctagtggcagctgtcag

SEQ ID NO: 3 - DNA, 47 bp, Artificial
  Primers used for PCR cloning of Human G2019 LRRK2 plasmids preparation LRRK2 wt-R1
gttcacgagatccactattcagtaagagttccaccaatttgggactg

SEQ ID NO: 4 - DNA, 23 bp, Artificial
  Primers used for PCR cloning of Human G2019 LRRK2 plasmids preparation LRRK2 wt-F2
gaatagtggatctcgtgaacaag

SEQ ID NO: 5 - DNA, 25 bp, Artificial
  Primers used for PCR cloning of Human G2019 LRRK2 plasmids preparation LRRK2 wt-R2
gtcagacaaactgcttggaaccagc

SEQ ID NO: 6 - DNA, 40 bp, Artificial
  Primers used for PCR cloning of Human G2019 LRRK2 plasmids preparation LRRK2 wt-F3
ctggttccaagcagtttgtctgaccacaggcctgtgatag

SEQ ID NO: 7 - DNA, 46 bp, Artificial
  Primers used for PCR cloning of Human G2019 LRRK2 plasmids preparation pHTBV-R
gttctagccaagcttggtaccctattactcaacagatgttcgtctc

SEQ ID NO: 8 - DNA, 7611 bp, Artificial
  G2019  Full length Flag-LRRK2 coding sequence
atggattacaaggatgacgacgataagatggctagtggcagctgtcaggggtgcgaagaggacgaggaaactctgaagaagttgatagtcaggctgaacaatgtccaggaaggaaaacagatagaaacgctggtccaaatcctggaggatctgctggtgttcacgtactccgagcacgcctccaagttatttcaaggcaaaaatatccatgtgcctctgttgatcgtcttggactcctatatgagagtcgcgagtgtgcagcaggtgggttggtcacttctgtgcaaattaatagaagtctgtccaggtacaatgcaaagcttaatgggaccccaggatgttggaaatgattgggaagtccttggtgttcaccaattgattcttaaaatgctaacagttcataatgccagtgtaaacttgtcagtgattggactgaagaccttagatctcctcctaacttcaggtaaaatcaccttgctgatactggatgaagaaagtgatattttcatgttaatttttgatgccatgcactcatttccagccaatgatgaagtccagaaacttggatgcaaagctttacatgtgctgtttgagagagtctcagaggagcaactgactgaatttgttgagaacaaagattatatgatattgttaagtgcgttaacaaattttaaagatgaagaggaaattgtgcttcatgtgctgcattgtttacattccctagcgattccttgcaataatgtggaagtcctcatgagtggcaatgtcaggtgttataatattgtggtggaagctatgaaagcattccctatgagtgaaagaattcaagaagtgagttgctgtttgctccataggcttacattaggtaattttttcaatatcctggtattaaacgaagtccatgagtttgtggtgaaagctgtgcagcagtacccagagaatgcagcattgcagatctcagcgctcagctgtttggccctcctcactgagactattttcttaaatcaagatttagaggaaaagaatgagaatcaagagaatgatgatgagggggaagaagataaattgttttggctggaagcctgttacaaagcattaacgtggcatagaaagaacaagcacgtgcaggaggccgcatgctgggcactaaataatctccttatgtaccaaaacagtttacatgagaagattggagatgaagatggccatttcccagctcatagggaagtgatgctctccatgctgatgcattcttcatcaaaggaagttttccaggcatctgcgaatgcattgtcaactctcttagaacaaaatgttaatttcagaaaaatactgttatcaaaaggaatacacctgaatgttttggagttaatgcagaagcatatacattctcctgaagtggctgaaagtggctgtaaaatgctaaatcatctttttgaaggaagcaacacttccctggatataatggcagcagtggtccccaaaatactaacagttatgaaacgtcatgagacatcattaccagtgcagctggaggcgcttcgagctattttacattttatagtgcctggcatgccagaagaatccagggaggatacagaatttcatcataagctaaatatggttaaaaaacagtgtttcaagaatgatattcacaaactggtcctagcagctttgaacaggttcattggaaatcctgggattcagaaatgtggattaaaagtaatttcttctattgtacattttcctgatgcattagagatgttatccctggaaggtgctatggattcagtgcttcacacactgcagatgtatccagatgaccaagaaattcagtgtctgggtttaagtcttataggatacttgattacaaagaagaatgtgttcataggaactggacatctgctggcaaaaattctggtttccagcttataccgatttaaggatgttgctgaaatacagactaaaggatttcagacaatcttagcaatcctcaaattgtcagcatctttttctaagctgctggtgcatcattcatttgacttagtaatattccatcaaatgtcttccaatatcatggaacaaaaggatcaacagtttctaaacctctgttgcaagtgttttgcaaaagtagctatggatgattacttaaaaaatgtgatgctagagagagcgtgtgatcagaataacagcatcatggttgaatgcttgcttctattgggagcagatgccaatcaagcaaaggagggatcttctttaatttgtcaggtatgtgagaaagagagcagtcccaaattggtggaactcttactgaatagtggatctcgtgaacaagatgtacgaaaagcgttgacgataagcattgggaaaggtgacagccagatcatcagcttgctcttaaggaggctggccctggatgtggccaacaatagcatttgccttggaggattttgtataggaaaagttgaaccttcttggcttggtcctttatttccagataagacttctaatttaaggaaacaaacaaatatagcatctacactagcaagaatggtgatcagatatcagatgaaaagtgctgtggaagaaggaacagcctcaggcagcgatggaaatttttctgaagatgtgctgtctaaatttgatgaatggacctttattcctgactcttctatggacagtgtgtttgctcaaagtgatgacctggatagtgaaggaagtgaaggctcatttcttgtgaaaaagaaatctaattcaattagtgtaggagaattttaccgagatgccgtattacagcgttgctcaccaaatttgcaaagacattccaattccttggggcccatttttgatcatgaagatttactgaagcgaaaaagaaaaatattatcttcagatgattcactcaggtcatcaaaacttcaatcccatatgaggcattcagacagcatttcttctctggcttctgagagagaatatattacatcactagacctttcagcaaatgaactaagagatattgatgccctaagccagaaatgctgtataagtgttcatttggagcatcttgaaaagctggagcttcaccagaatgcactcacgagctttccacaacagctatgtgaaactctgaagagtttgacacatttggacttgcacagtaataaatttacatcatttccttcttatttgttgaaaatgagttgtattgctaatcttgatgtctctcgaaatgacattggaccctcagtggttttagatcctacagtgaaatgtccaactctgaaacagtttaacctgtcatataaccagctgtcttttgtacctgagaacctcactgatgtggtagagaaactggagcagctcattttagaaggaaataaaatatcagggatatgctcccccttgagactgaaggaactgaagattttaaaccttagtaagaaccacatttcatccctatcagagaactttcttgaggcttgtcctaaagtggagagtttcagtgccagaatgaattttcttgctgctatgcctttcttgcctccttctatgacaatcctaaaattatctcagaacaaattttcctgtattccagaagcaattttaaatcttccacacttgcggtctttagatatgagcagcaatgatattcagtacctaccaggtcccgcacactggaaatctttgaacttaagggaactcttatttagccataatcagatcagcatcttggacttgagtgaaaaagcatatttatggtctagagtagagaaactgcatctttctcacaataaactgaaagagattcctcctgagattggctgtcttgaaaatctgacatctctggatgtcagttacaacttggaactaagatcctttcccaatgaaatggggaaattaagcaaaatatgggatcttcctttggatgaactgcatcttaactttgattttaaacatataggatgtaaagccaaagacatcataaggtttcttcaacagcgattaaaaaaggctgtgccttataaccgaatgaaacttatgattgtgggaaatactgggagtggtaaaaccaccttattgcagcaattaatgaaaaccaagaaatcagatcttggaatgcaaagtgccacagttggcatagatgtgaaagactggcctatccaaataagagacaaaagaaagagagatctcgtcctaaatgtgtgggattttgcaggtcgtgaggaattctatagtactcatccccattttatgacgcagcgagcattgtaccttgctgtctatgacctcagcaagggacaggctgaagttgatgccatgaagccttggctcttcaatataaaggctcgcgcttcttcttcccctgtgattctcgttggcacacatttggatgtttctgatgagaagcaacgcaaagcctgcatgagtaaaatcaccaaggaactcctgaataagcgagggttccctgccatacgagattaccactttgtgaatgccaccgaggaatctgatgctttggcaaaacttcggaaaaccatcataaacgagagccttaatttcaagatccgagatcagcttgttgttggacagctgattccagactgctatgtagaacttgaaaaaatcattttatcggagcgtaaaaatgtgccaattgaatttcccgtaattgaccggaaacgattattacaactagtgagagaaaatcagctgcagttagatgaaaatgagcttcctcacgcagttcactttctaaatgaatcaggagtccttcttcattttcaagacccagcactgcagttaagtgacttgtactttgtggaacccaagtggctttgtaaaatcatggcacagattttgacagtgaaagtggaaggttgtccaaaacaccctaagggaattatttcgcgtagagatgtggaaaaatttctttcaaagaaaaggaaatttccaaagaactacatgtcacagtattttaagctcctagaaaaattccagattgctttgccaataggagaagaatatttgctggttccaagcagtttgtctgaccacaggcctgtgatagagcttccccattgtgagaactctgaaattatcatccgactatatgaaatgccttattttccaatgggattttggtcaagattaatcaatcgattacttgagatttcaccttacatgctttcagggagagaacgagcacttcgcccaaacagaatgtattggcgacaaggcatttacttaaattggtctcctgaagcttattgtctggtaggatctgaagtcttagacaatcatccagagagtttcttaaaaattacagttccttcttgtagaaaaggctgtattcttttgggccaagttgtggaccacattgattctctcatggaagaatggtttcctgggttgctggagattgatatttgtggtgaaggagaaactctgttgaagaaatgggcattatatagttttaatgatggtgaagaacatcaaaaaatcttacttgatgacttgatgaagaaagcagaggaaggagatctcttagtaaatccagatcaaccaaggctcaccattccaatatctcagattgcccctgacttgattttggctgacctgcctagaaatattatgttgaataatgatgagttggaatttgaacaagctccagagtttctcctaggtgatggcagttttggatcagtttaccgagcagcctatgaaggagaagaagtggctgtgaagatttttaataaacatacatcactcaggctgttaagacaagagcttgtggtgctttgccacctccaccaccccagtttgatatctttgctggcagctgggattcgtccccggatgttggtgatggagttagcctccaagggttccttggatcgcctgcttcagcaggacaaagccagcctcactagaaccctacagcacaggattgcactccacgtagctgatggtttgagatacctccactcagccatgattatataccgagacctgaaaccccacaatgtgctgcttttcacactgtatcccaatgctgccatcattgcaaagattgctgactacggcattgctcagtactgctgtagaatggggataaaaacatcagagggcacaccagggtttcgtgcacctgaagttgccagaggaaatgtcatttataaccaacaggctgatgtttattcatttggtttactactctatgacattttgacaactggaggtagaatagtagagggtttgaagtttccaaatgagtttgatgaattagaaatacaaggaaaattacctgatccagttaaagaatatggttgtgccccatggcctatggttgagaaattaattaaacagtgtttgaaagaaaatcctcaagaaaggcctacttctgcccaggtctttgacattttgaattcagctgaattagtctgtctgacgagacgcattttattacctaaaaacgtaattgttgaatgcatggttgctacacatcacaacagcaggaatgcaagcatttggctgggctgtgggcacaccgacagaggacagctctcatttcttgacttaaatactgaaggatacacttctgaggaagttgctgatagtagaatattgtgcttagccttggtgcatcttcctgttgaaaaggaaagctggattgtgtctgggacacagtctggtactctcctggtcatcaataccgaagatgggaaaaagagacataccctagaaaagatgactgattctgtcacttgtttgtattgcaattccttttccaagcaaagcaaacaaaaaaattttcttttggttggaaccgctgatggcaagttagcaatttttgaagataagactgttaagcttaaaggagctgctcctttgaagatactaaatataggaaatgtcagtactccattgatgtgtttgagtgaatccacaaattcaacggaaagaaatgtaatgtggggaggatgtggcacaaagattttctccttttctaatgatttcaccattcagaaactcattgagacaagaacaagccaactgttttcttatgcagctttcagtgattccaacatcataacagtggtggtagacactgctctctatattgctaagcaaaatagccctgttgtggaagtgtgggataagaaaactgaaaaactctgtggactaatagactgcgtgcactttttaagggaggtaatggtaaaagaaaacaaggaatcaaaacacaaaatgtcttattctgggagagtgaaaaccctctgccttcagaagaacactgctctttggataggaactggaggaggccatattttactcctggatctttcaactcgtcgacttatacgtgtaatttacaacttttgtaattcggtcagagtcatgatgacagcacagctaggaagccttaaaaatgtcatgctggtattgggctacaaccggaaaaatactgaaggtacacaaaagcagaaagagatacaatcttgcttgaccgtttgggacatcaatcttccacatgaagtgcaaaatttagaaaaacacattgaagtgagaaaagaattagctgaaaaaatgagacgaacatctgttgagtaa

SEQ ID NO: 9 - protein, 2536 aa, Artificial
  Translated protein sequence for human G2019 Full length LRRK2 flag tagged protein
MDYKDDDDKMASGSCQGCEEDEETLKKLIVRLNNVQEGKQIETLVQILEDLLVFTYSEHASKLFQGKNIHVPLLIVLDSYMRVASVQQVGWSLLCKLIEVCPGTMQSLMGPQDVGNDWEVLGVHQLILKMLTVHNASVNLSVIGLKTLDLLLTSGKITLLILDEESDIFMLIFDAMHSFPANDEVQKLGCKALHVLFERVSEEQLTEFVENKDYMILLSALTNFKDEEEIVLHVLHCLHSLAIPCNNVEVLMSGNVRCYNIVVEAMKAFPMSERIQEVSCCLLHRLTLGNFFNILVLNEVHEFVVKAVQQYPENAALQISALSCLALLTETIFLNQDLEEKNENQENDDEGEEDKLFWLEACYKALTWHRKNKHVQEAACWALNNLLMYQNSLHEKIGDEDGHFPAHREVMLSMLMHSSSKEVFQASANALSTLLEQNVNFRKILLSKGIHLNVLELMQKHIHSPEVAESGCKMLNHLFEGSNTSLDIMAAVVPKILTVMKRHETSLPVQLEALRAILHFIVPGMPEESREDTEFHHKLNMVKKQCFKNDIHKLVLAALNRFIGNPGIQKCGLKVISSIVHFPDALEMLSLEGAMDSVLHTLQMYPDDQEIQCLGLSLIGYLITKKNVFIGTGHLLAKILVSSLYRFKDVAEIQTKGFQTILAILKLSASFSKLLVHHSFDLVIFHQMSSNIMEQKDQQFLNLCCKCFAKVAMDDYLKNVMLERACDQNNSIMVECLLLLGADANQAKEGSSLICQVCEKESSPKLVELLLNSGSREQDVRKALTISIGKGDSQIISLLLRRLALDVANNSICLGGFCIGKVEPSWLGPLFPDKTSNLRKQTNIASTLARMVIRYQMKSAVEEGTASGSDGNFSEDVLSKFDEWTFIPDSSMDSVFAQSDDLDSEGSEGSFLVKKKSNSISVGEFYRDAVLQRCSPNLQRHSNSLGPIFDHEDLLKRKRKILSSDDSLRSSKLQSHMRHSDSISSLASEREYITSLDLSANELRDIDALSQKCCISVHLEHLEKLELHQNALTSFPQQLCETLKSLTHLDLHSNKFTSFPSYLLKMSCIANLDVSRNDIGPSVVLDPTVKCPTLKQFNLSYNQLSFVPENLTDVVEKLEQLILEGNKISGICSPLRLKELKILNLSKNHISSLSENFLEACPKVESFSARMNFLAAMPFLPPSMTILKLSQNKFSCIPEAILNLPHLRSLDMSSNDIQYLPGPAHWKSLNLRELLFSHNQISILDLSEKAYLWSRVEKLHLSHNKLKEIPPEIGCLENLTSLDVSYNLELRSFPNEMGKLSKIWDLPLDELHLNFDFKHIGCKAKDIIRFLQQRLKKAVPYNRMKLMIVGNTGSGKTTLLQQLMKTKKSDLGMQSATVGIDVKDWPIQIRDKRKRDLVLNVWDFAGREEFYSTHPHFMTQRALYLAVYDLSKGQAEVDAMKPWLFNIKARASSSPVILVGTHLDVSDEKQRKACMSKITKELLNKRGFPAIRDYHFVNATEESDALAKLRKTIINESLNFKIRDQLVVGQLIPDCYVELEKIILSERKNVPIEFPVIDRKRLLQLVRENQLQLDENELPHAVHFLNESGVLLHFQDPALQLSDLYFVEPKWLCKIMAQILTVKVEGCPKHPKGIISRRDVEKFLSKKRKFPKNYMSQYFKLLEKFQIALPIGEEYLLVPSSLSDHRPVIELPHCENSEIIIRLYEMPYFPMGFWSRLINRLLEISPYMLSGRERALRPNRMYWRQGIYLNWSPEAYCLVGSEVLDNHPESFLKITVPSCRKGCILLGQVVDHIDSLMEEWFPGLLEIDICGEGETLLKKWALYSFNDGEEHQKILLDDLMKKAEEGDLLVNPDQPRLTIPISQIAPDLILADLPRNIMLNNDELEFEQAPEFLLGDGSFGSVYRAAYEGEEVAVKIFNKHTSLRLLRQELVVLCHLHHPSLISLLAAGIRPRMLVMELASKGSLDRLLQQDKASLTRTLQHRIALHVADGLRYLHSAMIIYRDLKPHNVLLFTLYPNAAIIAKIADYGIAQYCCRMGIKTSEGTPGFRAPEVARGNVIYNQQADVYSFGLLLYDILTTGGRIVEGLKFPNEFDELEIQGKLPDPVKEYGCAPWPMVEKLIKQCLKENPQERPTSAQVFDILNSAELVCLTRRILLPKNVIVECMVATHHNSRNASIWLGCGHTDRGQLSFLDLNTEGYTSEEVADSRILCLALVHLPVEKESWIVSGTQSGTLLVINTEDGKKRHTLEKMTDSVTCLYCNSFSKQSKQKNFLLVGTADGKLAIFEDKTVKLKGAAPLKILNIGNVSTPLMCLSESTNSTERNVMWGGCGTKIFSFSNDFTIQKLIETRTSQLFSYAAFSDSNIITVVVDTALYIAKQNSPVVEVWDKKTEKLCGLIDCVHFLREVMVKENKESKHKMSYSGRVKTLCLQKNTALWIGTGGGHILLLDLSTRRLIRVIYNFCNSVRVMMTAQLGSLKNVMLVLGYNRKNTEGTQKQKEIQSCLTVWDINLPHEVQNLEKHIEVRKELAEKMRRTSVE

SEQ ID NO: 10 - protein, 15 aa, Artificial
  ′LRRKtide′ peptide
RLGRDKYKTLRQIRQ